SEQUENCE LISTING (178 sequences)

SEQ ID NO: 1 - protein, 30 aa, Homo sapiens
ALFSVLNYERARRPGLLGASVLGLDDIHRA

SEQ ID NO: 2 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 3 - protein, 1314 aa, Clostridium tetani
PITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 4 - protein, 456 aa, Clostridium tetani
PITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTA

SEQ ID NO: 5 - protein, 858 aa, Clostridium tetani
SLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 6 - protein, 8 aa, Clostridium tetani
GITELKKL

SEQ ID NO: 7 - protein, 18 aa, Clostridium tetani
FIGITELKKLESKINKVF

SEQ ID NO: 8 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 9 - protein, 1531 aa, Homo sapiens
MEVSPLQPVNENMQVNKIKKNEDAKKRLSVERIYQKKTQLEHILLRPDTYIGSVELVTQQMWVYDEDVGINYREVTFVPGLYKIFDEILVNAADNKQRDPKMSCIRVTIDPENNLISIWNNGKGIPVVEHKVEKMYVPALIFGQLLTSSNYDDDEKKVTGGRNGYGAKLCNIFSTKFTVETASREYKKMFKQTWMDNMGRAGEMELKPFNGEDYTCITFQPDLSKFKMQSLDKDIVALMVRRAYDIAGSTKDVKVFLNGNKLPVKGFRSYVDMYLKDKLDETGNSLKVIHEQVNHRWEVCLTMSEKGFQQISFVNSIATSKGGRHVDYVADQIVTKLVDVVKKKNKGGVAVKAHQVKNHMWIFVNALIENPTFDSQTKENMTLQPKSFGSTCQLSEKFIKAAIGCGIVESILNWVKFKAQVQLNKKCSAVKHNRIKGIPKLDDANDAGGRNSTECTLILTEGDSAKTLAVSGLGVVGRDKYGVFPLRGKILNVREASHKQIMENAEINNIIKIVGLQYKKNYEDEDSLKTLRYGKIMIMTDQDQDGSHIKGLLINFIHHNWPSLLRHRFLEEFITPIVKVSKNKQEMAFYSLPEFEEWKSSTPNHKKWKVKYYKGLGTSTSKEAKEYFADMKRHRIQFKYSGPEDDAAISLAFSKKQIDDRKEWLTNFMEDRRQRKLLGLPEDYLYGQTTTYLTYNDFINKELILFSNSDNERSIPSMVDGLKPGQRKVLFTCFKRNDKREVKVAQLAGSVAEMSSYHHGEMSLMMTIINLAQNFVGSNNLNLLQPIGQFGTRLHGGKDSASPRYIFTMLSSLARLLFPPKDDHTLKFLYDDNQRVEPEWYIPIIPMVLINGAEGIGTGWSCKIPNFDVREIVNNIRRLMDGEEPLPMLPSYKNFKGTIEELAPNQYVISGEVAILNSTTIEISELPVRTWTQTYKEQVLEPMLNGTEKTPPLITDYREYHTDTTVKFVVKMTEEKLAEAERVGLHKVFKLQTSLTCNSMVLFDHVGCLKKYDTVLDILRDFFELRLKYYGLRKEWLLGMLGAESAKLNNQARFILEKIDGKIIIENKPKKELIKVLIQRGYDSDPVKAWKEAQQKVPDEEENEESDNEKETEKSDSVTDSGPTFNYLLDMPLWYLTKEKKDELCRLRNEKEQELDTLKRKSPSDLWKEDLATFIEELEAVEAKEKQDEQVGLPGKGGKAKGKKTQMAEVLPSPRGQRVIPRITIEMKAEAEKKNKKKIKNENTEGSPQEDGVELEGLKQRLEKKQKREPGTKTKKQTTLAFKPIKKGKKRNPWSDSESDRSSDESNFDVPPRETEPRRAATKTKFTMDLDSDEDFSDFDEKTDDEDFVPSDASPPKTKTSPKLSNKELKPQKSVVSDLEADDVKGSVPLSSSPPATHFPDETEITNPVPKKNVTVKKTAAKSQSSTSTTGAKKRAAPKGTKRDPALNSGVSQKPDPAKTKNRRKRKPSTSDDSDSNFEKIVSKAVTSKKSKGESDDFHMDFDSAVAPRAKSVRAKKPIKYLEESDEDDLF

SEQ ID NO: 10 - protein, 543 aa, Homo sapiens
MGTSLSPNDPWPLNPLSIQQTTLLLLLSVLATVHVGQRLLRQRRRQLRSAPPGPFAWPLIGNAAAVGQAAHLSFARLARRYGDVFQIRLGSCPIVVLNGERAIHQALVQQGSAFADRPAFASFRVVSGGRSMAFGHYSEHWKVQRRAAHSMMRNFFTRQPRSRQVLEGHVLSEARELVALLVRGSADGAFLDPRPLTVVAVANVMSAVCFGCRYSHDDPEFRELLSHNEEFGRTVGAGSLVDVMPWLQYFPNPVRTVFREFEQLNRNFSNFILDKFLRHCESLRPGAAPRDMMDAFILSAEKKAAGDSHGGGARLDLENVPATITDIFGASQDTLSTALQWLLLLFTRYPDVQTRVQAELDQVVGRDRLPCMGDQPNLPYVLAFLYEAMRFSSFVPVTIPHATTANTSVLGYHIPKDTVVFVNQWSVNHDPLKWPNPENFDPARFLDKDGLINKDLTSRVMIFSVGKRRCIGEELSKMQLFLFISILAHQCDFRANPNEPAKMNFSYGLTIKPKSFKVNVTLRESMELLDSAVQNLQAKETCQ

SEQ ID NO: 11 - protein, 491 aa, Homo sapiens
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 12 - protein, 14 aa, Homo sapiens
ALFSVLNYERARRP

SEQ ID NO: 13 - protein, 14 aa, Homo sapiens
LFSVLNYERARRPG

SEQ ID NO: 14 - protein, 14 aa, Homo sapiens
FSVLNYERARRPGL

SEQ ID NO: 15 - protein, 14 aa, Homo sapiens
SVLNYERARRPGLL

SEQ ID NO: 16 - protein, 14 aa, Homo sapiens
VLNYERARRPGLLG

SEQ ID NO: 17 - protein, 14 aa, Homo sapiens
LNYERARRPGLLGA

SEQ ID NO: 18 - protein, 14 aa, Homo sapiens
NYERARRPGLLGAS

SEQ ID NO: 19 - protein, 14 aa, Homo sapiens
YERARRPGLLGASV

SEQ ID NO: 20 - protein, 14 aa, Homo sapiens
ERARRPGLLGASVL

SEQ ID NO: 21 - protein, 14 aa, Homo sapiens
RARRPGLLGASVLG

SEQ ID NO: 22 - protein, 14 aa, Homo sapiens
ARRPGLLGASVLGL

SEQ ID NO: 23 - protein, 14 aa, Homo sapiens
RRPGLLGASVLGLD

SEQ ID NO: 24 - protein, 14 aa, Homo sapiens
RPGLLGASVLGLDD

SEQ ID NO: 25 - protein, 14 aa, Homo sapiens
PGLLGASVLGLDDI

SEQ ID NO: 26 - protein, 14 aa, Homo sapiens
GLLGASVLGLDDIH

SEQ ID NO: 27 - protein, 14 aa, Homo sapiens
LLGASVLGLDDIHR

SEQ ID NO: 28 - protein, 14 aa, Homo sapiens
LGASVLGLDDIHRA

SEQ ID NO: 29 - protein, 15 aa, Homo sapiens
SVLNYERARRPGLLG

SEQ ID NO: 30 - protein, 15 aa, Homo sapiens
FSVLNYERARRPGLL

SEQ ID NO: 31 - protein, 15 aa, Homo sapiens
ARRPGLLGASVLGLD

SEQ ID NO: 32 - protein, 15 aa, Homo sapiens
RARRPGLLGASVLGL

SEQ ID NO: 33 - protein, 15 aa, Homo sapiens
VLNYERARRPGLLGA

SEQ ID NO: 34 - protein, 15 aa, Homo sapiens
RPGLLGASVLGLDDI

SEQ ID NO: 35 - protein, 15 aa, Homo sapiens
VLNYERARRPGLLGA

SEQ ID NO: 36 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 37 - protein, 386 aa, Homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 38 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 39 - protein, 15 aa, Homo sapiens
GQDLFGIWSKVYDPL

SEQ ID NO: 40 - protein, 15 aa, Homo sapiens
TEDTMTKLRELSELS

SEQ ID NO: 41 - protein, 15 aa, Homo sapiens
GKVFRGNKVKNAQLA

SEQ ID NO: 42 - protein, 15 aa, Homo sapiens
TGNFSTQKVKMHIHS

SEQ ID NO: 43 - protein, 15 aa, Homo sapiens
NYTLRVDCTPLMYSL

SEQ ID NO: 44 - protein, 15 aa, Homo sapiens
RQIYVAAFTVQAAAE

SEQ ID NO: 45 - protein, 27 aa, Homo sapiens
GARGPESRLLEFYLAMPFATPMEAELA

SEQ ID NO: 46 - protein, 29 aa, Artificial Sequence
  Synthetic
FIGITELKKLESKINKVFAEKYARVRAKC

SEQ ID NO: 47 - protein, 34 aa, Artificial Sequence
  Synthetic
ARWWNYARTEDFFQQQPPPGQDLFGIWSKVYDPL

SEQ ID NO: 48 - protein, 31 aa, Artificial Sequence
  Synthetic
ARWWLLHETDSAVAAARQIYVAAFTVQAAAE

SEQ ID NO: 49 - protein, 31 aa, Artificial Sequence
  Synthetic
ARWWSLSLGFLFLAAAGKVFRGNKVKNAQLA

SEQ ID NO: 50 - protein, 28 aa, Artificial Sequence
  Synthetic
ARWWGMPEGDLVYTGNFSTQKVKMHIHS

SEQ ID NO: 51 - protein, 28 aa, Artificial Sequence
  Synthetic
ARWWKVFRGNKVKNYTLRVDCTPLMYSL

SEQ ID NO: 52 - protein, 15 aa, Homo sapiens
RTFVLRVRAQDPPPE

SEQ ID NO: 53 - protein, 15 aa, Homo sapiens
AERLTSRVKALFSVL

SEQ ID NO: 54 - protein, 20 aa, Homo sapiens
AVGALEGSRNQDWLGVPRQL

SEQ ID NO: 55 - protein, 27 aa, Artificial Sequence
  Synthetic
  X (18)..(18): Cyclohexyl-Ala
  u (27)..(27): D-Ala
ARWWWMHHNMDLIGGAKXVAAWTLKAX

SEQ ID NO: 56 - protein, 4 aa, Homo sapiens
ARWW

SEQ ID NO: 57 - protein, 9 aa, Homo sapiens
WMHHNMDLI

SEQ ID NO: 58 - protein, 12 aa, Artificial Sequence
  Synthetic
  X (3)..(3): Cyclohexyl-Ala
  U (12)..(12): D-Ala
AKXVAAWTLKAX

SEQ ID NO: 59 - protein, 12 aa, Homo sapiens
FIGITELKKLES

SEQ ID NO: 60 - protein, 12 aa, Homo sapiens
GITELKKLESKI

SEQ ID NO: 61 - protein, 14 aa, Homo sapiens
SKFIGITELKKLES

SEQ ID NO: 62 - protein, 14 aa, Homo sapiens
FIGITELKKLESKI

SEQ ID NO: 63 - protein, 16 aa, Homo sapiens
ANSKFIGITELKKLES

SEQ ID NO: 64 - protein, 16 aa, Homo sapiens
SKFIGITELKKLESKI

SEQ ID NO: 65 - protein, 18 aa, Homo sapiens
IKANSKFIGITELKKLES

SEQ ID NO: 66 - protein, 18 aa, Homo sapiens
ANSKFIGITELKKLESKI

SEQ ID NO: 67 - protein, 20 aa, Homo sapiens
QYIKANSKFIGITELKKLES

SEQ ID NO: 68 - protein, 20 aa, Homo sapiens
IKANSKFIGITELKKLESKI

SEQ ID NO: 69 - protein, 22 aa, Homo sapiens
QYIKANSKFIGITELKKLESKI

SEQ ID NO: 70 - protein, 20 aa, Homo sapiens
FIGITELKKLESKINKVFST

SEQ ID NO: 71 - protein, 19 aa, Homo sapiens
FIGITELKKLESKINKVFS

SEQ ID NO: 72 - protein, 18 aa, Homo sapiens
FIGITELKKLESKINKVF

SEQ ID NO: 73 - protein, 17 aa, Homo sapiens
FIGITELKKLESKINKV

SEQ ID NO: 74 - protein, 16 aa, Homo sapiens
FIGITELKKLESKINK

SEQ ID NO: 75 - protein, 15 aa, Homo sapiens
FIGITELKKLESKIN

SEQ ID NO: 76 - protein, 14 aa, Homo sapiens
IGITELKKLESKIN

SEQ ID NO: 77 - protein, 14 aa, Homo sapiens
GITELKKLESKINK

SEQ ID NO: 78 - protein, 14 aa, Homo sapiens
  K (8)..(8): Formylated Lys8
FIGITELKKLESKI

SEQ ID NO: 79 - protein, 14 aa, Homo sapiens
  K (9)..(9): Ly9 formylated
FIGITELKKLESKI

SEQ ID NO: 80 - protein, 14 aa, Homo sapiens
  K (13)..(13): Lys13 formylated
FIGITELKKLESKI

SEQ ID NO: 81 - protein, 14 aa, Homo sapiens
  K (8)..(8): Lys8 formylated
  K (9)..(9): Lys9 formylated
FIGITELKKLESKI

SEQ ID NO: 82 - protein, 14 aa, Homo sapiens
  K (9)..(9): Formylated Lys9
  K (13)..(13): Formylated Lys13
FIGITELKKLESKI

SEQ ID NO: 83 - protein, 18 aa, Homo sapiens
AIGITELKKLESKINKVF

SEQ ID NO: 84 - protein, 18 aa, Homo sapiens
FAGITELKKLESKINKVF

SEQ ID NO: 85 - protein, 18 aa, Homo sapiens
FIAITELKKLESKINKVF

SEQ ID NO: 86 - protein, 18 aa, Homo sapiens
FIGITALKKLESKINKVF

SEQ ID NO: 87 - protein, 18 aa, Homo sapiens
FIGITEAKKLESKINKVF

SEQ ID NO: 88 - protein, 18 aa, Homo sapiens
FIGITELAKLESKINKVF

SEQ ID NO: 89 - protein, 18 aa, Homo sapiens
FIGITELKALESKINKVF

SEQ ID NO: 90 - protein, 18 aa, Homo sapiens
FIGITELKKAESKINKVF

SEQ ID NO: 91 - protein, 18 aa, Homo sapiens
FIGITELKKLASKINKVF

SEQ ID NO: 92 - protein, 18 aa, Homo sapiens
FIGITELKKLEAKINKVF

SEQ ID NO: 93 - protein, 18 aa, Homo sapiens
FIGITELKKLESAINKVF

SEQ ID NO: 94 - protein, 18 aa, Homo sapiens
FIGITELKKLESKANKVF

SEQ ID NO: 95 - protein, 18 aa, Homo sapiens
FIGITELKKLESKIAKVF

SEQ ID NO: 96 - protein, 18 aa, Homo sapiens
FIGITELKKLESKINAVF

SEQ ID NO: 97 - protein, 18 aa, Homo sapiens
FIGITELKKLESKINKAF

SEQ ID NO: 98 - protein, 18 aa, Homo sapiens
FIGITELKKLESKINKVA

SEQ ID NO: 99 - protein, 18 aa, Homo sapiens
YIGITELKKLESKINKVF

SEQ ID NO: 100 - protein, 18 aa, Homo sapiens
FLGITELKKLESKINKVF

SEQ ID NO: 101 - protein, 18 aa, Homo sapiens
FISITELKKLESKINKVF

SEQ ID NO: 102 - protein, 18 aa, Homo sapiens
FIGLTELKKLESKINKVF

SEQ ID NO: 103 - protein, 18 aa, Homo sapiens
FIGISELKKLESKINKVF

SEQ ID NO: 104 - protein, 18 aa, Homo sapiens
FIGITDLKKLESKINKVF

SEQ ID NO: 105 - protein, 18 aa, Homo sapiens
FIGITEIKKLESKINKVF

SEQ ID NO: 106 - protein, 18 aa, Homo sapiens
FIGITELRKLESKINKVF

SEQ ID NO: 107 - protein, 18 aa, Homo sapiens
FIGITELKRLESKINKVF

SEQ ID NO: 108 - protein, 18 aa, Homo sapiens
FIGITELKKIESKINKVF

SEQ ID NO: 109 - protein, 18 aa, Homo sapiens
FIGITELKKLETKINKVF

SEQ ID NO: 110 - protein, 18 aa, Homo sapiens
FIGITELKKLESRINKVF

SEQ ID NO: 111 - protein, 18 aa, Homo sapiens
FIGITELKKLESKLNKVF

SEQ ID NO: 112 - protein, 18 aa, Homo sapiens
FIGITELKKLESKIQKVF

SEQ ID NO: 113 - protein, 18 aa, Homo sapiens
FIGITELKKLESKINRVF

SEQ ID NO: 114 - protein, 18 aa, Homo sapiens
FIGITELKKLESKINKLF

SEQ ID NO: 115 - protein, 18 aa, Homo sapiens
FIGITELKKLESKINKVY

SEQ ID NO: 116 - protein, 30 aa, Homo sapiens
LSEAEVRQHREARPALLTSRLRFIPKPDGL

SEQ ID NO: 117 - protein, 30 aa, Homo sapiens
HREARPALLTSRLRFIPKPDGLRPIVNMDY

SEQ ID NO: 118 - protein, 30 aa, Homo sapiens
LYFVKVDVTGAYDTIPQDRLTRVIASIIKP

SEQ ID NO: 119 - protein, 11 aa, Homo sapiens
NPILLWQPIPV

SEQ ID NO: 120 - protein, 41 aa, Homo sapiens
HREARPALLTSRLRFIPKPDGLRPIVNMDYVVGARTFRREK

SEQ ID NO: 121 - protein, 28 aa, Homo sapiens
EARPALLTSRLRFIPKPDGLRPIVNMDY

SEQ ID NO: 122 - protein, 29 aa, Homo sapiens
EARPALLTSRLRFIPKPDGLRPIVNMDYV

SEQ ID NO: 123 - protein, 27 aa, Homo sapiens
EARPALLTSRLRFIPKPDGLRPIVNMD

SEQ ID NO: 124 - protein, 27 aa, Homo sapiens
AEVRQHREARPALLTSRLRFIPKPDGL

SEQ ID NO: 125 - protein, 41 aa, Homo sapiens
ALFSVLNYERARRPGLLGASVLGLDDIHRANPILLWQPIPV

SEQ ID NO: 126 - protein, 16 aa, Homo sapiens
EARPALLTSRLRFIPK

SEQ ID NO: 127 - protein, 10 aa, Artificial Sequence
  Synthetic
AEKYARVRAK

SEQ ID NO: 128 - protein, 10 aa, Artificial Sequence
  Synthetic
AAKYARVRAK

SEQ ID NO: 129 - protein, 11 aa, Artificial Sequence
  Synthetic
AAKYARVRAKC

SEQ ID NO: 130 - protein, 16 aa, Homo sapiens
LKRVQLRELSEAEVRQ

SEQ ID NO: 131 - protein, 15 aa, Homo sapiens
EAEVRQHREARPALL

SEQ ID NO: 132 - protein, 15 aa, Homo sapiens
RPALLTSRLRFIPKP

SEQ ID NO: 133 - protein, 15 aa, Homo sapiens
TSRLRFIPKPDGLRP

SEQ ID NO: 134 - protein, 15 aa, Homo sapiens
FIPKPDGLRPIVNMD

SEQ ID NO: 135 - protein, 15 aa, Homo sapiens
PDGLRPIVNMDYVVG

SEQ ID NO: 136 - protein, 15 aa, Homo sapiens
FVLRVRAQDPPPELY

SEQ ID NO: 137 - protein, 14 aa, Homo sapiens
PELYFVKVDVTGAY

SEQ ID NO: 138 - protein, 15 aa, Homo sapiens
ELYFVKVDVTGAYDT

SEQ ID NO: 139 - protein, 15 aa, Homo sapiens
LYFVKVDVTGAYDTI

SEQ ID NO: 140 - protein, 15 aa, Homo sapiens
VTGAYDTIPQDRLTE

SEQ ID NO: 141 - protein, 15 aa, Homo sapiens
PQDRLTEVIASIIKP

SEQ ID NO: 142 - protein, 28 aa, Homo sapiens
FIGITELKKLESKINKVFSSAFADVEAA

SEQ ID NO: 143 - protein, 31 aa, Artificial Sequence; identical to SEQ ID NO: 48
  Synthetic

SEQ ID NO: 144 - protein, 27 aa, Artificial Sequence
  Synthetic
LLHETDSAVAAARQIYVAAFTVQAAAE

SEQ ID NO: 145 - protein, 28 aa, Artificial Sequence
  Synthetic
FIGITELKKLESKINKVFAEKYARVRAK

SEQ ID NO: 146 - protein, 29 aa, Artificial Sequence
  Synthetic
FIGITELKKLESKINKVFAEKYARVRAKC

SEQ ID NO: 147 - protein, 9 aa, Homo sapiens
LLGASVLGL

SEQ ID NO: 148 - protein, 10 aa, Homo sapiens
GLLGASVLGL

SEQ ID NO: 149 - protein, 10 aa, Homo sapiens
VLGLDDIHRA

SEQ ID NO: 150 - protein, 15 aa, Homo sapiens
FSVLNYERARRPGLL

SEQ ID NO: 151 - protein, 15 aa, Homo sapiens
RARRPGLLGASVLGL

SEQ ID NO: 152 - protein, 15 aa, Homo sapiens
VLNYERARRPGLLGA

SEQ ID NO: 153 - protein, 15 aa, Homo sapiens
RPGLLGASVLGLDDI

SEQ ID NO: 154 - protein, 10 aa, Homo sapiens
SSAFADVEAA

SEQ ID NO: 155 - protein, 9 aa, Homo sapiens
NYARTEDFF

SEQ ID NO: 156 - protein, 9 aa, Homo sapiens
LLHETDSAV

SEQ ID NO: 157 - protein, 9 aa, Homo sapiens
SLSLGFLFL

SEQ ID NO: 158 - protein, 9 aa, Homo sapiens
GMPEGDLVY

SEQ ID NO: 159 - protein, 9 aa, Homo sapiens
KVFRGNKVK

SEQ ID NO: 160 - DNA, 90 bp, Homo sapiens
gcactgttcagcgtgctcaactacgagcgggcgcggcgccccggcctcctgggcgcctctgtgctgggcctggacgatatccacagggcc

SEQ ID NO: 161 - DNA, 93 bp, Homo sapiens
ctgtcggaagcagaggtcaggcagcatcgggaagccaggcccgccctgctgacgtccagactccgcttcatccccaagcctgacgggctgcgg

SEQ ID NO: 162 - DNA, 90 bp, Homo sapiens
catcgggaagccaggcccgccctgctgacgtccagactccgcttcatccccaagcctgacgggctgcggccgattgtgaacatggactac

SEQ ID NO: 163 - protein, 9 aa, Homo sapiens
NYERARRPG

SEQ ID NO: 164 - protein, 9 aa, Homo sapiens
RRPGLLGAS

SEQ ID NO: 165 - protein, 12 aa, Homo sapiens
LLGASVLGLDDI

SEQ ID NO: 166 - protein, 20 aa, Homo sapiens
EARPALLTSRLRFIPKPDGL

SEQ ID NO: 167 - protein, 15 aa, Homo sapiens
QHLKRVQLRELSEAE

SEQ ID NO: 168 - protein, 15 aa, Homo sapiens
LSEAEVRQHREARPA

SEQ ID NO: 169 - protein, 15 aa, Homo sapiens
EARPALLTSRLRFIP

SEQ ID NO: 170 - protein, 15 aa, Homo sapiens
LRFIPKPDGLRPIVN

SEQ ID NO: 171 - protein, 9 aa, Homo sapiens
LLTSRLRFI

SEQ ID NO: 172 - protein, 10 aa, Homo sapiens
ALLTSRLRFI

SEQ ID NO: 173 - protein, 15 aa, Homo sapiens
REARPALLTSRLRFI

SEQ ID NO: 174 - protein, 15 aa, Homo sapiens
RAERLTSRVKALFSV

SEQ ID NO: 175 - protein, 15 aa, Homo sapiens
ALFSVLNYERARRPG

SEQ ID NO: 176 - protein, 15 aa, Homo sapiens
VLGLDDIHRAWRTFV

SEQ ID NO: 177 - protein, 5 aa, Homo sapiens
RPIVN

SEQ ID NO: 178 - protein, 4 aa, Homo sapiens
PDGL

(19) United States
(12) Patent Application Publication
(10) Pub. No.: US 2025/0152688 A1
(43) Pub. Date: May 15, 2025

(54) A Conjugate

(71) Applicant: ULTIMOVACS AB, Uppsala (SE)

(72) Inventors: Dana Michel, Borås (SE); Mikael Granath, Borås (SE); Ulf Tedebark, Borås (SE); Gustav Gaudernack, Oslo (NO); Roald Skurtveit, Oslo (NO); Sara Mangsbo, Uppsala (SE)

(21) Appl. No.: 18/568,716

(22) PCT Filed: Jun. 9, 2022
(86) PCT No.: PCT/EP2022/065777

§ 371 Date: Dec. 8, 2023

(30) Foreign Application Priority Data
Jun. 9, 2021 (EP) 21178648.8

(51) Int. Cl. A61K 39/00; A61K 39/385; A61K 47/64; A61P 35/00; G01N 33/68

(52) U.S. Cl. CPC A61K 39/001157; A61K 39/385; A61K 47/646; A61P 35/00; G01N 33/6854; A61K 2039/575; A61K 2039/6037; G01N 2333/33

(57) ABSTRACT

A conjugate comprising at least one polypeptide comprising a sequence of a B-cell epitope and at least one polypeptide comprising a sequence of a CD4+ T-cell epitope is provided. The CD4+ T-cell epitope comprises a region of at least 12 amino acids of a universal tumour antigen or a sequence having at least 80% sequence identity to the region and the CD4+ T-cell epitope is immunogenic in at least 50% of the population. The at least one polypeptide comprising the sequence of the CD4+ T-cell epitope is equal to or less than 500 amino acids in length. The sequence of the B-cell epitope is different from the sequence of the CD4+ T-cell epitope and an antibody specific for the B-cell epitope binds to the conjugate.

### RELATED APPLICATIONS

This application is a § 371 National Phase Application of International Application No. PCT/EP2022/065777, filed on Jun. 9, 2022, which claims priority to European Patent Application No. 21178648.8, filed on Jun. 9, 2021, each of which are incorporated herein by reference in their entirety.

### REFERENCE TO AN ELECTRONIC SEQUENCE LISTING

The contents of the electronic sequence listing (0436-0001US1_Sequence Listing.txt; Size: 105,241 bytes; and Date of Creation: Jun. 9, 2021) is herein incorporated by reference in its entirety.

### FIELD OF THE INVENTION

The present invention relates to a conjugate, a polypeptide, a nucleic acid molecule and a vector. The present invention also relates to a combination comprising a first product and a second product and a cocktail of conjugates. The present invention also relates to a pharmaceutical composition comprising the conjugate, the cocktail of conjugates, the nucleic acid molecule, the polypeptide or the combination. In addition, the present invention relates to the conjugate, the cocktail of conjugates, the nucleic acid molecule, the polypeptide, the combination, the first product or the second product for use in medicine. Furthermore, the present invention relates to a core, an intermediate conjugate and a process for manufacturing a conjugate. The present invention also relates to a method of determining the presence of a CD4+ T-cell response to a CD4+ T-cell epitope in a subject to whom a conjugate has been administered.

### BACKGROUND OF THE INVENTION

Cancer is a heterogeneous disease that is characterised by the new, abnormal and/or uncontrolled proliferation of cells within an individual. Typically, there is a high degree of diversity between different types of cancer as well as between individuals having the same cancer type. Thus the provision of a cancer treatment that is effective across a patient population is challenging.

Numerous approaches have been proposed for the treatment of cancer. One approach is the provision of cancer vaccines, such as those including antigenic peptides which comprise fragments of tumour-associated antigens. Such antigenic peptides, when administered to an individual, can elicit a CD8+ (an MHC class I restricted) or CD4+ (an MHC class II restricted) T-cell response against cells expressing the tumour-associated antigens.

WO 2011/101173 discloses various polypeptides from human telomerase reverse transcriptase (hTERT) for the treatment of cancer. WO 2017/207814 further discloses polypeptides from hTERT in combination with an immune checkpoint inhibitor for the treatment of cancer.

There remains a need to provide further anti-cancer treatments, including further antigenic polypeptides (and nucleic acid molecules which encode such peptides).

WO 2011/115483 relates to a vaccine conjugate comprising a peptide derived from tetanus toxin (TTx), conjugated to an antigen, immunogen or to a vehicle comprising an antigen or immunogen.

Circulating antibodies are frequently present against TTx in the human population because the majority of people are vaccinated early in life against tetanus, particularly in the Western world. According to the World Health Organisation, in 2015 approximately 86% of all infants worldwide were vaccinated against tetanus. The tetanus vaccine comprises the tetanus toxoid (TTd), which is prepared by detoxifying TTx by treatment of the protein with formalin. Although TTd is a modified form of TTx (and is thus a different protein) TTd is able to induce protective immunity against tetanus, which implies that antibodies raised by TTd vaccination recognise TTx. TTd is used in the childhood DTP combination vaccine against three infectious diseases (diphtheria, pertussis (whooping cough) and tetanus). In addition, many people have been challenged later in life with TTd, since anti-tetanus vaccination is a common procedure after injuries suspected of being potential cause of a tetanus infection, and as it is also in some countries advised to get a booster every 10 years. As a consequence, anti-TTx/TTd antibodies are present in a significant proportion of the human population of industrialised countries.

It is proposed that circulating antibodies in the individual will bind to the peptide derived from TTx and target the conjugate to antigen-presenting cells (APCs) in order to provide a robust immune response. WO 2011/115483 discloses various peptides derived from TTx. No specific examples or data are presented of a conjugate comprising an antigen associated with cancer.

Mangsbo et al. Mol Immunol. 2018 January; 93:115-124 discloses peptides derived from TTx, including an 18-mer peptide sequence. Peptide conjugates comprising the 18-mer tetanus epitope conjugated to a CD8+ T-cell epitope derived from ovalbumin (OVA) or a CD8+ T-cell epitope derived from human glycoprotein 100 (hgp 100) were constructed.

Fletcher et al. J Immunol. 2018 Jul. 1; 201 (1): 87-97 discloses the 18-mer peptide sequence derived from TTx as described in Mangsbo et al. 2018. Conjugates comprising the 18-mer tetanus epitope conjugated to synthetic long peptides comprising a CD8+ T-cell epitope from either CMV or influenza were constructed.

It is to be appreciated that in order for CD8+ and/or CD4+ T-cell responses to occur, the antigenic polypeptide must be presented on an MHC molecule. MHC class I molecules are found on the surface of most cells and typically bind polypeptides which are between 8 and 10 amino acid residues in length. MHC class I molecules present polypeptides, which are derived from cytosolic proteins by proteolysis, to CD8+ T cells (also known as cytotoxic T cells or CTLs) in order to elicit a CD8+ T cell response. In contrast, MHC class II molecules are found on the surface of antigen presenting cells and activated T cells and bind polypeptides that are generally longer, typically between 12 and 24 amino acids in length. MHC class II molecules present polypeptides, which are derived from extracellular proteins that have been internalised by endocytosis and digested, to CD4+ T cells (otherwise known as helper T cells or Th cells) in order to elicit a CD4+ T cell response.

There is a wide range of variability in MHC molecules in human populations. In particular, different individuals have different HLA alleles (i.e. which encode the human MHC molecules) and these have varying binding affinity for polypeptides, depending on the amino acid sequence of the polypeptides. Thus an individual who has one particular HLA allele may have MHC molecules that will bind a polypeptide of a particular sequence whereas other individuals lacking the HLA allele will have MHC molecules unable to bind and present the polypeptide (or, at least, their MHC molecules will have a very low affinity for the polypeptide and so present it at a relatively low level).

There remains a need for antigenic polypeptides (and nucleic acid molecules which encode such polypeptides) as well as further approaches involving such components that elicit effective immune responses in individuals and/or across a greater proportion of the population. Furthermore, there remains a need for methods for monitoring such immune responses.

The present invention seeks to alleviate one or more of the above problems.

### SUMMARY OF THE INVENTION

Aspects of the present invention arise from identifying properties of a conjugate that comprises a polypeptide comprising a sequence of a B-cell epitope and a polypeptide comprising a sequence of a CD4+ T-cell epitope from a universal tumour antigen such as hTERT. When such a conjugate is administered to an individual, antibodies (either pre-existing (e.g. from previous vaccination with the B cell epitope) or arising from administration of the conjugate) which recognise the B cell epitope result in antibody-mediated uptake of the conjugate. This has a dual effect: firstly a positive feedback mechanism involving further antibody production specific for the B cell epitope of the conjugate; and secondly presentation of the CD4+ T cell epitope by dendritic cells to CD4+ helper T cells. The activated CD4+ T cells are specific to tumour cells expressing the universal tumour antigen and the positive feedback mechanism drives the mechanism of CD4+ T cell activation which thereby is particularly effective in delivering an anti-tumour CD4+ T cell immune response.

Other aspects of the present invention arise from the observation that another result of this dual effect is that the antibody titre of antibodies specific for the B cell epitope of the conjugate (which can be determined relatively easily for an individual) is indicative of the levels of activated CD4+ T cells specific for the T cell epitope of the conjugate (which is otherwise difficult to determine for an individual).

Other aspects of the present invention arise from the identification of certain structures of a core that can be used to link the B-cell epitope and the CD4+ T cell epitope of the conjugate described above.

Other aspects of the present invention arise from the observation that certain polypeptides of the hTERT protein sequence contain a particularly high number of clinically-relevant epitopes and thus would be suitable for use in a vaccine that can induce an immune response in a wide proportion of the population.

According to a first aspect of the present invention, there is provided a conjugate comprising:
(a) at least one polypeptide comprising a sequence of a B-cell epitope; and
(b) at least one polypeptide comprising a sequence of a CD4+ T-cell epitope,
wherein the CD4+ T-cell epitope comprises a region of at least 12 amino acids of a universal tumour antigen or a sequence having at least 80% sequence identity to the region and wherein the CD4+ T-cell epitope is immunogenic in at least 50% of the population, wherein the at least one polypeptide comprising the sequence of the CD4+ T-cell epitope is equal to or less than 500 amino acids in length,
wherein the sequence of the B-cell epitope is different from the sequence of the CD4+ T-cell epitope, and
wherein an antibody specific for the B-cell epitope binds to the conjugate.

Conveniently, the at least one polypeptide comprising a sequence of a B-cell epitope and the at least one polypeptide comprising a sequence of a CD4+ T-cell epitope are linked via a core, wherein the core comprises, prior to linkage:
a body portion;
   one or more first linking groups attached to the body portion; and
   one or more second linking groups attached to the body portion,
   wherein the first linking group and second linking group are orthogonal to each other;
and wherein the first linking group is linked to the at least one polypeptide comprising a sequence of a B-cell epitope to form a first connecting element, and the second linking group is linked to the at least one polypeptide comprising a sequence of a CD4+ T-cell epitope to form a second connecting element.

Preferably, the first linking group and the second linking group are independently selected from an alkyne (e.g. a terminal alkyne), an alkene (e.g. a terminal alkene, norbornene), a cycloalkyne, a trans-cycloalkene, a tetrazine, a conjugated diene, a maleimide, an α-halocarbonyl, a thiol and an azide; and/or the first connecting element and the second connecting element are independently selected from a 1,2,3-triazole linkage, a dihydropyridazine linkage, a pyridazine linkage and a sulfide linkage.

According to a second aspect of the present invention, there is provided a method of determining the presence of a CD4+ T-cell response to a CD4+ T-cell epitope in a subject to whom a conjugate comprising at least one polypeptide comprising a sequence of a B-cell epitope and at least one polypeptide comprising a sequence of the CD4+ T-cell epitope has been administered, comprising the steps of:
(a) detecting a quantity or an absence of an antibody specific to the B-cell epitope in a sample derived from the subject prior to an administration of the conjugate, wherein the quantity or the absence of the antibody is detected at a first level; and
(b) detecting a quantity or an absence of an antibody specific to the B-cell epitope in a sample derived from the subject subsequent to one or more administrations of the conjugate, wherein the quantity or the absence of the antibody is detected at a second level, and
wherein an increase in the quantity of the antibody at the second level relative to the quantity or absence of the antibody at the first level is indicative of the presence of a CD4+ T-cell response to the CD4+ T-cell epitope in the subject.

Preferably, step (b) comprises detecting a quantity or absence of an antibody specific to the B-cell epitope in a sample derived from a subject subsequent to two or more administrations of the conjugate, preferably three or more administrations of the conjugate, more preferably four administrations of the conjugate.

Conveniently, the at least one polypeptide comprising a sequence of a B-cell epitope is a first and a second polypeptide comprising a sequence of a B-cell epitope, preferably a first, a second and a third polypeptide comprising a sequence of a B-cell epitope.

Advantageously, the B-cell epitope comprises a sequence selected from:
(i) a sequence comprising at least 10 amino acids which are contiguous in SEQ ID NO: 3, preferably a sequence comprising at least 10 amino acids which are contiguous in SEQ ID NO: 5; or
(ii) a sequence having at least 70% sequence identity to (i).

Preferably, the B-cell epitope comprises a sequence selected from:
(i) a sequence comprising at least 10 amino acids which are contiguous in SEQ ID NO: 5 and comprise the amino acid sequence GITELKKL as represented by SEQ ID NO: 6 in the Sequence Listing; or
(i) a sequence having at least 70% sequence identity to (i)

Conveniently, the B-cell epitope comprises a sequence selected from:
(i) SEQ ID NO: 7; or
(ii) a sequence having at least 70% sequence identity to (i).

Advantageously, in the second aspect of the present invention, the CD4+ T-cell epitope comprises a region of at least 12 amino acids of a self-antigen or a tumour-associated antigen, or a sequence having at least 80% sequence identity to the region and wherein the at least one polypeptide comprising the sequence of the CD4+ T-cell epitope is equal to or less than 500 amino acids in length, preferably, wherein the self-antigen or the tumour-associated antigen is a universal tumour antigen.

Alternatively, in the second aspect of the present invention, the CD4+ T-cell epitope comprises a region of at least 12 amino acids of an endogenous protein or a viral or a bacterial protein. Preferably the CD4+ T-cell epitope comprises a region of at least 12 amino acids of an intracellular protein.

Conveniently, the universal tumour antigen is selected from the group consisting of telomerase reverse transcriptase, survivin, DNA topoisomerase 2-alpha, cytochrome P450 1B1 and E3 ubiquitin-protein ligase Mdm2.

Preferably, the universal tumour antigen is telomerase reverse transcriptase and wherein the CD4+ T-cell epitope comprises one or more sequences selected from:
(i) SEQ ID NO: 1;
(ii) SEQ ID NO: 116;
(iii) SEQ ID NO: 117;
(iv) the sequence of an immunogenic fragment of (i), (ii) or (iii) comprising at least 12 amino acids; and/or
(v) a sequence having at least 80% sequence identity to (i), (ii), (iii) or (iv).

Advantageously, the at least one polypeptide comprising the sequence of the CD4+ T-cell epitope comprises the sequence of a further T-cell epitope, wherein the further T-cell epitope is a further CD4+ T-cell epitope and/or a CD8+ T-cell epitope.

Conveniently, the conjugate comprises a further substance.

Preferably, the further substance is a further polypeptide comprising the sequence of an epitope, more preferably wherein the epitope is a further T-cell epitope, more preferably wherein the further T-cell epitope is a further CD4+ T-cell epitope and/or a CD8+ T-cell epitope.

Advantageously, the CD4+ T-cell epitope comprises a sequence selected from:
(i) SEQ ID NO: 1;
(ii) the sequence of an immunogenic fragment of (i) comprising at least 12 amino acids; or
(iii) a sequence having at least 80% sequence identity to (i) or (ii),
and wherein the further CD4+ T-cell epitope comprises a sequence selected from:
(iv) SEQ ID NO: 116;
(v) the sequence of an immunogenic fragment of (iv) comprising at least 12 amino acids; or
(vi) a sequence having at least 80% sequence identity to (iv) or (v).

According to a third aspect of the present invention, there is provided a cocktail of conjugates comprising first and second different conjugates, wherein the first conjugate and/or the second conjugate is each a conjugate of the invention.

Conveniently, the first conjugate comprises a CD4+ T-cell epitope comprising a sequence selected from:
(i) SEQ ID NO: 1;
(ii) the sequence of an immunogenic fragment of (i) comprising at least 12 amino acids; or
(iii) a sequence having at least 80% sequence identity to (i) or (ii),
and wherein the second conjugate comprises a CD4+ T-cell epitope comprising a sequence selected from:
(iv) SEQ ID NO: 116;
(v) the sequence of an immunogenic fragment of (iv) comprising at least 12 amino acids; or
(vi) a sequence having at least 80% sequence identity to (iv) or (v).

Advantageously, the CD4+ T-cell epitope is immunogenic in at least 60% of the population, preferably at least 65% of the population.

Conveniently, the CD4+ T-cell epitope is bound by one or more of the following HLA alleles present in the population: HLA-DRB1*15, HLA-DRB1*07, HLA-DRB1*04, HLA-DQB1*06, HLA-DQB1*03, HLA-DQB1*05, HLA-DPB1*04 and/or HLA-DPB1*01.

According to a fourth aspect of the present invention, there is provided a conjugate comprising:
(a) at least one polypeptide comprising a sequence of a B-cell epitope; and
(b) at least one polypeptide comprising a sequence of a CD4+ T-cell epitope,
wherein the CD4+ T-cell epitope comprises one or more sequences selected from:
(i) SEQ ID NO: 1;
(ii) SEQ ID NO: 116;
(iii) SEQ ID NO: 117;
(iv) the sequence of an immunogenic fragment of (i), (ii) or (iii) comprising at least 12 amino acids; and/or
(v) a sequence having at least 80% sequence identity to (i), (ii), (iii) or (iv),
wherein the sequence of the B-cell epitope is different from the sequence of the CD4+ T-cell epitope, and
wherein an antibody specific for the B-cell epitope binds to the conjugate.

According to a fifth aspect of the present invention, there is provided a molecule comprising a first nucleotide sequence encoding a polypeptide comprising a sequence of a B-cell epitope of the invention and/or a second nucleotide sequence encoding a polypeptide comprising a sequence of a CD4+ T-cell epitope of the invention.

Preferably, the molecule is a nucleic acid molecule.

According to a sixth aspect of the present invention, there is provided a polypeptide comprising a sequence selected from:
(i) SEQ ID NO: 116;
(ii) a sequence having at least 91% sequence identity to (i);
(iii) SEQ ID NO: 117; or
(iv) a sequence having at least 95% sequence identity to (iii),
wherein the polypeptide comprising the sequence selected from (i) or (ii) is equal to or less than 170 amino acids in length and wherein the polypeptide comprising the sequence selected from (iii) or (iv) is equal to or less than 40 amino acids in length.

Advantageously, the polypeptide comprising the sequence selected from (i) or (ii) is equal to or less than 150, 125, 100, 75 or 50 amino acids in length.

Alternatively, the polypeptide comprising the sequence selected from (iii) or (iv) is equal to or less than 35, 34, 33, 32 or 31 amino acids in length.

According to a seventh aspect of the present invention, there is provided a nucleic acid molecule consisting of a nucleotide sequence encoding a polypeptide of the invention.

According to an eighth aspect of the present invention, there is provided a vector comprising a nucleic acid molecule of the invention.

According to an ninth aspect of the present invention, there is provided a combination comprising a first product and a second product wherein the first product is selected from the following (i) to (v):
(i) a polypeptide comprising the sequence of SEQ ID NO: 1;
(ii) a polypeptide comprising an immunogenic fragment of (i) comprising at least eight amino acids;
(iii) a polypeptide comprising a sequence having at least 80% sequence identity to (i) or (ii);
(iv) a conjugate comprising the polypeptide defined in any one of (i) to (iii);
(v) a nucleic acid molecule consisting of a nucleotide sequence encoding a polypeptide as defined in any one of (i) to (iii),
and wherein the second product is selected from the following (vi) to (x):
(vi) a polypeptide comprising the sequence of SEQ ID NO: 116;
(vii) a polypeptide comprising an immunogenic fragment of (vi) comprising at least seventeen amino acids;
(viii) a polypeptide comprising a sequence having at least 80% sequence identity to (vi) or (vii);
(ix) a conjugate comprising the polypeptide defined in any one of (vi) to (viii);
(x) a nucleic acid molecule consisting of a nucleotide sequence encoding a polypeptide as defined in any one of (vi) to (viii),
wherein the first product and the second product are optionally a single product subject to the following provisos:
(a) where the first product and the second product are a single polypeptide and the first product is as defined in any one of (i) to (iii) and the second product is as defined in any one of (vi) to (viii) then the single polypeptide is equal to or less than 170 amino acids in length;
(b) where the first product and the second product are a single product and the first product is as defined in (v) and the second product is as defined in (x) then the single nucleic acid molecule is less than 1500 nucleotides in length.

Preferably, the combination comprises a cocktail of polypeptides wherein the first product is a polypeptide as defined in part (i), (ii) or (iii) above and wherein the second product is a polypeptide as defined part (vi), (vii) or (viii) above.

Advantageously, the combination comprises a cocktail of nucleic acid molecules wherein the first product is a nucleic acid molecule as defined in part (v) above and the second product is a nucleic acid molecule as defined in part (x) above.

Conveniently, the or each polypeptide or the or each nucleic acid molecule is linked to a further substance.

According to a tenth aspect of the present invention, there is provided a first product for use in medicine by simultaneous, separate or sequential administration with a second product, wherein the first product is selected from the following (i) to (v):
(i) a polypeptide comprising the sequence of SEQ ID NO: 1;
(ii) a polypeptide comprising an immunogenic fragment of (i) comprising at least eight amino acids;
(iii) a polypeptide comprising a sequence having at least 80% sequence identity to (i) or (ii);
(iv) a conjugate comprising the polypeptide defined in any one of (i) to (iii);
(v) a nucleic acid molecule consisting of a nucleotide sequence encoding a polypeptide as defined in any one of (i) to (iii),
and wherein the second product is selected from the following (vi) to (x):
(vi) a polypeptide comprising the sequence of SEQ ID NO: 116;
(vii) a polypeptide comprising an immunogenic fragment of (vi) comprising at least seventeen amino acids;
(viii) a polypeptide comprising a sequence having at least 80% sequence identity to (vi) or (vii);
(ix) a conjugate comprising the polypeptide defined in any one of (vi) to (viii);
(x) a nucleic acid molecule consisting of a nucleotide sequence encoding a polypeptide as defined in any one of (vi) to (viii), wherein the first product and the second product are optionally a single product subject to the following provisos:
(a) where the first product and the second product are a single polypeptide and the first product is as defined in any one of (i) to (iii) and the second product is as defined in any one of (vi) to (viii) then the single polypeptide is equal to or less than 170 amino acids in length;

(b) where the first product and the second product are a single product and the first product is as defined in (v) and the second product is as defined in (x) then the single nucleic acid molecule is equal to or less than 1500 nucleotides in length.

According to a eleventh aspect of the present invention, there is provided a second product for use in medicine by simultaneous, separate or sequential administration with a first product, wherein the second product is selected from the following (i) to (v):
(i) a polypeptide comprising the sequence of SEQ ID NO: 116;
(ii) a polypeptide comprising an immunogenic fragment of (i) comprising at least seventeen amino acids;
(iii) a polypeptide comprising a sequence having at least 80% sequence identity to (i) or (ii);
(iv) a conjugate comprising the polypeptide defined in any one of (i) to (iii);
(v) a nucleic acid molecule consisting of a nucleotide sequence encoding a polypeptide as defined in any one of (i) to (iii),
and wherein the first product is selected from the following (vi) to (x):
(vi) a polypeptide comprising the sequence of SEQ ID NO: 1;
(vii) a polypeptide comprising an immunogenic fragment of (vi) comprising at least eight amino acids;
(viii) a polypeptide comprising a sequence having at least 80% sequence identity to (vi) or (vii);
(ix) a conjugate comprising the polypeptide defined in any one of (vi) to (viii);
(x) a nucleic acid molecule consisting of a nucleotide sequence encoding a polypeptide as defined in any one of (vi) to (viii),
wherein the second product and the first product are optionally a single product subject to the following provisos:
(a) where the second product and the first product are a single polypeptide and the second product is as defined in any one of (i) to (iii) and the first product is as defined in any one of (vi) to (viii) then the single polypeptide is equal to or less than 170 amino acids in length;
(b) where the second product and the first product are a single product and the second product is as defined in (v) and the first product is as defined in (x) then the single nucleic acid molecule is equal to or less than 1500 nucleotides in length.

According to twelfth aspect of the present invention, there is provided a conjugate of the invention, a cocktail of conjugates of the invention, a nucleic acid molecule of the invention, a polypeptide of the invention, or a combination of the invention for use in medicine.

Preferably, the first product, the second product, the conjugate, the cocktail of conjugates, the nucleic acid molecule, the polypeptide or the combination is for use in the treatment or prophylaxis of cancer.

According to a thirteenth aspect of the present invention, there is provided a pharmaceutical composition comprising the conjugate of the invention, the cocktail of conjugates of the invention, the nucleic acid molecule of the invention, the polypeptide of the invention, or the combination of the invention and a pharmaceutically acceptable diluent, excipient or adjuvant and optionally another therapeutic ingredient.

Conveniently, the pharmaceutical composition is for use in medicine, preferably for the treatment or prophylaxis of cancer.

Preferably, the conjugate of the invention, the cocktail of conjugates of the invention, a combination or a pharmaceutical composition of the invention comprising the aforementioned or a nucleic acid molecule of the invention encoding any of the aforementioned is for use in a subject to whom a vaccine to induce a B-cell response to the B-cell epitope has been administered.

Advantageously, the vaccine to induce a B-cell response to the B-cell epitope has been administered to the subject at least twice.

Conveniently, the vaccine to induce a B-cell response to the B-cell epitope is a tetanus vaccine.

According to a fourteenth aspect of the present invention, there is provided a method of treatment or prophylaxis of cancer in a subject, comprising administering, in a therapeutically effective amount, the conjugate of the invention, the cocktail of conjugates of the invention, the nucleic acid molecule of the invention, the polypeptide of the invention, the combination of the invention and/or the pharmaceutical composition of the invention.

According to a fifteenth aspect of the present invention, there is provided a method of treatment or prophylaxis of cancer in a patient comprising administering a first product simultaneously, separately or sequentially with a second product, wherein the first product is selected from the following (i) to (v):
(i) a polypeptide comprising the sequence of SEQ ID NO: 1;
(ii) a polypeptide comprising an immunogenic fragment of (i) comprising at least eight amino acids;
(iii) a polypeptide comprising a sequence having at least 80% sequence identity to (i) or (ii):
(iv) a conjugate comprising the polypeptide defined in any one of (i) to (iii);
(v) a nucleic acid molecule consisting of a nucleotide sequence encoding a polypeptide as defined in any one of (i) to (iii),
and wherein the second product is selected from the following (vi) to (x):
(vi) a polypeptide comprising the sequence of SEQ ID NO: 116;
(vii) a polypeptide comprising an immunogenic fragment of (vi) comprising at least seventeen amino acids;
(viii) a polypeptide comprising a sequence having at least 80% sequence identity to (vi) or (vii);
(ix) a conjugate comprising the polypeptide defined in any one of (vi) to (viii);
(x) a nucleic acid molecule consisting of a nucleotide sequence encoding a polypeptide as defined in any one of (vi) to (viii),
wherein the first product and the second product are optionally a single product subject to the following provisos:
(a) where the first product and the second product are a single polypeptide and the first product is as defined in any one of (i) to (iii) and the second product is as defined in any one of (vi) to (viii) then the single polypeptide is equal to or less than 170 amino acids in length;
(b) where the first product and the second product are a single product and the first product is as defined in (v) and the second product is as defined in (x) then the single nucleic acid molecule is equal to or less than 1500 nucleotides in length, wherein the first product and/or the second product are each administered in a therapeutically effective amount. In some embodiments the first product is administered before the second product; in other embodiments the first product is administered after the second product.

Preferably, the at least one polypeptide comprising a sequence of a B-cell epitope and the at least one polypeptide comprising a sequence of a CD4+ T-cell epitope are covalently linked.

Advantageously, prior to linkage, at least one of the first linking group and the second linking group comprises two or more first linking groups or second linking groups.

Preferably, prior to linkage, the first linking group and the second linking group are independently selected from an alkyne (e.g. a terminal alkyne), an alkene (e.g. a terminal alkene, norbornene), a cycloalkyne, a trans-cycloalkene, a tetrazine, a conjugated diene, a maleimide, an α-halocarbonyl, a thiol and an azide; more preferably wherein the first linking group and the second linking group are independently selected from an alkyne (e.g. a terminal alkyne), a cycloalkyne, a maleimide and an α-halocarbonyl; more preferably wherein the first linking group and the second linking group are independently selected from an alkyne (e.g. a terminal alkyne), a cycloalkyne and an α-halocarbonyl.

Conveniently, prior to linkage, the at least one polypeptide comprising a sequence of a B-cell epitope and the at least one polypeptide comprising a sequence of a CD4+ T-cell epitope independently comprise a thiol group, an azide group, an alkyne (e.g. a terminal alkyne), an alkene (e.g. a terminal alkene, norbornene), a cycloalkyne, a trans-cycloalkene, a tetrazine, a conjugated diene, a maleimide and an α-halocarbonyl.

Preferably, the at least one polypeptide comprising a sequence of a B-cell epitope and the at least one polypeptide comprising a sequence of a CD4+ T-cell epitope independently comprise a thiol group or an azide group.

Advantageously, the first connecting element and the second connecting element are independently selected from a 1,2,3-triazole linkage, a dihydropyridazine linkage, a pyridazine linkage and a sulfide linkage (e.g. formed from thiol-ene reactions). It is especially preferred that the first connecting element and the second connecting element are independently selected from a 1,2,3-triazole linkage and a sulfide linkage.

Preferably, the first connecting element and the second connecting element are independently selected from:

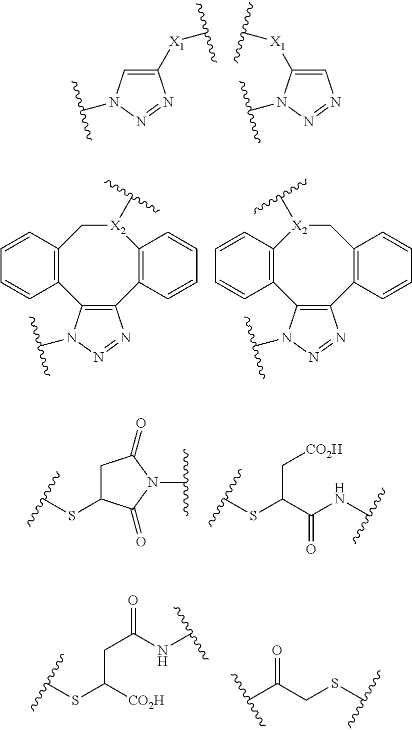

wherein:
X₁ is selected from —(CH₂)ₐₓ₁₁— and —(CH₂)ₐₓ₁₂—X₁₂—;
wherein X₁₂ is selected from O, NR₁₂ or S;
R₁₂ is selected from hydrogen, optionally substituted alkyl, optionally substituted cycloalkyl, optionally substituted heterocycloalkyl, optionally substituted aryl and optionally substituted heteroaryl; preferably hydrogen and optionally substituted alkyl;
X₂ is selected from N or CH;
ax11 and ax12 are independently selected from 0 to 12, and
the wavy line represents a connection point to the at least one polypeptide comprising a sequence of a B-cell epitope or the at least one polypeptide comprising a sequence of a CD4+ T-cell epitope and/or the core.

According to a sixteenth aspect of the present invention, there is provided a core comprising:
a body portion;
one or more first linking groups attached to the body portion; and
one or more second linking groups attached to the body portion,
wherein the first linking group and second linking group are orthogonal to each other,
at least one of the first linking group and the second linking group comprises two or more first linking groups or second linking groups, and
the first linking group and the second linking group are independently selected from an alkyne (e.g. a terminal alkyne), an alkene (e.g. a terminal alkene, norbornene), a cycloalkyne, a trans-cycloalkene, a tetrazine, a conjugated diene, a maleimide, an α-halocarbonyl, a thiol and an azide; preferably wherein the first linking group and the second linking group are independently selected from an alkyne (e.g. a terminal alkyne), a cycloalkyne, a maleimide and an α-halocarbonyl; more preferably wherein the first linking group and the second linking group are independently selected from an alkyne (e.g. a terminal alkyne), a cycloalkyne and an α-halocarbonyl;
wherein the core is not:

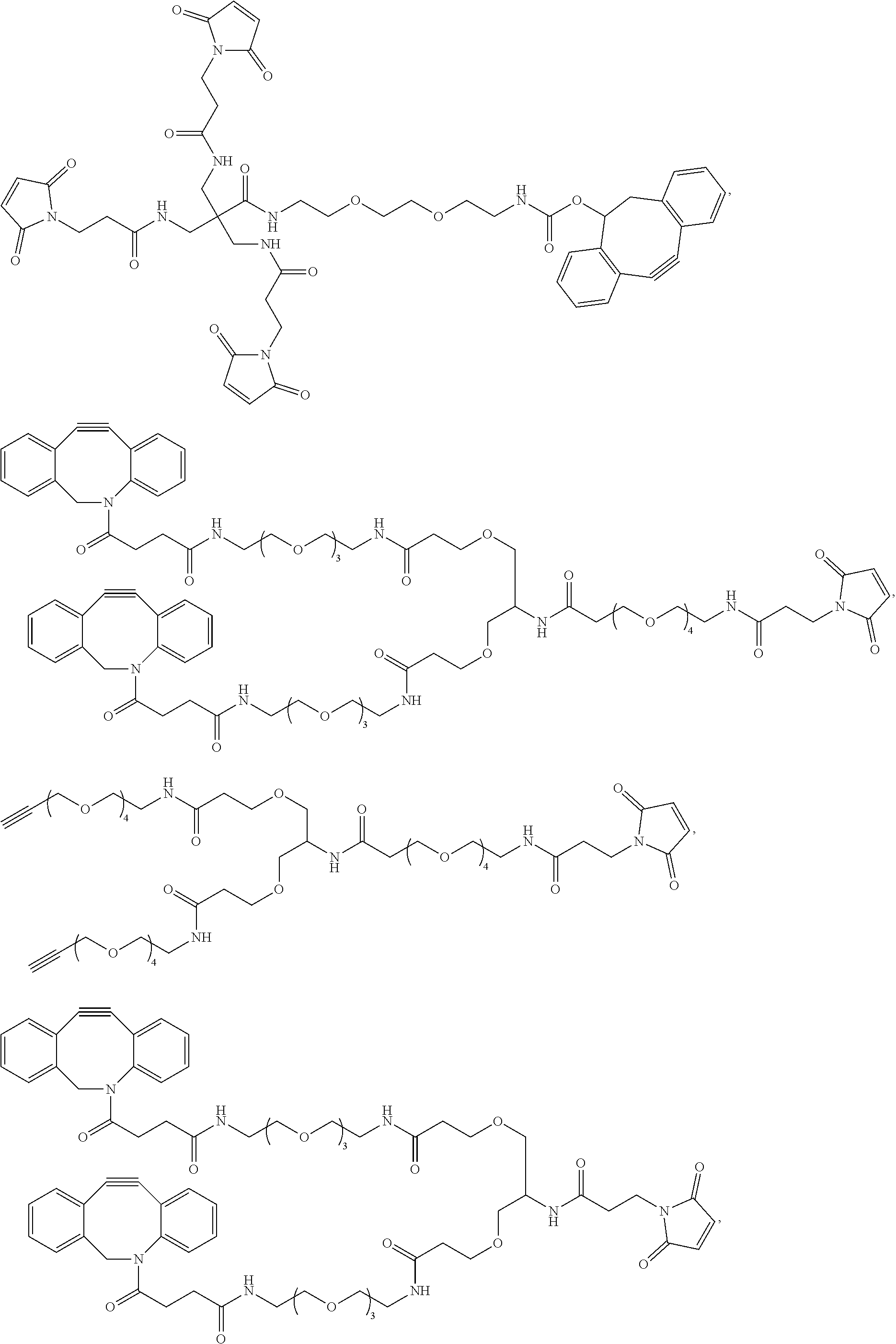

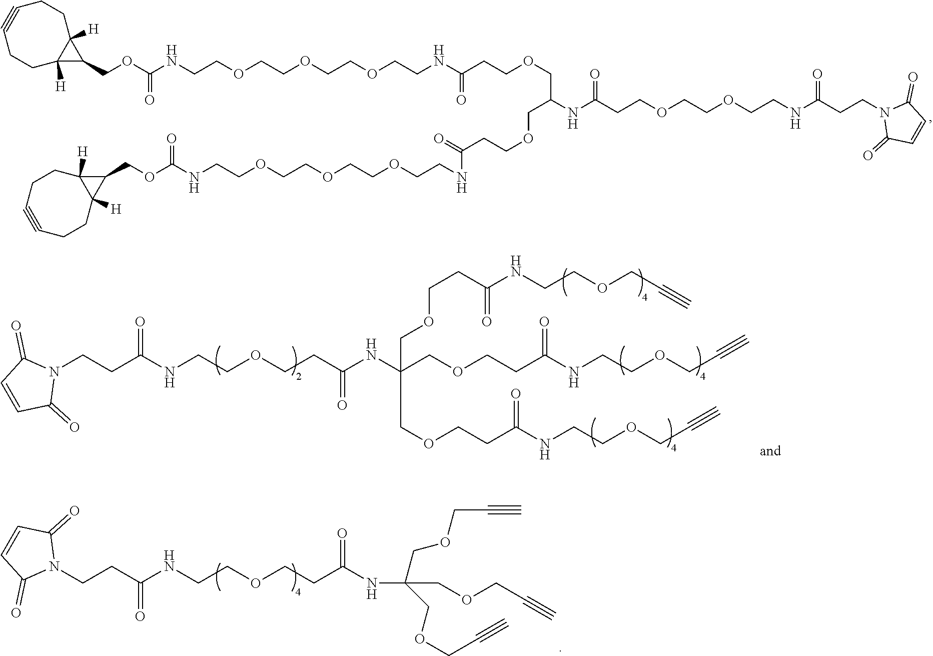

Preferably, the core comprises two or three first linking groups, preferably three first linking groups.

Conveniently, the core comprises one or two second linking groups, preferably one second linking group.

Advantageously, the first linking group and the second linking group are independently selected from:

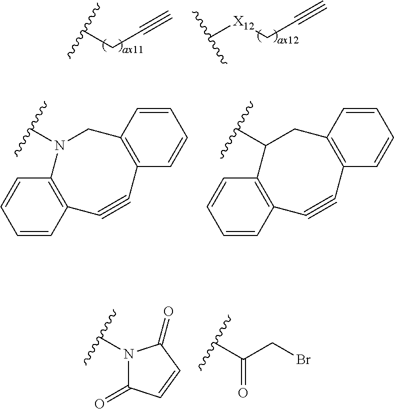

wherein X₁₂ is O, NR₁₂ or S;
R₁₂ is selected from hydrogen, optionally substituted alkyl, optionally substituted cycloalkyl, optionally substituted heterocycloalkyl, optionally substituted aryl and optionally substituted heteroaryl; preferably hydrogen and optionally substituted alkyl; and
ax11 and ax12 are independently selected from 0 to 12.

Preferably, the body portion is represented by one of Formulae 1 and 2:

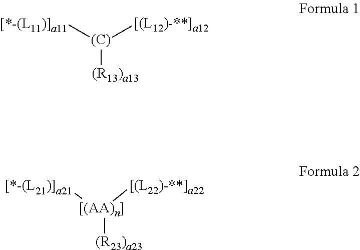

wherein in Formula 1:
L₁₁ and L₁₂ are linkers;
R₁₃ is selected from hydrogen, hydroxy, optionally substituted amino, halogen, optionally substituted alkyl, —S-(optionally substituted alkyl), optionally substituted cycloalkyl, optionally substituted heterocycloalkyl, optionally substituted alkoxy, optionally substituted alkanoyl, optionally substituted aryl and optionally substituted heteroaryl;
a11 represents the number of [(L₁₁)-*] groups attached to the carbon atom and is selected from 1, 2 or 3;
a12 represents the number of [(L₁₂)-**] groups attached to the carbon atom and is selected from 1, 2 or 3;
a13 represents the number of R₁₃ groups attached to the carbon atom and is selected from 0 or 1;
a11+a12+a13 is 4;
* represents a connection point to the first linking group;
** represents a connection point to the second linking group; and
wherein in Formula 2:
AA represents an amino acid;
n represents the number of independently selected AA groups and is selected from 1 to 12;
L₂₁ and L₂₂ are linkers;
R₂₃ is selected from hydrogen, hydroxy, optionally substituted amino, optionally substituted alkyl, —S-(optionally substituted alkyl), optionally substituted cycloalkyl, optionally substituted heterocycloalkyl, optionally substituted alkoxy, optionally substituted alkanoyl, optionally substituted aryl and optionally substituted heteroaryl;
a21 represents the number of [(L₂₁)-*] groups attached to [(AA)], and is selected from 1, 2 or 3;
a22 represents the number of [(L₂₂)-**] groups attached to [(AA)], and is selected from 1, 2 or 3;
a23 represents the number of R₂₃ groups attached to [(AA)] n at the C-terminus and/or the N-terminus, and is selected from 0, 1 or 2;
* represents a connection point to the first linking group via the N-terminus, C-terminus or a side-chain of one of the AA groups;
** represents a connection point to the second linking group via the N-terminus, C-terminus or a side-chain of one of the AA groups.

According to a seventeenth aspect of the present invention, there is provided a conjugate according to the first aspect of the invention or the core according to the second aspect of the invention, wherein the body portion is represented by one of Formulae 1 and 2:

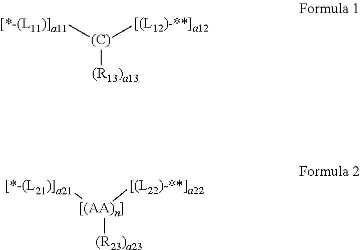

wherein in Formula 1:
L₁₁ and L₁₂ are linkers;
R₁₃ is selected from hydrogen, hydroxy, optionally substituted amino, halogen, optionally substituted alkyl, —S-(optionally substituted alkyl), optionally substituted cycloalkyl, optionally substituted heterocycloalkyl, optionally substituted alkoxy, optionally substituted alkanoyl, optionally substituted aryl and optionally substituted heteroaryl;
a11 represents the number of [(L₁₁)-*] groups attached to the carbon atom and is selected from 1, 2 or 3;
a12 represents the number of [(L₁₂)-**] groups attached to the carbon atom and is selected from 1, 2 or 3;
a13 represents the number of R₁₃ groups attached to the carbon atom and is selected from 0 or 1;
a11+a12+a13 is 4;
* represents a connection point to the first linking group;
** represents a connection point to the second linking group; and
wherein in Formula 2:
AA represents an amino acid;
n represents the number of independently selected AA groups and is selected from 1 to 12;
L₂₁ and L₂₂ are linkers;
R₂₃ is selected from hydrogen, hydroxy, optionally substituted amino, optionally substituted alkyl, —S-(optionally substituted alkyl), optionally substituted cycloalkyl, optionally substituted heterocycloalkyl, optionally substituted alkoxy, optionally substituted alkanoyl, optionally substituted aryl and optionally substituted heteroaryl;
a21 represents the number of [(L₂₁)-*] groups attached to [(AA)], and is selected from 1, 2 or 3;
a22 represents the number of [(L₂₂)-**] groups attached to [(AA)], and is selected from 1, 2 or 3;
a23 represents the number of R₂₃ groups attached to [(AA)] n at the C-terminus and/or the N-terminus, and is selected from 0, 1 or 2;
* represents a connection point to the first linking group via the N-terminus, C-terminus or a side-chain of one of the AA groups;
** represents a connection point to the second linking group via the N-terminus, C-terminus or a side-chain of one of the AA groups.

Conveniently, the body portion is represented by Formula 2.

Preferably, L₁₁, L₁₂, L₂₁ and L₂₂ are independently selected from -(optionally substituted alkylene)-, —O—, —(CONH)—, —(NHCO)—, —(CH₂CH₂O)_{w}, —(CO)—, -(optionally substituted alkylene)-O—, -(optionally substituted alkylene)-(CONH)—, -(optionally substituted alkylene)-(NHCO)—, -(optionally substituted alkylene)-(CH₂CH₂O)_{w}, -(optionally substituted alkylene)-(CO)—, —O-(optionally substituted alkylene)-, —O—(CONH)—, —O—(NHCO)—, —O—(CH₂CH₂O)_{w}, —O—(CO)—, —(CONH)-(optionally substituted alkylene)-, —(CONH)—O—, —(CONH)—(NHCO)—, —(CONH)—(CH₂CH₂O)_{w}—, —(CONH)—(CO)—, —(NHCO)-(optionally substituted alkylene)-, —(NHCO)—O—, —(NHCO)—(CONH)—, —(NHCO)—(CH₂CH₂O)_{w}, —(NHCO)—(CO)—, —(CH₂CH₂O)-(optionally substituted alkylene)-, —(CH₂CH₂O)_{w}—O—, —(CH₂CH₂O)_{w}—(CONH)—, —(CH₂CH₂O)_{w}—(NHCO)—, —(CH₂CH₂O)_{w}—(CO)—, —(CO)-(optionally substituted alkylene), —(CO)—O—, —(CO)—(CONH)—, —(CO)—(NHCO)—, —(CO)—(CH₂CH₂O)_{w}, —(CO)-(optionally substituted alkylene)-(CO)—, -(optionally substituted alkylene)-(NHCO)-(optionally substituted alkylene)-, -(optionally substituted alkylene)-(CONH)-(optionally substituted alkylene)-, -(optionally substituted alkylene)-(NHCO)—(CH₂CH₂O)_{w}-(optionally substituted alkylene)-, -(optionally substituted alkylene)-(CONH)—(CH₂CH₂O)_{w}-(optionally substituted alkylene)-, -(optionally substituted alkylene)-O-(optionally substituted alkylene)-(NHCO)—(CH₂CH₂O)_{w}-(optionally substituted alkylene)-, -(optionally substituted alkylene)-O-(optionally substituted alkylene)-(CONH)—(CH₂CH₂O)_{w}-(optionally substituted alkylene)-, —(CONH)—(CH₂CH₂O)_{w}-(optionally substituted alkylene)-(CONH)—O—, —(NHCO)—(CH₂CH₂O)_{w}-(optionally substituted alkylene)-(CONH)—O—, —(CONH)—(CH₂CH₂O)_{w}-(optionally substituted alkylene)-(NHCO)—O—, —(NHCO)—(CH₂CH₂O)_{w}-(optionally substituted alkylene)-(NHCO)—O—, —(CONH)—(CH₂CH₂O)_{w}-(optionally substituted alkylene)-(CONH)-(optionally substituted alkylene)-(CO)—, —(NHCO)—(CH₂CH₂O)_{w}-(optionally substituted alkylene)-(CONH)-(optionally substituted alkylene)-(CO)—, —(CONH)—(CH₂CH₂O)_{w}-(optionally substituted alkylene)-(NHCO)-(optionally substituted alkylene)-(CO)—, —(NHCO)—(CH₂CH₂O)_{w}-(optionally substituted alkylene)-(NHCO)-(optionally substituted alkylene)-(CO)—.

Conveniently, the body portion according to Formula 2 comprises one or more AA groups having a sidechain comprising an optionally substituted amino group. Preferably, the sidechain comprising an amino group may be a -(optionally substituted alkylene)-(optionally substituted amino) group.

Advantageously, the sidechain comprising an amino group may comprise one or more lysine groups, preferably three lysine groups.

Preferably, n is 2 to 10, preferably 2 to 6, more preferably 3 or 4, even more preferably 3.

Advantageously, an R₂₃ group connected to the C-terminus is selected from hydroxy, optionally substituted amino and optionally substituted alkoxy; preferably hydroxy and optionally substituted amino; more preferably optionally substituted amino.

Preferably, the connection point to the first linking group is via the side chain of one of the AA groups.

Conveniently, the connection point for each of the first linking groups is via the side chain of independently selected AA groups.

Advantageously, the connection point to the second linking group is via the N-terminus.

Preferably, the core further comprises a third linking group attached to the body portion, wherein the third linking group is orthogonal to the first linking group and the second linking group; more preferably wherein the third linking group is independently selected from an alkyne (e.g. a terminal alkyne), an alkene (e.g. a terminal alkene, norbornene), a cycloalkyne, a trans-cycloalkene, a tetrazine, a conjugated diene, a maleimide, an α-halocarbonyl, a thiol and an azide.

Conveniently, the connection point to the third linking group is via the side chain of one of the AA groups (e.g. at a serine group), or via the C-terminus. Preferably, the connection point to the third linking group is via the C-terminus.

According to an eighteenth aspect of the present invention, there is provided a conjugate comprising:
(a) at least one polypeptide comprising a sequence of a B-cell epitope; or
(b) at least one polypeptide comprising a sequence of a CD4+ T-cell epitope;
   wherein the at least one polypeptide comprising a sequence of a B-cell epitope or the at least one polypeptide comprising a sequence of a CD4+ T-cell epitope is linked to a core, wherein the core comprises, prior to linkage:
      a body portion;
      one or more first linking groups attached to the body portion; and
      one or more second linking groups attached to the body portion,
      wherein the first linking group and second linking group are orthogonal to each other,
      wherein the core is not:

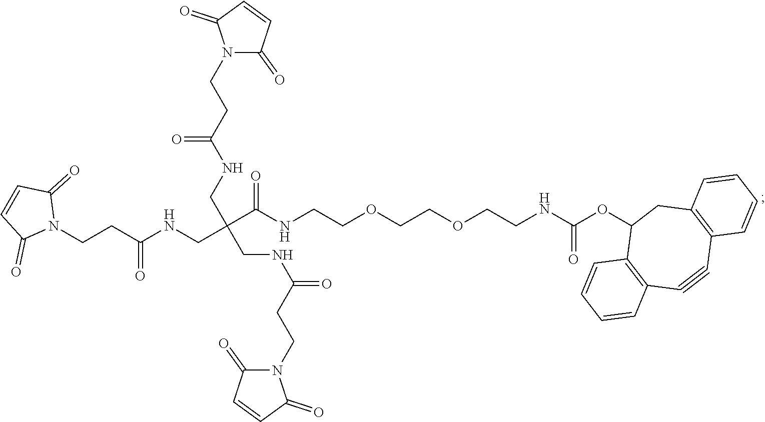 [SMILES: O=C(CCN1C(=O)C=CC1=O)NCC(CNC(=O)CCN1C(=O)C=CC1=O)(CNC(=O)CCN1C(=O)C=CC1=O)C(=O)NCCOCCOCCNC(=O)OC1Cc2ccccc2C#Cc2ccccc21]

and wherein the first linking group is linked to the at least one polypeptide comprising a sequence of a B-cell epitope to form a first connecting element, or the second linking group is linked to the at least one polypeptide comprising a sequence of a CD4+ T-cell epitope to form a second connecting element.

Preferably, there is provided a conjugate comprising:
(a) at least one polypeptide comprising a sequence of a B-cell epitope; and
(b) at least one polypeptide comprising a sequence of a CD4+ T-cell epitope;
and wherein the first linking group is linked to the at least one polypeptide comprising a sequence of a B-cell epitope to form a first connecting element, and the second linking group is linked to the at least one polypeptide comprising a sequence of a CD4+ T-cell epitope to form a second connecting element.

Preferably, the core is as defined in any one of the above.

Conveniently, the B-cell epitope and/or the CD4+ T-cell epitope is as defined in any one of the above.

According to a nineteenth aspect of the present invention, there is provided a process for manufacturing a conjugate, comprising the steps of:
(a) providing at least one polypeptide comprising a sequence of a B-cell epitope; and
(b) providing at least one polypeptide comprising a sequence of a CD4+ T-cell epitope and linking the at least one polypeptide comprising a sequence of a CD4+ T-cell epitope to the at least one polypeptide comprising a sequence of a B-cell epitope,
   wherein the CD4+ T-cell epitope comprises a region of at least 12 amino acids of a universal tumour antigen or a sequence having at least 80% sequence identity to the region and wherein the CD4+ T-cell epitope is immunogenic in at least 50% of the population, wherein the at least one polypeptide comprising the sequence of the CD4+ T-cell epitope is equal to or less than 500 amino acids in length,
   wherein the sequence of the B-cell epitope is different from the sequence of the CD4+ T-cell epitope, and
   wherein an antibody specific for the B-cell epitope binds to the conjugate.

Conveniently, the at least one polypeptide comprising a sequence of a B-cell epitope and the at least one polypeptide comprising a sequence of a CD4+ T-cell epitope are linked via a core,
wherein the core comprises, prior to linkage:
   a body portion;
   one or more first linking groups attached to the body portion; and
   one or more second linking groups attached to the body portion,
   wherein the first linking group and second linking group are orthogonal to each other;
and wherein the first linking group is linked to the at least one polypeptide comprising a sequence of a B-cell epitope to form a first connecting element, and the second linking group is linked to the at least one polypeptide comprising a sequence of a CD4+ T-cell epitope to form a second connecting element.

According to a twentieth aspect of the present invention, there is provided a process for manufacturing a conjugate, comprising the steps of:
(a) providing a core comprising:
   a body portion;
   one or more first linking groups attached to the body portion; and
   one or more second linking groups attached to the body portion,
   wherein the first linking group and second linking group are orthogonal to each other,
   at least one of the first linking group and the second linking group comprises two or more first linking groups or second linking groups, and
   the first linking group and the second linking group are independently selected from an alkyne (e.g. a terminal alkyne), an alkene (e.g. a terminal alkene, norbornene), a cycloalkyne, a trans-cycloalkene, a tetrazine, a conjugated diene, a maleimide, an α-halocarbonyl, a thiol or an azide; preferably wherein the first linking group and the second linking group are independently selected from an alkyne (e.g. a terminal alkyne), a cycloalkyne, a maleimide and an α-halocarbonyl; more preferably wherein the first linking group and the second linking group are independently selected from an alkyne (e.g. a terminal alkyne), a cycloalkyne and an α-halocarbonyl;
   wherein the core is not:

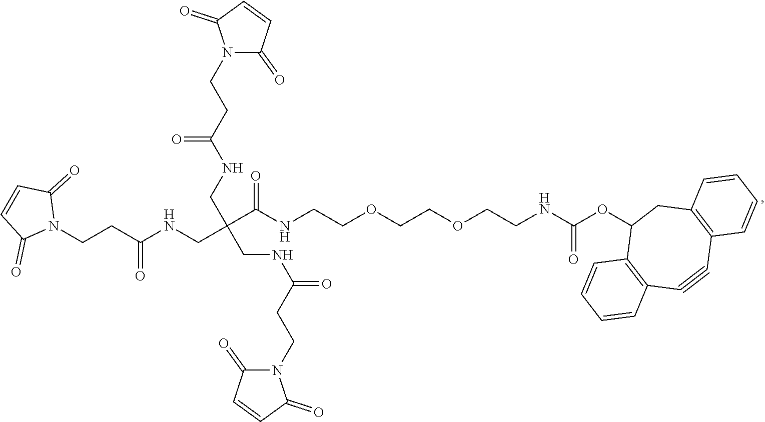 [SMILES: O=C(CCN1C(=O)C=CC1=O)NCC(CNC(=O)CCN1C(=O)C=CC1=O)(CNC(=O)CCN1C(=O)C=CC1=O)C(=O)NCCOCCOCCNC(=O)OC1Cc2ccccc2C#Cc2ccccc21]

preferably wherein the core is as defined in any one of the above;
(b) providing at least one polypeptide comprising a sequence of a B-cell epitope, or at least one polypeptide comprising a sequence of a CD4+ T-cell epitope; and reacting the core with the at least one polypeptide comprising a sequence of a B-cell epitope to form a first connecting element, or reacting the core with the at least one polypeptide comprising a sequence of a CD4+ T-cell epitope to form a second connecting element.

Preferably, the process further comprises the step of:
(c) providing the other of at least one polypeptide comprising a sequence of a CD4+ T-cell epitope, or at least one polypeptide comprising a sequence of a B-cell epitope, not provided in step (b); and reacting the core with the at least one polypeptide comprising a sequence of a CD4+ T-cell epitope to form a second connecting element if the first connecting element was formed in step (b); or reacting the core with the at least one polypeptide comprising a sequence of a B-cell epitope to form a first connecting element if the second connecting element was formed in step (b).

According to a twenty-first aspect of the present invention, there is provided a polypeptide comprising a sequence selected from:
(i) the sequence of UV36 (SEQ ID NO: 166), UV57 (SEQ ID NO: 167), UV58 (SEQ ID NO: 168), UV59 (SEQ ID NO: 169), UV60 (SEQ ID NO: 170), UV64 (SEQ ID NO: 171), UV65 (SEQ ID NO: 172) or UV66 (SEQ ID NO: 173);
(ii) the sequence of an immunogenic fragment of (i) comprising at least 8 amino acids; or
(iii) a sequence having at least 80% sequence identity to (i) or (ii),
wherein the polypeptide is equal to or less than 25, 22, 20, 18, 15, or 10 amino acids in length.

Preferably, the polypeptide does not consist of or comprise the sequence of SEQ ID NO: 126 or 171.

According to a twenty-second aspect of the present invention, there is provided a nucleic acid molecule consisting of a nucleotide sequence encoding a polypeptide in accordance with the twenty-first aspect of the present invention.

According to a twenty-third aspect of the present invention, there is provided the polypeptide of the twenty-first aspect of the present invention or the nucleic acid molecule of the twenty-second aspect of the present invention for use in medicine, preferably for inducing an immune response and more preferably for the treatment or prophylaxis of cancer.

The terms “polypeptide”, “peptide” and “protein” are used interchangeably herein to refer to a polymer of amino acid residues (including linear, cyclic, partially cyclic or multiply cyclic). The terms apply to amino acid polymers in which one or more amino acid residues is a modified residue, or a non-naturally occurring residue, such as an artificial chemical mimetic of a corresponding naturally occurring amino acid, as well as to naturally occurring amino acid polymers. In one embodiment, the term also encompasses amino acid polymers comprising sidechain elongation. In one embodiment, the backbone and/or the sidechain elongation comprises a linkage other than an amide linkage. In one embodiment, an interrupting group in the polypeptide or a fragment thereof is not an amino acid.

The term “amino acid” as used herein refers to naturally occurring and synthetic amino acids, as well as amino acid analogues and amino acid mimetics that have a function that is similar to the naturally occurring amino acids. Naturally occurring amino acids are those encoded by the genetic code, as well as those modified after translation in cells (e.g. hydroxyproline, gamma-carboxyglutamate, and O-phosphoserine). The phrase “amino acid analogue” refers to compounds that have the same basic chemical structural elements (an alpha carbon bound to a hydrogen, a carboxy group, an amino group, and an R group) as a naturally occurring amino acid but have a modified R group or modified backbones (e.g. homoserine, beta-alanine, PNA, norleucine, methionine sulfoxide, methionine methyl sulphonium). The phrase “amino acid mimetic” refers to chemical compounds that have different structures from but similar functions to naturally occurring amino acids. Thus in one embodiment, the amino acid analogue and/or amino acid mimetic provides a similar biological property to that of a naturally occurring amino acid despite having a difference in structure.

The term “fragment” as used herein in relation to a polypeptide means a consecutive series of amino acids that form part of the polypeptide. An “immunogenic fragment” of a polypeptide is a fragment as previously defined which is capable of eliciting an immune response, such as a T-cell response, when administered to a subject. In one embodiment, the “immunogenic fragment” is capable of eliciting a CD4+ T-cell response when administered to a subject. In one embodiment, the “immunogenic fragment” is capable of eliciting a CD4+ and/or CD8+ T-cell immune response when administered to a subject. In some embodiments, the immunogenic fragment comprises at least 8, 9, 10, 11, 12, 13, 14, 15, 16, 17, 18, 19, 20, 21, 22, 23, 24, 25, 26, 27, 28 or 29 amino acids of the polypeptide from which it is derived.

The terms “gene”, “oligonucleotide”, “polynucleotide”, and “nucleic acid molecule” are used interchangeably herein to refer to a polymer of multiple nucleotides. The nucleic acid molecules may comprise naturally occurring nucleic acids or may comprise artificial nucleic acids such as peptide nucleic acids (PNA), morpholino (PMO) and locked nucleic acid as well as glycol nucleic acid and threose nucleic acid. The “oligonucleotide” may also comprise non-nucleic acid derivatives in the e.g., polymer backbone, non-backbone modification(s).

The term “nucleotide” as used herein refers to naturally occurring nucleotides and synthetic nucleotide analogues that are recognised by e.g. cellular enzymes.

The terms “cancer” and “tumour” as used herein refer to the presence of cells in a subject that exhibit new, abnormal and/or uncontrolled proliferation. In one embodiment, the cells have the capacity to invade adjacent tissues and/or to spread to other sites in the body (i.e. the cells are capable of metastasis). In one embodiment, the cancer cells are in the form of a tumour (i.e. an abnormal mass of tissue). The term “tumour” as used herein includes both benign and malignant neoplasms.

The term “treatment” as used herein refers to any partial or complete treatment and includes: inhibiting the disease or symptom, i.e. arresting its development; and relieving the disease or symptom, i.e. causing regression of the disease or symptom.

The term “prophylaxis” as used herein refers to a measure taken to prevent the onset of a disease. In one embodiment, the disease is cancer.

The term “antigen” as used herein refers to a molecule capable of eliciting an immune response in a subject. In one embodiment, the “antigen” is a protein or a polypeptide or is derived from a protein or a polypeptide. In one embodiment, an “antigen” is capable of being recognised by an antibody, a B-cell receptor and/or a T-cell receptor.

The term “epitope” as used herein refers to the part of the antigen that is recognised by an antibody, a B-cell receptor and/or a T-cell receptor. The term “epitope” as used herein comprises conformational epitopes (i.e. which are composed of amino acid residues that are separated in sequence but which are brought together by protein folding) and linear epitopes (i.e. which are linear sequence of amino acid residues).

The term “antibody” as used herein refers to an immunoglobulin molecule or a fragment thereof that is capable of binding specifically to a particular antigen. The term “antibody” as used herein includes an antibody selected from any one of the five classes of immunoglobulin: immunoglobulin G (IgG), IgM, IgA, IgD and IgE.

The term “B-cell” (also known as “B-lymphocyte”) as used herein refers to a cell of the immune system which has a cell surface B-cell receptor.

The term “B-cell epitope” as used herein refers to a site within an antigen that is capable of being bound by an antibody and/or a B-cell receptor. In one embodiment, the B-cell epitope comprises the sequence of SEQ ID NO. 7 (also referred to as a “Minimal Tetanus Toxin Epitope” or “MTTE”).

The term “T-cell” (also known as “T lymphocyte”) as used herein refers to a cell of the immune system which has a cell surface T-cell receptor. In one embodiment, the term “T-cell” comprises different types of T cell, such as: CD4+ T-cells (also known as helper T-cells or Th cells), CD8+ T-cells (also known as cytotoxic T-cells or CTLs), memory T-cells and regulatory T-cells (Tregs). The term “CD4+ T-cell” as used herein refers to a T-cell comprising a CD4 glycoprotein on its cell surface. The term “CD8+ T-cell” as used herein refers to a T-cell comprising a CD8 glycoprotein on its cell surface.

The term “T-cell epitope” as used herein refers to a site within an antigen that is capable of being recognised by a T-cell receptor. The T-cell receptor recognises epitopes in the context of major histocompatibility complex (MHC) molecules.

The term “MHC molecule” as used herein refers to a protein structure which assembles with a polypeptide and which is capable of displaying the polypeptide at a cell surface to a T-cell. MHC molecules are encoded by genes within the major histocompatibility complex. In some embodiments, the term “MHC molecule” refers to an MHC class I molecules and/or an MHC class II molecule.

The term “human leukocyte antigen (HLA)” as used herein refers to the human major histocompatibility complex (MHC). The main human HLA class I genes include HLA-A, HLA-B and HLA-C. The main human HLA class II genes include HLA-DPA, HLA-DPB, HLA-DQA, HLA-DQB, HLA-DRA and HLA-DRB. The term “HLA allele” as used herein refers to an alternative form of the gene present at an HLA locus.

The term “CD4+ T-cell epitope” as used herein refers to an epitope that is capable of being recognised by a CD4+ T-cell. The CD4+ T-cell epitope or a portion thereof is capable of being presented by an MHC Class II molecule and being bound by a T-cell receptor of the CD4+ T-cell. In one embodiment, the CD4+ T-cell epitope comprises one or more sequences selected from SEQ ID NO: 1, 116 and/or 117 or a sequence having at least 80% sequence identity thereto. In a further embodiment, the CD4+ T-cell epitope comprises an immunogenic fragment of SEQ ID NO: 1, 116 and/or 117 comprising at least 12 amino acids thereof.

The term “CD8+ T-cell epitope” as used herein refers to an epitope that is capable of being recognised by a CD8+ T-cell. The CD8+ T-cell epitope or a portion thereof is capable of being presented by an MHC Class I molecule and being bound by a T-cell receptor of the CD8+ T-cell. In one embodiment, the CD8+ T-cell epitope is derived from prostatic acid phosphatase (PAP). In one embodiment, the CD8+ T-cell epitope comprises the sequence “NPILLWQPIPV” (SEQ ID NO: 119). In a further embodiment, the CD8+ T-cell epitope comprises a sequence selected from SEQ ID NOS: 155 to 159.

The term “conjugate” as used herein refers to the coupling or linking of two or more components, e.g. between a polypeptide comprising a sequence of a B-cell epitope and a polypeptide comprising a sequence of a CD4+ T-cell epitope. In one embodiment, a linkage between any two components may be a direct linkage. In an alternative embodiment, a linkage between any two components may be an indirect linkage, for example by a core as defined herein. The conjugation may be via a non-covalent linkage (e.g. by one or more interactions of ionic bonds, hydrogen bonds, hydrophobic interactions, IT-IT interactions, van der Waals interactions, affinity interactions and host-guest interactions) or a covalent linkage. In preferred embodiments, the linkage is a covalent linkage.

The term “cocktail” as used herein refers to a mixture of two or more compounds. In one embodiment, the compounds in the mixture are not linked or coupled to each other. In some embodiments, the cocktail comprises a mixture of two or more different polypeptides, two or more different nucleic acid molecules, two or more different conjugates and/or any combination thereof. In one embodiment, the polypeptides, nucleic acid molecules and/or conjugates are different in the sense of comprising different amino acid or nucleotide sequences.

The term “universal tumour antigen” as used herein refers to an antigen that is expressed in a high proportion of tumour types. In one embodiment, the universal tumour antigen is expressed in at least 50%, 60% or 70% or all tumour types, more preferably in at least 80%, 85% or 90% of all tumour types. In a further embodiment, the universal tumour antigen is also expressed in a high proportion of patients within each tumour type. In one embodiment, the universal tumour antigen is generally expressed in at least 40%, 50%, 60%, 70%, 80% or 90% of patients within each tumour type. In one embodiment, the universal tumour antigen has a direct role in oncogenesis. In one embodiment the universal tumour antigen is selected from the group consisting of telomerase reverse transcriptase, survivin, DNA topoisomerase 2-alpha (Top2α), cytochrome P450 1B1 (CYP1B1) and E3 ubiquitin-protein ligase Mdm2. Preferably, the universal tumour antigen is human telomerase reverse transcriptase (hTERT).

The phrase “the CD4+ T-cell epitope is immunogenic in at least 50% of the population” as used herein refers to the proportion of individuals within a population in whom the CD4+ T-cell epitope is capable of eliciting an immune response. In one embodiment, the population is a worldwide population. That is to say, the population is not limited to a particular geographic region. In one embodiment, the population is a population from a particular geographic region. In one embodiment, the population is any one or more of a European population, a North American population, a South and/or a Central American population, a North African, a South African, an East African, a West African, Central African and/or a Sub-Saharan African population, a West Indies population, a Western Asian, an East Asian, North-East Asian, South Asian, South-East Asian and/or South-West Asian population, an Oceania population and/or an Australian population. In a further embodiment, the population is the general population. That is to say the population comprises both healthy individuals and individuals with a disease such as cancer. In an alternative embodiment, the population consists of individuals who are cancer patients. In a further embodiment, the cancer patients are patients having non-small-cell lung carcinoma, prostate cancer and/or malignant melanoma. In one embodiment, the population further comprises cancer patients with pancreatic cancer. In one embodiment, a T-cell immune response is measured by a T-cell proliferation assay (³H-Thymidine) using blood samples from the individuals (as previously described in Inderberg-Suso et al. Oncoimmunology. 2012 Aug. 1; 1 (5): 670-686). In one embodiment, the T-cell immune response is considered positive if the response to the CD4+ epitope is at least 2 times the background (Stimulation Index, SI ≥2). In an alternative embodiment, the T-cell immune response is considered positive if the response to the CD4+ epitope is at least 3 times the background (Stimulation Index, SI ≥3). In one embodiment, the proportion of individuals within a population in whom the CD4+ T-cell epitope is immunogenic is determined by measuring T-cell immune responses to the CD4+ epitope in a sample of at least 50 randomly selected individuals from the population. In a further embodiment, the sample comprises at least 100 randomly selected individuals from the population. The T-cell immune responses as measured in the sample is taken to be representative of the population as a whole. In one embodiment, an alternative method is used to measure the T-cell immune response. In one embodiment, the method comprises a delayed-type hypersensitivity (DTH) assay, an ELISpot assay and/or flow cytometry.

The phrase “an antibody specific for the B-cell epitope binds to the conjugate” as used herein refers to the ability of an antibody specific for the B-cell epitope to interact with the epitope when comprised within the conjugate. In one embodiment, the antibody is a monoclonal or a polyclonal antibody, preferably a polyclonal antibody. In one embodiment, the B-cell epitope comprises the sequence of SEQ ID NO: 7 (MTTE) and the antibody is an anti-MTTE antibody. In one embodiment, the ability of an antibody specific for the B-cell epitope to bind to the conjugate is determined using an enzyme-linked immunosorbent assay (ELISA) protocol. In one embodiment, an indirect or a sandwich ELISA protocol is used. In one embodiment, the ELISA protocol as set out in Example 9 or 13 is used.

The phrase “a vaccine to induce a B-cell response to the B-cell epitope” as used herein refers to a vaccine that is capable of eliciting the production (by B-cells) of antibodies specific to the B-cell epitope in the subject to whom it has been administered. In one embodiment, the B-cell response to the B-cell epitope is determined by measuring a quantity of antibody in a sample derived from the subject using an ELISA protocol. A suitable ELISA protocol has been described previously, for example, in Fletcher et al. J Immunol. 2018 Jul. 1; 201 (1): 87-97, which is incorporated herein by reference.

The term “tetanus vaccine” as used herein refers to a vaccine that is capable of eliciting an immune response in a subject against the tetanus toxin. In one embodiment, the “tetanus vaccine” comprises the tetanus toxoid, a fragment thereof and/or a fragment of the tetanus toxin. The tetanus toxoid is an inactivated form of the tetanus toxin. In one embodiment, the tetanus vaccine is provided in combination with further antigens and/or toxoids. In one embodiment, the “tetanus vaccine” is a diphtheria, tetanus and pertussis (DTP) combination vaccine or any tetanus toxoid containing vaccine regimen.

The phrase “the vaccine to induce a B-cell response to the B-cell epitope has been administered to the subject at least twice” as used herein refers to a prime-boost immunization strategy. That is to say, the subject has received at least a first (priming) dose of the vaccine followed by at least a second (booster) dose of the vaccine. In one embodiment, the particular composition of the vaccine used for the priming dose is the same as that used for the one or more booster doses. In an alternative embodiment, the particular composition of the vaccine used for the priming dose is different from that used for one or more of the booster doses, provided that the vaccine is still capable of inducing a B-cell response to the B-cell epitope. In one embodiment, a tetanus toxoid containing vaccine is used to boost anti-tetanus (e.g. anti-MTTE) antibody titres prior to a prime-boost immunization strategy with a conjugate of the present invention.

The phrase “the presence of a CD4+ T-cell response to a CD4+ T-cell epitope” as used herein refers to the existence of a CD4+ T-cell that is capable of recognising the CD4+ T-cell epitope. The CD4+ T-cell recognises and is capable of binding the CD4+ T-cell epitope or a portion thereof when presented on an appropriate MHC molecule. In one embodiment, “the presence of a CD4+ T-cell response to a CD4+ T-cell epitope” can be confirmed by a T-cell proliferation assay (3H-Thymidine) as described above.

The phrase “detecting a quantity or an absence of an antibody specific to the B-cell epitope” as used herein refers to determining an amount or an absence of an antibody which is present in a sample and which is capable of binding to the B-cell epitope. In one embodiment, an enzyme-linked immunosorbent assay (ELISA) is used to detect the quantity or absence of the antibody. In one embodiment, an antibody titre is calculated.

The term “a sample derived from the subject” as used herein refers to a sample that has been obtained from the subject (i.e. it is ex vivo). In one embodiment, the sample is a blood sample. In one embodiment, the sample is a plasma sample or a diluted plasma sample.

The term “self-antigen” as used herein refers to an antigen that is derived from a naturally-occurring protein within the human body. In general, under normal conditions, the immune system does not react to self-antigens due to negative selection of T cells in the thymus. However, in a subject with cancer, a self-antigen may be recognised as foreign by the immune system (for example, as a result of the cancer cell overexpressing the protein from which the self-antigen is derived or expressing it inappropriately given the tissue in which the cancer developed) and a T cell immune response is mounted against the self-antigen. In one embodiment, the “self-antigen” is derived from telomerase reverse transcriptase.

The term “tumour-associated antigen” as used herein refers to an antigen that is associated with a tumour or cancer cell as well as a normal cell. In some embodiments, the “self-antigen” is a “tumour-associated antigen”.

The phrase “a clinically relevant response to the conjugate” as used herein refers an improved clinical outcome in a cancer patient. In one embodiment, an improved clinical outcome is a partial or a complete response (also known as a partial or a complete remission) or stable disease. A complete response refers to the disappearance of detectable tumour or cancer in the body in response to treatment; a partial response refers to a decrease in tumour size, or in the extent of cancer in the body, in response to treatment; and stable disease means that the tumour or cancer in the body is neither decreasing nor increasing in extent or severity.

In this specification, the percentage “identity” between two sequences is determined using EMBOSS Needle Pairwise Sequence Alignment (Rice et al., Trends Genet. 2000 June; 16 (6): 276-7; Nucleic Acids Res. 2019 Jul. 2: 47 (W1): W636-W641) using default parameters. In particular, EMBOSS Needle can be accessed on the internet using the URL: https://www.ebi.ac.uk/Tools/psa/emboss_needle/.

The term “body portion” as used herein refers to any backbone that is able to attach to one or more first linking groups as defined herein and one or more second linking groups as defined herein. The body portion is not particularly limited as long as it connects the first linking group(s) to the second linking group(s). Non-limiting examples of the body portion include body portions according to Formula 1, amino acids, peptides (e.g. body portions according to Formula 2), carbohydrates, other scaffolds, other biomolecules or a combination thereof.

The term “(AA)ₙ” as used herein refers to the following diradical structure.

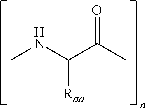

wherein Rₐₐ represents an amino acid side-chain (e.g. side-chains of natural (including glycine, i.e. H) or non-natural amino acids), wherein individual AA groups may be connected in any orientation to the next AA group (e.g. a carbonyl moiety of one AA group connected to an amine moiety of another AA group, a carbonyl moiety of one AA group connected via a side chain of another AA group, an amine moiety of one AA group connected via a side chain of another AA group, or a side chain of one AA group connected via a side chain of another AA group), and wherein individual AA groups are optionally spaced from each other by a linker; or, in the case of an individual AA group being based on an amino acid having a secondary amine (e.g. AA being proline), then Rₐₐ and the N atom of an AA group together with the carbon atom to which they are attached form a cyclic ring, with the structure:

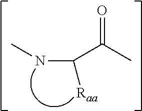

The term “C-terminus” as used in the context of the term “(AA)ₙ” refers to any end carbonyl group of the structure of “(AA)ₙ”. Thus, when a group is connected to the C-terminus, it is connected via an end carbonyl group that is not attached to a linker or other AA groups.

The term “N-terminus” as used in the context of the term “(AA)ₙ” refers to an end amine group of the structure of “(AA)ₙ”. Thus, when a group is connected to the N-terminus, it is connected via an end amine group that is not attached to a linker or other AA groups.

The term “first linking group” as used herein refers to a functional group which is able to undergo a coupling reaction with a first reactant (e.g. a polypeptide comprising a sequence of a B-cell epitope) to form a non-covalent linkage or a covalent linkage, and thus is able to form a first connecting element. In preferred embodiments, the linkage is a covalent linkage, such as by a cycloaddition reaction (e.g. 1,3-dipolar cycloaddition, including copper-catalysed azide-alkyne cycloaddition and strain-promoted azide-alkyne cycloaddition or azide-alkene cycloaddition; tetrazine ligation, such as with tetrazines and cycloalkynes or trans-cycloalkenes; Diels-Alder cycloaddition reactions), nucleophilic substitution, a Michael reaction, or other examples of click or click-like reactions. Non-limiting examples of first linking groups include alkynes (e.g. terminal alkynes), alkenes (e.g. terminal alkenes, norbornenes), cycloalkynes, trans-cycloalkenes, tetrazines, conjugated dienes, maleimides, α-halocarbonyls, thiols and azides. When one or more first linking groups are referred to, each of the first linking groups may be the same or different to each other. In some cases, each of the first linking groups are the same as each other.

The term “second linking group” as used herein refers to a functional group which is able to undergo a coupling reaction with a second reactant (e.g. a polypeptide comprising a sequence of a CD4+ T-cell epitope) to form a non-covalent linkage or a covalent linkage, and thus is able to form a second connecting element. In preferred embodiments, the linkage is a covalent linkage, such as by a cycloaddition reaction (e.g. 1,3-dipolar cycloaddition, including copper-catalysed azide-alkyne cycloaddition and strain-promoted azide-alkyne cycloaddition or azide-alkene cycloaddition; tetrazine ligation, such as with tetrazines and cycloalkynes or trans-cycloalkenes; Diels-Alder cycloaddition reactions), nucleophilic substitution, a Michael reaction or other examples of click or click-like reactions. Non-limiting examples of second linking groups include alkynes (e.g. terminal alkynes), alkenes (e.g. terminal alkenes, norbornenes), cycloalkynes, trans-cycloalkenes, tetrazines, conjugated dienes, maleimides, α-halocarbonyls, thiols and azides. When one or more second linking groups are referred to, each of the second linking groups may be the same or different to each other. In some cases, each of the second linking groups are the same as each other. The second linking group may be different from the first linking group. The second linking group may be orthogonal to the first linking group.

The term “third linking group” as used herein refers to a functional group which is able to undergo a coupling reaction with a third reactant (e.g. a further substance as defined herein) to form a non-covalent linkage or a covalent linkage, and thus is able to form a third connecting element. In preferred embodiments, the linkage is a covalent linkage, such as by a cycloaddition reaction (e.g. 1,3-dipolar cycloaddition, including copper-catalysed azide-alkyne cycloaddition and strain-promoted azide-alkyne cycloaddition or azide-alkene cycloaddition; tetrazine ligation, such as with tetrazines and cycloalkynes or trans-cycloalkenes; Diels-Alder cycloaddition reactions), nucleophilic substitution, a Michael reaction or other examples of click or click-like reactions. Non-limiting examples of third linking groups include alkynes (e.g. terminal alkynes), alkenes (e.g. terminal alkenes, norbornenes), cycloalkynes, trans-cycloalkenes, tetrazines, conjugated dienes, maleimides, α-halocarbonyls, thiols and azides. When one or more third linking groups are referred to, each of the third linking groups may be the same or different to each other. In some cases, each of the third linking groups are the same as each other. The third linking group may be different from the first linking group. The third linking group may be orthogonal to the first linking group. The third linking group may be different from the second linking group. The third linking group may be orthogonal to the second linking group. The third linking group may be different from the first linking group and the second linking group. The third linking group may be orthogonal to the first linking group and the second linking group.

The term “first connecting element” as used herein refers to a non-covalent linkage or a covalent linkage that has formed between a first linking group and a first reactant (e.g. a polypeptide comprising a sequence of a B-cell epitope). Non-limiting examples of first connecting elements include 1,2,3-triazole linkages, dihydropyridazine linkages, pyridazine linkages and sulfide linkages (e.g. formed from thiol-ene reactions). Where regioisomers of the first connecting elements are formed (e.g. regioisomers of 1,2,3-triazoles and/or ring-opened maleimide-thiol adducts), the present invention encompasses mixtures of regioisomeric products, as well as purified separate regioisomers.

The term “second connecting element” as used herein refers to a non-covalent linkage or a covalent linkage that has formed between a second linking group and a second reactant (e.g. a polypeptide comprising a sequence of a CD4+ T-cell epitope). Non-limiting examples of second connecting elements include 1,2,3-triazoles, dihydropyridazine linkages, pyridazine linkages and sulfide linkages (e.g. formed from thiol-ene reactions). Where regioisomers of the second connecting elements are formed (e.g. regioisomers of 1,2,3-triazoles and/or ring-opened maleimide-thiol adducts), the present invention encompasses mixtures of regioisomeric products, as well as purified separate regioisomers.

The term “third connecting element” as used herein refers to a non-covalent linkage or a covalent linkage that has formed between a third linking group and a third reactant (e.g. a further substance as defined herein). Non-limiting examples of third connecting elements include 1,2,3-triazoles, dihydropyridazine linkages, pyridazine linkages and sulfide linkages (e.g. formed from thiol-ene reactions). Where regioisomers of the third connecting elements are formed (e.g. regioisomers of 1,2,3-triazoles and/or ring-opened maleimide-thiol adducts), the present invention encompasses mixtures of regioisomeric products, as well as purified separate regioisomers.

The term “orthogonal” as used herein refers to a functional group (e.g. a first linking group as a nucleophile/electrophile, dienophile/diene or dipolarophile/dipole) which may react selectively with a reactant (e.g. with a first reactant, such as an electrophile/nucleophile, diene/dienophile or dipole/dipolarophile) in the presence of another different functional group (e.g. a second linking group and/or a third linking group), where the reactant does not substantially react with the another functional group (e.g. as a result of different types of functional group pairs, differences in reaction conditions, differences in electron-rich and electron-poor functional groups). For example, the functional group may have a 10-fold or greater selectivity for the reactant than compared to the another different functional group (e.g. in terms of the % conversion of the reactant relative to the % conversion of the another different functional group), preferably a 15-fold or greater selectivity, more preferably a 20-fold or greater selectivity, even more preferably a 25-fold or greater selectivity, yet even more preferably a 30-fold or greater selectivity, yet even more preferably a 40-fold or greater selectivity, yet even more preferably a 50-fold or greater selectivity, yet even more preferably a 60-fold or greater selectivity, yet even more preferably a 70-fold or greater selectivity, yet even more preferably a 80-fold or greater selectivity, yet even more preferably a 90-fold or greater selectivity, most preferably a 100-fold or greater selectivity.

The term “core” as used herein refers to a compound that comprises a body portion, one or more first linking groups attached to the body portion, and one or more second linking groups attached to the body portion, wherein the first linking group and second linking group are orthogonal to each other. The core is not particularly limited as long as it is able to connect two or more components (e.g. a polypeptide comprising a sequence of a B-cell epitope and a polypeptide comprising a sequence of a CD4+ T-cell epitope) via a non-covalent or covalent linkage.

The term “via a side chain of one of the AA groups” refers to where an amino acid side-chain residue (e.g. —NH₂, OH including —COOH, SH) is connected via the equivalent radical form of the amino acid side-chain residue (e.g. —NH—, —O—, —S—).

The notation as used herein:

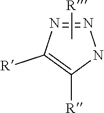

refers to a regioisomeric mixture of the following regioisomers:

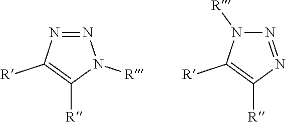

The notation as used herein:

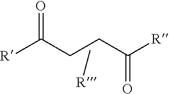

refers to a regioisomeric mixture of the following regioisomers:

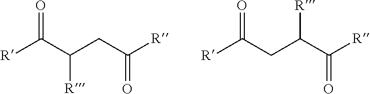

The term “terminal alkyne” as used herein refers to a group comprising a —C≡C—H moiety.

The term “cycloalkyne” as used herein refers to a closed non-aromatic monocyclic, bicyclic or tricyclic ring comprising from 8 to 12 carbon atoms in the ring, for example, 8 to 10 carbon atoms, and which contains at least one endocyclic carbon-carbon triple bond. A carbon atom in the ring may be replaced with a heteroatom, e.g. a nitrogen atom. Non-limiting examples of the cycloalkyne may include a cyclooctyne, bicyclononyne (e.g. bicyclo[6.1.0]non-4-yne), dibenzocyclooctyne and azadibenzocyclooctyne.

The term “maleimide” as used herein refers to the following structure:

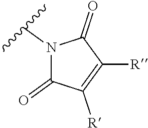

wherein R′ and R″ are independently selected from hydrogen, halogen, optionally substituted alkyl, optionally substituted cycloalkyl, optionally substituted heterocycloalkyl, optionally substituted aryl and optionally substituted heteroaryl; and
the wavy line represents a connection to the remainder of the compound.

The term “α-halocarbonyl” refers to the following structure:

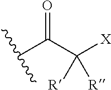

wherein R′ and R″ are independently selected from hydrogen, halogen, optionally substituted alkyl, optionally substituted cycloalkyl, optionally substituted heterocycloalkyl, optionally substituted aryl and optionally substituted heteroaryl;
X is halogen; and
the wavy line represents a connection to the remainder of the compound.

The term “halo” or “halogen” as used herein refers to any radical of fluorine, chlorine, bromine or iodine.

The term “alkyl” as used herein, by itself or as part of another group, refers to both straight and branched chain radicals of up to twelve carbons. For example, an alkyl group may contain 1, 2, 3, 4, 5, 6, 7, 8, 9, 10, 11 or 12 carbon atoms. Non-limiting examples of C₁-C₁₂ alkyl groups include methyl, ethyl, propyl, isopropyl, butyl, sec-butyl, tert-butyl, 3-pentyl, hexyl and octyl groups. Preferably, the term “alkyl” as used herein, by itself or as part of another group, may refer to a straight or branched chain radical comprising from one to eight carbon atoms, more preferably one to six carbon atoms and even more preferably one to four carbon atoms. An “optionally substituted alkyl” group may include the substituents as described below for the term “optionally substituted”. For example, an “optionally substituted alkyl” group may include a “haloalkyl” group.

The term “haloalkyl” as used herein, by itself or as part of another group, refers to both straight and branched chain radicals of up to twelve carbon atoms, comprising at least one halogen atom. For example, a haloalkyl group may contain 1, 2, 3, 4, 5, 6, 7, 8, 9, 10, 11 or 12 carbon atoms. Preferably, the term “haloalkyl” as used herein, by itself or as part of another group, may refer to a straight or branched chain radical comprising from one to eight carbon atoms, more preferably one to six carbon atoms and even more preferably one to four carbon atoms, and comprising at least one halogen atom.

For example, a “haloalkyl” group may be a fluoroalkyl or perfluoroalkyl group.

Preferably, a “haloalkyl” group may be a C₁-C₆ fluoroalkyl group, or a C₁-C₆ perfluoroalkyl group.

Even more preferably, a “haloalkyl” group may be a C₁-C₄ fluoroalkyl group, or a C₁-C₄ perfluoroalkyl group. For example, a “haloalkyl” group may include difluoromethyl, trifluoromethyl or pentafluoroethyl.

The term “cycloalkyl” as used herein refers to an alkyl group comprising a closed ring comprising from 3 to 8 carbon atoms, for example, 3 to 6 carbon atoms. For example, a cycloalkyl group may contain 3, 4, 5, 6, 7 or 8 carbon atoms. Non-limiting examples of cycloalkyl groups include cyclopropyl, cyclobutyl, cyclopentyl, cyclohexyl, cycloheptyl, (cyclohexyl)methyl, and (cyclohexyl)ethyl. An “optionally substituted cycloalkyl” group may include the substituents as described below for the term “optionally substituted”.

The term “heterocycloalkyl” as used herein refers to a saturated or partially saturated 3 to 7 membered monocyclic, or 7 to 10 membered bicyclic ring system, which consists of carbon atoms and from one to four heteroatoms independently selected from the group consisting of O, N, and S, wherein the nitrogen and sulfur heteroatoms may be optionally oxidised, the nitrogen may be optionally quaternised, and includes any bicyclic group in which any of the above-defined rings is fused to a benzene ring, and wherein the ring may be substituted on carbon or on a nitrogen atom if the resulting compound is stable. Non-limiting examples of common saturated or partially saturated heterocycloalkyl groups include azetinyl, oxetanyl, tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups. An “optionally substituted heterocycloalkyl” group may include the substituents as described below for the term “optionally substituted”.

The term “alkoxy” as used herein, by itself or as part of another group, refers to an alkyl group, as defined herein, appended to the parent molecular moiety through an oxygen atom. For example, an alkoxy group may contain 1, 2, 3, 4, 5, 6, 7, 8, 9, 10, 11 or 12 carbon atoms. Preferably, the “alkoxy” as used herein, by itself or as part of another group, may refer to a straight or branched chain radical comprising from one to eight carbon atoms, more preferably one to six carbon atoms and even more preferably one to four carbon atoms, appended to the parent molecular moiety through an oxygen atom. Non-limiting examples of alkoxy groups include methoxy, ethoxy, propoxy, 2-propoxy, butoxy, tert-butoxy, pentyloxy, and hexyloxy. An “optionally substituted alkoxy” group may include the substituents as described below for the term “optionally substituted”. For example, an “optionally substituted alkoxy” group may include a “haloalkoxy” group.

The term “haloalkoxy” as used herein, by itself or as part of another group, refers to both straight and branched chain radicals of up to twelve carbon atoms, comprising at least one halogen atom and being appended to the parent molecular moiety through an oxygen atom. For example, a haloalkoxy group may contain 1, 2, 3, 4, 5, 6, 7, 8, 9, 10, 11 or 12 carbon atoms. Preferably, the term “haloalkoxy” as used herein, by itself or as part of another group, may refer to a straight or branched chain radical comprising from one to eight carbon atoms, more preferably one to six carbon atoms and even more preferably one to four carbon atoms, comprising at least one halogen atom and being appended to the parent molecular moiety through an oxygen atom.

For example, a “haloalkoxy” group may be a fluoroalkoxy or perfluoroalkoxy group.

Preferably, a “haloalkoxy” group may be a C₁-C₆ fluoroalkoxy group, or a C₁-C₆ perfluoroalkoxy group.

Even more preferably, a “haloalkoxy” group may be a C₁-C₄ fluoroalkoxy group, or a C₁-C₄ perfluoroalkoxy group. For example, a “haloalkoxy” group may include difluoromethoxy, trifluoromethoxy or pentafluoroethoxy.

The term “alkanoyl” as used herein by itself or as part of another group, refers to an alkyl group, as defined herein, and appended to the parent molecular moiety through an R^{x}—C(═O)O— group via the oxygen atom, where R^{x} represents the alkyl group. For example, an alkanoyl group may contain 2, 3, 4, 5, 6, 7, 8, 9, 10, 11, 12 or 13 carbon atoms. Preferably, the term “alkanoyl” as used herein, by itself or as part of another group, may refer to a straight or branched chain radical comprising from two to eight carbon atoms, more preferably two to six carbon atoms and even more preferably two to four carbon atoms, and being appended to the parent molecular moiety through an R^{x}—C(═O)O— group via the oxygen atom, where R^{x} represents the alkyl group. Non-limiting examples of alkanoyl groups include acetoxy, propionyloxy, butyryloxy and pentanoyloxy. An “optionally substituted alkanoyl” group may include the substituents as described below for the term “optionally substituted”.

The term “aryl” as used herein by itself or as part of another group refers to monocyclic, bicyclic or tricyclic aromatic groups containing from 6 to 14 carbon atoms in the ring. Common aryl groups include C₆-C₁₄ aryl, for example, C₆-C₁₀ aryl. Non-limiting examples of C₆-C₁₄ aryl groups include phenyl, naphthyl, phenanthrenyl, anthracenyl, indenyl, azulenyl, biphenyl, biphenylenyl and fluorenyl groups. An “optionally substituted aryl” group may include the substituents as described below for the term “optionally substituted”.

The term “heteroaryl” as used herein refers to aromatic groups having 5 to 14 ring atoms (for example, 5 to 10 ring atoms) and containing carbon atoms and 1, 2 or 3 oxygen, nitrogen or sulfur heteroatoms. Examples of heteroaryl groups include thienyl (thiophenyl), benzo[b] thienyl, naphtho[2,3-b] thienyl, thianthrenyl, furyl (furanyl), pyranyl, isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, phthalazin-1-one, 1,4-dihydroquinoxaline-2,3-dione, 7-aminoisocoumarin, pyrido[1,2-α]pyrimidin-4-one, pyrazolo[1,5-α]pyrimidinyl, including without limitation pyrazolo[1,5-α]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide. An “optionally substituted heteroaryl” group may include the substituents as described below for the term “optionally substituted”.

The term “alkylene” as used herein refers to divalent straight and branched chain groups having from 1 to 12 carbon atoms. Preferably, the alkylene groups are straight or branched alkylene groups having from 1 to 6 carbon atoms, more preferably straight or branched alkylene groups having from 1 to 4 carbon atoms. An alkylene group may optionally comprise one or more “substituents”, as described herein.

As described herein, compounds may contain “optionally substituted” moieties. In general, the term “substituted”, whether preceded by the term “optionally” or not, means that one or more hydrogen atoms of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisaged by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

For example, the term “optionally substituted” as used herein may refer to when at least one substituent is selected from non-limiting examples such as oxo, hydroxy, halogen, cyano, optionally substituted alkyl, haloalkyl, optionally substituted cycloalkyl, optionally substituted heterocycloalkyl, optionally substituted alkoxy, haloalkoxy, optionally substituted alkanoyl, optionally substituted amino, optionally substituted aryl and optionally substituted heteroaryl.

Preferably, the term “optionally substituted” as used herein may refer to when at least one substituent is selected from halogen, hydroxy, a C₁-C₆ alkyl group, a C₁-C₆ haloalkyl group, a C₁-C₆ alkoxy group and a C₁-C₆ haloalkoxy group.

More preferably, the term “optionally substituted” as used herein may refer to when at least one substituent is selected from halogen, hydroxy, a C₁-C₄ alkyl group, a C₁-C₄ haloalkyl group, a C₁-C₄ alkoxy group and a C₁-C₄ haloalkoxy group.

Even more preferably, the term “optionally substituted” as used herein may refer to when at least one substituent is selected from fluoro, hydroxy, a methyl group, a trifluoromethyl group, a methoxy group and a trifluoromethoxy group.

The conjugates of the present invention may be provided in “salt” form. The term “salt” as used herein refers to salts of the compounds as described herein that are derived from suitable inorganic and organic acids and bases. Examples of salts of a basic group include those formed with inorganic acids such as hydrochloric acid, hydrobromic acid, phosphoric acid, sulfuric acid and perchloric acid or with organic acids such as trifluoroacetic acid, acetic acid, oxalic acid, maleic acid, tartaric acid, citric acid, succinic acid or malonic acid or by using other methods used in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁-C₄ alkyl)₄ salts. Representative alkali or alkaline earth metal salts include sodium, lithium, potassium, calcium, magnesium, and the like. Further salts include, when appropriate, nontoxic ammonium, quaternary ammonium, and amine cations formed using counterions such as halide, hydroxide, carboxylate, sulfate, phosphate, nitrate, alkyl sulfonate and aryl sulfonate.

Certain compounds of the present disclosure may exist in unsolvated forms as well as solvated forms, including hydrated forms. “Hydrate” refers to a complex formed by combination of water molecules with molecules or ions of the solute. “Solvate” refers to a complex formed by combination of solvent molecules with molecules or ions of the solute. The solvent may be an organic compound, an inorganic compound, or a mixture of both. Solvate is meant to include hydrate. Some examples of solvents include, but are not limited to, methanol, acetonitrile, N,N-dimethylformamide, tetrahydrofuran, dimethylsulfoxide, and water. In general, the solvated forms are equivalent to unsolvated forms and are encompassed within the scope of the present disclosure. Certain compounds of the present disclosure may exist as solid material in e.g. multiple crystalline or amorphous forms. In general, all physical forms are equivalent for the uses contemplated by the present disclosure and are intended to be within the scope of the present disclosure.

“Tautomer” means compounds produced by the phenomenon wherein a proton of one atom of a molecule shifts to another atom (See, Jerry March, Advanced Organic Chemistry: Reactions, Mechanisms and Structures, Fourth Edition, John Wiley & Sons, pages 69-74 (1992)). The tautomers also refer to one of two or more structural isomers that exist in equilibrium and are readily converted from one isomeric form to another. Examples include keto-enol tautomers, such as acetone/propen-2-ol, imine-enamine tautomers and the like, ring-chain tautomers, such as glucose/2,3,4,5,6-pentahydroxy-hexanal and the like, the tautomeric forms of heteroaryl groups containing a —N═C(H)—NH— ring atom arrangement, such as pyrazoles, imidazoles, benzimidazoles, triazoles, and tetrazoles. Where the compound contains, for example, a keto or oxime group or an aromatic moiety, tautomeric isomerism (‘tautomerism’) may occur. The compounds described herein may have one or more tautomers and therefore include various isomers. A skilled person would recognise that other tautomeric ring atom arrangements are possible. All such isomeric forms of these compounds are expressly included in the present disclosure.

“Isomers” mean compounds having identical molecular formulae but differ in the nature or sequence of bonding of their atoms or in the arrangement of their atoms in space. Isomers that differ in the arrangement of their atoms in space are termed “stereoisomers”. “Stereoisomer” and “stereoisomers” refer to compounds that exist in different stereoisomeric forms if they possess one or more asymmetric centres or a double bond with asymmetric substitution and, therefore, may be produced as individual stereoisomers or as mixtures. Stereoisomers include enantiomers and diastereomers. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer may be characterised by the absolute configuration of its asymmetric centre and is described by the R- and S-sequencing rules of Cahn and Prelog, or by the manner in which the molecule rotates the plane of polarised light and designated as dextrorotatory or laevorotatory (i.e., as (+) or (−)-isomers respectively). A chiral compound may exist as either individual enantiomers or as a mixture thereof. A mixture containing equal proportions of the enantiomers is called a “racemic mixture”. Unless otherwise indicated, the description is intended to include individual stereoisomers as well as mixtures. The methods for the determination of stereochemistry and the separation of stereoisomers are well-known in the art (see discussion in Chapter 4 of Advanced Organic Chemistry, 6th edition J. March, John Wiley and Sons, New York, 2007) differ in the chirality of one or more stereocentres.

The disclosure also embraces isotopically-labelled compounds of the present disclosure which are identical to those recited herein, but for the fact that one or more atoms are replaced by an atom having an atomic mass or mass number different from the atomic mass or mass number usually found in nature. Examples of isotopes that may be incorporated into compounds of the disclosure include isotopes of hydrogen, carbon, nitrogen, oxygen, phosphorus, fluorine, and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, and ¹²⁵I. Unless otherwise stated, when a position is designated specifically as “H” or “hydrogen”, the position is understood to have hydrogen at its natural abundance isotopic composition or its isotopes, such as deuterium (D) or tritium (H). Certain isotopically-labelled compounds of the present disclosure (e.g., those labelled with ³H and ¹⁴C) are useful in compound and/or substrate tissue distribution assays. Tritiated (i.e., ³H) and carbon-14 (i.e., ¹⁴C) and fluorine-18 (i.e., ¹⁸F) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of the present disclosure may generally be prepared by following procedures analogous to those described herein, by substituting an isotopically labelled reagent for a non-isotopically labelled reagent.

### BRIEF DESCRIPTION OF THE FIGURES

FIG. 1A is a schematic showing a conjugate according to one embodiment of the present invention. In this embodiment, the conjugate comprises three polypeptides each of which comprises the MTTE sequence of SEQ ID NO: 7 (1) linked via a spacer sequence (2) to a core (3). This enables linkage of the MTTE sequence (SEQ ID NO: 7) to a polypeptide comprising a CD4+ T-cell epitope derived from a universal tumour antigen (4). In one embodiment, the CD4+ T-cell epitope is SEQ ID NO: 1. In other embodiments, the universal tumour antigen is from an alternative hTERT peptide with broad HLA coverage. In a further embodiment, the polypeptide comprising the CD4+ T-cell epitope comprises a further epitope. FIG. 1B is a schematic showing a proposed mechanism by which an embodiment of the present invention elicits an immune response. Firstly, there is (1) parenteral administration of a synthetic peptide conjugate, then (2) the peptide conjugate is taken up by dendritic cells via scavenger receptors and antibody-mediated uptake of pre-existing anti-MTTE antibodies. (3) Through epitope processing and CD4+ epitope presentation to CD4+ helper T cells, additional feedback is provided through activation of MTTE-specific B-cells to become plasma cells which produce soluble anti-tetanus antibodies. (4) A positive feedback mechanism potentiates antibody production and immune complex formation via the CD4+ T-helper epitope in the conjugate. (5) Activated CD4+ T-cells are then tumour-specific (e.g. telomerase-specific) and can migrate to the tumour to potentiate the immune response in the tumour (epitope spreading).

FIGS. 2A to 2E are Venn diagrams showing comparisons of covered HLA class I (FIGS. 2A, 2B and 2D) or HLA class II alleles (FIGS. 2C and 2E) between specific polypeptide combinations. FIG. 2A shows a comparison between SEQ ID NO: 116 (GV_LSE) and 1 (P719-20). FIGS. 2B and 2C show a comparison between SEQ ID NO: 116 (GV_LSE) and 118 (C-term peptide). FIGS. 2D and 2E show a comparison between SEQ ID NO: 116 (GV_LSE) and 126 (GV1001).

FIGS. 3A and 3B are graphs showing the predicted population coverage provided by the polypeptides of SEQ ID NOS: 1 (P719-20) and 116 (GV_LSE) based on HLA class I epitopes and HLA class II epitopes respectively.

FIGS. 4A and 4B are graphs showing the predicted population coverage provided by the polypeptides of SEQ ID NO: 126 (GV1001) and 116 (GV_LSE) based on HLA class I epitopes and HLA class II epitopes respectively.

FIGS. 5A and 5B are graphs showing the predicted population coverage provided by the polypeptides of SEQ ID NOS: 126 (GV1001). 1 (P719-2) and 116 (GV_LSE) based on HLA class I epitopes and HLA class II epitopes respectively.

FIGS. 6A and 6B are graphs showing the predicted population coverage provided by the polypeptides of SEQ ID NOS: 126 (GV1001), 1 (P719-20), 116 (GV_LSE) and 118 (C-term-pep) based on HLA class I epitopes and HLA class II epitopes respectively.

FIG. 7A is a graph showing anti-MTTE IgG1 antibody titres as measured by ELISA in blood samples from female C57BL/6 mice that had been administered a conjugate comprising a synthetic long peptide (SLP) having the amino acid sequence AVGALEGSRNQDWLGVPRQL (SEQ ID NO. 54), which does not include a known active CD4+ T-cell epitope in mice (“SC injected mouse (conjugate 2 nmol)”) or that had been administered monoclonal anti-MTTE IgG1 antibodies as a control (“SC injected mouse (aMTTE IgG1)”). FIG. 7B is a graph showing anti-MTTE IgG1 antibody titres as measured by ELISA in blood samples from mice that had been administered a conjugate comprising an SLP the having amino acid sequence ARWWWMHHNMDLIGGAKxVAAWTLKAu (SEQ ID NO. 55), which includes the universal CD4+ T-cell epitope “PADRE” (“Conjugate: MTTE3-HY-PADRE”) or that had been administered the SLP alone, without conjugation (“SLP: HY-PADRE”).

FIG. 8A is a schematic of the prime-boost vaccination schedule used in the experiment of Example 8. FIG. 8B is a graph showing anti-MTTE antibody titres in male rabbits that were subcutaneously vaccinated four times with a tetanus toxoid (TTd) vaccine (at weeks 1, 3, 5 and 7) followed by four subcutaneous vaccinations with the cancer vaccine TENDU at either a low, an intermediate or a high dose. Plasma samples for serology analysis were collected at week 8 and week 15. The rabbit anti-MTTE antibody titres were measure by Capra Science antibodies AB in an in-house-ELISA. **P<0.01 using a paired t-test; ns=not significant. FIG. 8C is a table providing detail on the conjugates of LUG1-6 as comprised within the “TENDU” vaccine.

FIG. 9 shows the binding of GMP LUG1-6 constructs to human anti-MTTE antibodies. FIG. 9A shows binding of conjugates to monoclonal human IgG1 anti-MTTE antibodies. Conjugates were coated onto an ELISA plate at a range of concentrations (from 0.000457-1 nmol/ml). Human recombinant anti-MTTE IgG1 antibody was used as primary antibody and detection was performed using an anti-human kappa light chain secondary antibody. FIG. 9B shows binding of conjugates to serum containing polyclonal human anti-MTTE antibodies. Conjugates were coated onto an ELISA plate at a range of concentrations (from 0.004-1 nmol/ml) and incubated with diluted donor plasma. Detection was performed using an anti-human kappa light chain secondary antibody.

FIG. 10 shows the effects on anti-MTTE titre of vaccination of animals with LUG2 conjugates (A) (known to harbour a DR4 CD4+ T-cell epitope), and (B) ELISPOT analysis of T-cell responses after HLA-DR4 mice had been vaccinated with LUG2 as set out in Example 10. ELISPOT was performed by incubation of the splenocytes with the synthetic long peptides UV02 (SEQ ID NO: 143) and UV08 (SEQ ID NO: 144) for 48 h. SEB was used as positive control and untreated splenocytes as negative control.

FIG. 11A shows a schematic of the experimental design of Example 11 in which the polypeptide of SEQ ID NO: 1 was administered to HLA-DR4 transgenic mice. FIG. 11B is a graph of the results from an ELISpot assay in which T-cell responses were measured following stimulation of splenocytes with SEQ ID NO: 1. SEQ ID NO: 1 combined with an anti-CD4 antibody or cells alone as a negative control. Statistical analysis was performed using Kruskal-Wallis test with Dunn's multiple comparisons test; “**” refers to p<0.01 and “ns” is non-significant.

FIG. 12A is a graph showing the percentage of GFP-expressing T-cells specific for SEQ ID NO: 1 when exposed in co-cultures with EBV cells as antigen-presenting cells to 0.5, 1.5 and 5 μM concentrations of SEQ ID NO: 1, SEQ ID NO: 52 or the conjugate CD12B. Each bar represents the mean value+/−SD of duplicates for SEQ ID NO: 1 and CD12B while SEQ ID NO: 52 was run as a single sample for each concentration. FIG. 12B is a graph showing the percentage of GFP-expressing T-cells specific for SEQ ID NO: 1 when exposed to 0.5, 1.5 and 3 UM concentrations of the conjugates CD12B or CD20B or when exposed to 0.5, 1.5 and 5 μM concentrations of SEQ ID NO: 1 or SEQ ID NO: 52.

FIGS. 13A and 13B are a graph and table respectively showing the results from a sandwich ELISA experiment in which conjugate binding to anti-MTTE polyclonal antibodies from the blood plasma product TetaQuin was assessed.

FIGS. 14A and 14B are a graph and table respectively showing the results from an indirect ELISA experiment in which conjugate binding to human serum containing polyclonal antibodies were assessed (the serum was derived from a donor that had recently been vaccinated with tetanus toxoid prior to blood sampling).

FIGS. 15A and 15B are a graph and table respectively showing the results from an indirect ELISA experiment in which conjugate binding human serum containing polyclonal antibodies were assessed (the serum was derived from a donor that had recently been vaccinated with tetanus toxoid prior to blood sampling).

FIG. 16A is a schematic of the experimental design of Example 14. FIG. 16B is a graph showing the results from an ELISpot assay in which splenocytes, obtained from transgenic HLA-A2/HLA-DR1 mice that had previously been vaccinated with SEQ ID NO: 1/CpG or the conjugate CD12B, were stimulated with the polypeptides of SEQ ID NO: 7 (MTTE) or SEQ ID NO: 1 (P719-20). Statistical analysis was performed using Sidak's multiple comparisons test (*p<0.05; “ns”=not significant). FIGS. 16C and 16D are graphs showing the results from an ELISpot assay in which splenocytes from the CD12B exposed animals were stimulated with polypeptides from SEQ ID NO: 1 comprising predicted MHC class I or class II epitopes in the presence or absence of an anti-CD8 (FIG. 16C) or anti-CD4 antibody (FIG. 16D) respectively. Statistical analysis was performed using graph pad and using the Sidak's multiple comparisons test (*p<0.05; **p<0.01; “ns”=non-significant).

FIG. 17 relates to the in vivo assessment of the immunogenicity of the CD20B conjugate in seropositive or seronegative HLA-DR4 mice. FIG. 17A is a graph of optical density (OD) measured to detect anti-MTTE antibody levels in mice at the end of the experiment as set out in Example 15. Significance was assessed by a Mann Whitney test *p=0.0286. FIG. 17B is a graph showing a correlation analysis between anti-MTTE OD values pre and post CD20B exposure (x-axis) for seropositive animals and T-cell responses in response to stimulation with the peptide mix of UV18 and UV19 combined (y-axis). Pearson r=0.9348. The plotted curve is a simple regression line with the 95% confidence bands of the best-fit line. Analyses were performed using Graph pad prism. FIGS. 17C and 17D are graphs showing the results from ELISpot assays to measure T-cell responses after stimulation of splenocytes from mice administered either the conjugate CD20B or the polypeptide of SEQ ID NO: 1 formulated in IFA and CpG. Stimulations were performed using a combination of UV18/19 (FIG. 17C) or UV16 alone (FIG. 17D).

FIG. 18 shows a schematic structure of a conjugate according to an embodiment of the invention. A core comprising a body portion (oval), three first linking groups (hollow rounded rectangles) and a second linking group (hollow pentagon) is shown on the top of the figure. The core can be linked to three B-cell epitopes, in this case MTTE (dark filled rounded rectangle); and an SLP comprising a CD4+ T-cell epitope (dark filled pentagon). This forms a 3+1 conjugate as shown on the bottom of the figure, attached to the B-cell epitopes by first connecting elements (lightly shaded rounded rectangle) and attached to the SLP comprising the CD4+ T-cell epitope by a second connecting element (lightly shaded pentagon).

FIG. 19 shows a reverse phase chromatographic trace for Synthesis Example 17 (Reprosil Gold 200 C18, column temp. 40° C., 1 mL/min flow rate; eluent A=0.1% TFA in water, eluent B=0.1 TFA in acetonitrile, gradient of 95:5 (A:B) up to 2:98).

FIG. 20 shows a LCMS trace for Synthesis Example 17. Peaks labelled with “#” correspond to product peaks at various charges (e.g. 1462.2 for [M+10H]¹⁰⁺, 1329.4 for [M+11H]¹¹⁺, 1218.6 for [M+12H]¹²⁺, etc.); expected mass is 14614.

FIG. 21 shows a schematic of the vaccination schedule of Example 16. CD29 conjugate or 3+1+1 conjugate was administered to groups of HLA2-DR1 mice using a prime-boost-boost vaccination schedule. The third groups of mice was immunised with UV34 polypeptide (GVExt5-SEQ ID NO: 116) in an emulsion with IFA using a prime-boost-boost vaccination schedule. The conjugates and peptides were immunized with one week between each injection. The animals were sacrificed and splenocytes and draining lymph nodes were collected for in vitro assay.

FIG. 22 shows the results of ELISpot assays. Spleens and lymph nodes were used to perform ex-vivo ELISpot assay to identify the frequency of peptide specific responding T-cells and IFN-gamma released into the medium. ELISpot protocol similar to previous ELISpot assay. FIG. 22A shows results for CD29 conjugate; FIG. 22B shows results for 3+1+1 conjugate; and FIG. 22C shows results for the polypeptide of SEQ ID NO: 116 (UV 34-GVExt5)+IFA.

FIG. 23 shows a graph of T cell responses against individual peptides following administration of (SEQ ID NO: 126-GV1001). The horizontal dashed line marks the “cut off” which is the response level of “cells alone” which is to be considered the baseline. Bars of peptides above the cut off line/baseline are considered as T cell responses against the specific peptide.

FIG. 24 shows a graph of T cell responses against individual peptides following administration of (SEQ ID NO: 116-GVExt5). The horizontal dashed line marks the “cut off” which is the response level of “cells alone” which is to be considered the baseline. Bars of peptides above the cut off line/baseline are considered as T cell responses against the specific peptide.

FIG. 25 shows a tabulation of the individual results for FIGS. 23 and 24.

FIG. 26 is a graph showing the results of a sandwich ELISA assay in which conjugate binding to anti-MTTE polyclonal antibodies from the human blood plasma product TetaQuin was assessed. Capture antibody was anti-p719-20 (SEQ ID NO: 1) rabbit polyclonal.

FIG. 27 is a graph showing the results of a sandwich ELISA assay in which conjugate binding to anti-MTTE polyclonal antibodies from the human blood plasma product TetaQuin was assessed. Capture antibody was anti-GVExt5 (SEQ ID NO: 116) rabbit polyclonal.

FIG. 28 is a graph showing the results of binding of UVC1-017 (p719-20 containing conjugate), in 3 different salt forms, to monoclonal anti-MTTE (SEQ ID NO: 7) antibodies in indirect ELISA. Conjugate UVC1-017 in TFA salt form showed slightly better binding.

FIG. 29 is a graph showing the results of binding of UVC1-017 (p719-20 containing conjugate), in 3 different salt forms, to polyclonal anti-MTTE (SEQ ID NO: 7) antibodies in indirect ELISA. Conjugate UVC1-017 in TFA salt form showed slightly better binding.

FIG. 30 is a graph showing the results of binding of UVC2-001 (GvExt5 containing conjugate), in 3 different salt forms, to monoclonal anti-MTTE (SEQ ID NO: 7) antibodies in indirect ELISA. Similar binding is observed for all 3 salt forms.

FIG. 31 is a graph showing the results of binding of UVC2-001 (GvExt5 containing conjugate), in 3 different salt forms, to polyclonal anti-MTTE (SEQ ID NO: 7) antibodies in indirect ELISA. Similar binding is observed for all 3 salt forms.

FIG. 32 is a graph showing the concentration of anti-MTTE IgG measured in serum from patients enrolled in the TENDU vaccination trial.

FIG. 33 is a schematic representation of the vaccination schedule for the in vivo assessment of the immunogenicity of the GVExt5 peptide (SEQ ID NO: 116) in the form of a conjugate and peptide+IFA in transgenic HLA-A2/HLA-DR1 animals.

FIG. 34 shows the results of the serum anti-MTTE antibody study following the vaccination schedule of FIG. 33. A. is a graph showing anti-MTTE antibody titers in mice at the end of the experiment. B. is a graph showing anti-MTTE antibody titers at various time points during the course immunization in mice of individual groups.

FIG. 35 shows the results of the study following the vaccination schedule of FIG. 33. Each graph shows T-cell responses following stimulation with individual peptides: A. UV36; B. UV58; C. UV59; D. UV60; E. UV64; F. UV65; and G. UV66.

FIG. 36 shows the results of the study following the vaccination schedule of FIG. 33. A. is a graph showing the sum of T cell responses of mice exposed to CD29 towards polypeptides sequences of varying lengths within the UV36 peptide. B. is a graph showing the T cell responses of individual mice.

### DETAILED DESCRIPTION OF THE INVENTION

In embodiments of the present invention, there is provided a conjugate which comprises:
(a) at least one polypeptide comprising a sequence of a B-cell epitope; and
(b) at least one polypeptide comprising a sequence of a CD4+ T-cell epitope,
wherein the CD4+ T-cell epitope comprises a region of at least 12 amino acids of a universal tumour antigen or a sequence having at least 80% sequence identity to the region. The CD4+ T-cell epitope is immunogenic in at least 50% of the population, and the at least one polypeptide comprising the sequence of the CD4+ T-cell epitope is equal to or less than 500 amino acids in length. The sequence of the B-cell epitope is different from the sequence of the CD4+ T-cell epitope and an antibody specific for the B-cell epitope binds to the conjugate. The at least one polypeptide comprising a sequence of the B-cell epitope and the at least one polypeptide comprising a sequence of the CD4+ T-cell epitope are optionally linked via core as defined herein.

In other embodiments of the present invention, there is provided a polypeptide and a nucleic acid molecule which do not form part of a conjugate.

Further details of the components of the above embodiments of the present invention are described below.

### A Polypeptide

In accordance with one aspect of the invention, there is provided a polypeptide of the human telomerase reverse transcriptase (hTERT) protein which comprises a sequence selected from SEQ ID NO: 116 (i.e. LSEAEVRQHREARPALLTSRLRFIPKPDGL) and 117 (i.e. HREARPALLTSRLRFIPKPDGLRPIVNMDY). In one embodiment, the polypeptide comprising the sequence of SEQ ID NO: 116 is equal to or less than 170 amino acids in length. In a further embodiment, the polypeptide comprising the sequence of SEQ ID NO: 116 is equal to or less than 150, 140, 130, 125, 120, 110, 100, 90, 80, 75, 60, 50, 40 or 35 amino acids in length. In one embodiment, the polypeptide comprising the sequence of SEQ ID NO: 117 is equal to or less than 40 amino acids in length. In a further embodiment, the polypeptide comprising the sequence of SEQ ID NO: 117 is equal to or less than 38, 36, 35, 34, 33, 32 or 31 amino acids in length.

In some embodiments, the polypeptide consists of the sequence set out in SEQ ID NO: 116 or 117. In other embodiments, the polypeptide comprises a sequence set out in SEQ ID NO: 116 or 117. In one embodiment, any additional amino acids at the N- and/or C-termini are present in the naturally occurring hTERT protein. In an alternative embodiment, any additional amino acids at the N- and/or C-termini are different from those present in the naturally occurring hTERT protein.

In other embodiments, there are provided immunogenic fragments of the aforementioned polypeptides; the fragments comprise at least 8, 9, 10, 11, 12, 13, 14, 15, 16, 17, 18, 19, 20, 21, 22, 23, 24, 25, 26, 27, 28 or 29 amino acids of SEQ ID NO: 116 or 117.

Telomerase is an enzyme that has the function of replicating the 3′ end of the telomere regions of linear DNA strands in eukaryotic cells as these regions cannot be extended by the enzyme DNA polymerase in the normal way. The telomerase enzyme comprises the TERT subunit (“hTERT” in humans) and telomerase RNA. By using the telomerase RNA as a template, the TERT subunit adds a repeating sequence to the 3′ end of chromosomes in eukaryotic cells in order to extend the 3′ end of the DNA strand. The full-length hTERT mRNA sequence is set out in GenBank accession no. AF015950.1; the amino acid sequence is set out at UniProt accession number 014746 and is also set forth in SEQ ID NO: 2. SEQ ID NOS: 116 and 117 each consist of a 30 amino acid fragment of the hTERT protein.

In further embodiments, the polypeptide does not have the exact sequence identity to one of the aforementioned polypeptides or immunogenic fragments thereof. Instead, the polypeptide comprises a sequence having at least 91% sequence identity to that of SEQ ID NO: 116 or an immunogenic fragment thereof. It is particularly preferred that the sequence has at least 92%, 93%, 94%, 95%, 96%, 97%, 98% or 99% sequence identity to that set out above. In one embodiment, the polypeptide comprises a sequence having at least 95% sequence identity to that of SEQ ID NO: 117 or an immunogenic fragment thereof. It is particularly preferred that the sequence has at least 96%, 97%, 98% or 99% sequence identity to that set out above. It is also preferred that any addition or substitution of amino acid sequence results in the conservation of the properties of the original amino acid side chain. That is to say the substitution or modification is “conservative”.

Conservative substitution tables providing functionally similar amino acids are well known in the art. Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side chain (S, T, Y); a sulphur atom containing side-chain (C, M); a carboxylic acid and amide containing side-chain (D, N, E, Q); a base containing side-chain (R, K, H); and an aromatic containing side-chain (H, F, Y, W). In addition, the following eight groups each contain amino acids that are conservative substitutions for one another (see e.g. Creighton, Proteins (1984):
1) Alanine (A), Glycine (G);
2) Aspartic acid (D), Glutamic acid (E);
3) Aspargine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M).

In some embodiments, a cocktail (i.e. a mixture) of polypeptides is provided wherein the cocktail comprises at least two different polypeptides comprising sequences from SEQ ID NOS: 1, 116 and 117. In some embodiments, the cocktail comprises immunogenic fragments of said polypeptides, wherein the immunogenic fragments comprise at least eight amino acids. In further embodiments, the or each polypeptide in the cocktail comprises a sequence having at least 80% sequence identity to the sequence of SEQ ID NO: 1, 116 or 117 or the immunogenic fragment thereof. It is particularly preferred that the sequence has at least 85%, 90%, 91%, 92%, 93%, 94% 95%, 96%, 97%, 98% or 99% sequence identity to that set out above. In a preferred embodiment, the polypeptides in the cocktail comprise the sequences of SEQ ID NO: 1 and 116 or an immunogenic fragment thereof. In such embodiments, it is preferred than an immunogenic fragment of SEQ ID NO: 1 comprises at least eight amino acids thereof, more preferably 12 amino acids thereof and an immunogenic fragment of SEQ ID NO: 116 comprises at least 17 amino acids thereof. In one embodiment, the polypeptides are equal to or less than 500 amino acids in length, preferably equal to or less than 400, 300, 200, 170, 150, 125, 100, 90, 80, 70, 75, 60, 50, 40 or 30 amino acids in length. It is preferred that the at least two polypeptides are different in the sense of being based on different sequences selected from SEQ ID NOS: 1, 116 and 117.

In a further embodiment, the or each polypeptide as described above in comprised within a conjugate. Thus in one embodiment, a cocktail (i.e. a mixture) of conjugates is provided. In one embodiment, the conjugate comprising a polypeptide of the invention is a conjugate as described in further detail below.

In one embodiment, the at least two different polypeptides comprising sequences from SEQ ID NOS: 1, 116 and 117 are provided within a single polypeptide. That is to say, a single polypeptide chain is provided which comprises at least two different sequences selected from SEQ ID NOS: 1, 116 and 117. In some embodiments, the single polypeptide comprises immunogenic fragments of said polypeptides, wherein the immunogenic fragments comprise at least eight amino acids. In further embodiments, the single polypeptide comprises a sequence having at least 80% sequence identity to a sequence selected from one or more of SEQ ID NO: 1, 116 or 117 or the immunogenic fragment thereof. It is particularly preferred that the sequence has at least 85%, 90%, 91%, 92%, 93%, 94%, 95%, 96%, 97%, 98% or 99% sequence identity to that set out above.

In a preferred embodiment, the single polypeptide comprises the sequences of SEQ ID NO: 1 and 116 or an immunogenic fragment thereof. In such embodiments, it is preferred than an immunogenic fragment of SEQ ID NO: 1 comprises at least eight amino acids thereof, more preferably 12 amino acids thereof and an immunogenic fragment of SEQ ID NO: 116 comprises at least 17 amino acids thereof.

In some embodiments, the single polypeptide as described above is equal to or less than 170 amino acids in length. Preferably, equal to or less than 150, 140, 130, 125, 120, 110, 100, 90, 80, 75 or 60 amino acids in length. In some embodiments, an intervening sequence between the two different polypeptides or immunogenic fragments (e.g. SEQ ID NO: 1 and 116) comprised within the single polypeptide is provided. In one embodiment, the intervening sequence is the same as that found in the naturally occurring hTERT protein. In alternative embodiments, the intervening sequence is different (i.e. has less than 100% sequence identity) to that found in the naturally occurring hTERT protein. In one embodiment, the intervening sequence is not derived from hTERT. In one embodiment, the intervening sequence is a proteasomal cleavage site.

It is particularly preferred that the at least two different polypeptides in the cocktail or single polypeptide comprise epitopes that are capable of being recognised by a broad range of HLA class I and/or HLA class II alleles. It is also to be understood that in some embodiments, the cocktail or the single polypeptide comprises more than two polypeptides having different sequences. In one embodiment, 3, 4, 5 or more different polypeptides are provided.

In some embodiments, the sequence of a polypeptide as described above is altered in order to change (e.g. increase) the binding affinity of a polypeptide to an MHC molecule of a particular HLA allele. In other embodiments, the polypeptide has further amino acids, in addition to those set out above, at the N- and/or C-terminal thereof. Such additional amino acids can also be used to alter (e.g. increase) the binding affinity of a polypeptide to an MHC molecule.

The polypeptide (in particular a polypeptide whose sequence has been altered as set out above) is capable of eliciting a CD4+ and/or a CD8+ T-cell response. That is to say the polypeptide should be able to induce CD4+ and/or CD8+ T-cells when the polypeptide is presented by antigen presenting cells (e.g. dendritic cells). In one embodiment, a CD4+ T-cell immune response is measured by a T-cell proliferation assay (3H-Thymidine) (as previously described in Inderberg-Suso et al. Oncoimmunology. 2012 Aug. 1; 1 (5): 670-686). In one embodiment, the CD4+ T-cell immune response is considered positive if the response to the CD4+ epitope is at least 2 or 3 times the background (Stimulation Index, SI ≥2 or 3). In one embodiment, confirmation of CD8+ T-cell inducibility can be accomplished by inducing antigen-presenting cells carrying human MHC antigens (for example, B-lymphocytes, macrophages or dendritic cells) or more specifically dendritic cells derived from human peripheral blood mononuclear leukocytes, and after stimulation with the polypeptides, mixing with CD8+ cells, and then measuring the IFN-gamma produced and released by CD8+ T-cells against the target cells. As the reaction system, transgenic animals that have been produced to express a human HLA antigen (for example, those described in BenMohamed L, Krishnan R, Longmate J, Auge C, Low L, Primus J, Diamond D J, Hum Immunol 61 (8): 764-79, 2000 August, Related Articles, Books, Linkout Induction of CTL response by a minimal epitope vaccine in HLA A*0201/DR1 transgenic mice: dependence on HLA class II restricted T(H) response) can be used. For example, the target cells can be radiolabeled with ⁵¹Cr, and cytotoxic activity can be calculated from radioactivity released from the target cells.

In one embodiment, a transgenic animal that expresses an HLA antigen is used to assess whether or not a polypeptide is capable of eliciting a CD4+ and/or CD8+ T-cell response. In one embodiment, an HLA-DR4 mouse model is used to measure a CD4+ T-cell immune response. In an alternative embodiment, an HLA-A2/HLA-DR1 mouse model is used to measure a CD4+ and/or CD8+ T-cell immune response.

In a further embodiment, CD4+ and/or CD8+ T-cell inducibility can be examined using an ELISpot assay. In one embodiment, IFN-gamma that is produced and released by CD4+ and/or CD8+ T-cells in the presence of antigen-presenting cells that carry immobilized peptides is measured, and the inhibition zone on the media is visualised using anti-IFN-gamma monoclonal antibodies. More specifically, T-cells are cultured on a membrane containing immobilised antibodies against IFN-gamma. These antibodies capture interferon gamma produced by the relevant T-cells, which then can be visualized by a second antibody against IFN-gamma. This antibody is enzyme labelled and when a substrate is added a spot is visualized on the site containing an antigen reactive cell. Further examples of ELISpot assays are described below (e.g. in Examples 10, 11, 14, 15).

In some further embodiments of the present invention, the polypeptide is linked (e.g. covalently) to a further substance, while retaining its capability of inducing a CD4+ and/or CD8+ T-cell response. Such further substances include a lipid, a sugar or a sugar chain, an acetyl group, a natural or synthetic polymers and the like. In one embodiment, the polypeptide is linked to a further peptide in the form of a conjugate as described in detail below. In one embodiment, a longer antigen-rich polypeptide chain (i.e. a single polypeptide) is provided which comprises a polypeptide or immunogenic fragment as defined herein and a further polypeptide. In one embodiment, the further polypeptide is a further hTERT-derived sequence (e.g. of at least 10, 15, 20, 25, 30 or 35 amino acids in length). That is to say, in some embodiments, two or more different hTERT-derived polypeptide sequences are comprised together within a single polypeptide chain. In one embodiment an intervening sequence (e.g. a linker element) is provided between the two polypeptides. In one embodiment, the intervening sequence is not derived from hTERT or has less than 100% sequence identity with a naturally occurring hTERT sequence. In one embodiment, the linker element is a proteasomal cleavage site. The polypeptide, in certain embodiments, contains modifications such glycosylation, side chain oxidation or phosphorylation.

In some embodiments, the polypeptide is produced by conventional processes known in the art. Alternatively, the polypeptide is a fragment of a telomerase protein produced by cleavage, for example, using cyanogen bromide, and subsequent purification.

Enzymatic cleavage may also be used. In further embodiments, the polypeptide is in the form of a recombinant expressed polypeptide. For example, a suitable vector comprising a polynucleotide encoding the polypeptide in an expressible form (e.g. downstream of a regulatory sequence corresponding to a promoter sequence) is prepared and transformed into a suitable host cell. The host cell is then cultured to produce the polypeptide of interest. In other embodiments, the polypeptide is produced in vitro using in vitro translation systems.

In some embodiments, the polypeptide does not consist of the sequence of any one of:

```
  (SEQ ID NO: 120)
HREARPALLTSRLRFIPKPDGLRPIVNMDYVVGARTFRREK,
  (SEQ ID NO: 121)
EARPALLTSRLRFIPKPDGLRPIVNMDY,
  (SEQ ID NO: 122)
EARPALLTSRLRFIPKPDGLRPIVNMDYV,
  (SEQ ID NO: 123)
EARPALLTSRLRFIPKPDGLRPIVNMD
or
  (SEQ ID NO: 124)
AEVRQHREARPALLTSRLRFIPKPDGL.
```

### A Nucleic Acid Molecule

In an alternative embodiment of the present invention, there is provided a nucleic acid molecule consisting of a nucleotide sequence encoding a polypeptide as set out above. In one embodiment, the nucleotide sequence encoding SEQ ID NOS: 1, 116 or 117 is as set forth in SEQ ID NO: 160 to 162 respectively.

In some embodiments, the nucleic acid molecule consisting of the nucleotide sequence encoding a polypeptide as set out above is equal to or less than 1,500 nucleotides in length. Preferably, equal to or less than 1,200, 900, 600, 510, 450, 420, 390, 375, 360, 330, 300, 270, 240, 225, 180, 150, 120, 114, 108, 105, 102, 96 or 90 nucleotides in length.

In some embodiments, the nucleotide sequence encoding the polypeptide has less than 100% sequence identity to that of the naturally occurring hTERT sequence (e.g. as set out at GenBank accession no. AF015950.1). In some embodiments, codon optimisation is used to alter the nucleotide sequence, for example, for expression within a suitable host cell. In such embodiments, it is to be understood that the codon optimisation results in changes to the nucleotide sequence as compared with the naturally occurring hTERT sequence. In further embodiments, additional nucleotides are incorporated at the 3′ or 5′ end of the nucleotide sequence that encodes the polypeptide of the present invention. In some embodiments, such additional nucleotides are different from those found in the naturally occurring hTERT sequence. In one embodiment, the nucleic acid molecule comprises a 5′ cap. In one embodiment, a nucleic acid molecule is provided comprising a nucleotide sequence encoding a polypeptide or immunogenic fragment as defined herein and a further nucleotide sequence encoding a further polypeptide. In one embodiment, the further nucleotide sequence encodes a further hTERT-derived sequence (e.g. encoding at least 10 to 200 amino acids in length such that the further nucleotide sequence is at least 30 to 600 nucleotides in length). That is to say, in some embodiments, two or more different nucleotide sequences encoding hTERT-derived polypeptide sequences are comprised together within a single nucleic acid molecule. In one embodiment an intervening sequence (e.g. encoding a linker element) is provided between the two nucleotide sequences. In one embodiment, the intervening sequence is not derived from hTERT or has less than 100% sequence identity with a naturally occurring hTERT sequence. In one embodiment, the encoded linker element is a proteasomal cleavage site.

In some embodiments, the nucleic acid molecule is linked (e.g. covalently) to a further substance. In one embodiment, the nucleic acid molecule is linked to a carrier moiety. In one embodiment, the carrier moiety is an immunogenic carrier. In one embodiment, the nucleic acid molecule is comprised within a lipid nanoparticle.

In further embodiments, a cocktail (i.e. a mixture) of nucleic acid molecules is provided wherein the cocktail comprises at least two different nucleic acid molecules comprising a nucleotide sequence encoding a polypeptide comprising the sequence of SEQ ID NO: 1, 116 or 117. In alternative embodiments, the encoded polypeptide is an immunogenic fragment of SEQ ID NO: 1, 116 or 117, wherein the immunogenic fragment comprises at least eight amino acids. In alternative variants, the sequence of the encoded polypeptide is not identical to the aforementioned but instead has at least 80% sequence identity thereto. Preferably, at least 85%, 90%, 91%, 92%, 93%, 94%, 95%, 96%, 97%, 98% or 99% sequence identity to the aforementioned. In a preferred embodiment, the encoded polypeptides in the cocktail comprise the sequences of SEQ ID NOS: 1 and 116 or an immunogenic fragment thereof. In such embodiments, it is preferred that the immunogenic fragment of SEQ ID NO: 1 that is encoded comprises at least eight amino acids thereof, more preferably 12 amino acids thereof and that the immunogenic fragment of SEQ ID NO: 116 that is encoded comprises at least 17 amino acids thereof. In one embodiment, the encoded polypeptides are equal to or less than 500, 400, 300, 200, 170, 150, 125, 100, 90, 80, 70, 75, 60, 50, 40 or 30 amino acids in length. It is preferred that the at least two nucleic acid molecules are different in the sense of being based on different sequences selected from SEQ ID NOS: 1, 116 and 117.

In one embodiment, the at least two different nucleic acid molecules comprising a nucleotide sequence encoding a polypeptide comprising the sequence of SEQ ID NO: 1, 116 or 117 are provided within a single molecule. That is to say, a single molecule is provided which comprises at least two different nucleotide sequences, each of which encodes a polypeptide selected from the sequence of SEQ ID NO: 1, 116 and 117. In some embodiments, the encoded polypeptide is an immunogenic fragment of SEQ ID NO: 1, 116 or 117, wherein the immunogenic fragment comprises at least eight amino acids. In further embodiments, the sequence of the encoded polypeptide is not identical to the aforementioned but instead has at least 80% sequence identity thereto. Preferably, at least 85%, 90%, 91%, 92%, 93%, 94%, 95%, 96%, 97%, 98% or 99% sequence identity to the aforementioned.

In a preferred embodiment, the single molecule comprises nucleotide sequences, each of which encode a polypeptide comprising the sequence of SEQ ID NO: 1 and 116 or an immunogenic fragment thereof. In such embodiments, it is preferred that the immunogenic fragment of SEQ ID NO: 1 that is encoded comprises at least eight amino acids thereof, more preferably 12 amino acids thereof and that the immunogenic fragment of SEQ ID NO: 116 that is encoded comprises at least 17 amino acids thereof.

In one embodiment, the single molecule is a single nucleic acid molecule. In some embodiments, the single nucleic acid molecule is equal to or less than 1,500 nucleotides in length. Preferably, equal to or less than 1,200, 900, 600, 510, 450, 420, 390, 375, 360, 330, 300, 270, 240, 225 or 180 nucleotides in length.

In some embodiments, an intervening nucleotide sequence between the two different nucleotide sequences (e.g. those encoding SEQ ID NO: 1 and 116) comprised within the single molecule or nucleic acid molecule is provided. In one embodiment, the intervening sequence is the same as that found in the nucleotide sequence encoding the naturally occurring hTERT protein. In alternative embodiments, the intervening sequence is different (i.e. has less than 100% sequence identity) to that found in the nucleotide sequence encoding the naturally occurring hTERT protein. In one embodiment, the intervening sequence is not derived from hTERT. In one embodiment, the intervening sequence encodes a proteasomal cleavage site.

It is also to be understood that in some embodiments, the cocktail comprises more than two different nucleic acid molecules having different sequences or the single nucleic acid molecule comprises more than two different nucleotide sequences. In one embodiment, 3, 4, 5 or more different nucleic acid molecules or different nucleotide sequences are provided.

In a further embodiment, a vector comprising a nucleic acid molecule as set out above or a fragment thereof is provided. The particular vector to be used may be dependent on the host organism or cell(s) in which the nucleic acid molecule is to be expressed, the method that will be used to transform the host cell(s), and/or the method that is to be used for protein expression (or any another intended use of the vector). In one embodiment, the vector includes a substance(s) facilitating endosomal escape of the nucleic acid molecule.

It is to be appreciated that, owing to the degeneracy of the genetic code, nucleic acid molecules encoding a particular polypeptide may have a range of polynucleotide sequences. For example, the codons GCA, GCC, GCG and GCT all encode the amino acid alanine.

The nucleic acid molecules may be either DNA or RNA or derivatives thereof.

In one embodiment, a combination of any one of the polypeptides and/or nucleic acid molecules as set out above is provided.

### A Conjugate

In accordance with further aspect of the invention, there is provided a conjugate comprising at least one polypeptide comprising a sequence of a B-cell epitope and at least one polypeptide comprising a sequence of a CD4+ T-cell epitope.

It is preferred that the at least one polypeptide comprising a sequence of a B-cell epitope is more than one polypeptide comprising a sequence of a B-cell epitope, 2 or 3 polypeptides comprising a sequence of a B-cell epitope being particularly preferred. In some embodiments where there is more than polypeptide comprising a sequence of a B-cell epitope, each of these polypeptides has the same sequence.

In a first embodiment, the B-cell epitope of the conjugate comprises the sequence of SEQ ID NO. 7 (i.e. FIGITELKKLESKINKVF). In a preferred embodiment, the B-cell epitope consists of the sequence of SEQ ID NO. 7. SEQ ID NO. 7 is 18 amino acids in length and is derived from the sequence of tetanus toxin (TTx). TTx is a neurotoxin produced by the vegetative spore of Clostridium tetani in anaerobic conditions and causes tetanus in humans. The TTx amino acid sequence is set out at UniProt accession number P04958 and in SEQ ID NO. 3 of the Sequence Listing. TTx is synthesised by C. tetani as a single polypeptide chain that is proteolysed to yield two fragments, the light chain (LC; also known as the alpha chain) derived from the amino terminus, and the heavy chain (HC; also known as the beta chain) derived from the carboxyl terminus. The TTx light chain and heavy chain are represented by SEQ ID NOS. 4 and 5 respectively in the Sequence Listing. In TTx, the light and heavy chains are linked by a disulphide bond. SEQ ID NO. 7 corresponds to amino acids 381 to 398 of the TTx heavy chain, i.e. amino acids 381 to 398 of SEQ ID NO. 5.

A function of the B-cell epitope of the conjugate (as described in further detail below) is to bind to circulating antibodies in the subject in order to direct the conjugate to antigen-presenting cells (APCs). Having a B-cell epitope derived from TTx is advantageous because the majority of individuals (in the Western world) are immunised in childhood against tetanus and so a significant proportion of the population is expected to have circulating antibodies against TTx.

It is to be appreciated that SEQ ID NO. 7 comprises the amino acid sequence “GITELKKL” (as represented by SEQ ID NO. 6 in the Sequence Listing); more specifically, SEQ ID NO. 6 is located at amino acid positions 3 to 10 of SEQ ID NO. 7. SEQ ID NO. 6 corresponds to amino acids 383 to 390 of the TTx heavy chain, i.e. amino acids 383 to 390 of SEQ ID NO 5.

In the first embodiment, the at least one polypeptide comprising the sequence of SEQ ID NO. 7 is a first, a second and a third polypeptide comprising the sequence of SEQ ID NO. 7. In other words, the conjugate comprises three copies of the polypeptide comprising the sequence of SEQ ID NO. 7. In an alternative embodiment, the at least one polypeptide comprising the sequence of SEQ ID NO. 7 is a single polypeptide or a first and a second polypeptide. In one embodiment, the conjugate comprises a further substance. That is to say, the conjugate comprises a further substance in addition to the at least one polypeptide comprising the sequence of the B-cell epitope and the at least one polypeptide comprising the sequence of the CD4+ T-cell epitope. In one embodiment, the further substance is a further polypeptide. In a preferred embodiment, the further polypeptide comprises the sequence of an epitope. In one embodiment, the epitope is any sequence of an antigen capable of eliciting an immune response. In one embodiment, the sequence of the epitope is a further T-cell epitope, preferably a further CD4+ T-cell epitope and/or a CD8+ T-cell epitope. In a further embodiment, the conjugate comprises 4, 5, 6, 7, 8, 9, 10 or more polypeptides comprising the sequence of SEQ ID NO. 7.

In an alternative embodiment, the B-cell epitope of the conjugate has a different sequence to that of SEQ ID NO. 7. In one embodiment, the B-cell epitope comprises a different sequence comprising at least 10 amino acids which are contiguous in SEQ ID NO. 5 (i.e. the TTx heavy chain) and which comprise the amino acid sequence “GITELKKL” as represented by SEQ ID NO. 6 in the Sequence Listing. In one embodiment, the different sequence comprises at least 12, 15 or 18 amino acids which are contiguous in SEQ ID NO. 5 and which comprise the amino acid sequence “GITELKKL” as represented by SEQ ID NO. 6 in the Sequence Listing. In a further embodiment, the B-cell epitope comprises a sequence comprising at least 20, 25, 30, 35, 40, 45 or 50 amino acids which are contiguous in SEQ ID NO. 5 and which comprise the amino acid sequence “GITELKKL” as represented by SEQ ID NO. 6 in the Sequence Listing. In one embodiment, the B-cell epitope consists of any one of the sequences as described above.

In one embodiment, the B-cell epitope comprises a sequence selected from any one of SEQ ID NOS. 59 to 115. In a further embodiment, the B-cell epitope consists of a sequence selected from any one of SEQ ID NOS. 59 to 115.

In a further alternative embodiment, the B-cell epitope comprises at least 10 amino acids which are contiguous in SEQ ID NO. 5 other than those comprising the amino acid sequence “GITELKKL” as represented by SEQ ID NO. 6 in the Sequence Listing. That is to say the B-cell epitope is derived from a different region of the sequence of SEQ ID NO. 5. In yet a further embodiment, the B-cell epitope comprises at least 10 amino acids which are contiguous in SEQ ID NO. 3.

In one embodiment, the at least one polypeptide comprising the sequence of the B-cell epitope does not comprise the sequence of SEQ ID NO. 5. That is to say, the at least one polypeptide comprising the B-cell epitope does not comprise the complete sequence of the TTx heavy chain. In one embodiment, the at least one polypeptide comprising the B-cell epitope is equal to or less than 500, 400, 300, 200 or 100 amino acids or less in length. In a further embodiment, the at least one polypeptide comprising the B-cell epitope is 90, 80, 75, 70, 60, 50, 40 or 30 amino acids or less in length.

In a further embodiment, the polypeptide comprising the sequence of the B-cell epitope and/or the B-cell epitope does not have the exact sequence identity to one of the aforementioned sequences. Instead, the polypeptide and/or B-cell epitope has at least 70% sequence identity to that as set out above. Preferably, at least 75%, 80%, 85%, 90%, 95%, 96%, 97%, 98% or 99% sequence identity to that set out above. It is also preferred that any addition or substitution of amino acid sequence results in the conservation of the properties of the original amino acid side chain. That is to say the substitution or modification is “conservative” (as described above).

In one embodiment, although a B-cell epitope comprises or consists of a sequence having less than 100% sequence identity to that of SEQ ID NO. 7, the amino acids at positions corresponding to positions 3 to 5 and 11 of SEQ ID NO. 7 are unchanged from the amino acids at positions 3 to 5 and 11 of SEQ ID NO. 7.

A B-cell epitope that does not have 100% sequence identity to one of the aforementioned B-cell epitopes is referred to as a “variant B-cell epitope”. It is important that a variant B-cell epitope that is derived from TTx (for example, derived from any one of SEQ ID NOS. 3 to 5) is capable of being bound by an anti-TTx antibody. In one embodiment, confirmation that a variant B-cell epitope derived from any one of SEQ ID NOS. 3 to 5 binds to an anti-TTx antibody is accomplished using a Tettox ELISA. A “Tettox ELISA” is an ELISA assay specific for anti-TTx antibodies. A person skilled in the art will understand how to perform an ELISA assay to identify whether anti-TTx antibodies bind a particular variant B-cell epitope of interest derived from any one of SEQ ID NOS. 3 to 5. Such a variant B-cell epitope may be generated by any method known in the art, e.g. chemical synthesis. Anti-TTx antibodies may be obtained as a polyclonal antibody serum from a human donor who has received the tetanus toxoid vaccine. An exemplary Tettox ELISA protocol is described in detail in WO 2011/115483 as incorporated herein by reference.

In an alternative embodiment, the B-cell epitope is derived from a protein or polypeptide that is not the tetanus toxin.

The conjugate of the present invention also comprises at least one polypeptide comprising a sequence of a CD4+ T-cell epitope. In one embodiment, the CD4+ T-cell epitope comprises a region of at least 12 amino acids of a universal tumour antigen.

A universal tumour antigen is an antigen expressed in a high proportion of tumour types. In one embodiment, the universal tumour antigen is not widely expressed in normal (i.e. non-cancerous) tissue. In one embodiment, it is expressed in a limited subset of normal tissue. In a further embodiment, the universal tumour antigen is expressed in a high proportion of patients within each tumour type. Cancer is a heterogeneous disease and there is high degree of diversity between different tumour types as well as between patients within the same tumour type. By targeting universal tumour antigens, the applicability of the cancer therapy is improved across the patient population (i.e. within and between cancer and/or tumour types).

In a first embodiment of the invention, the universal tumour antigen is the telomerase reverse transcriptase subunit (“TERT” or “hTERT” for humans) of the telomerase enzyme (as described above). Telomerase is expressed in certain normal tissue such as stem cells in the bone marrow and gastrointestinal tract. However, it has been observed that the telomerase enzyme is activated in the vast majority of all human cancers (for example, Kim et al., Science. 1994 266 (5193): 2011-5; Shay & Wright, FEBS Lett. 2010 584 (17): 3819-25). It is believed that telomerase is activated in the vast majority of human cancers because, without the expression of the telomerase enzyme, the telomeres of cells are gradually lost, and the integrity of the chromosomes decline with each round of cell division of a cell, which ultimately results in apoptosis of the cells. Thus, expression of the telomerase enzyme is generally necessary for a cancer cell to develop because without such expression, programmed cell death will usually occur by default. In view of the role of telomerase activation in cancer, polypeptides from TERT/hTERT are regarded as universal tumour antigens.

In a preferred embodiment, the CD4+ T-cell epitope comprises the sequence of SEQ ID NO: 1 (i.e. ALFSVLNYERARRPGLLGASVLGLDDIHRA), SEQ ID NO: 116 (i.e. LSEAEVRQHREARPALLTSRLRFIPKPDGL) and/or SEQ ID NO: 117 (i.e. HREARPALLTSRLRFIPKPDGLRPIVNMDY). It is preferred that the CD4+ T-cell epitope consists of the sequence of SEQ ID NO: 1, 116 and/or 117. More preferably, the CD4+ T-cell epitope comprises or consists of the sequence of SEQ ID NO: 1 or 116. The polypeptide of SEQ ID NO: 1, 116 or 117 is each 30 amino acids in length. SEQ ID NO: 1, 116 and 117 each consists of a 30 amino acid fragment of hTERT.

In other embodiments, there are provided immunogenic fragments of SEQ ID NOS. 1, 116 or 117 which comprise at least 12 amino acids thereof. In one embodiment, the immunogenic fragment consists of 12 amino acids of SEQ ID NO: 1, 116 or 117. In an alternative embodiment, the immunogenic fragment comprises at least 13, 14, 15, 16, 17, 18, 19, 20, 21, 22, 23, 24, 25, 26, 27, 28 or 29 amino acids of SEQ ID NO: 1, 116 or 117. In a further embodiment, immunogenic fragment consists of 13, 14, 15, 16, 17, 18, 19, 20, 21, 22, 23, 24, 25, 26, 27, 28 or 29 amino acids of SEQ ID NO: 1, 116 or 117. Exemplary immunogenic fragments of SEQ ID NO: 1 include those set out in SEQ ID NOs. 12 to 35.

In the first embodiment, the at least one polypeptide comprising the sequence of SEQ ID NO: 1, 116 or 117 is a single polypeptide (i.e. the conjugate contains one polypeptide comprising the sequence of SEQ ID NO. 1). In an alternative embodiment, the at least one polypeptide comprising the sequence of SEQ ID NO: 1, 116 or 117 is a first polypeptide and the conjugate comprises a plurality of polypeptides, each of which encodes a sequence selected from SEQ ID NOS: 1, 116 or 117. In one embodiment, the conjugate comprises a polypeptide comprising the sequence of SEQ ID NO: 1 and a polypeptide comprising the sequence of SEQ ID NO: 116.

In a further embodiment, the conjugate comprises a further substance. That is to say, the conjugate comprises a further substance in addition to the at least one polypeptide comprising the sequence of the B-cell epitope and the at least one polypeptide comprising the sequence of the CD4+ T-cell epitope. In one embodiment, the further substance is a further polypeptide. In a preferred embodiment, the further polypeptide comprises the sequence of an epitope. In one embodiment, the epitope is any sequence of an antigen capable of eliciting an immune response. In one embodiment, the sequence of the epitope is a further T-cell epitope, preferably a further CD4+ T-cell epitope and/or a CD8+ T-cell epitope. It is to be appreciated that, in some embodiments, the further CD4+ T-cell epitope is different from that comprising the sequence of SEQ ID NO: 1, 116 or 117. In one embodiment, the further CD4+ T-cell epitope and/or the CD8+ T-cell epitope is any sequence that is capable of being recognised by a CD4+ T-cell or a CD8+ T-cell respectively. In one embodiment, the conjugate comprises the polypeptide comprising the sequence of SEQ ID NO: 1, 116 or 117 and a further 1, 2, 3, 4, 5, 6, 7, 8, 9, 10 or more polypeptides, each of which comprises an epitope.

Thus in some embodiments, the conjugate comprises a further substance, in addition to the at least one polypeptide comprising the sequence of a B-cell epitope and the at least one polypeptide comprising the sequence of the CD4+ T-cell epitope.

In one embodiment, the at least one polypeptide comprising the sequence of SEQ ID NO: 1, 116 or 117 comprises the sequence of a further T-cell epitope. In one embodiment, the further T-cell epitope is a further CD4+ T-cell epitope and/or a CD8+ T-cell epitope. In one embodiment, the at least one polypeptide comprises the sequence of SEQ ID NO: 1 and the further T-cell epitope comprises the sequence of SEQ ID NO: 116 or 117, preferably SEQ ID NO: 116. In an alternative embodiment, the further T-cell epitope comprises a different sequence from that of SEQ ID NO: 1, 116 or 117. Thus in some embodiments, the further CD4+ T-cell epitope and/or the CD8+ T-cell epitope is any sequence capable of being recognised by a CD4+ T-cell or a CD8+ T-cell respectively. In certain embodiments comprising a further CD4+ T-cell epitope, the sequence of SEQ ID NO: 1, 116 or 117 and sequence of the further CD4+ T-cell epitope are arranged sequentially and/or overlapping within the at least one polypeptide. In certain embodiments comprising a CD8+ T-cell epitope, the CD8+ T-cell epitope is comprised within the sequence of SEQ ID NO: 1, 116 or 117; that is to say, the sequence of SEQ ID NO: 1, 116 or 117 encompasses the sequence of the CD8+ T-cell epitope. In alternative embodiments, the sequence of SEQ ID NO: 1, 116 or 117 and the CD8+ T-cell epitope are arranged sequentially and/or overlapping within the at least one polypeptide. In embodiments in which epitopes are arranged sequentially, an intervening sequence between the two epitopes may or may not be present.

In some embodiments, the further CD4+ T-cell epitope and/or the CD8+ T-cell epitope (either comprised within the same polypeptide of SEQ ID NO: 1, 116 or 117 or comprised within a further polypeptide in the conjugate) is derived from a self-antigen, a tumour-associated antigen and/or a universal tumour antigen. In one embodiment, the CD8+ T-cell epitope is derived from a prostate cancer-associated antigen, preferably from prostatic acid phosphatase (PAP). In one embodiment, the CD8+ T-cell epitope comprises the sequence “NPILLWQPIPV” (SEQ ID NO: 119) which is derived from PAP. In a preferred embodiment, the at least one polypeptide comprises the sequence of SEQ ID NO: 1 and SEQ ID NO: 119 arranged sequentially. In one embodiment, the sequence of SEQ ID NO: 1 and SEQ ID NO: 119 are arranged sequentially as follows: ALFSVLNYERARRPGLLGASVLGLDDIHRANPILLWQPIPV (SEQ ID NO: 125), in a further embodiment, the order of SEQ ID NO: 1 and SEQ ID NO: 119 is reversed.

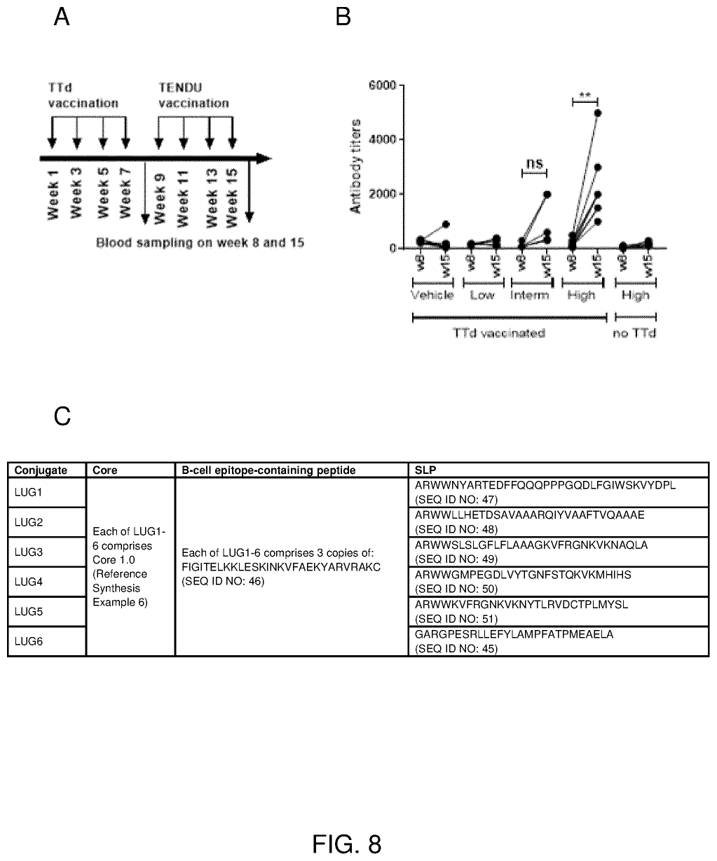

In a further embodiment, the further CD4+ T-cell epitope and/or the CD8+ T-cell epitope (either comprised within the same polypeptide of SEQ ID NO: 1, 116 or 117 or comprised within a further polypeptide in the conjugate) are comprised within any one of SEQ ID NOS: 47 to 51 and/or 45. In one embodiment, the further CD4+ T-cell epitope comprises one or more of SEQ ID NOS: 39 and 41 to 45. In one embodiment, the further CD8+ T-cell epitope comprises one or more of SEQ ID NOS: 155 to 159. In one embodiment, the further CD4+ T-cell epitope and/or the CD8+ T-cell epitope comprises a sequence or a fragment thereof as set out in FIG. 8C (“SLP”).

It is to be appreciated that different lengths of polypeptide elicit different T cell responses. A polypeptide comprising at least 12 amino acids of a universal tumour antigen as described above is of an appropriate length to be presented on an MHC class II molecule and thus to elicit a CD4+ T-cell response (i.e. as polypeptides that are presented on MHC class II molecules are typically between 12 and 24 amino acids in length). In contrast, in order to elicit a CD8+ T-cell response, the polypeptide must be presented on an MHC class I molecule, which will typically only bind shorter polypeptides that are between 8 and 10 amino acid residues in length.

It is to be noted that certain of the CD4+ T-cell epitopes of the present invention (for example, SEQ ID NO: 1, 116 and 117) are longer than would normally be accommodated on an MHC class II molecule. Polypeptides of this length, which are synthetic long peptides (SLPs), have been shown to induce more robust immune responses (for example, Welters et al., Clin Cancer Res. 2008 Jan. 1; 14 (1): 178-87; Rosalia et al., Eur J Immunol. 2013 October; 43 (10): 2554-65). Without wishing to be bound by theory, it is believed that such polypeptides in certain embodiments, following their administration to a subject are endocytosed/taken up by cells, subjected to proteolytic degradation in the proteasome and then presented on an MHC class II (and/or class I) molecule. Thus such polypeptides are capable of giving rise to an MHC class II (and/or an MHC class I) restricted T-cell response. In such an embodiment, it to be understood that a portion of the CD4+ T-cell epitope of the present invention is presented by the MHC class II molecule and bound by the T-cell receptor of the CD4+ T-cell in order to elicit the CD4+ T-cell response. It is also to be appreciated that longer polypeptides remain extant within a subject for a greater period of time than shorter polypeptides and therefore there is a longer period of time during which they may elicit an immune response. This is particularly significant as regards those polypeptides which have a relatively low MHC binding affinity.

In the embodiments described above, the CD4+ T-cell epitope comprises a region of at least 12 amino acids of hTERT. In an alternative embodiment, the CD4+ T-cell epitope comprises a region of at least 12 amino acids of a universal tumour antigen other than hTERT. In one embodiment, the universal tumour antigen is selected from the group consisting of: survivin, DNA topoisomerase 2-alpha (Top2a), cytochrome P450 1B1 (CYP1B1) and E3 ubiquitin-protein ligase Mdm2. In one embodiment, the universal tumour antigen is survivin (Sørensen et al., Cancer Biol Ther. 2008 7 (12): 1885-7; Wobser et al., Cancer Immunol Immunother. 2006 55 (10): 1294-8). Survivin (also known as Baculoviral IAP repeat-containing protein 5) is encoded by the BIRC5 gene in humans and is an inhibitor of apoptosis. A 142 amino acid isoform of Survivin is set out at UniProtKB reference O15392 (isoform 1) and is also set forth in SEQ ID NO. 8. In one embodiment, the universal tumour antigen is DNA topoisomerase 2-alpha (Top2a) (Park et al., Cancer Immunol Immunother. 2010 (5): 747-57). DNA topoisomerase 2-alpha is encoded by the TOP2A gene in humans and controls the topological states of DNA by transient breakage and subsequent rejoining of DNA strands. Topoisomerase II makes double-strand breaks. A 1,531 amino acid isoform of DNA topoisomerase 2-alpha is set out at UniProtKB reference P11388 (isoform 1) and is also set forth in SEQ ID NO. 9. In one embodiment, the universal tumour antigen is cytochrome P450 1B1 (CYP1B1) (Gribben et al., Clin Cancer Res. 2005 11 (12): 4430-6). Cytochrome P450 1B1 is encoded by the CYP1B1 gene in humans and is involved in the metabolism of a diverse range of xenobiotics and endogenous compounds. The 543 amino acid sequence of Cytochrome P450 1B1 is set out at UniProtKB reference Q16678 and is also set forth in SEQ ID NO. 10. In one embodiment, the universal tumour antigen is E3 ubiquitin-protein ligase Mdm2 (Gordan and Vonderheide, Cytotherapy. 2002; 4 (4): 317-27). E3 ubiquitin-protein ligase Mdm2 is encoded by the MDM2 gene in humans and is a negative regulator of the p53 tumour suppressor. A 491 amino acid isoform of E3 ubiquitin-protein ligase Mdm2 is set out at UniProtKB reference Q00987 (Isoform Mdm2) and is also set forth in SEQ ID NO. 11.

In a preferred embodiment, the CD4+ T-cell epitope comprises a region of at least 13, 14, 16, 17, 18, 19, 20, 21, 22, 23, 24, 25, 26, 27, 28, 29 or 30 amino acids of the universal tumour antigen. In a particularly preferred embodiment, the CD4+ T-cell epitope consists of a region of 13, 14, 16, 17, 18, 19, 20, 21, 22, 23, 24, 25, 26, 27, 28, 29 or 30 amino acids of the universal tumour antigen. As discussed above, SEQ ID NO: 1, 116 or 117 each consist of a region of 30 amino acids of hTERT. In one embodiment, the at least one polypeptide comprising the CD4+ T-cell epitope is 500 amino acids or less in length. In a further embodiment, the at least one polypeptide comprising the CD4+ T-cell epitope is equal to or less than 400, 300, 200, 170, 150, 125, 100, 90, 80, 70, 75, 60, 50, 40 or 30 amino acids or less in length.

In one embodiment, the “universal tumour antigen” is not cancer/testis antigen 1 (NY-ESO-1), prostatic acid phosphatase (PAP) and/or glutamate carboxypeptidase 2 (GCPII). It is to be understood that NY-ESO-1 is not encompassed by the term “universal tumour antigen” as used herein because it is not generally expressed in a high proportion of patients within each tumour type (Ishihara et al. BMC Cancer. 2020; 20:606). Furthermore, it is to be understood that PAP and GCPII are prostate cancer-associated proteins therefore are also not encompassed by the term “universal tumour antigen” as used herein. NY-ESO-1 is encoded by the CTAG1A gene in humans and has the UniProt accession number P78358. The amino acid sequence of NY-ESO-1 is set out in SEQ ID NO. 36. PAP is encoded by the ACPP gene in humans and has the UniProt access number P15309. The amino acid sequence of PAP is set out in SEQ ID NO. 37. GCPII (also known as prostate-specific membrane antigen [PSMA]) is encoded by the FOLH1 gene in humans and has the Uniprot accession number Q04609. The amino acid sequence of GCPII is set out in SEQ ID NO. 38.

In one embodiment, the CD4+ T-cell epitope comprising a region of at least 12 amino acids of a universal tumour antigen does not comprise the sequence of any one of SEQ ID NOS. 39 to 45. SEQ ID NOS. 39 (i.e. GQDLFGIWSKVYDPL) and 40 (i.e. TEDTMTKLRELSELS) are derived from PAP; SEQ ID NOS. 41 to 44 (i.e. GKVFRGNKVKNAQLA, TGNFSTQKVKMHIHS, NYTLRVDCTPLMYSL, RQIYVAAFTVQAAAE respectively) are derived from GCPII; and SEQ ID NO. 45 (i.e. GARGPESRLLEFYLAMPFATPMEAELA) is derived from NY-ESO-1.

In addition to comprising at least 12 amino acids of a universal tumour antigen, the CD4+ T-cell epitope is immunogenic in at least 50% of the population. That is to say, the CD4+ T-cell epitope is capable of eliciting an immune response in 50% or more of individuals within a population. In a preferred embodiment, the CD4+ T-cell epitope is immunogenic in 55% or more or 60% or more of the population. In a particularly preferred embodiment, the CD4+ T-cell immune response is immunogenic in 65% or more of the population. In one embodiment, the CD4+ T-cell epitope comprises the sequence of SEQ ID NO: 1 and is immunogenic in 65% or more of the population. In a further embodiment, the CD4+ T-cell epitope comprises the sequence of SEQ ID NO: 116 or 117 and is immunogenic in at least 50% of the population. It is to be appreciated that polypeptides comprising the sequences of SEQ ID NO: 116 or 117 comprise the sequence of EARPALLTSRLRFIPK (SEQ ID NO: 126) which has been reported to induce immune responses in at least 50% of vaccinated individuals (see, for example, Bernhardt et al. Br J Cancer. 2006 Dec. 4; 95 (11): 1474-82; Inderberg-Suso et al. 2012; and Kyte et al. Clin Cancer Res Jul. 1 2011 (17) (13) 4568-4580). These sequences are further analysed in Examples 5 and 6.

In a first embodiment, the population is the general population. That is to say the population comprises both healthy individuals and individuals with a disease such as cancer. In an alternative embodiment, the population consists of individuals who are cancer patients. In a further embodiment, the cancer patients are patients having non-small-cell lung carcinoma, prostate cancer and/or malignant melanoma. In one embodiment, the population further comprises cancer patients with pancreatic cancer.

It is to be understood that, in order to be immunogenic is a population, the CD4+ T-cell epitope (or a portion thereof) must be capable of being presented on (i.e. binding to) an HLA class II molecule that is present within the population. The frequency of HLA class II alleles in the different regions worldwide is shown in Table 1 (adapted from Dosset et al., Cancers. 2020 Jun. 25; 12 (6):E1687). By “frequency” is meant the proportion of individuals in the population who carry each allele. In one embodiment, the CD4+ T-cell epitope (or a portion thereof) is bound by one or more HLA class II allele that is present in the population at a frequency of 10% or more. In one embodiment, the HLA class II allele that is present in the population at a frequency of 10% or more is selected from the group consisting of: DRB1*01, DPA1*01, DPA1*02, DPB1*02, DPB1*03, DPB1*04, DQA1*01, DQA1*02, DQA1*03, DQB1*02 and DQB1*03. In a further embodiment, the CD4+ T-cell epitope (or a portion thereof) is bound by one or more HLA class II allele that is present in the population at a frequency of 40% or more, preferably 50% or more, more preferably 60%, 70%, 80%, 90%, 95% or more or 100%. In one embodiment, the HLA class II allele that is present in the population at a frequency of 40% or more is selected from the group consisting of: DPA1*01, DPA1*02, DPB1*02, DPB1*03, DPB1*04, DQA1*01, DQA1*03, DQB1*02 and DQB1*03.

In one embodiment, the CD4+ T-cell epitope (or a portion thereof) is bound by one or more HLA class II allele selected from the group consisting of: HLA-DRB1*15, HLA-DRB1*07, HLA-DRB1*04, HLA-DQB1*06, HLA-DQB1*03, HLA-DQB1*05, HLA-DPB1*04 and HLA-DPB1*01. In a particularly preferred embodiment, the CD4+ T-cell epitope (or a portion thereof) is bound by one or more HLA class II allele selected from the group consisting of: HLA-DRB1*15:01, HLA-DRB1*07:01, HLA-DRB1*04:01, HLA-DQB1*06:02, HLA-DQB1*03:02, HLA-DQB1*05:01, HLA-DPB1*04:01, HLA-DPB1*04:02 and HLA-DPB1*01:01. In a yet a further preferred embodiment, the CD4+ T-cell epitope (or a portion thereof) is bound by one or more HLA class II allele selected from the group consisting of: HLA-DRB1*15:01, HLA-DRB1*07:01, HLA-DRB1*04:01, HLA-DQB1*06:02, HLA-DQB1*05:01, HLA-DPB1*04:01, HLA-DPB1*04:02, even more preferably, HLA-DRB1*15:01 and/or HLA-DRB1*07:01. Individuals having the above-mentioned HLA-class II alleles have been shown to elicit an immune response to the polypeptide of SEQ ID NO: 1.

It is to be understood (for example, with reference to Table 1) that the frequency of an HLA class II allele in a population may differ depending on the geographical region. In one embodiment, the population is a European population. In an alternative embodiment, the population is a North American population, a South/Central American population, a North African population, a Sub-Saharan African population, a Western Asian population, a North-East Asian population, a South-East Asian population and/or an Australian population. In one embodiment, the CD4+ T-cell epitope (or a portion thereof) is bound by one or more HLA class II allele that is present in one of the aforementioned populations at a frequency of 10% or more, preferably 40% or 50% or more, more preferably 60%, 70%, 80%, 90%, 95% or more or 100%.

In one embodiment, where the population is a European population, the HLA class II allele is at least one selected from the group consisting of: DPA1*01, DPB1*03, DPB1*04, DQA1*01 and DQB1*02. In one embodiment, where the population is a North American population, the HLA class II allele is one or more selected from the group consisting of: DPA1*01, DPB1*04, DQA1*03 and DQB1*03. In one embodiment, where the population is a South/Central American population, the HLA class II allele is one or more selected from the group consisting of: DPA1*01, DPA1*02, DPB1*04, DQA1*01, DQA1*03 and DQB1*03. In one embodiment, where the population is a North African population, the HLA class II allele is DQB1*02. In one embodiment, where the population is a Sub-Saharan African population, the HLA class II allele is one or more selected from the group consisting of: DPA1*01, DPA1*02, DPB1*04, DQA1*01 and DQB1*03. In one embodiment, where the population is a Western Asian population, the HLA class II allele is one or more selected from the group consisting of: DPB1*04, DQA1*01, DQB1*02 and DQB1*03. In one embodiment, where the population is a North-East Asian population, the HLA class II allele is one or more selected from the group consisting of: DPA1*01, DPA1*02. DPB1*02, DPB1*04, DQA1*01, DQA1*03 and DQB1*03. In one embodiment, where the population is a South-East Asian population, the HLA class II allele is one or more selected from the group consisting of: DPA1*01, DPA1*02, DPB1*04, DQA1*01 and DQB1*03. It is to be understood from Table 1 that the aforementioned HLA class II alleles are generally present at a frequency of 40% or more in the aforementioned populations.

It is to be appreciated that the CD4+ T-cell epitope (or a portion thereof) may be bound by multiple different HLA class II alleles in a population. In one embodiment, the CD4+ T-cell epitope is bound by multiple (e.g. 2, 3, 4, 5, 6, 7, 8, 9, 10 or more or all) of the aforementioned different HLA class II alleles. Binding to one or multiple HLA class II allele(s) that are present at a higher frequency in the population is preferred as it suggests the CD4+ T-cell epitope will be immunogenic in a higher proportion of the population i.e. as a higher proportion of individuals within the population will carry an appropriate HLA class II allele on which the CD4+ T-cell epitope (or a portion thereof) can be presented in order to elicit a CD4+ T-cell response.

TABLE 1
Prevalence of the most common (≥10% frequency) HLA class II molecules
worldwide (adapted from Dosset et al., Cancers. 2020 Jun. 25; 12(6): E1687)

| | Region | | | | | | | | |
|---|---|---|---|---|---|---|---|---|---|
| HLA | | | South/ | | Sub- | | North- | South- | |
| Class II | | North | Central | North | Saharan | Western | East | East | |
| Allele | Europe | America | America | Africa | Africa | Asia | Asia | Asia | Australia |
| DRB1*01 | 17% | 17% | 15% | 13% | 10% | 15% | 24% | 7% | 11% |
| DPA1*01 | 87% | 100% | 72% | — | 45% | — | 77% | 64% | — |
| DPA1*02 | 19% | 12% | 71% | — | 56% | — | 48% | 59% | — |
| DPB1*02 | 35% | 17% | 19% | 31% | 18% | 29% | 66% | 34% | 16% |
| DPB1*03 | 49% | 17% | 18% | 14% | 9% | 15% | 16% | 10% | 3% |
| DPB1*04 | 52% | 91% | 89% | 34% | 70% | 68% | 42% | 43% | 11% |
| DQA1*01 | 44% | 31% | 59% | 39% | 50% | 50% | 41% | 70% | 36% |
| DQA1*02 | 31% | 13% | 25% | 22% | 12% | 27% | 26% | 39% | 4% |
| DQA1*03 | 24% | 70% | 75% | 18% | 27% | 24% | 66% | 32% | — |
| DQB1*02 | 59% | 24% | 33% | 43% | 37% | 46% | 29% | 33% | 7% |
| DQB1*03 | 47% | 94% | 78% | 32% | 43% | 48% | 67% | 55% | 9% |

In a further embodiment, the polypeptide comprising the sequence of the CD4+ T-cell epitope and/or the CD4+ T-cell epitope does not have the exact sequence identity to one of the aforementioned sequences. Instead, the polypeptide and/or the CD4+ T-cell epitope has at least 80% sequence identity to that as set out above. Preferably, at least 85%, 90%, 91%, 92%, 93%, 94%, 95%, 96%, 97%, 98% or 99% sequence identity to that set out above. It is also preferred that any addition or substitution of amino acid sequence results in the conservation of the properties of the original amino acid side chain. That is to say the substitution or modification is “conservative” (as described above).

The CD4+ T-cell epitope (in particular one whose sequence has been altered as set out above) is able to induce a CD4+ T-cell response. That is to say the CD4+ T-cell epitope should be able to induce CD4+ (helper) T-cells when presented by on an appropriate MHC class II molecule by antigen presenting cells (e.g. dendritic cells). Confirmation that a CD4+ T-cell immune response is induced can be accomplished using a T-cell proliferation assay (H-Thymidine) as described above.

The conjugate may contain any combination of the above-mentioned polypeptides, provided that it comprises at least one polypeptide comprising a sequence of a B-cell epitope and at least one polypeptide comprising a sequence of a CD4+ T-cell epitope as set out above. It is to be understood that the sequence of the B-cell epitope is different from the sequence of the CD4+ T-cell epitope. In one embodiment, the sequence of the B-cell epitope does not comprise the sequence of the CD4+ T-cell epitope. In a further embodiment, the sequence of the CD4+ T-cell epitope does not comprise the sequence of the B-cell epitope.

In a further embodiment, there is provided a cocktail (i.e. a mixture) of any one of the conjugates defined herein. In some embodiments, the cocktail of conjugates comprises at least two different conjugates comprising the sequences of CD4+ T-cell epitopes selected from SEQ ID NOS: 1, 116 and 117. In some embodiments, the cocktail comprises conjugates comprising immunogenic fragments of said CD4+ T-cell epitopes, wherein the immunogenic fragments comprise at least 12 amino acids. In further embodiments, the or each conjugate in the cocktail comprises the sequence of a CD4+ T-cell epitope having at least 80% sequence identity to the sequence of SEQ ID NO: 1, 116 or 117 or the immunogenic fragment thereof. It is particularly preferred that the sequence has at least 85%, 90%, 91%, 92%, 93%, 94%, 95%, 96%, 97%, 98% or 99% sequence identity to that set out above. In a preferred embodiment, the conjugates in the cocktail comprise the sequences of the CD4+ T-cell epitopes of SEQ ID NO: 1 and 116 or an immunogenic fragment thereof comprising at least 12 amino acids. In one embodiment, the polypeptides comprising the sequences of the CD4+ T-cell epitopes, which are comprised within each conjugate, are equal to or less than 500 amino acids in length, preferably equal to or less than 400, 300, 200, 170, 150, 125, 100, 90, 80, 70, 75, 60, 50, 40 or 30 amino acids in length. It is preferred that the at least two conjugates are different in the sense of being based on different CD4+ T-cell epitopes selected from SEQ ID NOS: 1, 116 and 117.

In some embodiments, there is provided a molecule comprising a first nucleotide sequence encoding the polypeptide comprising the sequence of the B-cell epitope and/or a second nucleotide sequence encoding the polypeptide comprising the sequence of the CD4+ T-cell epitope. In some embodiments, the first and/or a second nucleotide sequence encodes a fragment of the polypeptide comprising the sequence of the B-cell epitope and/or the CD4+ T-cell epitope. In one embodiment, the molecule is a nucleic acid molecule. In certain embodiments, the molecule or nucleic acid molecule comprises an intervening sequence between the first and second nucleotide sequences.

In a further embodiment, a vector comprising a nucleic acid molecule as set out above or a fragment thereof is provided. The particular vector to be used may be dependent on the host organism or cell(s) in which the nucleic acid molecule is to be expressed, the method that will be used to transform the host cell(s), and/or the method that is to be used for protein expression (or any another intended use of the vector). In one embodiment, the vector is used for delivery of the nucleic acid molecule into a cell and/or for administration of the nucleic acid molecule to a subject.

In one embodiment, the at least one polypeptide comprising a sequence of a B-cell epitope and the at least one polypeptide comprising a sequence of a CD4+ T-cell epitope may be non-covalently linked or covalently linked. Preferably, the at least one polypeptide comprising a sequence of a B-cell epitope and the at least one polypeptide comprising a sequence of a CD4+ T-cell epitope may be covalently linked.

In one embodiment, the at least one polypeptide comprising a sequence of a B-cell epitope and the at least one polypeptide comprising a sequence of a CD4+ T-cell epitope may be directly linked. In another embodiment, the at least one polypeptide comprising a sequence of a B-cell epitope and the at least one polypeptide comprising a sequence of a CD4+ T-cell epitope may be indirectly linked.

In one embodiment, the at least one polypeptide comprising a sequence of a B-cell epitope and the at least one polypeptide comprising a sequence of a CD4+ T-cell epitope may be linked via a core,
wherein the core comprises, prior to linkage:
   a body portion;
   one or more first linking groups attached to the body portion; and
   one or more second linking groups attached to the body portion,
   wherein the first linking group and second linking group are orthogonal to each other;
and wherein the first linking group is linked to the at least one polypeptide comprising a sequence of a B-cell epitope to form a first connecting element, and the second linking group is linked to the at least one polypeptide comprising a sequence of a CD4+ T-cell epitope to form a second connecting element.

In a preferred embodiment, the at least one polypeptide comprising a sequence of a B-cell epitope and the at least one polypeptide comprising a sequence of a CD4+ T-cell epitope are linked within the conjugate via a core structure as defined herein. That is to say, in a preferred embodiment, the sequence of the B-cell epitope and the sequence of the CD4+ T-cell epitope are not arranged sequentially and/or overlapping within a single polypeptide chain.

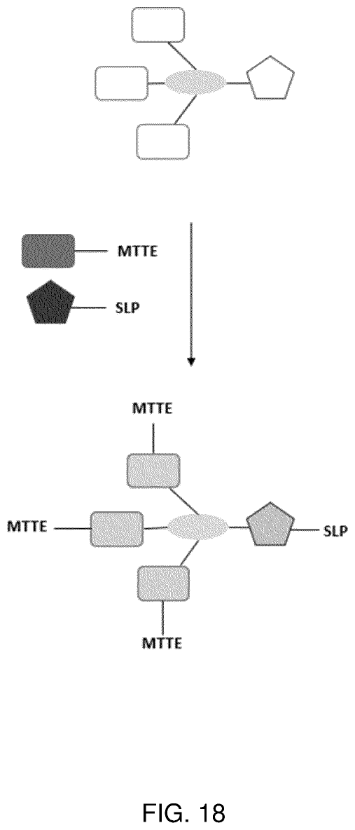

FIG. 18 shows a schematic structure of a conjugate according to an embodiment of the present invention.

In one embodiment, prior to linkage, the at least one polypeptide comprising a sequence of a B-cell epitope and the at least one polypeptide comprising a sequence of a CD4+ T-cell epitope may independently comprise a reactive functional group which is configured to undergo a coupling reaction with a first linking group as defined herein and a second linking group as defined herein respectively. For example, the reactive functional group may be present on natural amino acid residues (e.g. a thiol group of a cysteine residue). In other cases, a natural amino acid residue may be modified to include a reactive functional group (e.g. an azide group of an azidolysine residue, or an alkyne (e.g. a terminal alkyne), an alkene (e.g. a terminal alkene, norbornene), a cycloalkyne, a trans-cycloalkene, a tetrazine, a conjugated diene, a maleimide or an α-halocarbonyl). Preferred examples of reactive functional groups include thiol groups and azide groups.

In one embodiment, the reactive functional group may be provided at the C-terminus of the at least one polypeptide comprising a sequence of a B-cell epitope and/or the at least one polypeptide comprising a sequence of a CD4+ T-cell epitope. In another embodiment, the reactive functional group may be provided at the N-terminus of the at least one polypeptide comprising a sequence of a B-cell epitope and/or the at least one polypeptide comprising a sequence of a CD4+ T-cell epitope.

In one embodiment, the at least one polypeptide comprising a sequence of a B-cell epitope and the at least one polypeptide comprising a sequence of a CD4+ T-cell epitope may independently comprise a cysteine residue. In some embodiments, the at least one polypeptide comprising a sequence of a B-cell epitope and the at least one polypeptide comprising a sequence of a CD4+ T-cell epitope may independently comprise only one cysteine residue.

In one embodiment, the at least one polypeptide comprising a sequence of a B-cell epitope and the at least one polypeptide comprising a sequence of a CD4+ T-cell epitope may independently comprise an azidolysine (i.e. K(N₃)) residue. In some embodiments, the at least one polypeptide comprising a sequence of a B-cell epitope and the at least one polypeptide comprising a sequence of a CD4+ T-cell epitope may independently comprise only one azidolysine residue.

In one embodiment, prior to linkage, the at least one polypeptide comprising a sequence of a B-cell epitope and the at least one polypeptide comprising a sequence of a CD4+ T-cell epitope may independently comprise a spacer sequence. In an alternative embodiment, the spacer is comprised within the core (for example between a body portion and a first linking group, or a body portion and a second linking group). The at least one polypeptide comprising a sequence of a B-cell epitope or a sequence of a CD4+ T-cell epitope may comprise a spacer sequence either N-terminal and/or C-terminal to the B-cell epitope or the CD4+ T-cell epitope respectively. In one embodiment, the spacer sequence is between the reactive functional group and the epitope sequence. In some embodiments, the spacer sequence is comprised within the at least one polypeptide comprising a sequence of a B-cell epitope. In a preferred embodiment, the spacer sequence comprises an amino acid sequence. In one embodiment, the spacer sequence is 10 amino acids in length. In further embodiments, the spacer sequence comprises 1 to 20, 1 to 15 or 1 to 12 amino acids, preferably 3 to 12, 6 to 12 or 9 to 12 amino acids. In some embodiments, the spacer comprises or consists of the sequence of AEKYARVRAK (SEQ ID NO: 127), AAKYARVRAK (SEQ ID NO: 128), AAKYARVRAKC (SEQ ID NO: 129) or SSAFADVEAA (SEQ ID NO: 154) or a sequence having at least 70%, 75%, 80%, 85%, 90%, 95%, 98%, 99% sequence identity thereto. Preferably, the spacer sequence comprises or consists of the sequence of AEKYARVRAK (SEQ ID NO: 127). However, the spacer sequence is not limited to the aforementioned sequences and a person skilled in the art will be aware of alternative spacer sequences. In one embodiment, the spacer sequence contributes to a property of the conjugate (e.g. solubility and/or structural properties).

In one embodiment, the first connecting element and the second connecting element are independently selected from a 1,2,3-triazole linkage, a dihydropyridazine linkage, a pyridazine linkage and a sulfide linkage (e.g. formed from thiol-ene reactions). Preferably, the first connecting element and the second connecting element are independently selected from a 1,2,3-triazole linkage and a sulfide linkage. More preferably, the first connecting element and the second connecting element are independently selected from:

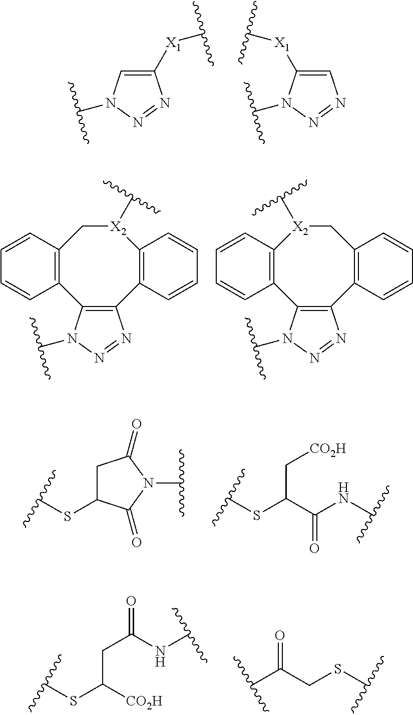

wherein:
X₁ is selected from —(CH₂)ₐₓ₁₁— and —(CH₂)ₐₓ₁₂—X₁₂—;
wherein X₁₂ is selected from O, NR₁₂ or S;
R₁₂ is selected from hydrogen, optionally substituted alkyl, optionally substituted cycloalkyl, optionally substituted heterocycloalkyl, optionally substituted aryl and optionally substituted heteroaryl; preferably hydrogen and optionally substituted alkyl;
X₂ is selected from N or CH;
ax11 and ax12 are independently selected from 0 to 12, and
the wavy line represents a connection point to the at least one polypeptide comprising a sequence of a B-cell epitope or the at least one polypeptide comprising a sequence of a CD4+ T-cell epitope and/or the core.

In one embodiment, the third connecting element is selected from a 1,2,3-triazole linkage, a dihydropyridazine linkage, a pyridazine linkage and a sulfide linkage (e.g. formed from thiol-ene reactions). Preferably, the third connecting element is selected from a 1,2,3-triazole linkage. More preferably, the third connecting element is independently selected from:

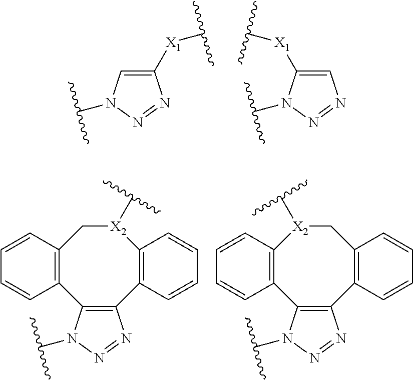

wherein:
X₁ is selected from —(CH₂)ₐₓ₁₁— and —(CH₂)ₐₓ₁₂—X₁₂—;
wherein X₁₂ is selected from O, NR₁₂ or S;
R₁₂ is selected from hydrogen, optionally substituted alkyl, optionally substituted cycloalkyl, optionally substituted heterocycloalkyl, optionally substituted aryl and optionally substituted heteroaryl; preferably hydrogen and optionally substituted alkyl;
X₂ is selected from N or CH:
ax11 and ax12 are independently selected from 0 to 12, and the wavy line represents a connection point to the at least one polypeptide comprising a sequence of a B-cell epitope or the at least one polypeptide comprising a sequence of a CD4+ T-cell epitope and/or the core.

Preferably, ax11 and ax12 are independently selected from 1 to 12. More preferably, ax11 and ax12 are independently selected from 1 to 6. Even more preferably, ax11 and ax12 are independently selected from 1 to 4.

Preferably, X₁₂ may be O or NR¹². More preferably, X₁₂ may be O.

Other preferred embodiments with regard to the core are described in further detail below and apply equally to the conjugate.

In accordance with another aspect of the invention, there is provided a conjugate comprising:
(a) at least one polypeptide comprising a sequence of a B-cell epitope; and
(b) at least one polypeptide comprising a sequence of a CD4+ T-cell epitope;
   wherein the at least one polypeptide comprising a sequence of a B-cell epitope or the at least one polypeptide comprising a sequence of a CD4+ T-cell epitope are linked via a core, wherein the core comprises, prior to linkage:
      a body portion;
      one or more first linking groups attached to the body portion; and
      one or more second linking groups attached to the body portion,
      wherein the first linking group and second linking group are orthogonal to each other,
      wherein the core is not:

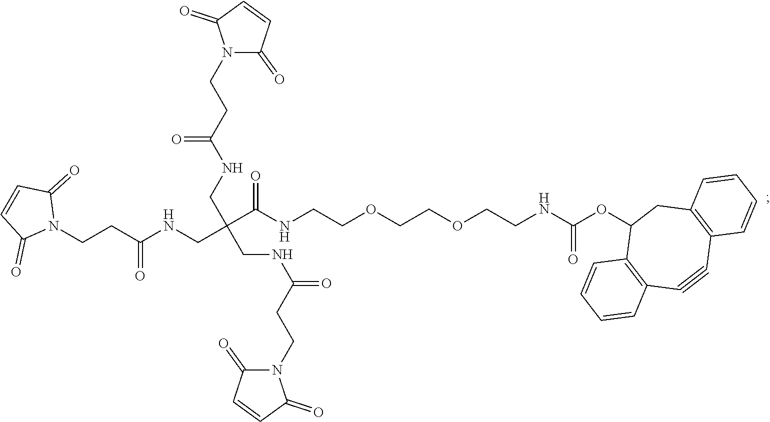 [SMILES: O=C(CCN1C(=O)C=CC1=O)NCC(CNC(=O)CCN1C(=O)C=CC1=O)(CNC(=O)CCN1C(=O)C=CC1=O)C(=O)NCCOCCOCCNC(=O)OC1Cc2ccccc2C#Cc2ccccc21]

and wherein the first linking group is linked to the at least one polypeptide comprising a sequence of a B-cell epitope to form a first connecting element, and the second linking group is linked to the at least one polypeptide comprising a sequence of a CD4+ T-cell epitope to form a second connecting element. Other preferred embodiments with regard to the conjugate have been described above and apply equally to this conjugate. Other preferred embodiments with regard to the core are described in further detail below and apply equally to this conjugate.

In accordance with another aspect of the invention, there is provided an intermediate conjugate comprising:
(a) at least one polypeptide comprising a sequence of a B-cell epitope; or
(b) at least one polypeptide comprising a sequence of a CD4+ T-cell epitope;
   wherein the at least one polypeptide comprising a sequence of a B-cell epitope or the at least one polypeptide comprising a sequence of a CD4+ T-cell epitope is linked to a core, wherein the core comprises, prior to linkage:
      a body portion;
      one or more first linking groups attached to the body portion; and
      one or more second linking groups attached to the body portion,
      wherein the first linking group and second linking group are orthogonal to each other,
      wherein the core is not:

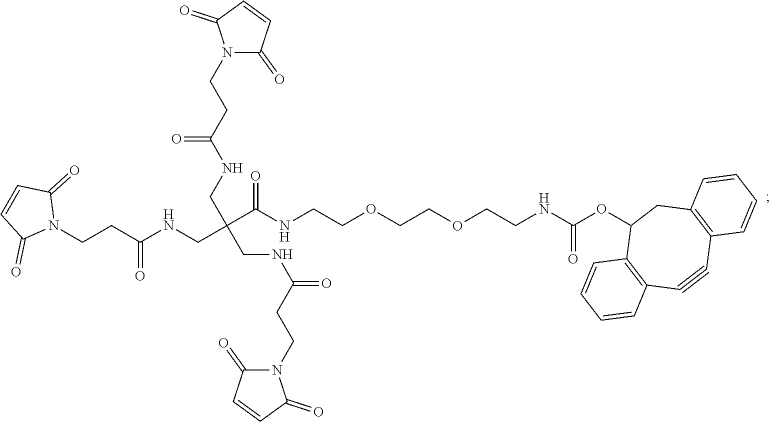 [SMILES: O=C(CCN1C(=O)C=CC1=O)NCC(CNC(=O)CCN1C(=O)C=CC1=O)(CNC(=O)CCN1C(=O)C=CC1=O)C(=O)NCCOCCOCCNC(=O)OC1Cc2ccccc2C#Cc2ccccc21]

and wherein the first linking group is linked to the at least one polypeptide comprising a sequence of a B-cell epitope to form a first connecting element, or the second linking group is linked to the at least one polypeptide comprising a sequence of a CD4+ T-cell epitope to form a second connecting element. Other preferred embodiments with regard to the conjugate have been described above and apply equally to the intermediate conjugate. Other preferred embodiments with regard to the core are described in further detail below and apply equally to the intermediate conjugate.

In one embodiment, the conjugate may be selected from:

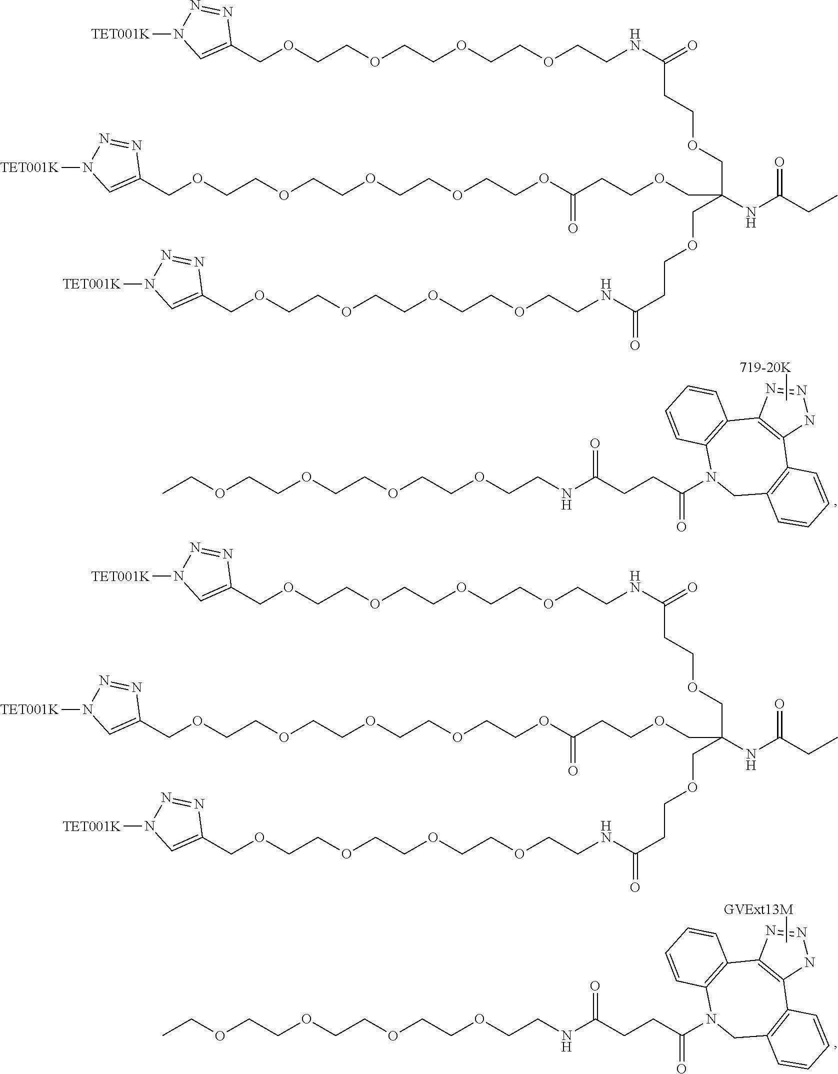

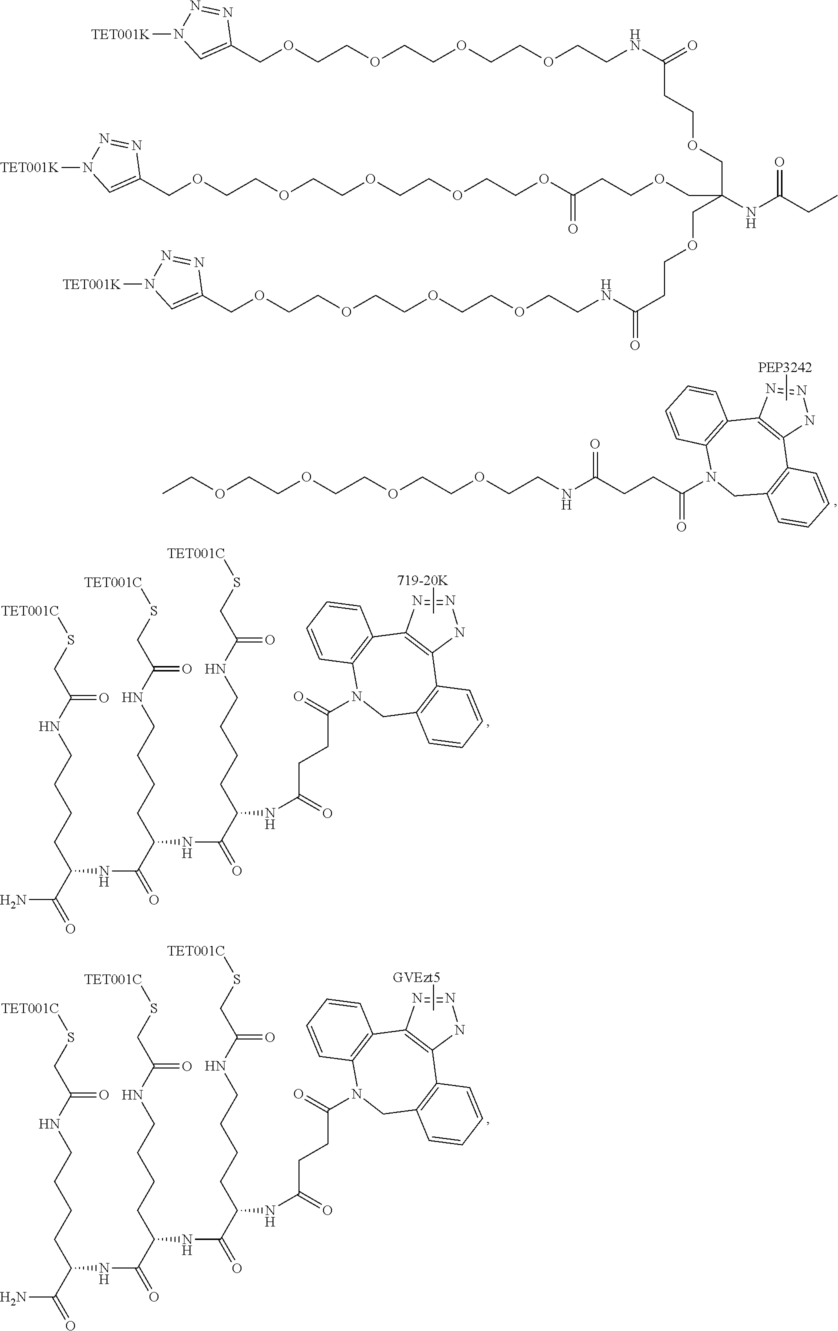

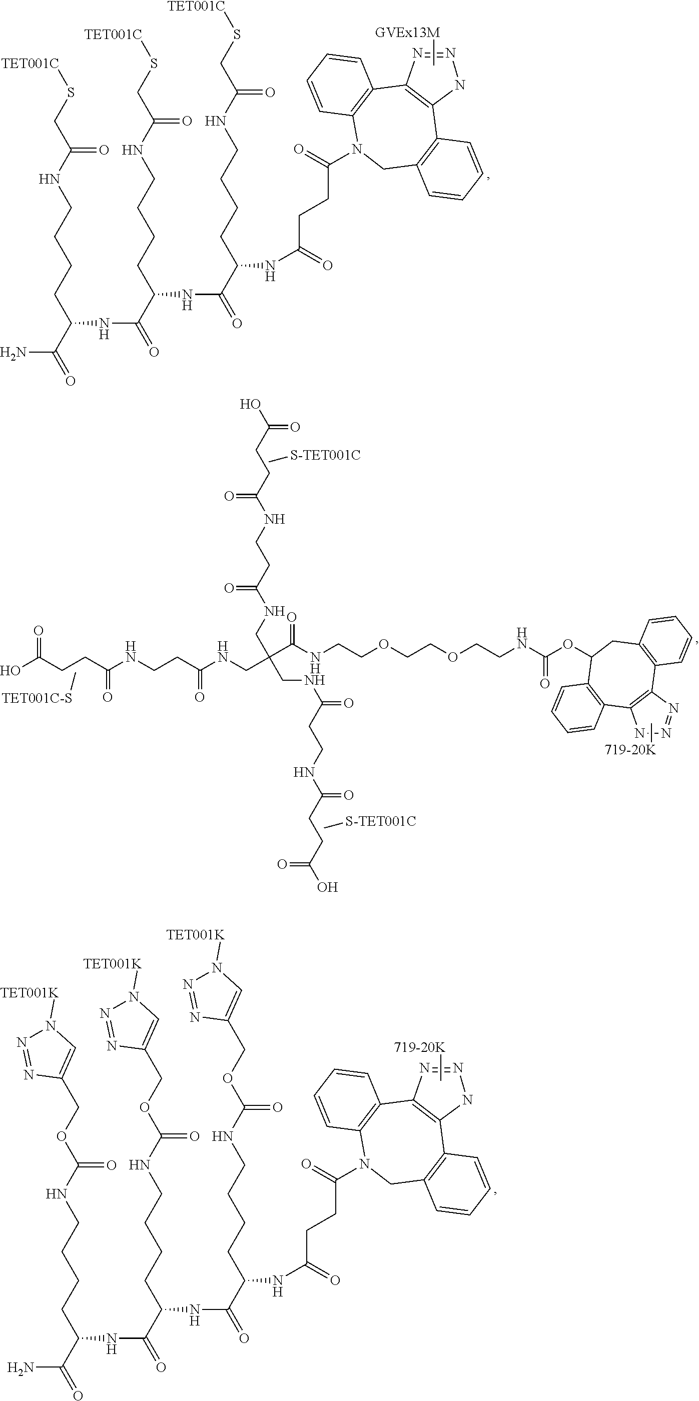

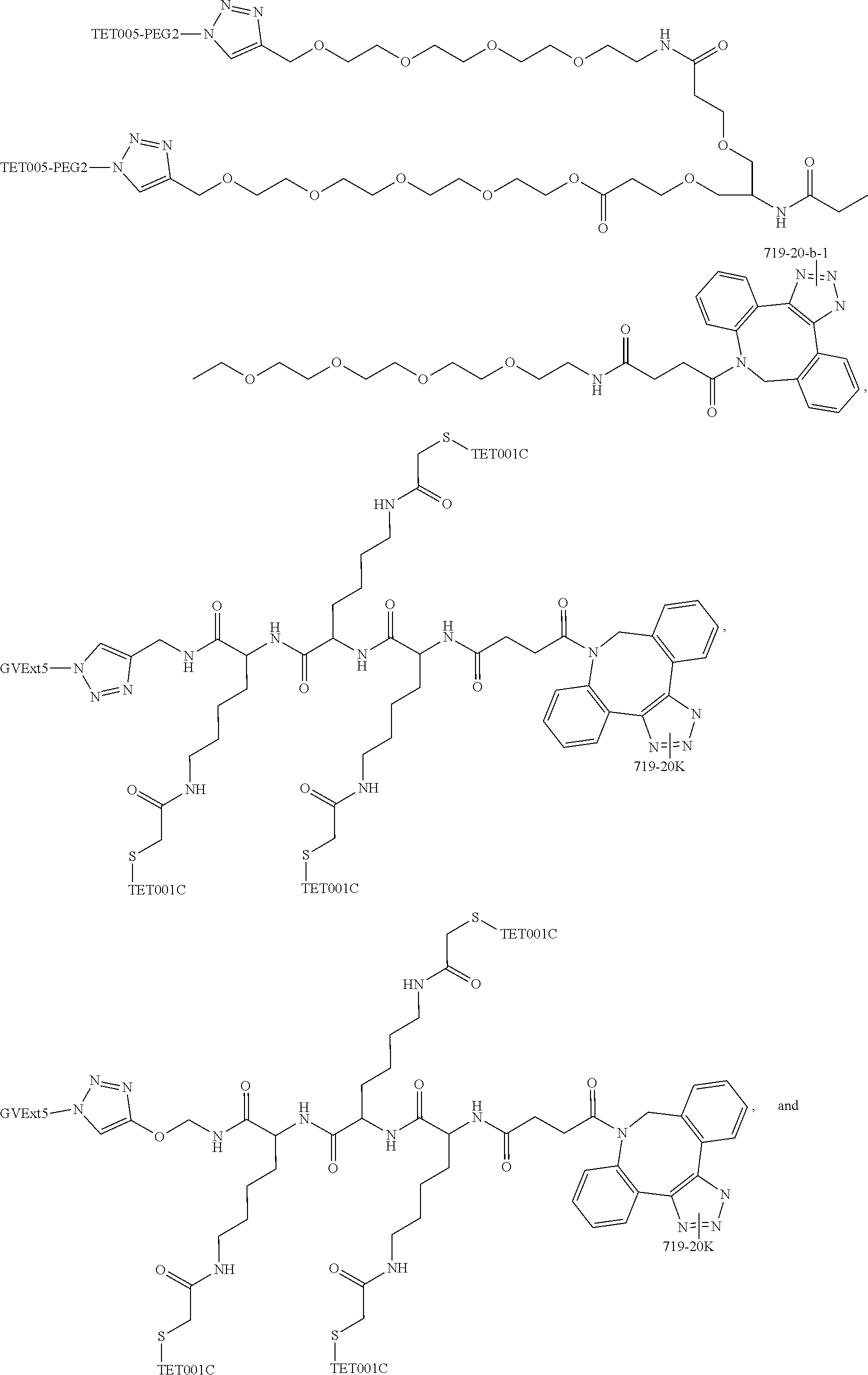

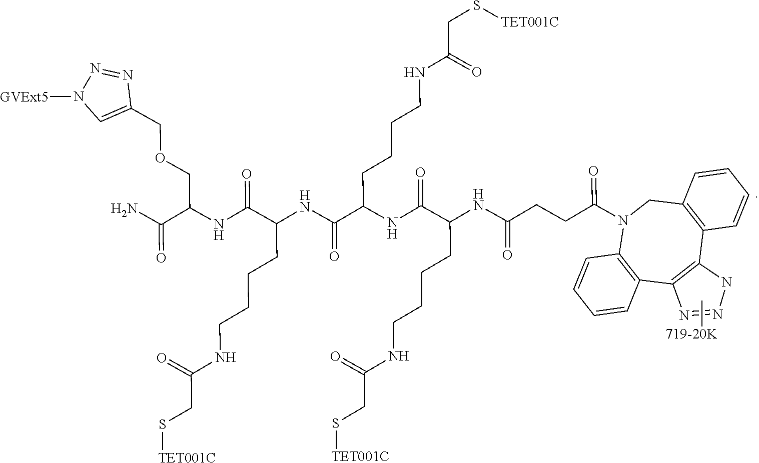

### A Core

In accordance with another aspect of the invention, there is provided a core comprising: a body portion;
one or more first linking groups attached to the body portion; and
one or more second linking groups attached to the body portion,
wherein the first linking group and second linking group are orthogonal to each
other.

In one embodiment, the body portion is configured such that when a polypeptide comprising a B-cell epitope is attached to the one or more first linking groups, an antibody specific for the B-cell epitope is able to bind to the B-cell epitope.

In one embodiment, the core is not:

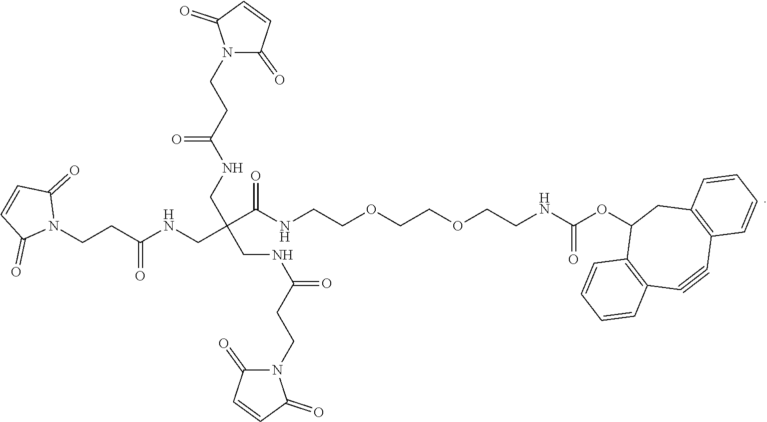 [SMILES: O=C(CCN1C(=O)C=CC1=O)NCC(CNC(=O)CCN1C(=O)C=CC1=O)(CNC(=O)CCN1C(=O)C=CC1=O)C(=O)NCCOCCOCCNC(=O)OC1Cc2ccccc2C#Cc2ccccc21]

Optionally, this may also apply to conjugates, intermediate conjugates or methods of the present invention; however, the conjugates, intermediate conjugates or methods of the present invention are not necessarily limited thereto.

In embodiments relating to the core per se, the core is not:

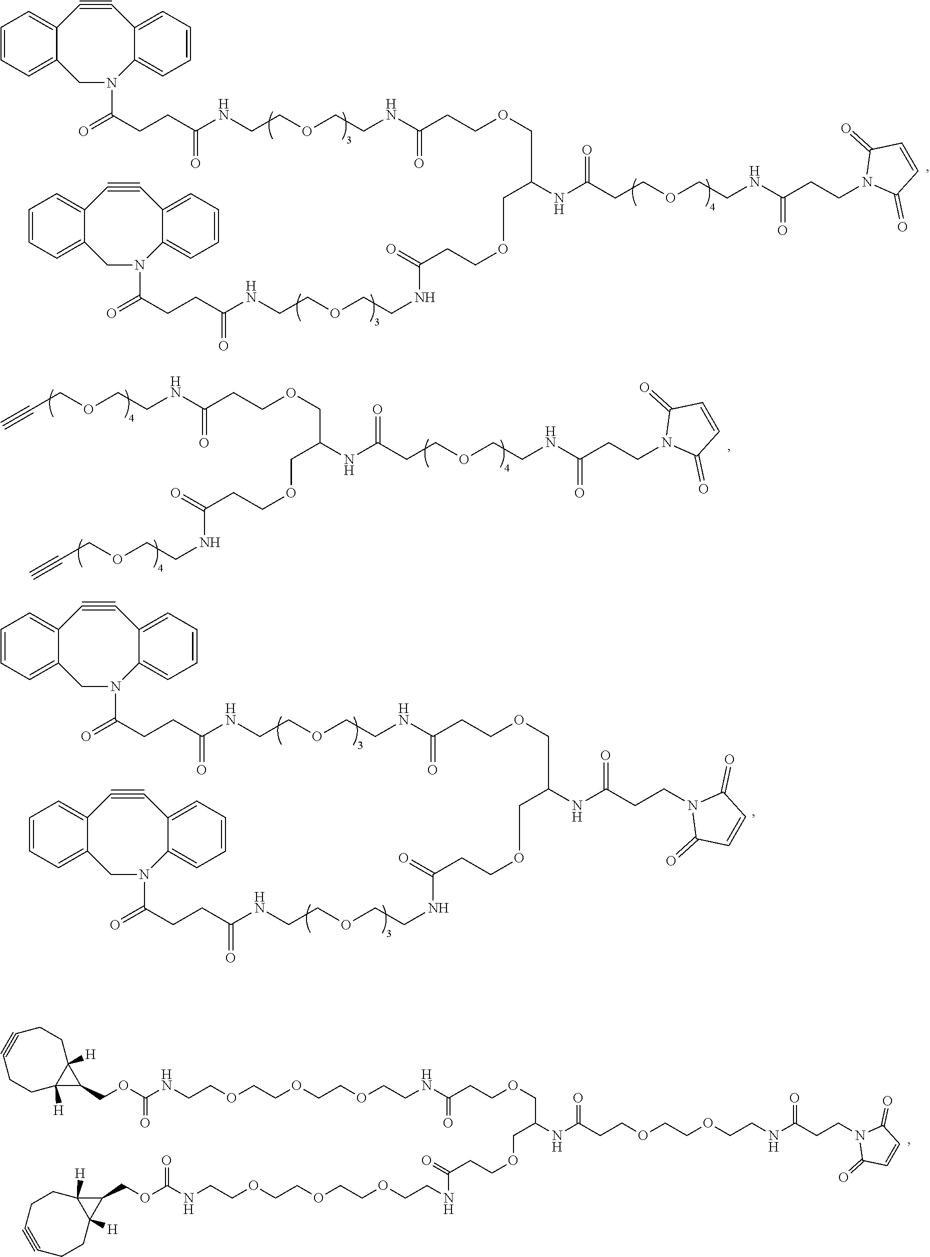

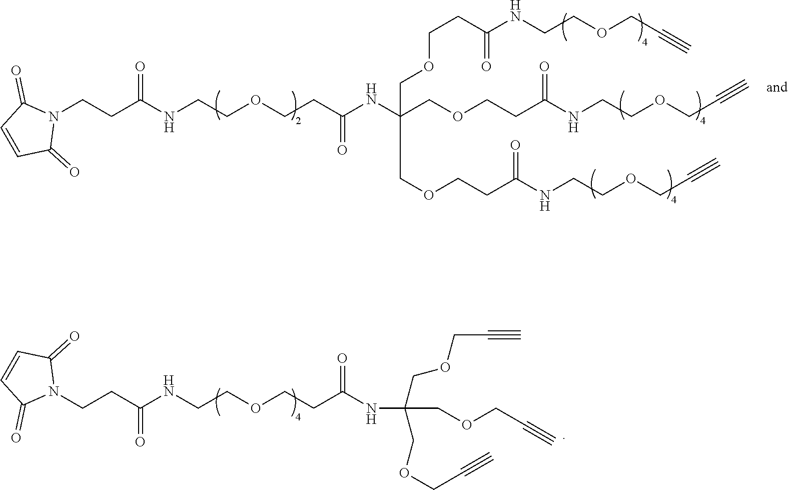

Optionally, this may also apply to conjugates, intermediate conjugates or methods of the present invention; however, the conjugates, intermediate conjugates or methods of the present invention are not necessarily limited thereto.

Cores according to the present invention are generally more efficiently synthesised than compared to other cores, such as the following core:

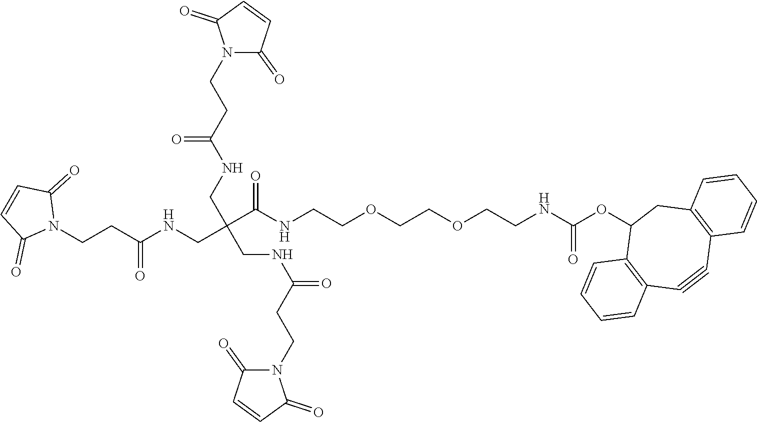 [SMILES: O=C(CCN1C(=O)C=CC1=O)NCC(CNC(=O)CCN1C(=O)C=CC1=O)(CNC(=O)CCN1C(=O)C=CC1=O)C(=O)NCCOCCOCCNC(=O)OC1Cc2ccccc2C#Cc2ccccc21]

In particular, the overall yield for the synthesis of the core shown above is approximately 0.5%, and takes approximately 14 days for completion of the synthesis.

By contrast, cores according to the present invention (particularly for compounds with body portions of Formula 2) can be synthesised in higher yields (generally up to 60 times more than 0.5%) and at a shorter time scale (generally around 5 days). Furthermore, cores according to the present invention can be synthesised using fewer reaction steps, as well as fewer purification steps (particularly chromatography, such as size exclusion chromatography, and crystallisation). In addition, the use of hazardous chemicals such as hydrazine hydrate, sodium azide and dichloromethane can be avoided using the present cores.

Furthermore, the cores according to the present invention allow higher binding efficiencies to be achieved.

Cores with body portions of Formula 2 may be based on natural amino acids.

In one embodiment, at least one of the first linking group and the second linking group may comprise two or more first linking groups or second linking groups.

In one embodiment, the first linking group may be configured to be linkable to a B-cell epitope. For example, the first linking group may be configured to be linkable to a polypeptide comprising a sequence of a B-cell epitope.

In one embodiment, the core may comprise two or more first linking groups. Preferably, the core may comprise two to four first linking groups. More preferably, the core may two or three first linking groups. Even more preferably, the core may comprise three first linking groups.

In one embodiment, the second linking group may be configured to be linkable to a T-cell epitope. For example, the second linking group may be configured to be linkable to a polypeptide comprising a sequence of a T-cell epitope. Preferably, the T-cell epitope is a CD4+ and/or CD8+ T-cell epitope.

In one embodiment, the core may comprise one to four second linking groups. Preferably, the core may comprise one or two second linking groups. More preferably, the core may comprise one second linking group.

In one embodiment, the core comprises three first linking groups and at least one second linking group. In one embodiment, the three first linking groups are each configured to be linkable to a polypeptide comprising a sequence of a B-cell epitope. In one embodiment, the at least one second linking group is configured to be linkable to a polypeptide comprising a sequence of a CD4+ T-cell epitope.

In one embodiment, the core may additionally comprise a third linking group attached to the body portion, configured to be linkable to a further substance as defined herein. The third linking group may be different to the first linking group and the second linking group. The third linking group may be orthogonal to the first linking group and the second linking group. The core may comprise one to four third linking groups, preferably one third linking group.

In one embodiment, the first linking group and the second linking group may be independently selected from an alkyne (e.g. a terminal alkyne), an alkene (e.g. a terminal alkene, norbornene), a cycloalkyne, a trans-cycloalkene, a tetrazine, a conjugated diene, a maleimide, an α-halocarbonyl, a thiol and an azide; preferably wherein the first linking group and the second linking group are independently selected from an alkyne (e.g. a terminal alkyne), a cycloalkyne, a maleimide and an α-halocarbonyl; more preferably wherein the first linking group and the second linking group are independently selected from an alkyne (e.g. a terminal alkyne), a cycloalkyne and an α-halocarbonyl.

In one embodiment, the third linking group may be independently selected from an alkyne (e.g. a terminal alkyne), an alkene (e.g. a terminal alkene, norbornene), a cycloalkyne, a trans-cycloalkene, a tetrazine, a conjugated diene, a maleimide, an α-halocarbonyl, a thiol and an azide; preferably wherein the first linking group and the second linking group are independently selected from an alkyne (e.g. a terminal alkyne), a cycloalkyne, a maleimide and an α-halocarbonyl; more preferably wherein the third linking group is selected from an alkyne (e.g. a terminal alkyne), a cycloalkyne and an α-halocarbonyl.

In one embodiment, the first linking group and the second linking group may be independently selected from:

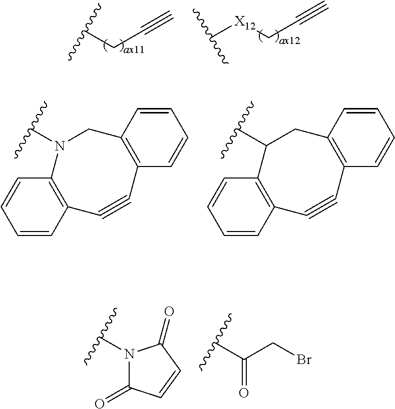

wherein X₁₂ is O, NR₁₂ or S;
R₁₂ is selected from hydrogen, optionally substituted alkyl, optionally substituted cycloalkyl, optionally substituted heterocycloalkyl, optionally substituted aryl and optionally substituted heteroaryl; preferably hydrogen and optionally substituted alkyl; and
ax11 and ax12 are independently selected from 0 to 12.

In one embodiment, the third linking group may be independently selected from:

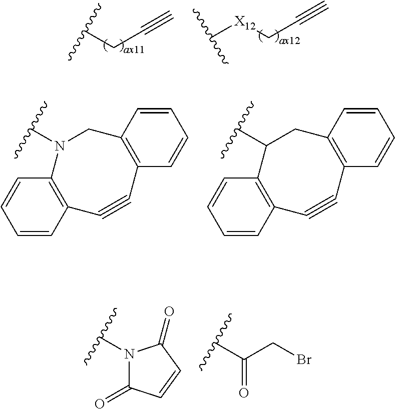

wherein X₁₂ is O, NR₁₂ or S;
R₁₂ is selected from hydrogen, optionally substituted alkyl, optionally substituted cycloalkyl, optionally substituted heterocycloalkyl, optionally substituted aryl and optionally substituted heteroaryl; preferably hydrogen and optionally substituted alkyl; and
ax11 and ax12 are independently selected from 0 to 12.

Preferably, ax11 and ax12 are independently selected from 1 to 12. More preferably, ax11 and ax12 are independently selected from 1 to 6. Even more preferably, ax11 and ax12 are independently selected from 1 to 4.

Preferably, X₁₂ may be O or NH. More preferably, X₁₂ may be O.

In one embodiment, the first linking group may be selected from an alkyne (e.g. a terminal alkyne), a maleimide and an α-halocarbonyl. Preferably, the first linking group may be selected from an alkyne (e.g. a terminal alkyne) and an α-halocarbonyl.

In one embodiment, the first linking group may be selected from:

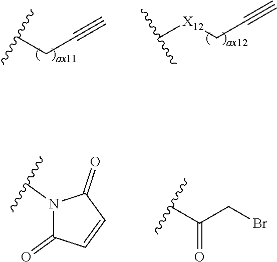

wherein X₁₂, ax11 and ax12 are as defined herein.

Preferably, the first linking group may be selected from

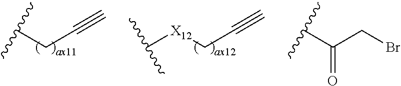

wherein X₁₂, ax11 and ax12 are as defined herein.

In one embodiment, the second linking group may be selected from an alkyne (e.g. a terminal alkyne) and a cycloalkyne.

In one embodiment, the second linking group may be selected from:

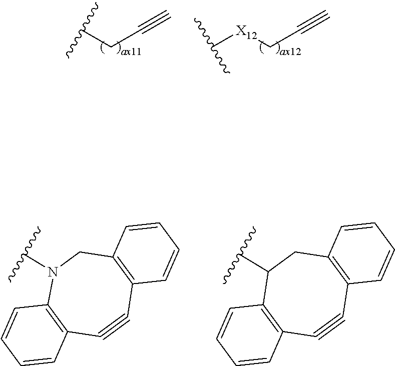

wherein X₁₂, ax11 and ax12 are as defined herein.

In one embodiment, the third linking group may be an alkyne (e.g. a terminal alkyne).

In one embodiment, the third linking group may be selected from:

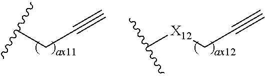

wherein X₁₂, ax11 and ax12 are as defined herein.

In one embodiment, the core may comprise a body portion according to Formula 1:

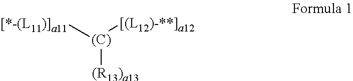

wherein in Formula 1:
Ln and L₁₂ are linkers;
R₁₃ is selected from hydrogen, hydroxy, optionally substituted amino, halogen, optionally substituted alkyl, —S-(optionally substituted alkyl), optionally substituted cycloalkyl, optionally substituted heterocycloalkyl, optionally substituted alkoxy, optionally substituted alkanoyl, optionally substituted aryl and optionally substituted heteroaryl;
a11 represents the number of [(L₁₁)-*] groups attached to the carbon atom and is selected from 1, 2 or 3:
a12 represents the number of [(L₁₂)-**] groups attached to the carbon atom and is selected from 1, 2 or 3;
a13 represents the number of R₁₃ groups attached to the carbon atom and is selected from 0 or 1;
a11+a12+a13 is 4;
* represents a connection point to the first linking group;
** represents a connection point to the second linking group.

The type of linker for L₁₁ and L₁₂ is not particularly limited provided that they are able to connect to the first linking group or the second linking group. Non-limiting examples include alkylene linkages, arylene linkages, PEG linkages, carbonyl-based linkages (e.g. ketones, esters, amides) or other combinations of linkages. In one embodiment, the linker is a non-cleavable linker. In another embodiment, the linker is a cleavable linker.

In one embodiment, L₁₁ and L₁₂ are each independently comprise 1 to 6 units, each unit being independently selected from:
-(optionally substituted alkylene)-, —O—, —(CONH)—, —(NHCO)—, —(CH₂CH₂O)_{w}, —(CO)—, -(optionally substituted alkylene-CO)— and —(CO-optionally substituted alkylene)-,
wherein w is selected from 1 to 6.

In one embodiment, L₁₁ and L₁₂ may each independently comprise 2 to 6 units. Preferably, L₁₁ and L₁₂ may each independently comprise 3 to 6 units. More preferably, L₁₁ and L₁₂ may each independently comprise 4 to 6 units.

In one embodiment, any two adjacent units in L₁₁ and L₁₂ may be different to each other.

In one embodiment, L₁₁ and L₁₂ may be independently selected from -(optionally substituted alkylene)-, —O—, —(CONH)—, —(NHCO)—, —(CH₂CH₂O)_{w}, —(CO)—, -(optionally substituted alkylene)-O—, -(optionally substituted alkylene)-(CONH)—, -(optionally substituted alkylene)-(NHCO)—, -(optionally substituted alkylene)-(CH₂CH₂O)_{w}—, -(optionally substituted alkylene)-(CO)—, —O-(optionally substituted alkylene)-, —O—(CONH)—, —O—(NHCO)—, —O—(CH₂CH₂O)_{w}, —O—(CO)—, —(CONH)-(optionally substituted alkylene)-, —(CONH)—O—, —(CONH)—(NHCO)—, —(CONH)—(CH₂CH₂O)_{w}, —(CONH)—(CO)—, —(NHCO)-(optionally substituted alkylene)-, —(NHCO)—O—, —(NHCO)—(CONH)—, —(NHCO)—(CH₂CH₂O)_{w}, —(NHCO)—(CO)—, —(CH₂CH₂O)-(optionally substituted alkylene)-, —(CH₂CH₂O)_{w}—O—, —(CH₂CH₂O)_{w}—(CONH)—, —(CH₂CH₂O)_{w}—(NHCO)—, —(CH₂CH₂O)_{w}—(CO)—, —(CO)-(optionally substituted alkylene), —(CO)—O—, —(CO)—(CONH)—, —(CO)—(NHCO)—, —(CO)—(CH₂CH₂O)_{w}—, —(CO)-(optionally substituted alkylene)-(CO)—, -(optionally substituted alkylene)-(NHCO)-(optionally substituted alkylene)-, -(optionally substituted alkylene)-(CONH)-(optionally substituted alkylene)-, -(optionally substituted alkylene)-(NHCO)—(CH₂CH₂O)_{w}-(optionally substituted alkylene)-, -(optionally substituted alkylene)-(CONH)—(CH₂CH₂O)_{w}-(optionally substituted alkylene)-, -(optionally substituted alkylene)-O-(optionally substituted alkylene)-(NHCO)—(CH₂CH₂O)_{w}-(optionally substituted alkylene)-, -(optionally substituted alkylene)-O-(optionally substituted alkylene)-(CONH)—(CH₂CH₂O)_{w}-(optionally substituted alkylene)-, —(CONH)—(CH₂CH₂O)_{w}-(optionally substituted alkylene)-(CONH)—O—, —(NHCO)—(CH₂CH₂O)_{w}-(optionally substituted alkylene)-(CONH)—O—, —(CONH)—(CH₂CH₂O)_{w}-(optionally substituted alkylene)-(NHCO)—O—, —(NHCO)—(CH₂CH₂O)_{w}-(optionally substituted alkylene)-(NHCO)—O—, —(CONH)—(CH₂CH₂O)_{w}-(optionally substituted alkylene)-(CONH)-(optionally substituted alkylene)-(CO)—, —(NHCO)—(CH₂CH₂O)_{w}-(optionally substituted alkylene)-(CONH)-(optionally substituted alkylene)-(CO)—, —(CONH)—(CH₂CH₂O)_{w}-(optionally substituted alkylene)-(NHCO)-(optionally substituted alkylene)-(CO)—, —(NHCO)—(CH₂CH₂O)_{w}-(optionally substituted alkylene)-(NHCO)-(optionally substituted alkylene)-(CO)—. Preferably, L₁₁ and L₁₂ may be independently selected from -(optionally substituted alkylene)-O-(optionally substituted alkylene)-(NHCO)—(CH₂CH₂O)_{w}-(optionally substituted alkylene)-, -(optionally substituted alkylene)-O-(optionally substituted alkylene)-(CONH)—(CH₂CH₂O)_{w}-(optionally substituted alkylene)-, —(CONH)—(CH₂CH₂O)_{w}-(optionally substituted alkylene)-(CONH)-(optionally substituted alkylene)-(CO)—, —(NHCO)—(CH₂CH₂O)_{w}-(optionally substituted alkylene)-(CONH)-(optionally substituted alkylene)-(CO)—, —(CONH)—(CH₂CH₂O)_{w}-(optionally substituted alkylene)-(NHCO)-(optionally substituted alkylene)-(CO)—, —(NHCO)—(CH₂CH₂O)_{w}-(optionally substituted alkylene)-(NHCO)-(optionally substituted alkylene)-(CO)—. More preferably, L₁₁ and L₁₂ may be independently selected from -(optionally substituted alkylene)-O-(optionally substituted alkylene)-(CONH)—(CH₂CH₂O)_{w}-(optionally substituted alkylene)-, and —(NHCO)—(CH₂CH₂O)_{w}-(optionally substituted alkylene)-(NHCO)-(optionally substituted alkylene)-(CO)—. The order of units for L₁₁ and L₁₂ from left to right as referred to herein may be read from the carbon atom to the connection point (*) to the first linking group and the connection point (**) to the second linking group respectively.

In one embodiment, L₁₁ may be selected from -(optionally substituted alkylene)-, —O—, —(CONH)—, —(NHCO)—, —(CH₂CH₂O)_{w}—, —(CO)—, -(optionally substituted alkylene)-O—, -(optionally substituted alkylene)-(CONH)—, -(optionally substituted alkylene)-(NHCO)—, -(optionally substituted alkylene)-(CH₂CH₂O)_{w}—, -(optionally substituted alkylene)-(CO)—, —O-(optionally substituted alkylene)-, —O—(CONH)—, —O—(NHCO)—, —O—(CH₂CH₂O)_{w}—, —O—(CO)—, —(CONH)-(optionally substituted alkylene)-, —(CONH)—O—, —(CONH)—(NHCO)—, —(CONH)—(CH₂CH₂O)_{w}, —(CONH)—(CO)—, —(NHCO)-(optionally substituted alkylene)-, —(NHCO)—O—, —(NHCO)—(CONH)—, —(NHCO)—(CH₂CH₂O)_{w}—, —(NHCO)—(CO)—, —(CH₂CH₂O)_{w}-(optionally substituted alkylene)-, —(CH₂CH₂O)_{w} O—, —(CH₂CH₂O)_{w}—(CONH)—, —(CH₂CH₂O)_{w}—(NHCO)—, —(CH₂CH₂O)_{w}—(CO)—, —(CO)-(optionally substituted alkylene), —(CO)—O—, —(CO)—(CONH)—, —(CO)—(NHCO)—, —(CO)—(CH₂CH₂O)_{w}, —(CO)-(optionally substituted alkylene)-(CO)—, -(optionally substituted alkylene)-(NHCO)-(optionally substituted alkylene)-, -(optionally substituted alkylene)-(CONH)-(optionally substituted alkylene)-, -(optionally substituted alkylene)-(NHCO)—(CH₂CH₂O)_{w}-(optionally substituted alkylene)-, -(optionally substituted alkylene)-(CONH)—(CH₂CH₂O)_{w}-(optionally substituted alkylene)-, -(optionally substituted alkylene)-O-(optionally substituted alkylene)-(NHCO)—(CH₂CH₂O)_{w}-(optionally substituted alkylene)-, (optionally substituted alkylene)-O-(optionally substituted alkylene)-(CONH)—(CH₂CH₂O)_{w}-(optionally substituted alkylene)-, —(CONH)—(CH₂CH₂O)_{w}-(optionally substituted alkylene)-(CONH)—O—, —(NHCO)—(CH₂CH₂O)_{w}-(optionally substituted alkylene)-(CONH)—O—, —(CONH)—(CH₂CH₂O)_{w}-(optionally substituted alkylene)-(NHCO)—O—, —(NHCO)—(CH₂CH₂O)_{w}-(optionally substituted alkylene)-(NHCO)—O—, —(CONH)—(CH₂CH₂O)_{w}-(optionally substituted alkylene)-(CONH)-(optionally substituted alkylene)-(CO)—, —(NHCO)—(CH₂CH₂O)_{w}-(optionally substituted alkylene)-(CONH)-(optionally substituted alkylene)-(CO)—, —(CONH)—(CH₂CH₂O)_{w}-(optionally substituted alkylene)-(NHCO)-(optionally substituted alkylene)-(CO)—, —(NHCO)—(CH₂CH₂O)_{w}-(optionally substituted alkylene)-(NHCO)-(optionally substituted alkylene)-(CO)—. Preferably, L₁₁ may be selected from -(optionally substituted alkylene)-O-(optionally substituted alkylene)-(NHCO)—(CH₂CH₂O)_{w}-(optionally substituted alkylene)-, and -(optionally substituted alkylene)-O-(optionally substituted alkylene)-(CONH)—(CH₂CH₂O)_{w}-(optionally substituted alkylene)-. More preferably, Lu may be -(optionally substituted alkylene)-O-(optionally substituted alkylene)-(CONH)—(CH₂CH₂O)_{w}-(optionally substituted alkylene)-.

In one embodiment, L₁₂ may be selected from -(optionally substituted alkylene)-, —O—, —(CONH)—, —(NHCO)—, —(CH₂CH₂O)_{w}, —(CO)—, -(optionally substituted alkylene)-O—, -(optionally substituted alkylene)-(CONH)—, -(optionally substituted alkylene)-(NHCO)—, -(optionally substituted alkylene)-(CH₂CH₂O)_{w}—, -(optionally substituted alkylene)-(CO)—, —O-(optionally substituted alkylene)-, —O—(CONH)—, —O—(NHCO)—, —O—(CH₂CH₂O)_{w}—, —O—(CO)—, —(CONH)-(optionally substituted alkylene)-, —(CONH)—O—, —(CONH)—(NHCO)—, —(CONH)—(CH₂CH₂O)_{w}, —(CONH)—(CO)—, —(NHCO)-(optionally substituted alkylene)-, —(NHCO)—O—, —(NHCO)—(CONH)—, —(NHCO)—(CH₂CH₂O)_{w}—, —(NHCO)—(CO)—, —(CH₂CH₂O)_{w}-(optionally substituted alkylene)-, —(CH₂CH₂O)_{w}—O—, —(CH₂CH₂O)_{w}—(CONH)—, —(CH₂CH₂O)_{w}—(NHCO)—, —(CH₂CH₂O)_{w}—(CO)—, —(CO)-(optionally substituted alkylene), —(CO)—O—, —(CO)—(CONH)—, —(CO)—(NHCO)—, —(CO)—(CH₂CH₂O)_{w}, —(CO)-(optionally substituted alkylene)-(CO)—, -(optionally substituted alkylene)-(NHCO)-(optionally substituted alkylene)-, -(optionally substituted alkylene)-(CONH)-(optionally substituted alkylene)-, -(optionally substituted alkylene)-(NHCO)—(CH₂CH₂O)_{w}-(optionally substituted alkylene)-, -(optionally substituted alkylene)-(CONH)—(CH₂CH₂O)_{w}-(optionally substituted alkylene)-, -(optionally substituted alkylene)-O-(optionally substituted alkylene)-(NHCO)—(CH₂CH₂O)_{w}-(optionally substituted alkylene)-, -(optionally substituted alkylene)-O-(optionally substituted alkylene)-(CONH)—(CH₂CH₂O)_{w}-(optionally substituted alkylene)-, —(CONH)—(CH₂CH₂O)_{w}-(optionally substituted alkylene)-(CONH)—O—, —(NHCO)—(CH₂CH₂O)_{w}-(optionally substituted alkylene)-(CONH)—O—, —(CONH)—(CH₂CH₂O)_{w}-(optionally substituted alkylene)-(NHCO)—O—, —(NHCO)—(CH₂CH₂O)_{w}-(optionally substituted alkylene)-(NHCO)—O—, —(CONH)—(CH₂CH₂O)_{w}-(optionally substituted alkylene)-(CONH)-(optionally substituted alkylene)-(CO)—, —(NHCO)—(CH₂CH₂O)_{w}-(optionally substituted alkylene)-(CONH)-(optionally substituted alkylene)-(CO)—, —(CONH)—(CH₂CH₂O)_{w}-(optionally substituted alkylene)-(NHCO)-(optionally substituted alkylene)-(CO)—, —(NHCO)—(CH₂CH₂O)_{w}-(optionally substituted alkylene)-(NHCO)-(optionally substituted alkylene)-(CO)—. Preferably, L₁₂ may be selected from —(CONH)—(CH₂CH₂O)_{w}-(optionally substituted alkylene)-(CONH)-(optionally substituted alkylene)-(CO)—, —(NHCO)—(CH₂CH₂O)_{w}-(optionally substituted alkylene)-(CONH)-(optionally substituted alkylene)-(CO)—, —(CONH)—(CH₂CH₂O)_{w}-(optionally substituted alkylene)-(NHCO)-(optionally substituted alkylene)-(CO)—, —(NHCO)—(CH₂CH₂O)_{w}-(optionally substituted alkylene)-(NHCO)-(optionally substituted alkylene)-(CO)—. More preferably, L₁₂ may be —(NHCO)—(CH₂CH₂O)_{w}-(optionally substituted alkylene)-(NHCO)-(optionally substituted alkylene)-(CO)—.

In one embodiment, w for L₁₁ and L₁₂ may be independently selected from 1 to 4.

In one embodiment, R₁₃ may be selected from hydrogen, halogen, optionally substituted alkyl and optionally substituted cycloalkyl. Preferably, R₁₃ may be selected from hydrogen, optionally substituted alkyl. More preferably, R₁₃ may be hydrogen.

In one embodiment, a11 may be 2 or 3. Preferably, a11 may be 3.

In one embodiment, a12 may be 1 or 2. Preferably, a12 may be 1.

In one embodiment, a13 may be 0. In another embodiment, a13 may be 1.

In one embodiment, the core may comprise a body portion according to Formula 2:

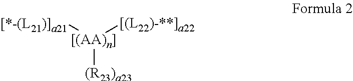

wherein in Formula 2:
AA represents an amino acid;
n represents the number of independently selected AA groups and is selected from 1 to 12;
L₂₁ and L₂₂ are linkers;
R₂₃ is selected from hydrogen, hydroxy, optionally substituted amino, optionally substituted alkyl, —S-(optionally substituted alkyl), optionally substituted cycloalkyl, optionally substituted heterocycloalkyl, optionally substituted alkoxy, optionally substituted aryl and optionally substituted heteroaryl;
a21 represents the number of [(L₂₁)-*] groups attached to [(AA)], and is selected from 1, 2 or 3;
a22 represents the number of [(L₂₂)-**] groups attached to [(AA)], and is selected from 1, 2 or 3;
a23 represents the number of R₂₃ groups attached to [(AA)] n at the C-terminus and/or the N-terminus, and is selected from 0, 1 or 2;
* represents a connection point to the first linking group via the N-terminus, C-terminus or a side-chain of one of the AA groups;
** represents a connection point to the second linking group via the N-terminus, C-terminus or a side-chain of one of the AA groups.

The type of linker for L₂₁ and L₂₂ is not particularly limited provided that they are able to connect to the first linking group or the second linking group. Non-limiting examples include alkylene linkages, arylene linkages, PEG linkages, carbonyl-based linkages (e.g. ketones, esters, amides) or other combinations of linkages. In one embodiment, the linker is a non-cleavable linker. In another embodiment, the linker is a cleavable linker.

In one embodiment, L₂₁ and L₂₂ are each independently comprise 0 to 6 units, each unit being independently selected from:
(optionally substituted alkylene)-, —O—, —(CONH)—, —(NHCO)—, —(CH₂CH₂O)_{w}, —(CO)—, -(optionally substituted alkylene-CO)— and —(CO-optionally substituted alkylene)-,
wherein w is selected from 1 to 6.

Where L₂₁ and/or L₂₂ comprise 0 units, this refers to when the first linking group or the second linking group is directly connected to the N-terminus, C-terminus or a side-chain of one of the AA groups.

In one embodiment, the body portion according to Formula 2 may comprise one or more AA groups having a sidechain comprising an optionally substituted amino group. Preferably, the sidechain comprising an amino group may be a -(optionally substituted alkylene)-(optionally substituted amino) group.

In one embodiment, the sidechain comprising an amino group may comprise one or more lysine groups. Preferably, the body portion according to Formula 2 may comprise one to four lysine groups. More preferably, the body portion according to Formula 2 may comprise three lysine groups. Even more preferably, the body portion according to Formula 2 may consist of three lysine groups (Lys-Lys-Lys), or consist of three lysine groups and a serine group (e.g. Lys-Lys-Lys-Ser).

In one embodiment, n may be selected from 2 to 10. Preferably, n may be selected from 2 to 6. More preferably, n may be selected from 3 to 6. Even more preferably, n may be 3 or 4. Yet even more preferably, n may be 3.

In one embodiment, any two AA groups may be separated by a linker. The type of linker between any two AA groups is not particularly limited provided that they are able to connect to the two AA groups. Non-limiting examples include alkylene linkages, arylene linkages, PEG linkages, carbonyl-based linkages (e.g. ketones, esters, amides) or other combinations of linkages. In one embodiment, the linker is a non-cleavable linker. In another embodiment, the linker is a cleavable linker.

The linker between any two AA groups may comprise 0 to 6 units, each unit being independently selected from:
-(optionally substituted alkylene)-, —O—, —(CONH)—, —(NHCO)—, —(CH₂CH₂O)_{w}, —(CO)—, -(optionally substituted alkylene-CO)— and —(CO-optionally substituted alkylene)-,
wherein w is selected from 1 to 6.

Preferably, the linker between any two AA groups may be selected from -(optionally substituted alkylene)-, —O—, —(CONH)—, —(NHCO)—, —(CH₂CH₂O)_{w}, —(CO)—, -(optionally substituted alkylene)-O—, -(optionally substituted alkylene)-(CONH)—, -(optionally substituted alkylene)-(NHCO)—, -(optionally substituted alkylene)-(CH₂CH₂O)_{w}—, -(optionally substituted alkylene)-(CO)—, —O-(optionally substituted alkylene)-, —O—(CONH)—, O—(NHCO)—, —O—(CH₂CH₂O)_{w}, —O—(CO)—, —(CONH)-(optionally substituted alkylene)-, —(CONH)—O—, —(CONH)—(NHCO)—, —(CONH)—(CH₂CH₂O)_{w}—, —(CONH)—(CO)_{w}—(NHCO)-(optionally substituted alkylene)-, —(NHCO)—O—, —(NHCO)—(CONH)—, —(NHCO)—(CH₂CH₂O)_{w}, —(NHCO)—(CO)—, —(CH₂CH₂O)_{w}-(optionally substituted alkylene)-, —(CH₂CH₂O)_{w}—O—, —(CH₂CH₂O)_{w}—(CONH)—, —(CH₂CH₂O)_{w}—(NHCO)—, —(CH₂CH₂O)_{w}—(CO)—, —(CO)-(optionally substituted alkylene), —(CO)—O—, —(CO)—(CONH)—, —(CO)—(NHCO)—, —(CO)—(CH₂CH₂O)_{w}—(CO)-(optionally substituted alkylene)-(CO)—, -(optionally substituted alkylene)-(NHCO)-(optionally substituted alkylene)-, -(optionally substituted alkylene)-(CONH)-(optionally substituted alkylene)-, -(optionally substituted alkylene)-(NHCO)—(CH₂CH₂O)_{w}-(optionally substituted alkylene)-, -(optionally substituted alkylene)-(CONH)—(CH₂CH₂O)_{w}-(optionally substituted alkylene)-, -(optionally substituted alkylene)-O-(optionally substituted alkylene)-(NHCO)—(CH₂CH₂O)_{w}-(optionally substituted alkylene)-, -(optionally substituted alkylene)-O-(optionally substituted alkylene)-(CONH)—(CH₂CH₂O)_{w}-(optionally substituted alkylene)-, —(CONH)—(CH₂CH₂O)_{w}-(optionally substituted alkylene)-(CONH)—O—, —(NHCO)—(CH₂CH₂O)_{w}-(optionally substituted alkylene)-(CONH)—O—, —(CONH)—(CH₂CH₂O)_{w}-(optionally substituted alkylene)-(NHCO)—O—, —(NHCO)—(CH₂CH₂O)_{w}-(optionally substituted alkylene)-(NHCO)—O—, —(CONH)—(CH₂CH₂O)_{w}-(optionally substituted alkylene)-(CONH)-(optionally substituted alkylene)-(CO)—, —(NHCO)—(CH₂CH₂O)_{w}-(optionally substituted alkylene)-(CONH)-(optionally substituted alkylene)-(CO)—, —(CONH)—(CH₂CH₂O)_{w}-(optionally substituted alkylene)-(NHCO)-(optionally substituted alkylene)-(CO)—, —(NHCO)—(CH₂CH₂O)_{w}-(optionally substituted alkylene)-(NHCO)-(optionally substituted alkylene)-(CO)—.

In one embodiment, any two AA groups may be directly connected to each other.

In one embodiment, any two AA groups in all of “(AA)ₙ” are directly connected to each other.

In one embodiment, a carbonyl moiety of one AA group may be connected to an amine moiety of another AA group, a carbonyl moiety of one AA group may be connected via a side chain of another AA group, an amine moiety of one AA group may be connected via a side chain of one another AA group, or a side chain of one AA group connected via a side chain of another AA group (optionally with a linker between the one AA group and the another AA group as defined herein).

In one embodiment, “(AA) n” may be a linear sequence of AA groups, wherein each AA group is connected to the next AA group such that a carbonyl moiety of one AA group is connected to an amine moiety of another AA group (optionally with a linker between the one AA group and the another AA group as defined herein). In some embodiments, the linear sequence of AA groups may be connected such that any two AA groups are directly connected to each other (i.e. without a linker between any two AA groups).

In one embodiment, L₂₁ and L₂₂ may each independently comprise 0 to 5 units. Preferably, L₂₁ and L₂₂ may each independently comprise 0 to 3 units.

In one embodiment, any two adjacent units in L₂₁ and L₂₂ may be different to each other.

In one embodiment, each unit in L₂₁ and L₂₂ may be independently selected from (optionally substituted alkylene)- and —(CO)—.

In one embodiment, L₂₁ and L₂₂ may be independently selected from -(optionally substituted alkylene)-, —O—, —(CONH)—, —(NHCO)—, —(CH₂CH₂O)_{w}, —(CO)—, -(optionally substituted alkylene)-O—, -(optionally substituted alkylene)-(CONH)—, -(optionally substituted alkylene)-(NHCO)—, -(optionally substituted alkylene)-(CH₂CH₂O)_{w}, -(optionally substituted alkylene)-(CO)—, —O-(optionally substituted alkylene)-, —O—(CONH)—, —O—(NHCO)—, —O—(CH₂CH₂O)_{w}—, —O—(CO)—, —(CONH)-(optionally substituted alkylene)-, —(CONH)—O—, —(CONH)—(NHCO)—, —(CONH)—(CH₂CH₂O)_{w}, —(CONH)—(CO)—, —(NHCO)-(optionally substituted alkylene)-, —(NHCO)—O—, —(NHCO)—(CONH)—, —(NHCO)—(CH₂CH₂O)_{w}—, —(NHCO)—(CO)—, —(CH₂CH₂O)_{w}-(optionally substituted alkylene)-, —(CH₂CH₂O)_{w}—O—, —(CH₂CH₂O)_{w}—(CONH)—, —(CH₂CH₂O)—(NHCO)—, —(CH₂CH₂O), —(CO)—, —(CO)-(optionally substituted alkylene), —(CO)—O—, —(CO)—(CONH)—, —(CO)—(NHCO)—, —(CO)—(CH₂CH₂O)_{w}—(CO)-(optionally substituted alkylene)-(CO)—, -(optionally substituted alkylene)-(NHCO)-(optionally substituted alkylene)-, -(optionally substituted alkylene)-(CONH)-(optionally substituted alkylene)-, -(optionally substituted alkylene)-(NHCO)—(CH₂CH₂O)_{w}-(optionally substituted alkylene)-, -(optionally substituted alkylene)-(CONH)—(CH₂CH₂O)_{w}-(optionally substituted alkylene)-, -(optionally substituted alkylene)-O-(optionally substituted alkylene)-(NHCO)—(CH₂CH₂O)_{w}-(optionally substituted alkylene)-, -(optionally substituted alkylene)-O-(optionally substituted alkylene)-(CONH)—(CH₂CH₂O)_{w}-(optionally substituted alkylene)-, —(CONH)—(CH₂CH₂O)_{w}-(optionally substituted alkylene)-(CONH)—O—, —(NHCO)—(CH₂CH₂O)_{w}-(optionally substituted alkylene)-(CONH)—O—, —(CONH)—(CH₂CH₂O)_{w}-(optionally substituted alkylene)-(NHCO)—O—, —(NHCO)—(CH₂CH₂O)_{w}-(optionally substituted alkylene)-(NHCO)—O—, —(CONH)—(CH₂CH₂O)_{w}-(optionally substituted alkylene)-(CONH)-(optionally substituted alkylene)-(CO)—, —(NHCO)—(CH₂CH₂O)_{w}-(optionally substituted alkylene)-(CONH)-(optionally substituted alkylene)-(CO)—, —(CONH)—(CH₂CH₂O)_{w}-(optionally substituted alkylene)-(NHCO)-(optionally substituted alkylene)-(CO)—, —(NHCO)—(CH₂CH₂O)_{w}-(optionally substituted alkylene)-(NHCO)-(optionally substituted alkylene)-(CO)—. Preferably, L₂₁ and L₂₂ may be independently selected from —(CO)—, —(CO)-(optionally substituted alkylene)-, and —(CO)-(optionally substituted alkylene)-(CO)—. The order of units for L₂₁ and L₂₂ from left to right as referred to herein may be read from the (AA)ₙ group to the connection point (*) to the first linking group and the connection point (**) to the second linking group respectively.

In one embodiment, L₂₁ may be selected from -(optionally substituted alkylene)-, —O—, —(CONH)—, —(NHCO)—, —(CH₂CH₂O)_{w}, —(CO)—, -(optionally substituted alkylene)-O—, -(optionally substituted alkylene)-(CONH)—, -(optionally substituted alkylene)-(NHCO)—, -(optionally substituted alkylene)-(CH₂CH₂O)_{w}—, -(optionally substituted alkylene)-(CO)—, —O-(optionally substituted alkylene)-, —O—(CONH)—, —O—(NHCO)—, —O—(CH₂CH₂O)_{w}—, —O—(CO)—, —(CONH)-(optionally substituted alkylene)-, —(CONH)—O—, —(CONH)—(NHCO)—, —(CONH)—(CH₂CH₂O)_{w}, —(CONH)—(CO)—, —(NHCO)-(optionally substituted alkylene)-, —(NHCO)—O—, —(NHCO)—(CONH)—, —(NHCO)—(CH₂CH₂O)_{w}—, —(NHCO)—(CO)—, —(CH₂CH₂O)_{w}-(optionally substituted alkylene)-, —(CH₂CH₂O)_{w}—O—, —(CH₂CH₂O)_{w}—(CONH)—, —(CH₂CH₂O)_{w}—(NHCO)—, —(CH₂CH₂O)_{w}—(CO)—, —(CO)-(optionally substituted alkylene), —(CO)—O—, —(CO)—(CONH)—, —(CO)—(NHCO)—, —(CO)—(CH₂CH₂O)_{w}—, —(CO)-(optionally substituted alkylene)-(CO)—, -(optionally substituted alkylene)-(NHCO)-(optionally substituted alkylene)-, -(optionally substituted alkylene)-(CONH)-(optionally substituted alkylene)-, -(optionally substituted alkylene)-(NHCO)—(CH₂CH₂O)_{w}-(optionally substituted alkylene)-, -(optionally substituted d alkylene)-(CONH)—(CH₂CH₂O)_{w}-(optionally substituted alkylene)-, -(optionally substituted alkylene)-O-(optionally substituted alkylene)-(NHCO)—(CH₂CH₂O)_{w}-(optionally substituted alkylene)-, -(optionally substituted alkylene)-O-(optionally substituted alkylene)-(CONH)—(CH₂CH₂O)_{w}-(optionally substituted alkylene)-, —(CONH)—(CH₂CH₂O)_{w}-(optionally substituted alkylene)-(CONH)—O—, —(NHCO)—(CH₂CH₂O)_{w}-(optionally substituted alkylene)-(CONH)—O—, —(CONH)—(CH₂CH₂O)_{w}-(optionally substituted alkylene)-(NHCO)—O—, —(NHCO)—(CH₂CH₂O)_{w}-(optionally substituted alkylene)-(NHCO)—O—, —(CONH)—(CH₂CH₂O)_{w}-(optionally substituted alkylene)-(CONH)-(optionally substituted alkylene)-(CO)—, —(NHCO)—(CH₂CH₂O)_{w}-(optionally substituted alkylene)-(CONH)-(optionally substituted alkylene)-(CO)—, —(CONH)—(CH₂CH₂O)_{w}-(optionally substituted alkylene)-(NHCO)-(optionally substituted alkylene)-(CO)—, —(NHCO)—(CH₂CH₂O)_{w}-(optionally substituted alkylene)-(NHCO)-(optionally substituted alkylene)-(CO)—. Preferably, L₂₁ may be selected from —(CO)— and —(CO)-(optionally substituted alkylene)-.

In one embodiment, L₂₂ may be selected from -(optionally substituted alkylene)-, —O—, —(CONH)—, —(NHCO)—, —(CH₂CH₂O)_{w}—, —(CO)—, -(optionally substituted alkylene)-O—, -(optionally substituted alkylene)-(CONH)—, -(optionally substituted alkylene)-(NHCO)—, -(optionally substituted alkylene)-(CH₂CH₂O)_{w}, -(optionally substituted alkylene)-(CO)—, —O-(optionally substituted alkylene)-, —O—(CONH)—, —O—(NHCO)—, —O—(CH₂CH₂O)_{w}, —O—(CO)—, —(CONH)-(optionally substituted alkylene)-, —(CONH)—O—, —(CONH)—(NHCO)—, —(CONH)—(CH₂CH₂O)_{w}—, —(CONH)—(CO)—, —(NHCO)-(optionally substituted alkylene)-, —(NHCO)—O—, —(NHCO)—(CONH)—, —(NHCO)—(CH₂CH₂O)_{w}, —(NHCO)—(CO)—, —(CH₂CH₂O)_{w}-(optionally substituted alkylene)-, —(CH₂CH₂O)_{w}—O—, —(CH₂CH₂O)_{w}—(CONH)—, —(CH₂CH₂O)_{w}—(NHCO)—, —(CH₂CH₂O)_{w}—(CO)—, —(CO)-(optionally substituted alkylene), —(CO)—O—, —(CO)—(CONH)—, —(CO)—(NHCO)—, —(CO)—(CH₂CH₂O)_{w}, —(CO)-(optionally substituted alkylene)-(CO)—, -(optionally substituted alkylene)-(NHCO)-(optionally substituted alkylene)-, -(optionally substituted alkylene)-(CONH)-(optionally substituted alkylene)-, -(optionally substituted alkylene)-(NHCO)—(CH₂CH₂O)_{w}-(optionally substituted alkylene)-, -(optionally substituted alkylene)-(CONH)—(CH₂CH₂O)_{w}-(optionally substituted alkylene)-, -(optionally substituted alkylene)-O-(optionally substituted alkylene)-(NHCO)—(CH₂CH₂O)_{w}-(optionally substituted alkylene)-, -(optionally substituted alkylene)-O-(optionally substituted alkylene)-(CONH)—(CH₂CH₂O)_{w}-(optionally substituted alkylene)-, —(CONH)—(CH₂CH₂O)_{w}-(optionally substituted alkylene)-(CONH)—O—, —(NHCO)—(CH₂CH₂O)_{w}-(optionally substituted alkylene)-(CONH)—O—, —(CONH)—(CH₂CH₂O)_{w}-(optionally substituted alkylene)-(NHCO)—O—, —(NHCO)—(CH₂CH₂O)_{w}-(optionally substituted alkylene)-(NHCO)—O—, —(CONH)—(CH₂CH₂O)_{w}-(optionally substituted alkylene)-(CONH)-(optionally substituted alkylene)-(CO)—, —(NHCO)—(CH₂CH₂O)_{w}-(optionally substituted alkylene)-(CONH)-(optionally substituted alkylene)-(CO)—, —(CONH)—(CH₂CH₂O)_{w}-(optionally substituted alkylene)-(NHCO)-(optionally substituted alkylene)-(CO)—, —(NHCO)—(CH₂CH₂O)_{w}-(optionally substituted alkylene)-(NHCO)-(optionally substituted alkylene)-(CO)—. Preferably, L₂₂ may be selected from —(CO)— and —(CO)-(optionally substituted alkylene)-(CO)—.

In one embodiment, w for L₂₁ and L₂₂ may be independently selected from 1 to 4.

In one embodiment, an R₂₃ group connected to the C-terminus may be selected from hydroxy, optionally substituted amino and optionally substituted alkoxy. Preferably, an R₂₃ group connected to the C-terminus may be selected from hydroxy and optionally substituted amino. More preferably, an R₂₃ group connected to the C-terminus may be optionally substituted amino. Even more preferably, an R₂₃ group connected to the C-terminus may be —NH₂.

In one embodiment, an R₂₃ group connected to the N-terminus may be selected from hydrogen, optionally substituted alkyl, optionally substituted cycloalkyl, optionally substituted heterocycloalkyl, optionally substituted aryl and optionally substituted heteroaryl. Preferably, an R₂₃ group connected to the N-terminus may be selected from hydrogen and optionally substituted alkyl. More preferably, an R₂₃ group connected to the N-terminus may be hydrogen.

In one embodiment, a21 may be 2 or 3. Preferably, a21 may be 3.

In one embodiment, a22 may be 1 or 2. Preferably, a22 may be 1.

In one embodiment, a23 may be 0. In another embodiment, a23 may be 1.

In one embodiment, the connection point to the first linking group may be via the side chain of one of the AA groups. In embodiments where more than one first linking group is present, at least one connection point to the first linking group(s) may be via the side chain of at least one of the AA groups. Preferably, where more than one first linking group is present, each of the connection points to the first linking groups is via the side chain of independently selected AA groups. In some embodiments, an individual AA group may be connected to up to one first linking group.

In one embodiment, the connection point to the second linking group may be via the C-terminus or the N-terminus. Preferably, the connection point to the second linking group may be via the N-terminus.

When present, a connection point to the third linking group may be via the side chain of one of the AA groups (e.g. at a serine group), or via the C-terminus. Preferably, the connection point to the third linking group may be via the C-terminus.

When present, the third linking group may be connected directly to the body portion. In other embodiments, the third linking group may be connected indirectly to the body portion via a linker. In such a case, the type of linker for the third linking group is not particularly limited provided that the linker is able to connect (covalently) the third linking group to the body portion. Non-limiting examples include alkylene linkages, arylene linkages, PEG linkages, carbonyl-based linkages (e.g. ketones, esters, amides) or other combinations of linkages. In one embodiment, the linker is a non-cleavable linker. In another embodiment, the linker is a cleavable linker.

In one embodiment, the linker for the third linking group may be selected from -(optionally substituted alkylene)-, —O—, —(CONH)—, —(NHCO)—, —(CH₂CH₂O)_{w}, —(CO)—, -(optionally substituted alkylene)-O—, —(NH)-(optionally substituted alkylene)-, -(optionally substituted alkylene)-(CONH)—, -(optionally substituted alkylene)-(NHCO)—, -(optionally substituted alkylene)-(CH₂CH₂O)_{w}, -(optionally substituted alkylene)-(CO)—, —O-(optionally substituted alkylene)-, —O—(CONH)—, —O—(NHCO)—, —O—(CH₂CH₂O)_{w}—, —O—(CO)—, —(CONH)-(optionally substituted alkylene)-, —(CONH)—O—, —(CONH)—(NHCO)—, —(CONH)—(CH₂CH₂O)_{w}, —(CONH)—(CO)—, —(NHCO)-(optionally substituted alkylene)-, —(NHCO)—O—, (NHCO)—(CONH)—, —(NHCO)—(CH₂CH₂O)_{w}, —(NHCO)—(CO)—, —(CH₂CH₂O)_{w}-(optionally substituted alkylene)-, —(CH₂CH₂O)_{w}—O—, —(CH₂CH₂O)_{w}—(CONH)—, —(CH₂CH₂O)_{w}—(NHCO)—, —(CH₂CH₂O)_{w}—(CO)—, —(CO)-(optionally substituted alkylene), —(CO)—O—, —(CO)—(CONH)—, (CO)—(NHCO)—, —(CO)—(CH₂CH₂O)_{w}—, —(CO)-(optionally substituted alkylene)-(CO)—, -(optionally substituted alkylene)-(NHCO)-(optionally substituted alkylene)-, -(optionally substituted alkylene)-(CONH)-(optionally substituted alkylene)-, -(optionally substituted alkylene)-(NHCO)—(CH₂CH₂O)_{w}-(optionally substituted alkylene)-, -(optionally substituted alkylene)-(CONH)—(CH₂CH₂O)_{w}-(optionally substituted alkylene)-, -(optionally substituted alkylene)-O-(optionally substituted alkylene)-(NHCO)—(CH₂CH₂O)_{w}-(optionally substituted alkylene)-, -(optionally substituted alkylene)-O-(optionally substituted alkylene)-(CONH)—(CH₂CH₂O)_{w}-(optionally substituted alkylene)-, (CONH)—(CH₂CH₂O)_{w}-(optionally substituted alkylene)-(CONH)—O—, —(NHCO)—(CH₂CH₂O)_{w}-(optionally substituted alkylene)-(CONH)—O—, —(CONH)—(CH₂CH₂O)_{w}-(optionally substituted alkylene)-(NHCO)—O—, —(NHCO)—(CH₂CH₂O)_{w}-(optionally substituted alkylene)-(NHCO)—O—, —(CONH)—(CH₂CH₂O)_{w}-(optionally substituted alkylene)-(CONH)-(optionally substituted alkylene)-(CO)—, —(NHCO)—(CH₂CH₂O)_{w}-(optionally substituted alkylene)-(CONH)-(optionally substituted alkylene)-(CO)—, —(CONH)—(CH₂CH₂O)_{w}-(optionally substituted alkylene)-(NHCO)-(optionally substituted alkylene)-(CO)—, —(NHCO)—(CH₂CH₂O)_{w}-(optionally substituted alkylene)-(NHCO)-(optionally substituted alkylene)-(CO)—. Preferably, the linker to the third linking group may be —(NH)-(optionally substituted alkylene)-. The order of units from left to right for the third linking group as referred to herein may be read from the (AA) n group to the connection point to the third linking group.

In one embodiment, the core is selected from:

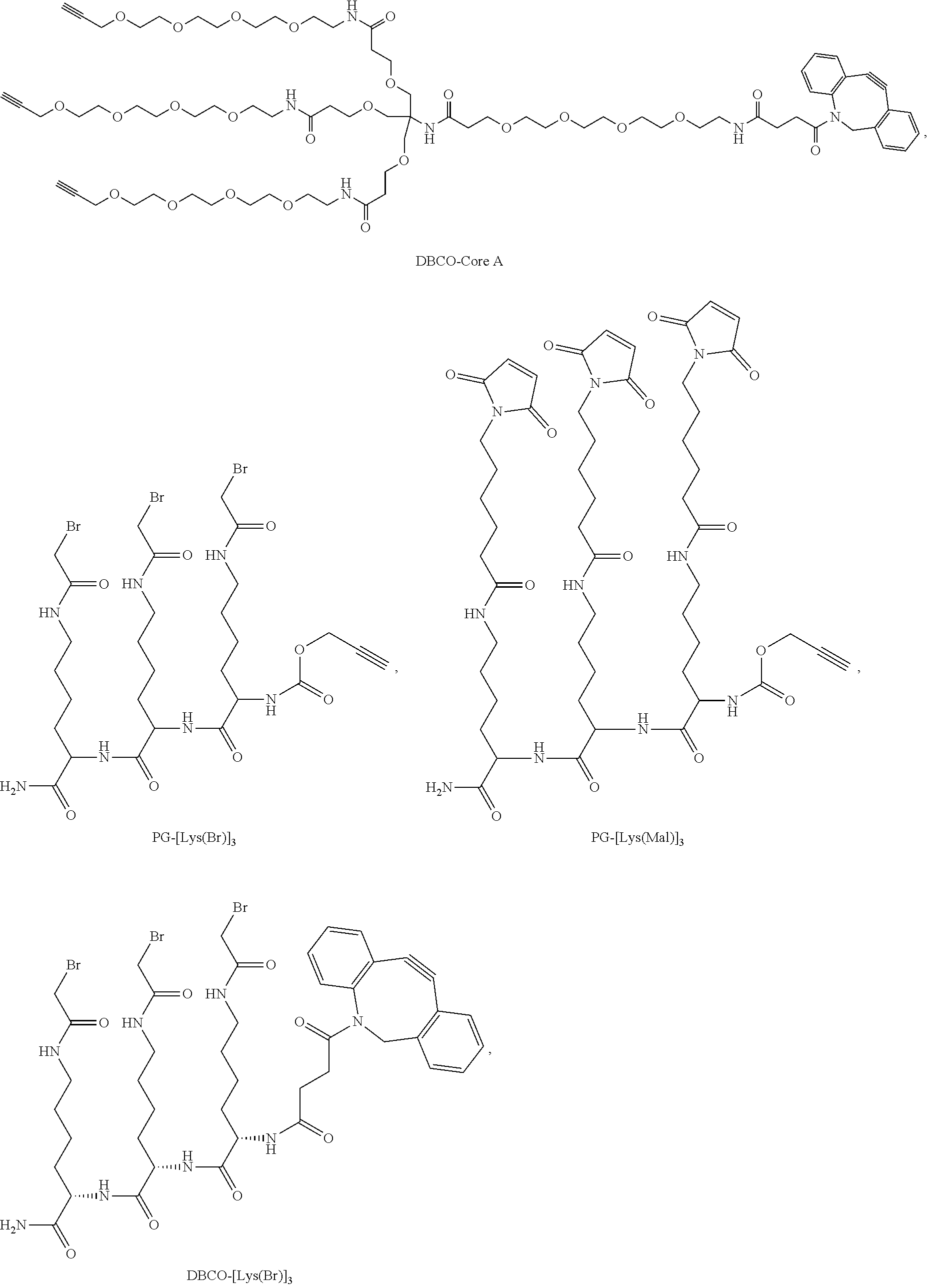 [SMILES: C#CCOCCOCCOCCOCCNC(=O)CCOCC(COCCC(=O)NCCOCCOCCOCCOCC#C)(COCCC(=O)NCCOCCOCCOCCOCC#C)NC(=O)CCOCCOCCOCCOCCNC(=O)CCC(=O)N1Cc2ccccc2C#Cc2ccccc21 ; C#CCOC(=O)NC(CCCCNC(=O)CBr)C(=O)NC(CCCCNC(=O)CBr)C(=O)NC(CCCCNC(=O)CBr)C(N)=O ; C#CCOC(=O)NC(CCCCNC(=O)CCCCCN1C(=O)C=CC1=O)C(=O)NC(CCCCNC(=O)CCCCCN1C(=O)C=CC1=O)C(=O)NC(CCCCNC(=O)CCCCCN1C(=O)C=CC1=O)C(N)=O ; NC(=O)[C@H](CCCCNC(=O)CBr)NC(=O)[C@H](CCCCNC(=O)CBr)NC(=O)[C@H](CCCCNC(=O)CBr)NC(=O)CCC(=O)N1Cc2ccccc2C#Cc2ccccc21]

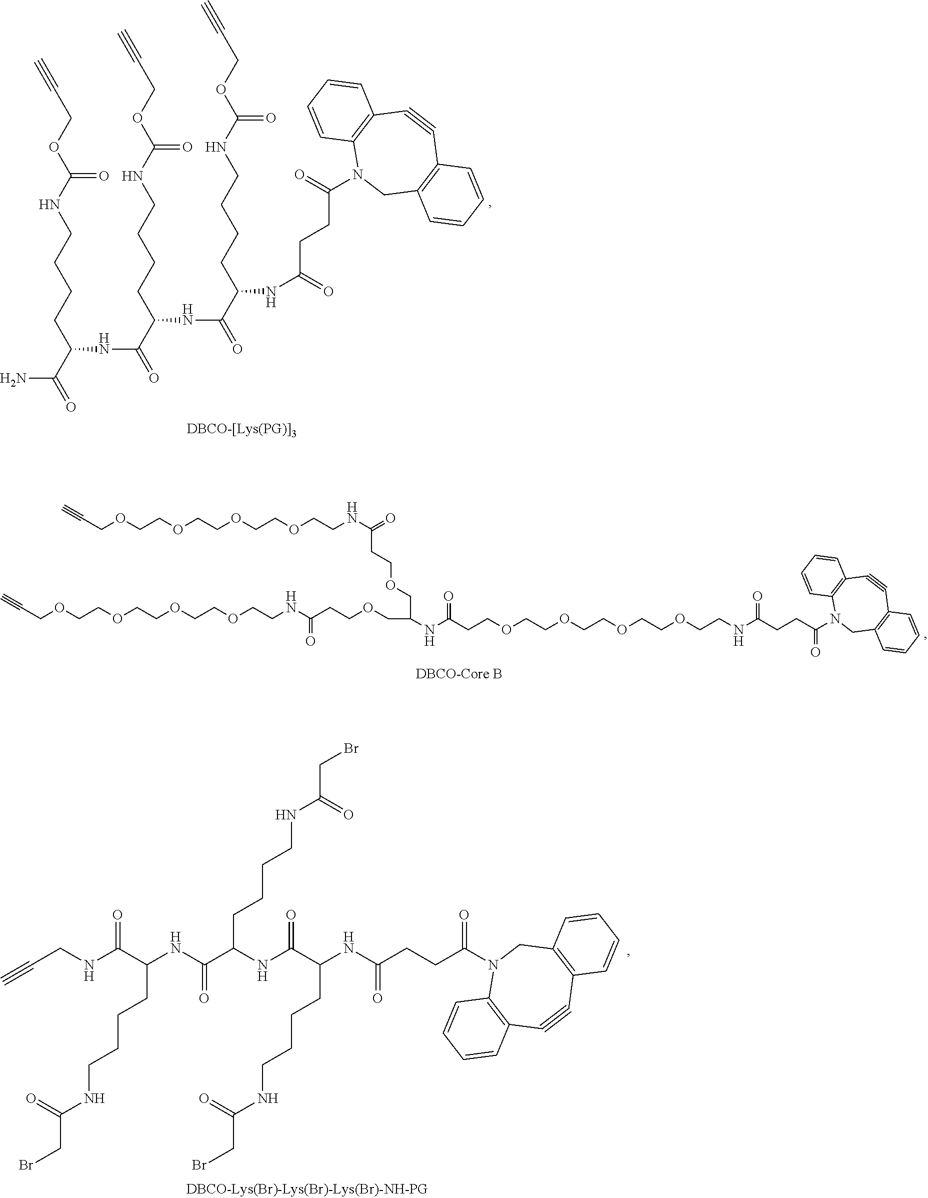 [SMILES: C#CCOC(=O)NCCCC[C@H](NC(=O)[C@H](CCCCNC(=O)OCC#C)NC(=O)[C@H](CCCCNC(=O)OCC#C)NC(=O)CCC(=O)N1Cc2ccccc2C#Cc2ccccc21)C(N)=O ; C#CCOCCOCCOCCOCCNC(=O)CCOCC(COCCC(=O)NCCOCCOCCOCCOCC#C)NC(=O)CCOCCOCCOCCOCCNC(=O)CCC(=O)N1Cc2ccccc2C#Cc2ccccc21 ; C#CCNC(=O)C(CCCCNC(=O)CBr)NC(=O)C(CCCCNC(=O)CBr)NC(=O)C(CCCCNC(=O)CBr)NC(=O)CCC(=O)N1Cc2ccccc2C#Cc2ccccc21]

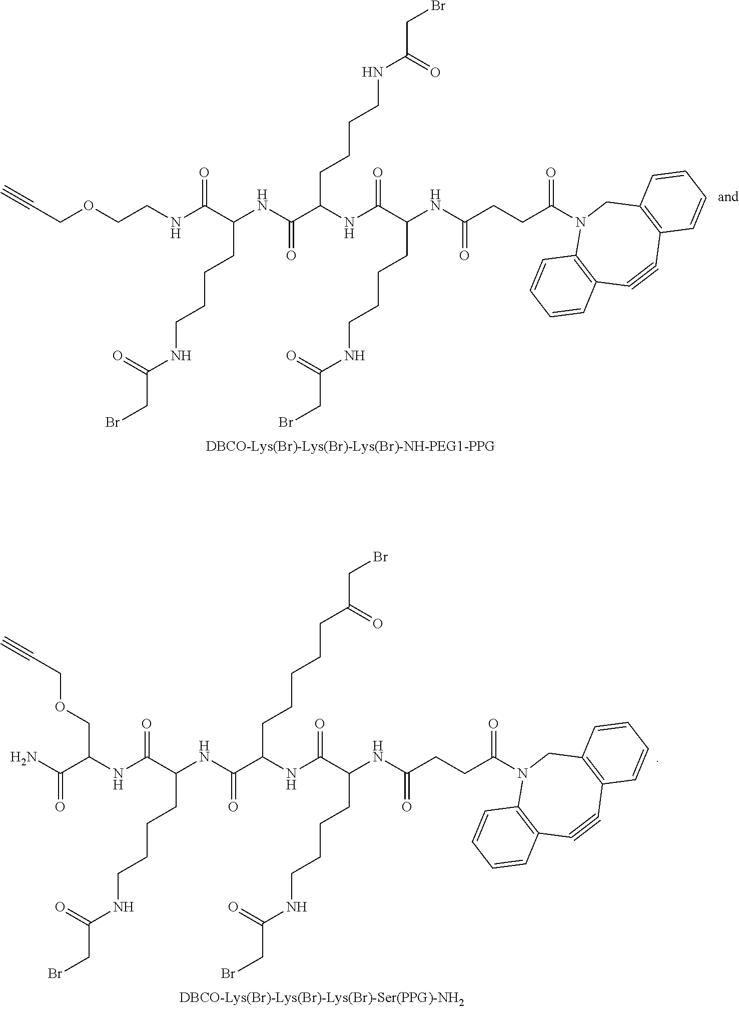 [SMILES: C#CCOCCNC(=O)C(CCCCNC(=O)CBr)NC(=O)C(CCCCNC(=O)CBr)NC(=O)C(CCCCNC(=O)CBr)NC(=O)CCC(=O)N1Cc2ccccc2C#Cc2ccccc21 ; C#CCOCC(NC(=O)C(CCCCNC(=O)CBr)NC(=O)C(CCCCCC(=O)CBr)NC(=O)C(CCCCNC(=O)CBr)NC(=O)CCC(=O)N1Cc2ccccc2C#Cc2ccccc21)C(N)=O]

### A Process for Manufacture

In accordance with another aspect of the invention, there is provided a process for manufacturing a conjugate, comprising the steps of:
(a) providing at least one polypeptide comprising a sequence of a B-cell epitope; and
(b) providing at least one polypeptide comprising a sequence of a CD4+ T-cell epitope and linking the at least one polypeptide comprising a sequence of a CD4+ T-cell epitope to the at least one polypeptide comprising a sequence of a B-cell epitope,
   wherein the CD4+ T-cell epitope comprises a region of at least 12 amino acids of a universal tumour antigen or a sequence having at least 80% sequence identity to the region and wherein the CD4+ T-cell epitope is immunogenic in at least 50% of the population, wherein the at least one polypeptide comprising the sequence of the CD4+ T-cell epitope is equal to or less than 500 amino acids in length,
   wherein the sequence of the B-cell epitope is different from the sequence of the CD4+ T-cell epitope, and
   wherein an antibody specific for the B-cell epitope binds to the conjugate.

In one embodiment, the at least one polypeptide comprising a sequence of a B-cell epitope and the at least one polypeptide comprising a sequence of a CD4+ T-cell epitope are linked via a core,
wherein the core comprises, prior to linkage:
   a body portion;
   one or more first linking groups attached to the body portion; and
   one or more second linking groups attached to the body portion,
   wherein the first linking group and second linking group are orthogonal to each other;
and wherein the first linking group is linked to the at least one polypeptide comprising a sequence of a B-cell epitope to form a first connecting element, and the second linking group is linked to the at least one polypeptide comprising a sequence of a CD4+ T-cell epitope to form a second connecting element.

In accordance with another aspect of the invention, there is provided a process for manufacturing a conjugate, comprising the steps of:
(a) providing a core comprising:
   a body portion;
   one or more first linking groups attached to the body portion; and
   one or more second linking groups attached to the body portion,
   wherein the first linking group and second linking group are orthogonal to each other,
   at least one of the first linking group and the second linking group comprises two or more first linking groups or second linking groups, and
   the first linking group and the second linking group are independently selected from an alkyne (e.g. a terminal alkyne), an alkene (e.g. a terminal alkene, norbornene), a cycloalkyne, a trans-cycloalkene, a tetrazine, a conjugated diene, a maleimide, an α-halocarbonyl, a thiol and an azide; preferably wherein the first linking group and the second linking group are independently selected from an alkyne (e.g. a terminal alkyne), a cycloalkyne, a maleimide and an α-halocarbonyl; more preferably wherein the first linking group and the second linking group are independently selected from an alkyne (e.g. a terminal alkyne), a cycloalkyne and an α-halocarbonyl;
   wherein the core is not:

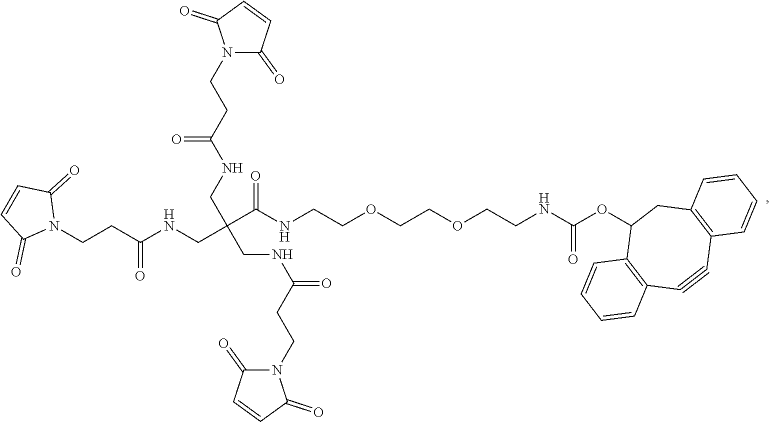 [SMILES: O=C(CCN1C(=O)C=CC1=O)NCC(CNC(=O)CCN1C(=O)C=CC1=O)(CNC(=O)CCN1C(=O)C=CC1=O)C(=O)NCCOCCOCCNC(=O)OC1Cc2ccccc2C#Cc2ccccc21]

preferably wherein the core is as defined herein;
(b) providing at least one polypeptide comprising a sequence of a B-cell epitope, or at least one polypeptide comprising a sequence of a CD4+ T-cell epitope; and reacting the core with the at least one polypeptide comprising a sequence of a B-cell epitope to form a first connecting element, or reacting the core with the at least one polypeptide comprising a sequence of a CD4+ T-cell epitope to form a second connecting element. Other preferred embodiments with regard to the at least one polypeptide comprising a sequence of a B-cell epitope, and/or the at least one polypeptide comprising a sequence of a CD4+ T-cell epitope have been described above and apply equally to the process. Other preferred embodiments with regard to the core are described in further detail above and apply equally to the process.

In one embodiment, the process may further comprise the step of:
(c) providing the other of at least one polypeptide comprising a sequence of a CD4+ T-cell epitope, or at least one polypeptide comprising a sequence of a B-cell epitope not provided in step (b); and reacting the core with the at least one polypeptide comprising a sequence of a CD4+ T-cell epitope to form a second connecting element if the first connecting element was formed in step (b); or reacting the core with the at least one polypeptide comprising a sequence of a B-cell epitope to form a first connecting element if the second connecting element was formed in step (b).

In one embodiment, step (b) may involve the formation of a non-covalent linkage or a covalent linkage. Preferably, step (b) may involve formation of a covalent linkage.

In one embodiment, step (b) may involve a click reaction. Preferably, the reaction involves a cycloaddition reaction (e.g. 1,3-dipolar cycloaddition, including copper-catalysed azide-alkyne cycloaddition and strain-promoted azide-alkyne cycloaddition or azide-alkene cycloaddition; tetrazine ligation, such as with tetrazines and cycloalkynes or trans-cycloalkenes; Diels-Alder cycloaddition reactions), nucleophilic substitution, a Michael reaction, or other examples of click reactions.

In one embodiment, step (c) may involve the formation of a non-covalent linkage or a covalent linkage. Preferably, step (c) may involve formation of a covalent linkage.

In one embodiment, step (c) may involve a click reaction. Preferably, the reaction involves a cycloaddition reaction (e.g. 1,3-dipolar cycloaddition, including copper-catalysed azide-alkyne cycloaddition and strain-promoted azide-alkyne cycloaddition or azide-alkene cycloaddition; tetrazine ligation, such as with tetrazines and cycloalkynes or trans-cycloalkenes; Diels-Alder cycloaddition reactions), nucleophilic substitution, a Michael reaction, or other examples of click reactions.

In one embodiment, where the core comprises a third linking group, the process may further comprise a step of:
(d) providing a further substance as defined herein and reacting the core with the further substance to form a third connecting element.

In one embodiment, step (d) may be conducted after step (c).

In one embodiment, step (d) may involve the formation of a non-covalent linkage or a covalent linkage. Preferably, step (d) may involve formation of a covalent linkage.

In one embodiment, step (d) may involve a click reaction. Preferably, the reaction involves a cycloaddition reaction (e.g. 1,3-dipolar cycloaddition, including copper-catalysed azide-alkyne cycloaddition and strain-promoted azide-alkyne cycloaddition or azide-alkene cycloaddition; tetrazine ligation, such as with tetrazines and cycloalkynes or trans-cycloalkenes; Diels-Alder cycloaddition reactions), nucleophilic substitution, a Michael reaction, or other examples of click reactions.

In one embodiment, the process may further comprise the step of opening a maleimide-thiol adduct ring (i.e. opening a succinimide ring). In one embodiment, the step of opening a maleimide-thiol adduct ring may be provided after step (b). In another embodiment, the step of opening a maleimide-thiol adduct ring may be provided after step (c). In another embodiment, the step of opening a maleimide-thiol adduct ring may be provided after step (d).

### Additional Components

In further embodiments, there is provided a composition, a pharmaceutical composition and/or a kit which comprises a polypeptide, a cocktail of polypeptides, a nucleic acid molecule, a cocktail of nucleic acid molecules, a conjugate, a cocktail of conjugates and/or a combination of the aforementioned as described above (hereafter referred to as the “medicament” or “medicaments”). In certain embodiments, the combination comprises a mixture of any one of the polypeptides and/or nucleic acid molecules and/or conjugates as set out above but in alternative embodiments the respective components are provided in separate receptacles in the form of a kit. In further embodiments, there is provided a first product and a second product as described below which are provided in a mixture or, alternatively, the first and second products are provided in separate receptacles in the form of a kit.

In one embodiment, the pharmaceutical composition and/or the kit comprise a pharmaceutically acceptable adjuvant, diluent and/or excipient. In a further embodiment, the pharmaceutical composition and/or the kit comprise a pharmaceutically acceptable diluent and/or excipient. It is to be appreciated that in some embodiments the pharmaceutically acceptable adjuvant, diluent and/or excipient is provided in formulation with the medicament whereas in other embodiments, the pharmaceutically acceptable adjuvant, diluent and/or excipient is provided in a separate receptacle from the medicament(s) in the form of a kit. It is preferred that the components are provided mixed in a single formulation.

It is also to be appreciated that, where components are provided in separate receptacles in a kit, then, in some embodiments, when the kit is used, the components are combined before administration to an individual whereas in other embodiments, when the kit is used, the components are administered separately to the individual.

Exemplary adjuvants include Poly I:C (Hiltonol), CpG, a liposome, a microsphere, a virus-like particle (an immune stimulating complex, ISCOMS), Incomplete Freund's Adjuvant (IFA), Complete Freund's Adjuvant (CFA), aluminium phosphate, aluminium hydroxide, alum and/or a bacterial toxin (for example, cholera toxin and/or Salmonella toxin) or nanoparticle formulations of any sort. In one embodiment, the adjuvant is a combination of one or more adjuvants. In one embodiment, the adjuvant is IFA and CpG. Further exemplary adjuvants include Imiquimod, glucopyranosyl and/or Lipid A. A preferred adjuvant is GM-CSF (granulocyte macrophage colony stimulating factor). In one embodiment, the GM-CSF is sargramostim. Exemplary adjuvants for use in vaccines targeting the T-cell arm of the immune system are detailed in Petrovsky & Aguilar Immunol Cell Biol. 2004 82 (5): 488-96, which is incorporated herein by reference.

Exemplary diluents and excipients include sterilised water, physiological saline, culture fluid and/or phosphate buffer or lipid and/or nanoparticle-based formulations.

It is also to be understood that, in specific embodiments, the medicament is a nucleic acid molecule and this is provided in a lipid or nanoparticle formulation.

The pharmaceutical composition and/or kit, in some embodiments, also comprises a further therapeutic ingredient. Exemplary further therapeutic ingredients include interleukin-2 (IL-2), interleukin-12 (IL-12), a further polypeptide (that is to say, a polypeptide aside from those discussed above), a chemotherapeutic, a pain killer, an anti-inflammatory agent and/or an anti-cancer agent.

Further details of additional components of the pharmaceutical composition and/or kit may be found in Remington's Pharmaceutical Sciences and US Pharmacopoeia, 1984, Mack Publishing Company, Easton, PA, USA.

### Methods of the Invention

In use, the conjugate, the cocktail of conjugates, the polypeptide, the cocktail of polypeptides, the nucleic acid molecule, the cocktail of nucleic acid molecules, the combination, the composition or the pharmaceutical composition as explained above (the “medicament”) is administered to a subject.

In embodiments where a kit comprising the conjugate, the polypeptide, the cocktail of polypeptides, the nucleic acid molecule, the cocktail of nucleic acid molecules and/or the combination of the present invention is provided, in use, each component of the kit is administered to the subject. The components of the kit as described above are administered simultaneously, separately or sequentially to the subject. In one embodiment, the kit comprises a further therapeutic ingredient as described above. In such embodiments, the conjugate, the polypeptide, the cocktail of polypeptides, the nucleic acid molecule, the cocktail of nucleic acid molecules and/or the combination is administered to the subject simultaneously, separately or sequentially with the further therapeutic ingredient.

In other embodiments, a first product and a second product are administered to the subject and in this regard the first product and the second product can together be considered to be the “medicament” albeit they are not necessarily a single product. The first product is selected from the following (i) to (v):
(i) a polypeptide comprising the sequence of SEQ ID NO: 1;
(ii) a polypeptide comprising an immunogenic fragment of (i) comprising at least eight amino acids;
(iii) a polypeptide comprising a sequence having at least 80% sequence identity to (i) or (ii);
(iv) a conjugate comprising the polypeptide defined in any one of (i) to (iii); and
(v) a nucleic acid molecule consisting of a nucleotide sequence encoding a polypeptide as defined in any one of (i) to (iii).

The second product is selected from the following (vi) to (x):
(vi) a polypeptide comprising the sequence of SEQ ID NO: 116;
(vii) a polypeptide comprising an immunogenic fragment of (vi) comprising at least seventeen amino acids;
(viii) a polypeptide comprising a sequence having at least 80% sequence identity to (vi) or (vii);
(ix) a conjugate comprising the polypeptide defined in any one of (vi) to (viii); and
(x) a nucleic acid molecule consisting of a nucleotide sequence encoding a polypeptide as defined in any one of (vi) to (viii).

In some variants of this embodiment, both the first product and the second product are administered simultaneously, sequentially or separately. In these variants, the first product may be administered before the second product or the second product may be administered before the first product. In some alternative variants of this embodiment, the first product and the second product are combined as a single product (i.e. a mixture) and are administered as the single product and in these alternative variants, the following provisos apply:
(a) where the first product and the second product are a single polypeptide and the first product is as defined in any one of (i) to (iii) and the second product is as defined in any one of (vi) to (viii) then the single polypeptide is equal to or less than 170 amino acids in length; and
(b) where the first product and the second product are a single product and the first product is as defined in (v) and the second product is as defined in (x) then the single nucleic acid molecule is less than 1500 nucleotides in length.

For example, in one embodiment, the first product is a polypeptide comprising the sequence of SEQ ID NO: 1 and the second product is a polypeptide comprising the sequence of SEQ ID NO: 116 and the first and second products are mixed but remain as separate molecules. In another embodiment, the first product is a first polypeptide comprising the sequence of SEQ ID NO: 1 and the second product is a second polypeptide comprising the sequence of SEQ ID NO: 116 and the first and second polypeptides together form a single fusion protein which is equal to or less than 170 amino acid residues in length. In another embodiment, the first product is a nucleic acid molecule encoding the polypeptide comprising the sequence of SEQ ID NO: 1 and the second product is a nucleic acid molecule encoding the polypeptide comprising the sequence of SEQ ID NO: 116 and wherein first and second products are mixed but remain as separate molecules. In another embodiment, the first product is a first nucleic acid molecule encoding the polypeptide comprising the sequence of SEQ ID NO: 1 and the second product is a second nucleic acid molecule encoding the polypeptide comprising the sequence of SEQ ID NO: 116 and the first and second nucleic acid molecules together form a single nucleic acid molecule which is less than 1500 nucleotides in length.

In one embodiment, the subject is a patient in need of treatment. In a further embodiment, the patient is a cancer patient. In an alternative embodiment, the medicament or the components of the kit are administered to a subject prior to any symptoms of disease in order to provide a prophylactic therapy. In one embodiment, the disease is cancer and the medicament or the components of the kit are administered to the subject prior to any symptoms of cancer in order to provide protective immunity against the cancer.

As described above, the conjugate of the present invention comprises at least one polypeptide comprising a sequence of a B-cell epitope. In one embodiment, the subject to whom the medicament or the components of the kit are to be administered has pre-existing, circulating antibodies specific to the B-cell epitope. In an alternative embodiment, the subject does not have pre-existing, circulating antibodies specific to the B-cell epitope. In a further embodiment, the subject has pre-existing circulating antibodies specific to the B-cell epitope but it is desired to increase the level of the antibodies. In embodiments where the subject does not have the pre-existing, circulating antibodies or it is desired to increase their level, the subject is administered a vaccine to induce a B-cell response (and thus to induce antibody production) to the B-cell epitope prior to the administration of the medicament or a component of the kit. In one embodiment, the presence and/or level of antibodies specific to the B-cell epitope in a subject to whom the conjugate is to be administered is determined prior to administration of the conjugate. In one embodiment, the vaccine to induce a B-cell response to the B-cell epitope is administered at least 1, 2, 3, 4, 5, 6, 7, 8, 9, 10, 20, 30 or more weeks prior to administration of the medicament or a component of the kit.

In an alternative embodiment, the subject is passively administered antibodies specific to the B-cell epitope to provide a passive humoral immune response to the B-cell epitope in the subject. In one embodiment, the antibodies specific to the B-cell epitope are administered to the subject simultaneously, separately or sequentially with the medicament or with a component of the kit. In one embodiment, the antibodies specific to the B-cell epitope are provided in a solution or serum.

In embodiments in which the B-cell epitope is derived from any one of SEQ ID NOS. 3 to 5, the vaccine to induce the B-cell response to the B-cell epitope is a tetanus vaccine. In one embodiment, the tetanus vaccine comprises the tetanus toxoid (TTd), a fragment thereof and/or a fragment of the tetanus toxin (TTx). In a further embodiment, the “tetanus vaccine” is a diphtheria, tetanus and pertussis (DTP) combination vaccine or any tetanus toxoid-containing vaccine. In an alternative embodiment, anti-TTx/TTd antibodies are passively administered to the subject simultaneously, separately or sequentially with the medicament or with a component of the kit. In one embodiment, the subject is administered a solution or serum comprising anti-TTx/TTd antibodies, e.g. Tetaquin or an equivalent anti-TTx/TTd antibody preparation. In one embodiment, the subject is administered an isolated IgG fraction from a high titre anti-TTx/TTd donor. In one embodiment, the presence and/or level of anti-TTx/TTd antibodies in a subject to whom the conjugate is to be administered is determined prior to administration of the conjugate. In a preferred embodiment, a Tettox ELISA as described above is used to determine the presence and/or level of anti-TTx/TTd antibodies.

In one embodiment, the vaccine to induce a B-cell response to the B-cell epitope is administered to the subject at least twice. Thus the subject receives at least a first (priming) dose and at least a second (booster) dose of the vaccine. In one embodiment, the subject has received at least a first (priming) dose and at least a second (booster) dose in childhood and is administered at least one further booster dose prior to administration of the conjugate. In one embodiment, the at least one booster dose is administered 1, 2, 3, 4, 5, 6, 7, 8, 9, 10, 20, 30 or more weeks prior to administering the medicament or a component of the kit.

Without wishing to be bound by theory, it is thought that upon administration of the conjugate, the circulating antibodies in the subject (i.e. either pre-existing, induced following vaccination or transferred passively) bind to the B-cell epitope to form an immune complex (IC), which directs/targets the conjugate to antigen-presenting cells (APC). It is therefore advantageous for each conjugate to comprise more than one polypeptide comprising a B-cell epitope since multiple antibodies are then able to bind to each conjugate simultaneously which thereby facilitates the formation of the immune complex. In one embodiment, the Fc portion of an antibody bound to the B-cell epitope binds to an Fc receptor on the APCs. In a further embodiment, complement and C1q binding promotes uptake of the conjugate by complement-receptor positive APCs or by scavenger receptors. The targeting and binding of the conjugate via Fc receptors and/or complement receptors to an APC (in one embodiment, the APC is a dendritic cell (DC)) promotes activation of the APC accompanied by internalisation and intracellular processing of the conjugate by the activated APC. In this way, the CD4+ T-cell epitope is presented to relevant CD4+ T-cells by MHC Class II molecules on the APC and elicits a CD4+ T-cell response. In one embodiment, the CD4+ T-cell epitope is presented to relevant CD4+ T-cells by MHC Class II molecules on the APCs in a lymph node draining the site of administration of the conjugate (in one embodiment, the site of administration is a vaccine injection site). Thus the conjugate enables efficient targeting to APCs, promoting the processing and presentation of the CD4+ T-cell epitope to the relevant CD4+ T-cells. In one embodiment, the conjugate circumvents the need for pre-stimulation and/or co-stimulation with an external adjuvant (such as GM-CSF).

In addition, a B-cell comprising a B-cell receptor specific for the B-cell epitope of the conjugate can bind and internalise the conjugate by B-cell receptor-mediated internalisation. In this way, the B-cell also processes and presents (on MHC Class II molecules) the CD4+ T-cell epitope of the conjugate to relevant CD4+ T-cells.

Through presentation on APCs to the relevant CD4+ T-cells, the at least one polypeptide comprising the CD4+ T-cell epitope is capable of generating a helper T-cell response against the universal tumour antigen from which the CD4+ T-cell epitope is derived. This supports the generation of specific immune responses against the CD4+ T-cell epitope of the universal tumour antigen. In one embodiment, the at least one polypeptide comprising the CD4+ T-cell epitope promotes a Th1 immune response. A Th1 immune response is important for an immune response directed against a cancer cell and thus contributes to the treatment or prophylaxis of cancer. In one embodiment, the at least one polypeptide comprising the CD4+ T-cell epitope generates a T-memory cell response. A T-memory cell response is important to promote the long-term surveillance of a cancer.

The T helper response that is promoted by the CD4+ T-cell epitope of the conjugate supports a feedback loop of antibody regulation in the subject to whom the conjugate has been administered. More specifically, the helper T cells that are stimulated by the CD4+ T-cell epitope of the conjugate are capable of co-stimulating the B-cells that are specific for the B-cell epitope of the conjugate. The stimulated B-cells generate antibodies specific to the B-cell epitope thus increasing the level of antibody specific to the B-cell epitope in the subject following administration of the conjugate. In one embodiment, the increased level of antibody specific to the B-cell epitope is an increased level of IgG antibody specific to the B-cell epitope. The increased level of antibody specific to the B-cell epitope improves IC formation and targeting of the conjugate to the APC. Thus this feedback loop of antibody regulation improves the adjuvant function of the at least one polypeptide comprising the B-cell epitope.

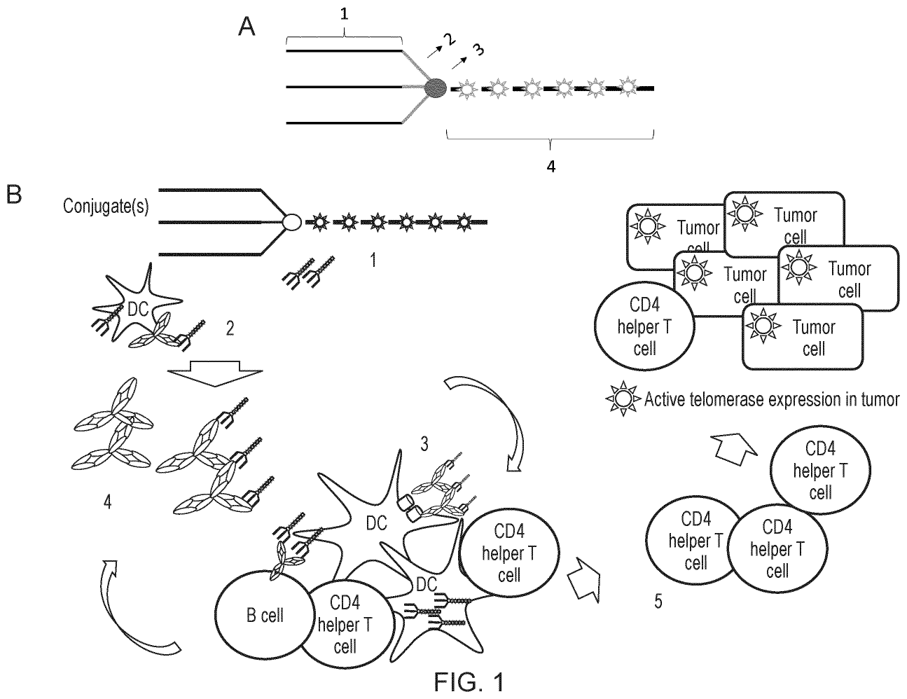

FIG. 1B illustrates the proposed mechanism by which an embodiment of the present invention elicits an immune response.

In embodiments in which the conjugate comprises a CD8+ T-cell epitope, it is to be appreciated that as a result of intracellular processing of the conjugate by the activated APC, the CD8+ T-cell epitope may be presented in a complex with an MHC class I molecule on the cell surface and thereby elicit a CD8+ T cell response.

In embodiments in which the medicament comprises a polypeptide, the polypeptide is endocytosed by antigen presenting cells, may be subject to antigen processing and is then presented in complex with an MHC class I or class II molecule on the cell surface.

Through interaction with T-cell receptors on the surface of T-cells, a CD4+ or CD8+ T-cell response is elicited. In embodiments in which the medicament comprises a nucleic acid molecule, the nucleic acid molecule is also endocytosed and is then transcribed (if the nucleic acid molecule is DNA), and the encoded polypeptide is synthesised through endogenous cellular pathways. Subsequently, if the target cell is an antigen presenting cell, the encoded polypeptide is processed and presented on an MHC molecule in order to elicit the T-cell response, as previously described. Alternatively, if the target cell is another cell, such as a muscle cell, fibroblast and other non-antigen presenting cells, the encoded polypeptide will be secreted and taken up by an antigen presenting cell. Thus the medicament may be used as a vaccine in order to elicit either CD4+ or CD8+ T-cell immunity.

In principle, any mode of administration of the medicament or the components of the kit may be used but injection is particularly preferred. In one embodiment, the medicament or a component of the kit is administered to the subject by a parenteral route, that is to say administration is by an intradermal, a subcutaneous, an intramuscular, an intravenous, an intraarterial, an intraperitoneal, or an intralesional route. In a preferred embodiment, the medicament or a component of the kit is administered by an intradermal, a subcutaneous or an intramuscular route. In one embodiment, administration as a bolus injection is useful.

In embodiment, in which the medicament is a polypeptide, a suitable dosage of the polypeptide is between 100 and 700 μg although dosages outside this range may occasionally be required (e.g. from 1-1500 μg). In one embodiment, the polypeptide is administered simultaneously, separately or sequentially with an adjuvant, preferably GM-CSF, most preferably sargramostim. A suitable dosage of GM-CSF, preferably sargramostim, is between 20 and 100 μg. In one embodiment, the dosage is 37.5 μg, in a preferred embodiment, the dosage is 75 μg.

In embodiments in which the medicament is a nucleic acid molecule, a suitable dosage of the nucleic acid molecule is between 10 and 1000 μg although dosages outside this range may occasionally be required (e.g. from 1-1500 μg).

In embodiments in which the medicament is a conjugate of the invention, a suitable dosage of the conjugate is between 100 and 2000 microgram although dosages outside this range may occasionally be required (e.g. from 1-5000 μg).

In one embodiment, an individual is administered a tetanus booster vaccine at least 7 days prior to administration of the conjugate of the invention to the individual. In this way, the individual's immune response to the Minimal Tetanus Toxin Epitope sequence is boosted, prior to receiving the conjugate of the invention.

In certain embodiments, multiple medicaments are provided and are administered simultaneously, separately or sequentially to an individual. That is to say, the medicaments may be administered at a different time, as well as in a substantially simultaneous manner. The term “simultaneously” as used herein refers to administration of more than one medicament at the same time. Simultaneously includes administration contemporaneously, that is during the same period of time. In certain embodiments, the medicaments are administered simultaneously in the same hour, or simultaneously in the same day. In some embodiments, the term “sequentially” refers to the medicaments being administered within 30 days of each other.

It is to be appreciated that, as the CD4+ T-cell epitope is derived from a universal tumour antigen, which are expressed in a high proportion of tumour types, the efficacy of the present invention is not limited to any particular type of tumour/cancer. In embodiments in which the universal tumour antigen is hTERT, in principle, the medicament or the components of the kit may be administered to a patient suffering from any type of cancer in which the telomerase gene is activated. Alternatively, the medicament or the components of the kit may be administered prophylactically to a subject who is at risk of any type of cancer in which the telomerase gene is activated. Cancers in which the telomerase gene is activated include but are not limited to breast cancer, prostate cancer, pancreatic cancer, colorectal cancer, lung cancer, bladder cancer, malignant melanoma, leukaemias, lymphomas, ovarian cancer, cervical cancer and biliary tract carcinomas. However, as the telomerase enzyme is expressed in the vast majority of cancers, it is to be understood the efficacy of the invention is not limited to any particular type of cancer.

That telomerase is expressed in the vast majority of cancers has been demonstrated in studies such as Kim et al. Science. 1994 Dec. 23; 266 (5193): 2011-5 and Bearss et al. Oncogene. 2000 Dec. 27; 19 (56): 6632-41 (both are incorporated herein by reference).

Kim et al. 1994 has demonstrated that, in cultured cells representing 18 different human tissues, 98 of 100 immortal and none of 22 mortal populations were positive for telomerase. The human tissues from which the immoral cell lines having telomerase activity were derived included: skin, connective, adipose, breast, lung, stomach, pancreas, ovary, cervix, kidney, bladder, colon, prostate, CNS, retina and blood. The present invention would therefore be suitable for use against cancers derived from these tissues. Similarly, 90 of 101 biopsies representing 12 human tumour types and none of 50 normal somatic tissues were positive for telomerase. The human tumour types which exhibited telomerase activity included: hepatocellular carcinoma, colon cancer, squamous cell carcinoma (head and neck), Wilms tumor, breast cancer (ductal and lobular, node positive), breast cancer (axillary node negative), prostate cancer, prostatic intraepithelial neoplasia type 3, benign prostatic hyperplasia, neuroblastoma, brain tumors, lung small-cell carcinoma, rhabdomyosarcoma, leiomyosarcoma, hematological malignancies (including acute lymphocytic leukaemia, chronic lymphocytic leukemia, lymphoma (adult)),

Bearss et al. 2000 has furthermore demonstrated the presence of telomerase activity in tumour cells taken directly from patients across a wide range of cancer types. These tumour types included: hematologic malignancies (including acute myeloid leukaemia, acute lymphoid leukaemia, chronic myeloid leukaemia, chronic lymphoid leukaemia (early), chronic lymphoid leukaemia (late), myeloma, low-grade lymphoma, high-grade lymphoma); breast; prostate; lung (including non-small cell and small cell); colon; ovarian; head and neck; kidney; melanoma; neuroblastoma; glioblastoma; hepatocellular carcinoma; gastric; and bladder.

It is to be understood that, as telomerase is activated in the above-mentioned cancer types, the present invention is suitable for use against any one of these types of cancer (and indeed any cancer type in which telomerase is activated). Furthermore, it is apparent that, as the activation of telomerase is a common property shared between cancer types, the present invention is not limited to any particular type of cancer.

It is also to be appreciated that subjects will generally have developed some degree of immunological tolerance to epitopes of self-antigens, such as telomerase reverse transcription, through a process whereby T-cells reactive with such epitopes are destroyed in the thymus of the subject during T-cell development. Thus in some embodiments of the present invention, epitopes with a relatively low MHC binding affinity are desired. This is because epitopes with lower MHC binding affinity will have been exposed to maturing T-cells at a lower rate and so it is less likely that all of the subject's T-cells reactive with the epitope will have been deleted from the subject's T-cell repertoire. Thus epitopes having a relatively low MHC binding affinity are, in some embodiments, able to overcome immunological tolerance more readily.

In some embodiments, the administration of the medicament or the components of the kit results in “epitope spreading” whereby the immune response is expanded to other, secondary epitopes (Gulley et al. J Natl Cancer Inst. 2017 Apr. 1; 109 (4)).

### An Assay Method

In accordance with a further aspect of the invention, there is provided a method of determining the presence of a CD4+ T-cell response to a CD4+ T-cell epitope in a subject to whom a conjugate comprising at least one polypeptide comprising a sequence of a B-cell epitope and at least one polypeptide comprising the sequence of the CD4+ T-cell epitope has been administered. The present inventors have surprisingly found that administration of such a conjugate results in an increase in the level of antibody specific to the conjugate (such as an antibody specific to the B-cell epitope in the conjugate) but only when the conjugate comprises a B-cell epitope and a CD4+ T-cell epitope. For example, this is shown in Example 7, where a conjugate comprising a B-cell epitope and a CD8+ T-cell epitope was not capable of driving an increase in the level of antibody specific to the B-cell epitope whereas a conjugate comprising a B-cell epitope and a CD4+ T-cell epitope was capable of doing so.

Without wishing to be bound by theory, it is thought that the increase in the level of antibody specific to the conjugate (such as an antibody specific to the B-cell epitope in the conjugate) results from CD4+ T-cell recognition of the CD4+ T-cell epitope of the conjugate. In particular, the CD4+ T-cells that are stimulated by the CD4+ T-cell epitope of the conjugate are capable of co-stimulating B-cells specific to the conjugate (such as antibodies specific to the B-cell epitope of the conjugate), resulting in the stimulated B-cells generating antibody specific to the conjugate and thus increasing the level of this antibody. The present inventors have made the surprising realisation that the level of antibody specific to the conjugate can be used as a biomarker for the presence of a CD4+ T-cell response to the CD4+ T-cell epitope of the conjugate in a subject to whom the conjugate has been administered.

In the method of the invention, a blood sample is taken from a subject. In a first embodiment, a blood sample is taken prior to an administration of a conjugate comprising at least one polypeptide comprising a sequence of a B-cell epitope and at least one polypeptide comprising a sequence of a CD4+ T-cell epitope. In a first embodiment, the B-cell epitope of the conjugate comprises the sequence of SEQ ID NO: 7 and the CD4+ T-cell epitope comprises the sequence of SEQ ID NO: 1. In the first embodiment, the at least one polypeptide comprising the sequence of SEQ ID NO. 7 is a first, a second and a third polypeptide comprising the sequence of SEQ ID NO. 7. The conjugate is then administered to the subject at least once. In a preferred embodiment, the subject receives one cycle of administration of the conjugate, wherein the cycle of administration comprises a plurality of injections of the conjugate, preferably 4 injections. Thus in some embodiment, it is to be understood that the term “an administration” encompasses a cycle of administrations. In some embodiments, the blood sample is taken from the subject prior to a first administration or cycle of administration of the conjugate (that is to say, the subject is a “naïve” subject). In alternative embodiments, the blood sample is taken prior to a further administration or cycle of administration of the conjugate (that is to say, the subject is not a “naïve” subject). In the first embodiment, a blood sample is taken from the subject subsequent to the administration of the conjugate at a first time point. In one embodiment, the blood sample is taken 1, 2, 3, 4, 5, 10, 15, 20, 25 or 30 days subsequent to the administration of the conjugate.

In a further embodiment, the blood sample taken prior to an administration of the conjugate is taken subsequent to at least one administration of a vaccine to induce a B-cell response to the B-cell epitope. That is to say, the subject receives at least one administration of a vaccine to induce a B-cell response and then the blood sample is taken. In a preferred embodiment, the subject receives 2, 3, 4, 5 or more administrations of the vaccine to induce the B-cell response. Thus the subject to whom the conjugate is administered is one that has an immune response to the B-cell epitope. It is to be understood that such a sample is included within the term “a sample derived from the subject prior to an administration of the conjugate”. In a preferred embodiment, the vaccine to induce a B-cell response to the B-cell epitope is administered at least twice before the blood sample is taken. In the first embodiment, where the B-cell epitope has the sequence of SEQ ID NO: 7, the vaccine to induce a B-cell response thereto is a tetanus vaccine (for example, as defined herein). In one embodiment, the blood sample is taken 1, 2, 3, 4, 5, 10, 15, 20, 25 or 30 days subsequent to the at least one administration of the vaccine to induce a B-cell response to the B-cell epitope.

In the first embodiment, the samples derived from the subject prior and subsequent to an administration or cycle of administration of the conjugate are each contacted with a plate onto which a polypeptide comprising the sequence of SEQ ID NO. 7 has been immobilised. In a one embodiment, the polypeptide comprising the sequence of SEQ ID NO. 7 is biotinylated and is immobilised on a streptavidin-coated plate. In the first embodiment, the quantity or absence of antibody specific to the sequence of SEQ ID NO. 7 in each blood sample is detected by ELISA. The ELISA procedure has been described previously, for example, in Fletcher et al. J Immunol. 2018 Jul. 1; 201 (1): 87-97, which is incorporated herein by reference. In brief, once each sample has been contacted with the plate, any antibodies which are specific for the sequence of SEQ ID NO. 7 and present in the sample bind to the immobilised polypeptide comprising the sequence of SEQ ID NO. 7. In one embodiment, the antibody specific for the sequence of SEQ ID NO. 7 is an IgG antibody. The plate is then washed and blocked to prevent any non-specific binding. A secondary antibody conjugated to an enzyme, which catalyses a reaction to produce a detectable signal, is contacted with the plate and any excess secondary antibody is removed by washing. In one embodiment, where the antibody to be detected is an IgG antibody, the secondary antibody is an anti-IgG antibody. In one embodiment, the enzyme conjugated to the secondary antibody is horseradish peroxidase (HRP) and a detectable signal is produced from the oxidation of tetramethylbenzidine (TMB), which produces a colour change. The signal is detected and used to determine the quantity or absence of the antibody specific to the sequence of SEQ ID NO. 7 in each sample. In one embodiment, the signal is detected by measuring absorbance on a spectrophotometer at 450 nm.

The quantity or absence of antibody specific to the sequence of SEQ ID NO. 7 in the sample derived from the subject prior to an administration of the conjugate is detected by the above method at a first level. The quantity or absence of antibody specific to the sequence of SEQ ID NO. 7 in the sample derived from the subject subsequent to the administration of the conjugate is detected by the above method at a second level. In the first embodiment, the second level and the first level are compared. An increase in the second level relative to the first level signifies that the quantity of antibody present in the sample derived from the subject subsequent to the administration of the conjugate is higher than that in the sample derived from the subject prior to the administration of the conjugate. In one embodiment, the increase is a statistically significant increase. The increase is indicative of the presence of a CD4+ T-cell response to the polypeptide of SEQ ID NO. 1 in the subject. This is thought to arise because the polypeptide of SEQ ID NO. 1, through stimulation of CD4+ T-cells has co-stimulated B-cells specific to the sequence of SEQ ID NO. 7, leading to the production of antibody specific to the sequence of SEQ ID NO. 7.

Conversely, if a comparison of the second level and the first level reveals no change, a less than 10% change and/or a non-statistically significant change then this indicates that a CD4+ T-cell response to the polypeptide of SEQ ID NO. 1 is not present in the subject.

In a further embodiment, a blood sample is taken from the subject at a second time point subsequent to the administration of the conjugate. In one embodiment, the second time point is prior to the first time point described above. That is to say, it is closer in time to the administration of the conjugate. In one embodiment, the second time point is less than 1 day or 1, 2, 3, 4 or 5 days subsequent to the administration of the conjugate. The quantity or absence of antibody specific to the sequence of SEQ ID NO. 7 in the sample derived from the subject at the second time point is detected by the above method at a third level. The third level and the second level are compared. An increase in the second level relative to the third level signifies that the quantity of antibody present in the sample derived from the subject at the first time point (i.e. which is further in time from the administration of the conjugate) is higher than that in the sample derived from the subject at the second time point (i.e. which is closer in time to the administration of the conjugate). In one embodiment, the increase is a statistically significant increase. The increase is indicative of the presence of a CD4+ T-cell response to the polypeptide of SEQ ID NO. 1 in the subject. This is thought to arise because during the intervening time period between the two time points, the polypeptide of SEQ ID NO. 1, through stimulation of CD4+ T-cells has co-stimulated B-cells specific to the sequence of SEQ ID NO. 7, leading to the production of antibody specific to the sequence of SEQ ID NO. 7.

Conversely, if a comparison of the second level and the third level reveals no change, a less than 10% change and/or a non-statistically significant change then this indicates that a CD4+ T-cell response to the polypeptide of SEQ ID NO. 1 is not present in the subject.

In the embodiment described above, a blood sample is taken at a first and a second time point subsequent to the administration of the conjugate. In a further embodiment, a blood sample is taken at 3, 4, 5, 6, 7, 8, 9 or 10 or more time points subsequent to the administration of the conjugate. It is to be understood that the quantity or absence of antibody may be compared between any two of these time points and/or the quantity or absence of antibody at each time point may be compared such that the level of antibody in the subject is plotted over time.

In a variant of the first embodiment, a blood sample is taken subsequent to an administration of the conjugate as described above but not prior to an administration of the conjugate. The quantity or absence of antibody specific to the sequence of SEQ ID NO. 7 in the sample derived from the subject subsequent to an administration of the conjugate is detected by the above method at a level. If the level is higher than a threshold value then it is indicative of the presence of a CD4+ T-cell response to the polypeptide of SEQ ID NO. 1 in the subject. In one embodiment, the threshold value is determined from a collation of data from a plurality of subjects.

In the embodiments described above, a blood sample is taken from the subject. In one embodiment, the blood sample is further processed before it is used in the method of the invention, for example, to obtain a plasma sample or a diluted plasma sample. Any suitable sample derived from the subject that contains antibody can be used in the method of the present invention.

In the embodiments described above, the antibody specific to the sequence of SEQ ID NO. 7 which is detected is an IgG antibody. In a preferred embodiment, the antibody is an IgG1 antibody or IgG4 antibody. In an alternative embodiment, the antibody to be detected is from a different immunoglobulin class (i.e. other than IgG).

In the embodiments described above, an ELISA procedure is used to detect antibodies specific to the B-cell epitope. Various modifications to the above-referenced ELISA procedure will be apparent to those skilled in the art. In an alternative embodiment, a procedure other than ELISA is used to detect the quantity or absence of antibody specific to the B-cell epitope. In one such alternative embodiment, a dipstick assay, a spot assay, a lateral flow assay and/or a Meso-Scale assay is used.

It is to be understood that the conjugate and/or the vaccine to induce a B-cell response to the B-cell epitope may have been administered the subject more than once, for example, the conjugate and/or vaccine to induce the B-cell response may have been administered to the subject twice (or more). Where it is planned for there to be multiple administrations of the conjugate and/or the vaccine to induce the B-cell response, the blood sample taken from the subject, in some embodiments, is taken subsequent to the final planned administration. Thus in some embodiments, the blood sample is taken 1, 2, 3, 4, 5, 10, 15, 20, 25 or 30 days subsequent to the final planned administration of the conjugate and/or the vaccine to induce the B-cell response. In an alternative embodiment, a blood sample is taken after each administration of the conjugate and/or the vaccine to induce the B-cell response. It is to be understood that such additional samples may be used to provide more detailed information on the developing immune response in the subject.

In the embodiments described above, an increase between two detected levels of the antibody, which is indicative of the presence of a CD4+ T-cell response, is a statistically significant increase. In one embodiment, the statistically significant increase is determined using a paired t-test and a p-value of ≤0.05, preferably a p-value of ≤0.01. In one embodiment, the increase between two detected levels is a two-fold increase. In some embodiments, the increase which is indicative of the presence of a CD4+ T-cell response is at least a 50% increase, preferably at least a 75%, 100%, 150%, 200% increase.

In the first embodiment described above, the B-cell epitope of the conjugate that has been administered to the subject comprises the sequence of SEQ ID NO. 7. In an alternative embodiment, the B-cell epitope comprises a different sequence. In one embodiment, the B-cell epitope comprises a sequence having at least 70% sequence identity to the sequence of SEQ ID NO. 7, preferably at least 75%, 80%, 85%, 90%, 95%, 96%, 97%, 98% or 99% sequence identity thereto. In a further embodiment, the B-cell epitope comprises a sequence derived from the sequence of the tetanus toxin (SEQ ID NO. 3) or the tetanus toxin heavy chain (SEQ ID NO. 5) other than that of the sequence of SEQ ID NO. 7. In one embodiment, the B-cell epitope comprises: (i) a sequence comprising at least 10 amino acids which are contiguous in SEQ ID NO. 5 and which comprise the amino acid sequence GITELKKL (as represented by SEQ ID NO. 6 in the Sequence Listing); or (ii) a sequence having at least 70% sequence identity to (i), preferably at least 75%, 80%, 85%, 90%, 95%, 96%, 97%, 98% or 99% sequence identity to (i). In one embodiment, the B-cell epitope does not comprise the complete sequence of the TTx heavy chain (SEQ ID NO: 5). In one embodiment, the B-cell epitope is selected from any one of the B-cell epitopes described in an aforementioned section.

In the first embodiment described above, the CD4+ T-cell epitope of the conjugate that has been administered to the subject comprises the sequence of SEQ ID NO. 1. In an alternative embodiment, the CD4+ T-cell epitope comprises a different sequence. In one embodiment, the CD4+ T-cell epitope comprises the sequence of SEQ ID NO: 116 or 117. In one embodiment, the CD4+ T-cell epitope comprises the sequence of an immunogenic fragment of SEQ ID NO. 1, 116 or 117 comprising at least 12 amino acids. In a further embodiment, the CD4+ T-cell epitope comprises a sequence having at least 80% sequence identity to the sequence of SEQ ID NO. 1, 116 or 117 or an immunogenic fragment thereof, preferably at least 85%, 90%, 95%, 96%, 97%, 98% or 99% sequence identity. In yet a further embodiment, the CD4+ T-cell epitope comprises a region of at least 12 amino acids of telomerase reverse transcriptase (other than the sequence of SEQ ID NO. 1, 116 or 117) or a sequence having at least 80% sequence identity to the region. In one embodiment, the CD4+ T-cell epitope comprises a region of at least 12 amino acids of a different self-antigen, a different tumour-associated antigen and/or a different universal tumour antigen (i.e. other than telomerase reverse transcriptase) or a sequence having at least 80% sequence identity to the region. In one embodiment, the universal tumour antigen is selected from the group consisting of survivin, DNA topoisomerase 2-alpha (Top2α), cytochrome P450 1B1 (CYP1B1) and E3 ubiquitin-protein ligase Mdm2. In a preferred embodiment, the sequence identity to any one of the above-mentioned regions is it at least 85%, 90%, 95%, 96%, 97%, 98% or 99%. In one embodiment, the polypeptide comprising the CD4+ T-cell epitope is 500 amino acids or less in length. In a further embodiment, the at least one polypeptide comprising the CD4+ T-cell epitope is equal to or less than 400, 300, 200, 170, 150, 125, 100, 90, 80, 70, 75, 60, 50, 40 or 30. In one embodiment, the CD4+ T-cell epitope is selected from any one of the CD4+ T-cell epitopes described in an aforementioned section.

It is particularly preferred that the CD4+ T-cell epitope is from a naturally-occurring protein such as an epitope from an endogenous protein or a viral or bacterial protein such as associated with the tumor micro-environment. It is also preferred that the CD4+ T-cell epitope is an intracellular peptide such as a peptide which is part of a tumor antigen or a bacterial or viral antigen of intracellular origin. In some embodiments, the CD4+ T-cell epitope is present as an endogenous tumour antigen. In this way, the measurement of the antibody specific for the conjugate acts as a biomarker in relation to the T cell response directed to the tumor or the (optionally tumor-associated) pathogen (virus or bacteria).

In the embodiments described above, the conjugate that has been administered to the subject comprises a first, a second and a third polypeptide comprising a B-cell epitope and a first polypeptide comprising a CD4+ T-cell epitope. In one embodiment, the method of the present invention is performed using a sample derived from a subject to whom a conjugate containing any combination of a first, a first and second, or a first, a second and a third polypeptide comprising a B-cell epitope and a first, a first and second, or a first, a second and a third polypeptide comprising a CD4+ T-cell epitope has been administered. In one embodiment, the at least one polypeptide comprising the sequence of the CD4+ T-cell epitope comprises a sequence of a further T-cell epitope. In one embodiment, the further T-cell epitope is a CD8+ T-cell and/or a further CD4+ T-cell epitope. In a further embodiment, the method of the present invention is performed using a sample derived from a subject to whom a conjugate comprising at least one polypeptide comprising a sequence of a B-cell epitope, at least one polypeptide comprising a sequence of a CD4+ T-cell epitope and at least one polypeptide comprising a sequence of a further epitope has been administered. Such conjugates are described in further detail above.

In one embodiment, the method of the present invention is performed using a sample derived from a subject to whom a vaccine comprising a plurality of conjugates has been administered. In one embodiment, the plurality of conjugates is a cocktail of conjugates as defined herein. In a further embodiment, a vaccine comprising a plurality of conjugates is the “TENDU” vaccine. The TENDU vaccine comprises six SLPs (termed “LUG1-6”) comprising the sequences of SEQ ID NOS. 47 to 51 and 45, which contain epitopes derived from PAP, GCPII/PSMA and NY-ESO-1 (as set out in FIG. 8C). Each of the six SLPs is conjugated to three copies of a sequence comprising the sequence of SEQ ID NO. 7, more specifically, to three copies of a sequence consisting of SEQ ID NO. 46 via a core (Core 1.0, Reference Synthesis Example 6).

It is to be understood that the method of the present invention is not limited to the aforementioned specific conjugates. Generally, the method of the present invention is applicable to any conjugate comprising at least one B-cell epitope and at least one CD4+ T-cell epitope where the CD4+ T-cell epitope is capable of promoting a T helper response that supports a feedback loop of antibody regulation in the subject to whom the conjugate has been administered. That is to say the CD4+ T-cell epitope is capable of eliciting a CD4+ T-cell immune response that co-stimulates B-cells specific to the B-cell epitope of the conjugate such that they generate antibody and thus increase the level of antibody specific for the conjugate in the subject. In this way, the quantity of antibody specific to the conjugate can be used as a biomarker for the presence of a CD4+ T-cell response in the subject to whom to conjugate has been administered.

It is to be appreciated that there are a number of advantages to measuring a quantity of antibody specific to the conjugate as a biomarker for the presence of a CD4+ T-cell response as opposed to measuring the CD4+ T-cell response directly. Typically, measuring a quantity of antibody requires a smaller volume of blood to be taken from the subject and methods to measure a quantity of antibody are quicker, less complex and less expensive to perform.

In one embodiment, the subject to whom the conjugate has been administered and from whom a sample is derived is a cancer patient. In embodiments in which the CD4+ T-cell epitope is a universal tumour antigen and thus is expected to be effective against a broad range of cancer types, the cancer from which the patient is suffering is not limited to any particular type of cancer (as described above). Where the subject has been administered the TENDU vaccine (as described above), the cancer patient is a prostate cancer patient.

The method of the present invention is capable of providing information on the presence (or absence) of a CD4+ T-cell response to the CD4+ T-cell epitope of the conjugate in the cancer patient. In one embodiment, this information is used to stratify the cancer patient within a population of patients and/or to inform the subsequent treatment and/or procedures received by the cancer patient. In one embodiment, the presence of a CD4+ T-cell response to the CD4+ T-cell epitope of the conjugate is indicative of a clinically relevant response to the conjugate in the cancer patient. That is to say a response that is associated with an improved clinical outcome for the cancer patient. In one embodiment, the improved clinical outcome is a partial or a complete response (also known as a partial or a complete remission) or stable disease. Thus the method of the present invention may be used to identity clinical situations in which an improved clinical outcome for the cancer patient is (or is not) to be expected. In one embodiment, the method of the present invention is used to investigate (e.g. in a clinical trial) a conjugate according to the present invention in combination with a further substance.

It is preferred that the antibody specific for the conjugate that is detected is an antibody specific for the B-cell epitope in the conjugate.

### EXAMPLES

Hereinafter, the invention will be specifically described with reference to the Examples. However, these Examples do not limit the technical scope of the invention.

### Example 1: SEQ ID NO: 1 is Immunogenic in 65% of Patients Vaccinated with the Cancer Vaccine UV1

### Background

The cancer vaccine UV1 is a cocktail of three hTERT polypeptides having the sequences of SEQ ID NO. 1 (ALFSVLNYERARRPGLLGASVLGLDDIHRA; p719-20), SEQ ID NO. 52 (RTFVLRVRAQDPPPE; p725) and SEQ ID NO. 53 (AERLTSRVKALFSVL; p728). UV1 specific immune responses have been investigated as a monotherapy in two clinical studies in patients with non-small-cell lung carcinoma (NSCLC) (UV1/hTERT-2012-L) or prostate cancer (UV1/hTERT-2012-P), and in combination with the anti-CTLA-4 antibody ipilimumab in patients with malignant melanoma (UV1/hTERT-MM). A total of 52 patients were treated in the studies, of which 51 were evaluable for monitoring of immune responses.

### Material and Methods

Vaccine specific immune responses were measured using a standard T-cell proliferation assay (measuring proliferation by 3H-thymidine incorporation; as previously described in Inderberg-Suso et al. Oncoimmunology. 2012 Aug. 1; 1 (5): 670-686) following re-stimulation of peripheral blood mononuclear cells (PBMCs) harvested before, during and after UV1 vaccination. Re-stimulation of the PBMCs was performed with the UV1 vaccine mix and the individual vaccine peptides (p719-20, p725 and p728). The specific T-cell response was considered positive if the peptide response was at least 3 times the background (stimulation index, SI ≥3) for at least one of the vaccine peptides or the UV1 vaccine mix.

### Results

Table 2 shows the immune responses against individual UV1 peptides (i.e. p719-20, p725 and p728), the UV1 vaccine (i.e. the mixture of p719-20, p725 and p728), or either the individual UV1 peptides or the UV1 vaccine. The data include follow-up for two years after the first UV1 vaccination.

TABLE 2
Immune responses against the UV1 vaccine and the individual
UV1 peptides in patients vaccinated with UV1

| | p719-20 | p728 | p725 | | p719-20, |
|---|---|---|---|---|---|
| | (SEQ ID | SEQ ID | SEQ ID | | p728, p725 or |
| Donor | NO. 1) | NO. 53) | NO. 52) | UV1 | UV1 |
| 1 | + | − | − | + | + |
| 2 | + | − | + | + | + |
| 3 | + | − | − | + | + |
| 4 | + | + | + | + | + |
| 5 | + | − | − | + | + |
| 6 | + | − | − | + | + |
| 7 | + | + | + | + | + |
| 8 | + | + | + | + | + |
| 9 | + | − | − | + | + |
| 10 | + | − | − | + | + |
| 11 | − | − | − | − | − |
| 12 | Not done | Not done | Not done | Not done | Not done |
| 13 | + | − | + | + | + |
| 14 | − | − | − | + | + |
| 15 | − | − | − | − | − |
| 16 | + | + | − | + | + |
| 17 | + | + | + | + | + |
| 18 | + | − | + | + | + |
| 19 | + | − | + | + | + |
| 20 | − | − | − | − | − |
| 21 | − | − | − | − | − |
| 22 | + | − | + | + | + |
| 23 | + | − | − | + | + |
| 24 | + | − | + | + | + |
| 25 | − | − | − | − | − |
| 26 | + | − | + | + | + |
| 27 | − | − | − | + | + |
| 28 | + | − | − | + | + |
| 29 | − | − | − | + | + |
| 30 | + | − | − | − | + |
| 31 | − | + | − | + | + |
| 32 | + | − | − | + | + |
| 33 | + | + | + | + | + |
| 34 | + | + | + | + | + |
| 35 | − | − | − | − | − |
| 36 | + | − | + | − | + |
| 37 | − | − | − | − | − |
| 38 | − | − | − | − | − |
| 39 | + | − | + | + | + |
| 40 | − | − | − | + | + |
| 41 | + | + | + | + | + |
| 42 | + | + | + | + | + |
| 43 | + | − | − | + | + |
| 44 | + | − | − | + | + |
| 45 | − | − | − | + | + |
| 46 | − | − | − | − | − |
| 47 | + | + | + | + | + |
| 48 | − | − | + | + | + |
| 49 | + | − | − | + | + |
| 50 | − | − | − | − | − |
| 51 | − | − | − | − | − |
| 52 | + | + | + | + | + |
| | 65% | 24% | 39% | 75% | 78% |
| | (33/51) | (12/51) | (20/51) | (38/51) | (40/51) |

Referring to Table 2, the immune monitoring data demonstrated a detectable UV1 specific immune response in 78% of patients (i.e. those patients that demonstrated an immune response to one of the individual peptides or the UV1 vaccine; see the column entitled “p719-20, p728, p725 or UV1”). At the individual peptide level, 65% of the patients recognised the p719-20 peptide (SEQ ID NO. 1). 24% recognised the p728 peptide (SEQ ID NO. 53) and 39% recognized the p725 peptide (SEQ ID NO. 52).

At the individual patient level, 20% of patients recognised all three individual UV1 peptides, 20% recognised two peptides and 40% recognised one peptide or the UV1 vaccine. Of the patients that recognised one peptide only. 13 recognised p719-20 (SEQ ID NO. 1), one recognized p728 (SEQ ID NO. 53), and one recognised p725 only (SEQ ID NO. 52).

### Discussion

The data shown in Table 2 demonstrate that a polypeptide having the sequence of SEQ ID NO. 1 is immunogenic in 65% of patients. The patient population was comprised of patients with NSCLC, prostate cancer or malignant melanoma who had been administered the UV1 cancer vaccine either as a monotherapy or in combination with the anti-CTLA-4 inhibitor ipilimumab.

### Example 2: The Frequency of HLA Allele Types in the UV1-Vaccinated Patient Population is Representative of a European Population

### Background

HLA allele typing of the patients vaccinated with the UV1 cancer vaccine (i.e. the population of 52 patients as described in Example 1) was performed in order to confirm that the frequencies of the HLA alleles present in this patient population were representative of a general European population and to investigate if there were any indications of bias in the use of HLA alleles for presentation of the T-cell epitopes of the UV1 peptides.

### Materials and Methods

HLA allele typing of all patients vaccinated with UV1 was determined by PCR-sequence specific oligonucleotides to resolve major allele groups to 4 digits. The analysis was performed in retrospect (i.e. following the inclusion of the patients into the clinical trials).

The analyses were conducted on PBMCs isolated from patient blood samples. HLA allele typing data were obtained from 48 patients out of a population of 52 non-HLA selected patients vaccinated with UV1.

### Results

Table 3 shows the frequency of the most common HLA alleles present in the population of patients vaccinated with UV1, together with the corresponding frequencies of these alleles across the European population (in parentheses, in bold). The HLA allele frequencies are obtained from The Allele Frequency (http://www.allelefrequencies.net/default.asp).

TABLE 3
Frequencies of the most common HLA alleles

| HLA class | | | | |
|---|---|---|---|---|
| I or II | HLA alleles | | | |
| HLA-A | *02:01 | *01:01 | *03:01 | *24:02 |
| | 50% (50%) | 35% (30%) | 20% (30%) | 18% (20%) |
| HLA-B | *07:02 | *08:01 | *44:02 | *40:01 |
| | 30% (20%) | 25% (21%) | 13% (25%) | 20% (10%) |
| HLA-DRB1 | *15:01 | *07:01 | *04:01 | *03:01 |
| | 35% (30%) | 10% (22%) | 25% (20%) | 28% (25%) |
| HLA-DQB1 | *06:02 | *03:02 | *05:01 | *02:01 |
| | 35% (25%) | 28% (20%) | 18% (20%) | 28% (20%) |
| HLA-DPB1 | *04:01 | *04:02 | 01:01 | *02:01 |
| | 65% (60%) | 15% (25%) | 15% (10%) | 30% (25%) |

Referring to Table 3, the HLA typing data demonstrated that a wide range of HLA allele types were present in the population of patients vaccinated with UV1. Furthermore, the observed frequencies within the patient population corresponded well to those recorded in a general Caucasian/European population (http://www.allelefrequencies.net/default.asp). These results were in line with the fact that recruitment of patients into the clinical trials was not based on HLA typing.

### Discussion

The data in Table 3 demonstrate that the frequency of HLA allele types in the UV1-vaccinated patient population is representative of a general European population.

### Example 3: Immune Responses Against a Polypeptide Having the Sequence of SEQ ID NO. 1 were Generated in Patients Having a Range of Common HLA Class II Alleles

### Background

The HLA allele typing data of Example 2 was further analysed to investigate the HLA class II allele distribution in UV1-vaccinated patients that had demonstrated an immune response to the p719-20 peptide (SEQ ID NO. 1).

### Results

Table 4 shows the number of patients (n=) and the corresponding percentage of patients having each of the three most common HLA-DR, -DQ and -DP haplotypes (alleles) (i.e. the upper set of figures shown in the three rows of the table). Table 4 also shows the number of patients carrying each allele who responded to a polypeptide having the sequence of SEQ ID NO. 1 (n=; underlined) and the percentage of patients carrying each allele who responded to a polypeptide having the sequence of SEQ ID NO. 1 (in parentheses) (i.e. the lower set of figures shown in the three rows of the table).

TABLE 4
Most frequently distributed HLA class-II alleles
combined with data on immune responses against
a polypeptide having the sequence of SEQ ID NO. 1

| | |
|---|---|
| | Patients having each HLA class II allele |
| | (upper row); |
| | Patients having each HLA class II allele and an |
| | immune response against SEQ ID NO. 1 |
| HLA class II | (lower row; underlined) |

| | | | |
|---|---|---|---|
| HLA-DRB1 | *15:01 | *07:01 | *04:01 |
| | n = 18 38% | n = 10 21% | n = 10 21% |
| | n = 16 (89%) | n = 9 (90%) | n = 5 (50%) |
| HLA-DQB1 | *06:02 | *03:02 | *05:01 |
| | n = 18 38% | n = 13 27% | n = 9 19% |
| | n = 13 (72%) | n = 6 (46%) | n = 6 (67%) |
| HLA-DPB1 | *04:01 | *04:02 | *01:01 |
| | n = 33 69% | n = 7 15% | n = 7 15% |
| | n = 21 (64%) | n = 5 (71%) | n = 1 (14%) |

Referring to the data in Table 4, the HLA allele determination and the immune response analysis for the polypeptide of SEQ ID NO. 1 performed on the individual patients demonstrated that immune responses against the polypeptide of SEQ ID NO. 1 were generated in patients across all of the most common HLA class II haplotypes, with >50% responders in 7 of the 9 most common haplotypes.

### Discussion

The data in Table 4 demonstrate that the polypeptide of SEQ ID NO. 1 is capable of being recognised by a range of HLA class II alleles that are common in a general European population. This observation, together with the fact that fully heterozygous patients express 9 different HLA class II haplotypes, explains the high proportion of immune responders against the polypeptide of SEQ ID NO. 1 across the 3 different patient populations (as described in Example 1). Therefore, the polypeptide of SEQ ID NO. 1, which is derived from the universal tumour antigen hTERT, is expected to provide broad population coverage in a wide range of (or all) cancer indications.

### Example 4: Different Regions within SEQ ID NO: 1 are Recognised by Different T-Cell Clones from UV1-Vaccinated Patients

### Background

A T-cell immune response in a human subject is determined by two cell types (APC and T-cell) and its specificity is determined by 3 molecules (HLA class-I/II, peptide and T-cell receptor (TCR)). In a single individual, 6 different HLA class-I and 6 HLA class-II molecules may be expressed, while at a population basis several thousand different polymorphic variants exist. The number of different peptides is virtually unlimited. The number of different TCRs in a single individual is also extremely high.

Processing of peptides for HLA class-I binding is highly regulated takes place in proteasomes and results in short peptides (8 to 10 mers). Proteasomal cleavage sites limit the number of 8 to 10-mers than can be produced from a given protein. Loading of peptides into HLA-class I is also highly regulated via transporters and chaperones etc. In contrast, processing of peptides for HLA-class II binding is very complex (Stern L J, Santambrogio L. Curr Opin Immunol. 2016; 40:70-77) and was originally thought to only take place in endosomes. Endosomes represent a highly dynamic and diversified system for protein sorting and degradation. One main task of the endosomes in protein degradation is to provide single amino acids to be re-used for protein synthesis. This goal is reached by the use of a combination of multiple endo- and exo-proteases. Class II molecules reach endosomes with their binding site protected by Invariant chain (li), which is cleaved in lyso-endosomes, leaving the binding site open for capture of peptides with an amino acid sequence/binding motif fitting the particular class II molecule. HLA class II molecules thus rescue such molecules from further degradation and are transported to the cell surface. The core binding motif of the peptide is necessary for binding, but peptide elution followed by mass spectrometry has shown that such peptides have “ragged ends” extending at both sites of the core binding motif (Muntasell et al. J Immunol. 2002 Nov. 1; 169 (9): 5052-60.). Recognition by individual CD4+ T cell clones requires binding to a relevant class II molecule through the core binding motif but is influenced by the flanking amino acids.

Thus different lengths of peptides derived from a polypeptide, such as SEQ ID NO: 1, and with the same binding motif may activate different T-cells. Promiscuity of peptide binding to different HLA class II molecules will further extend the number of different T-cell clones activated. Therefore, long peptides, such as SEQ ID NO: 1 with several core-binding motifs embedded will extend population coverage of a vaccine.

### Results

Table 5 shows the fine mapping of T-cell clones and the sequences within SEQ ID NO: 1 that are recognised by each clone. The data are based on T-cell clones derived from patients that responded to UV1 vaccination. All clones listed in Table 5 responded to the polypeptide of SEQ ID NO: 1 when tested in vitro (using a T-cell proliferation assay as described in Example 1). In addition, the T-cell clones were tested using a series of overlapping peptides derived from SEQ ID NO: 1. As is shown in Table 5, HLA-DR molecules preferentially presented peptides present in the N-terminal half of the 30-mer polypeptide of SEQ ID NO: 1 with at least 2 alleles (DR12 and DR7), while DQ2 and DQ7 alleles presented the C-terminal part. The data show that a range of 14-mer peptides derived from the 30-mer polypeptide of SEQ ID NO: 1 are presented by different HLA class II molecules and can be recognised by different T cell clones.

Individual T cell clones recognise different “frames” of SEQ ID NO: 1 represented by the 14-mer peptides shown in Table 5. Thus the three DR12 restricted clones only recognised the first N-terminal 14-mer. The reactivity was lost when the N-terminal alanine was removed and a C-terminal glycine was added. The DR7 restricted clones were split in their preference. The reactivity of clones 4, 12, 21 and 92 was lost when the N-terminal serine was removed and a C-terminal glycine was added. On the other hand, clone 15 still recognised this peptide but recognition was abolished when the N-terminal valine was removed and a C-terminal aspartic acid was added. Conversely, clone 109 from patient 5 recognised 6 different 14-mer peptides and only seemed to depend on the presence of the 9-mer core binding sequence of “NYERARRPG” (SEQ ID NO: 163. A tentative DR specificity for these clones is HLA-DR8. Similar results were seen with the HLA-DQ7 restricted T-cell clone; clone 8 seemed to require the 9-mer core binding sequence of “RRPGLLGAS” (SEQ ID NO: 164) whereas clone 10 did not recognise the first two peptides recognised by clone 8. Thus the C-terminal leucine need to be in place to obtain recognition by this clone. Clone 52 recognises a C-terminal frame consisting of the core-binding region “LLGASVLGLDDI” (SEQ ID NO: 165).

TABLE 5
Fine mapping of T-cell clones recognising
peptides from SEQ ID NO: 1

| Sequence (SEQ ID NO.) | HLA Class II allele, T-cell clone |
|---|---|
| ALFSVLNYERARRPGLLGASVLGLDDIHRA | All clones |
| (SEQ ID NO: 1) | |
| ALFSVLNYERARRP (SEQ ID NO: 12) | DR12, clone 44, 38, 6 |
| LESVLNYERARRPG (SEQ ID NO: 13) | p5-clone109 |
| FSVLNYERARRPGL (SEQ ID NO: 14) | p5-clone 109 |
| SVLNYERARRPGLL (SEQ ID NO: 15) | DR7, clone 4, 12, 21, 15, 92, |
| | p5-clone109 |
| VLNYERARRPGLIG (SEQ ID NO: 16) | DR7, clone 15, p5-clone109 |
| LNYERARRPGLLGD (SEQ ID NO: 17) | p5-clone109 |
| NYERARRPGLLGAS (SEQ ID NO: 18) | p5-clone 109 |
| NYERARRPGLLGAS (SEQ ID NO: 18) | DQ7, clone 8 |
| YERARRPGLLGASV (SEQ ID NO: 19) | DQ7, clone 8 |
| ERARRPGLLGASVL (SEQ ID NO: 20) | DQ7, clone 8,10 |
| RARRPGLLGASVLG (SEQ ID NO: 21) | DQ7, clone 8,10 |
| ARRPGLLGASVLGL (SEQ ID NO: 22) | DQ7, clone 8,10 |
| RRPGLLGASVLGLD (SEQ ID NO: 23) | DQ7, clone 8,10 |
| PGLLGASVLGLDDI (SEQ ID NO: 25) | DQ2, clone 52 |
| GLLGASVLGLDDIH (SEQ ID NO: 26) | DQ2, clone 52 |
| LLGASVLGLDDIHR (SEQ ID NO: 27) | DQ2, clone 52 |

### Discussion

The data point to the role of not only HLA binding of the peptides, where different HLA class II molecules select individual “frames” from a longer polypeptide based on the preference for “core binding” sequences, but also highlight the role of the composition of the flanking region in determining the efficacy of the immune response generated by polypeptides such as SEQ ID NO: 1 in cancer patients. Thus polypeptides such as SEQ ID NO: 1 comprise different regions within their overall sequence that are recognised by different T-cell clones, which may contribute to the high immunogenicity of this polypeptide (for example, as discussed in Example 1).

### Example 5: The Polypeptide of SEQ ID NO: 116 Contains a High Number of Epitopes Capable of Eliciting a T-Cell Immune Response

### Materials and Methods

Epitope analysis was performed on samples from cancer patients who had been vaccinated with GV1001 (n=4) or dendritic cells (DC) transfected with hTERT mRNA (n=3) and who were identified as clinical responders (as described in WO 2011/101173 and Inderberg Suso et al. Cancer Immunol Immunother. 2011 June; 60 (6): 809-18, incorporated herein by reference). The cancer patients included melanoma, lung cancer, colon cancer and pancreatic cancer patients. In brief, peripheral blood mononuclear cells (PMBCs) from various time points during vaccination were stimulated with an overlapping hTERT peptide library and then tested for specific T-cell responses in T-cell proliferation assays (as described in Bernhardt et al., 2006, incorporated herein by reference) using the peptides of 731, 713, 714, 730, 715, 729, 722, 709, 726, 727, 732, 723.

### Results

Table 6 shows the sequence in the active site region of hTERT that has been subject to epitope analysis. The individual peptides tested in the T-cell proliferation assays are numbered and underlined (i.e. 731, 713, 714, 730, 715, 729, 722, 709, 726, 727, 732, 723) and the sequences of GV1001 (SEQ ID NO: 126, a 16-mer polypeptide), SEQ ID NO: 116 (a 30-mer polypeptide) and a C-terminal reference sequence of SEQ ID NO: 118 (a 30-mer polypeptide) are highlighted and underlined. Polypeptides tested that gave a positive immune response, either as a result of epitope spreading after GV1001 vaccination (4 patients) or as a result of vaccination with hTERT mRNA transfected DC (3 patients) were considered to contain epitopes embedded in, for example, SEQ ID NO: 116 if they shared a minimal sequence of 8 amino acids (considered to be a minimal epitope/core binding region) with SEQ ID NO: 116. The number of such hits for each polypeptide is shown in Tables 6 and 7 below.

TABLE 6
Epitope analysis of active site region of hTERT

| | SEQ | | No. of |
|---|---|---|---|
| | ID | | positive |
| Peptide | NO: | Sequence | patients |
| | 116 | --------------------------------------- [symbol] --------------------------- | |
| GV1001 | 126 | -------------------------------------------------- [symbol] ---------------------------------- | |
| 731 | 130 | --------------------------------LKRVQLRELSEAEVRQ--------------------------------------------------- | 1 |
| 713 | 131 | ------------------------------------------EAEVRQHREARPALL------------------------------------------ | 4 |
| 714 | 132 | ----------------------------------------------------RPALLTSRLRFIPKP-------------------------------- | 7 |
| 730 | 133 | ---------------------------------------------------------TSRLRFIPKPDGLRP--------------------------- | 5 |
| 715 | 134 | --------------------------------------------------------------FIPKPDGLRPIVNMD---------------------- | 3 |
| 729 | 135 | ------------------------------------------------------------------PDGLRPIVNMDYVVG------------------ | |
| | | | Total = 20 |
| | | continued | |
| | 118 | -------------------------------------------- [symbol] ----- | |
| 722 | 136 | -------------------------------FVLRVRAQDPPPELY--------------------------------- | |
| 709 | 137 | ------------------------------------------PELYFVKVDVTGAY----------------------- | |
| 726 | 138 | -------------------------------------------ELYFVKVDVEGAYDT--------------------- | 2 |
| 727 | 139 | --------------------------------------------LYFVKVDVTGAYDTI-------------------- | 5 |
| 732 | 140 | ---------------------------------------------------VTGAYDTIPQDRLTE------------- | 1 |
| 723 | 141 | -----------------------------------------------------------PQDRLTEVIASIIKP----- | 1 |
| | | | Total = 9 |

TABLE 7
T-cell responses to individual polypeptides

| | Vaccination GV1001 + DC | | | | | | | | |
|---|---|---|---|---|---|---|---|---|---|
| | (n = 4 + 3) | | | Vaccination DC (n = 3) | | | Vaccination GV1001 (n = 4) | | |
| | SEQ ID | | SEQ ID | SEQ ID | | SEQ ID | SEQ ID | | SEQ ID |
| Peptide | NO: 116 | GV1001 | NO: 118 | NO: 116 | GV1001 | NO: 118 | NO: 116 | GV1001 | NO: 118 |
| SUM | 20 | 12 | 9 | 8 | 5 | 4 | 12 | 7 | 5 |
| 731 | 1 | | | | | | 1 | | |
| 713 | 4 | | | 1 | | | 3 | | |
| 714 | 7 | 7 | | 3 | 3 | | 4 | 4 | |
| 730 | 5 | 5 | | 2 | 2 | | 3 | 3 | |
| 715 | 3 | | | 2 | | | 1 | | |
| 729 | | | | | | | | | |
| 722 | | | | | | | | | |
| 709 | | | | | | | | | |
| 726 | | | 2 | | | 1 | | | 1 |
| 727 | | | 5 | | | 2 | | | 3 |
| 732 | | | 1 | | | | | | 1 |
| 723 | | | 1 | | | 1 | | | |

### Discussion

The data in Tables 6 and 7 demonstrate that SEQ ID NO: 116 contains the highest number of epitopes capable of mounting a T-cell immune response as compared to GV1001 and the C-terminal reference sequence of SEQ ID NO: 118 in DC- and GV1001-vaccinated patients. The use of two different types of patient material provides important validation of the epitopes. Notably, since both epitope spreading from GV1001 vaccination and mRNA vaccination rely on processing of individual epitopes from the hTERT protein, the data identify bona fide naturally processed hTERT epitopes. The observation that several of the epitopes are identified in different patients provides independent validation of these epitopes. The difference between number of responders for SEQ ID NO: 116 and GV1001 compared to the C-terminal reference sequence of SEQ ID NO: 118 is relatively higher in GV1001-vaccinated patients compared to DC-vaccinated patients due to overlap with direct vaccine responses to GV1001.

This analysis was performed on samples from patients who have been identified as clinical responders, indicating that the T-cell responses detected against the polypeptides analysed herein represent clinically-relevant epitopes. The polypeptide of SEQ ID NO: 116 therefore appears to contain a high number of clinically-relevant epitopes and, as shown by the data included in Tables 6 and 7, a higher number of epitopes than GV1001 or the C-terminal reference sequence of SEQ ID NO: 118.

### Example 6: Predicted T-Cell Epitope Counts and Population Coverage

### Background

The population coverage of therapeutic peptides used in cancer therapy can be estimated by looking at T-cell epitope content and population HLA frequencies. In many cases, peptides or peptide mixes contain peptides able to elicit activation of both CD4+ and CD8+ T-cells. This in turns means that both HLA class I and HLA class II binding is relevant. Population coverage was assessed for a number of peptides as set out in Table 8 below. In silico prediction of HLA-peptide binding was used together with population HLA allele frequency data in order to estimate coverage.

### Materials and Methods

### Population HLA Allele Frequencies

HLA frequency data from the Allele Frequency Net database (Gonzalez-Galarza et al., Nucleic Acids Res. 2020 Jan. 8; 48 (D1): D783-D788) was used. This database contains information for >1600 populations from >10 million donors. A challenge is the selection of representative high-level populations (e.g. European, Asian), based on smaller studies that in many cases are regional or focused on a certain ethnical group. Take Australia as an example, where AUS-European is substantially different from AUS-Aboriginal. However, comparing US-European with a German cohort shows very similar allele distribution. In in order to combine smaller studies into high-level population groups, the strategy used by Bui et al. (BMC Bioinformatics. 2006 Mar. 17; 7:153) was used, where the updated approach splits data into 16 geographical regions.

### In Silico Prediction of HLA-Peptide Binding/T-Cell Epitopes

Prediction of both HLA class I and HLA class II binding was done using SciCross in-house algorithms. For frequently occurring HLA alleles the accuracy of prediction models is in general high. This is due to a good availability of data for algorithm training. For rare alleles an approach similar to Tepitopepan was used for HLA class II prediction (Zhang et al., PLOS One. 2012; 7 (2): e30483).

### Allele Coverage

Allele coverage is calculated in two steps, with final analysis split into coverage of HLA class I and HLA class II separately: (i) HLA binding prediction is run for all alleles using the peptide of interest. An allele-specific binder is considered if the prediction score is among the top 5% percentile of the model's score distribution. A peptide is considered to be a binder for a given HLA allele if there is at least one 9-mer sub-peptide binder predicted. The output is a number of alleles where the peptide has at least one predicted binder. (ii) The alleles predicted are used to calculate coverage in different populations. The approach used in this step is an implementation of the method described by Bui et al. 2006. In addition, the population coverage of combination of peptides can be calculated by combining their individual alleles covered.

Peptide sequences used in the analysis Population coverage and total epitopes counts were calculated for five peptide/combined peptide groups as set out in Table 8 below. In addition, some specific comparisons of peptides in terms of HLA class I and class II allele coverage and population coverage were performed as set out at a) to c) below.

TABLE 8
Peptide sequences and combinations used
in the analysis

| Peptide/peptide | |
|---|---|
| combination name | SEQ ID NO./Sequence |
| GV1001 | 126 |
| | EARPALLTSRLRFIPK |
| P719-20 | 1 |
| GV_LSE | 116 |
| | LSEAEVRQHREARPALLTSRLRFIPKPDGL |
| P719-20 + GV_LSE | 1 and 116 |
| C-term-pep | 118 |
| | LYFVKVDVTGAYDTIPQDRLTRVIASIIKP |

### Specific Comparisons of Peptides:

a) SEQ ID NO: 1 vs SEQ ID NO: 116 and the combination thereof;
b) SEQ ID NO: 116 vs SEQ ID NO: 118;
c) SEQ ID NO: 116 vs SEQ ID NO: 126 (and vs SEQ ID NO: 1); and

### Results:

### Total Numbers of Predicted Epitopes

Table 9 shows the total number of predicted epitopes for the five peptide/peptide combinations as set out in Table 8 above. The total number of unique HLA alleles covered is given in parentheses. As is shown in Table 9, SEQ ID NO: 116 would appear to comprise a particularly high number of predicted class I epitopes. Furthermore, the combination of SEQ ID NO: 1 and SEQ ID NO: 116 appears to result in a high number of predicted class I and class II epitopes, suggesting the combination of these peptides would be expected to have a high level of efficacy.

TABLE 9
Total numbers of predicted epitopes

| | Class I No. | Class II No. |
|---|---|---|
| | Predicted Epitopes | Predicted Epitopes |
| Peptide(s) | (covered alleles) | (covered alleles) |
| GV1001 (SEQ ID NO: 126) | 28(25) | 24(22) |
| P719-20 (SEQ ID NO: 1) | 57 (32) | 150 (75) |
| GV_LSE (SEQ ID NO: 116) | 73(43) | 96(70) |
| P719-20 + GV_LSE | 130(51) | 243(86) |
| (SEQ ID NO: 1 and | | |
| SEQ ID NO: 116) | | |
| C-terminal peptide | 44(32) | 96(73) |
| (SEQ ID NO: 118) | | |

### Specific Comparisons

### a) SEQ ID NO: 1 vs SEQ ID NO: 116 and the Combination Thereof:

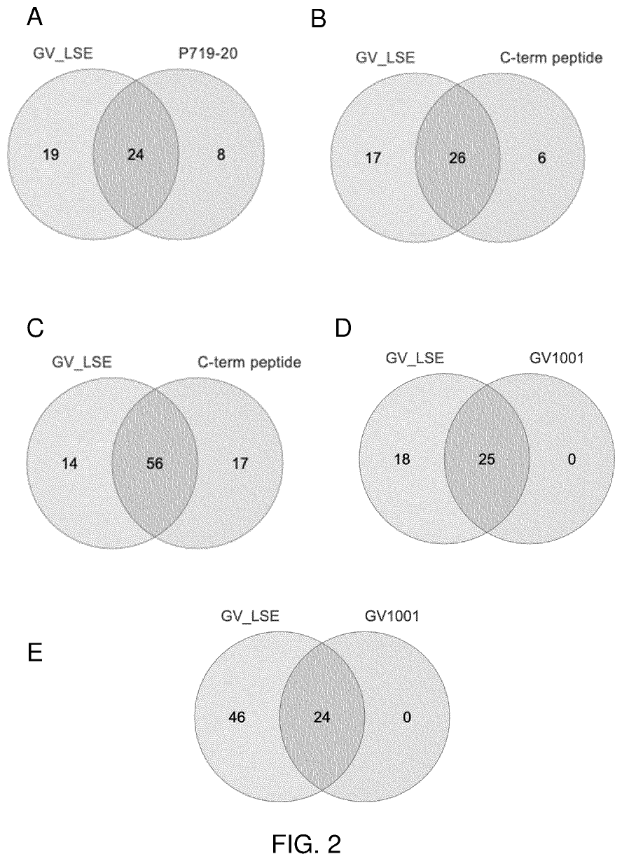

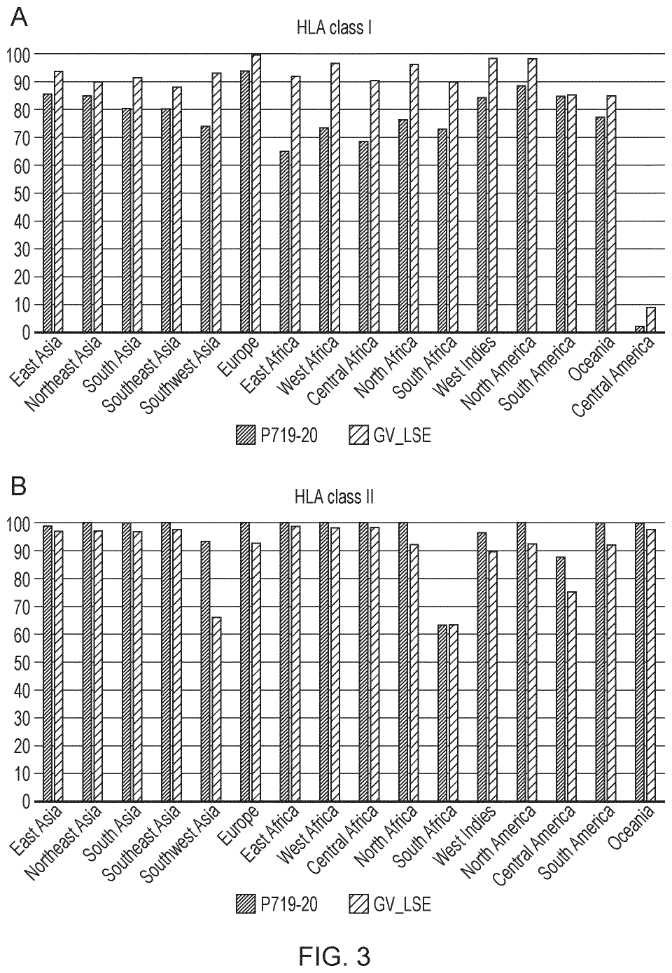

SEQ ID NO: 1 and SEQ ID NO: 116 differ in their predicted coverage of HLA class I and HLA class II alleles. SEQ ID NO: 116 has a higher coverage than SEQ ID NO: 1 for class I alleles, whereas SEQ ID NO: 1 has a higher coverage than SEQ ID NO: 116 for class II alleles (Table 9). FIG. 2A shows a comparison of the covered HLA class I alleles between SEQ ID NO: 1 and SEQ ID NO: 116. Combining SEQ ID NO: 1 plus SEQ ID NO: 116 would be expected to give a higher class I allele coverage. It is the peptide of SEQ ID NO: 116 that contributes the highest number of class I epitopes (it covers 19 unique alleles, see FIG. 2A). The combination of SEQ ID NO: 1 and SEQ ID NO: 116 gives a higher total predicted epitope count for both class I and class II epitopes (Table 9) as compared with each polypeptide alone. FIG. 3 shows the population coverage for SEQ ID NO: 1 and 116 based on HLA class I (FIG. 3A) and HLA class II alleles (FIG. 3B). Both polypeptides are expected to provide high population coverage based on HLA class II alleles (with SEQ ID NO: 1 generally providing slightly higher coverage than SEQ ID NO: 116). SEQ ID NO: 116 provides particularly high population coverage based on HLA class I alleles.

### b) SEQ ID NO: 116 vs SEQ ID NO: 118:

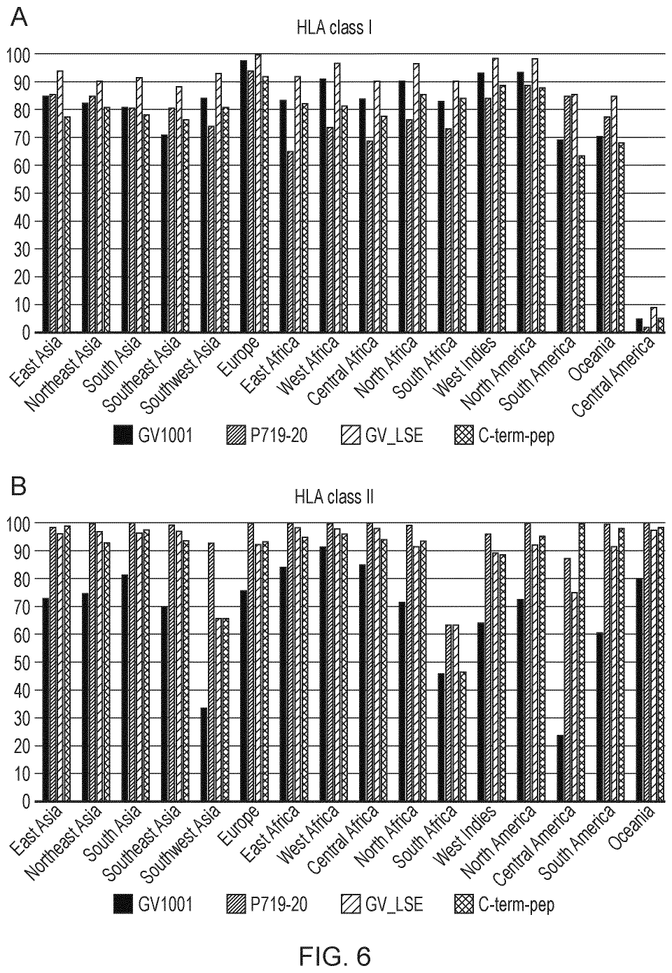

SEQ ID NO: 116 has a higher predicted epitope count and better coverage for HLA class I alleles, compared to SEQ ID NO: 118. FIG. 2B shows the overlap between covered class I alleles. Here it can be seen that most alleles covered by SEQ ID NO: 118 are also covered by SEQ ID NO: 116. The total coverage for HLA class II alleles between SEQ ID NO: 116 and 118 is rather similar. FIG. 2C shows a comparison of the covered HLA class II alleles between SEQ ID NO: 116 and SEQ ID NO: 118. There is a large overlap of covered alleles, but each individual peptide also covers a specific set of alleles. A similar pattern of results is also noted in relation to population coverage (FIGS. 6A and 6B, compare “GV_LSE” and “C-term-pep”), with SEQ ID NO: 116 expected to provide higher population coverage, in particular, based on HLA class I epitopes.

### c) SEQ ID NO: 116 vs SEQ ID NO: 126 (and vs SEQ ID NO: 1):

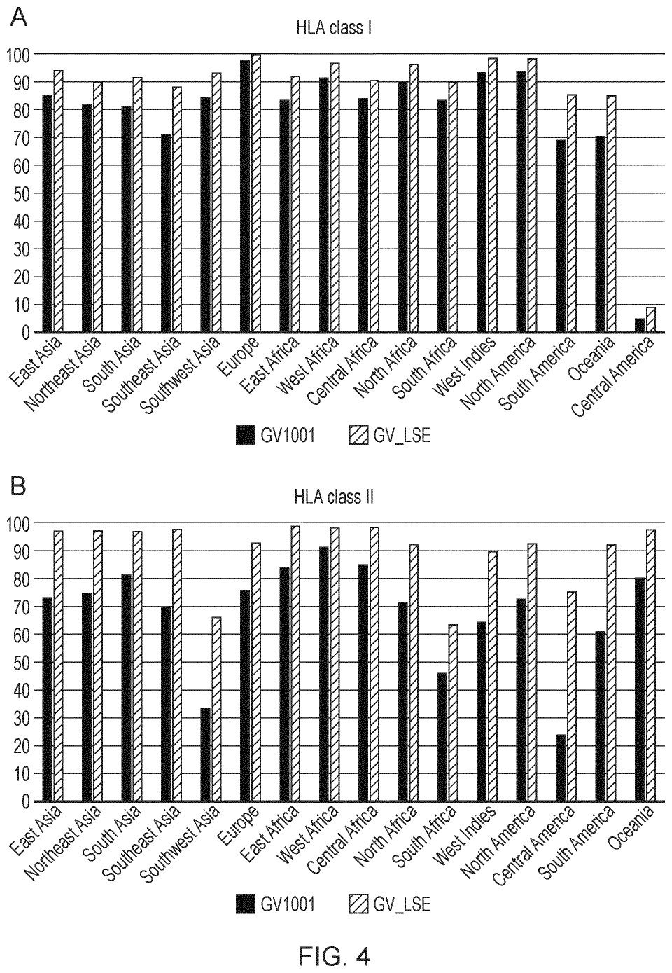

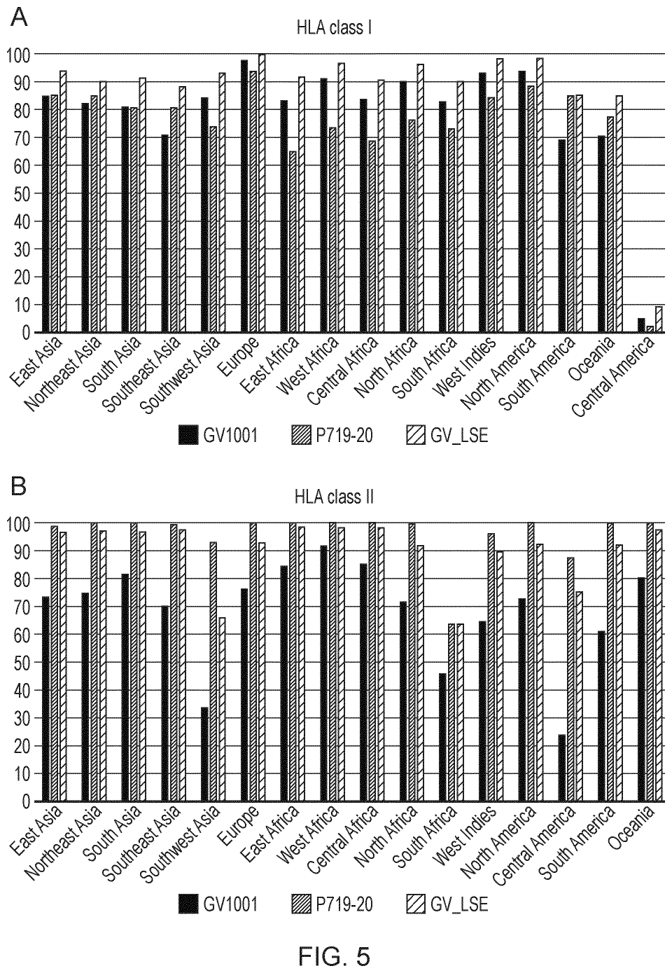

FIGS. 2D and 2E show comparisons of the covered HLA class I alleles and HLA class II alleles respectively between SEQ ID NO: 116 and SEQ ID NO: 126. Although there is some overlap between the covered HLA class I and II alleles, SEQ ID NO: 116 contributes a large number of unique alleles as compared to SEQ ID NO: 126, in particular in relation to HLA class II alleles. FIGS. 4A and 4B indicate that SEQ ID NO: 116 would provide higher population coverage based on HLA class I epitopes (FIG. 4A) and HLA class II epitopes (FIG. 4B) as compared to SEQ ID NO: 126. FIGS. 5A and 5B further show population coverage based on HLA class I and class II epitopes for SEQ ID NO: 116, 126 and also SEQ ID NO: 1. As discussed above, SEQ ID NO: 1 provides particularly high population coverage based on HLA class II alleles.

Note that very little HLA frequency data is available for Central America, meaning that no conclusions can be drawn from any seemingly low coverage.

### Discussion

The predicted T-cell epitopes and the population coverage data indicate that SEQ ID NO: 1 and SEQ ID NO: 116 and, in particular, the combination of these polypeptides, would be expected to provide a high level of efficacy over a broad range of the (worldwide) human population.

### Example 7: Administration of a Conjugate with or without a CD4+ T-Cell Epitope to Mice

### Example 7a): A Conjugate without a CD4+ Epitope does not Drive an Antibody Response to a B-Cell Epitope in Mice

### Materials and Methods

Female C57BL/6 mice were administrated a conjugate comprising a polypeptide comprising the sequence of a B-cell epitope (MTTE; SEQ ID NO. 7) and a polypeptide comprising the sequence of: AVGALEGSRNQDWLGVPRQL (SEQ ID NO. 54), containing the murine CD8+ T-cell epitope of mgp100. The conjugate did not comprise any known, complete CD4+ T-cell epitopes or any CD4+ T-cell epitopes from a universal tumour antigen. Administration of the conjugate was performed 3 times with a 1 week interval between each sub-cutaneous (SC) administration of 2 nmol of conjugate. 23 days post the last administration of the conjugate, blood was drawn from the mice and anti-MTTE IgG ELISA was performed with MTTE coated streptavidin plates to evaluate if the mice had developed anti-MTTE antibodies. Several mice were also injected with a monoclonal anti-MTTE IgG1 antibody (SC injection of 300 μg) as controls at the same time-points as the conjugates were injected. ELISA was performed by coating streptavidin plates with a biotinylated MTTE-containing peptide at a concentration of 1 nmol/ml overnight at 4° C. After this the plate was washed three times with a 0.05% Tween solution and then blocked with a solution containing 10% FBS and 0.05% Tween. Diluted plasma samples were incubated for 2 hours at room temperature and then washed again three times. A secondary polyclonal anti-mouse IgG1/HRP antibody was used to detect anti-MTTE antibodies bound to the plate. The secondary detection antibody was incubated for 1 hour in room temperature and subsequently the plate was washed and TMB (3,3′,5,5′-tetramethylbenzidine) was used to develop the plate. The reaction was stopped using 1 M H₂SO₄. Absorbance was read at 450 nm.

### Results

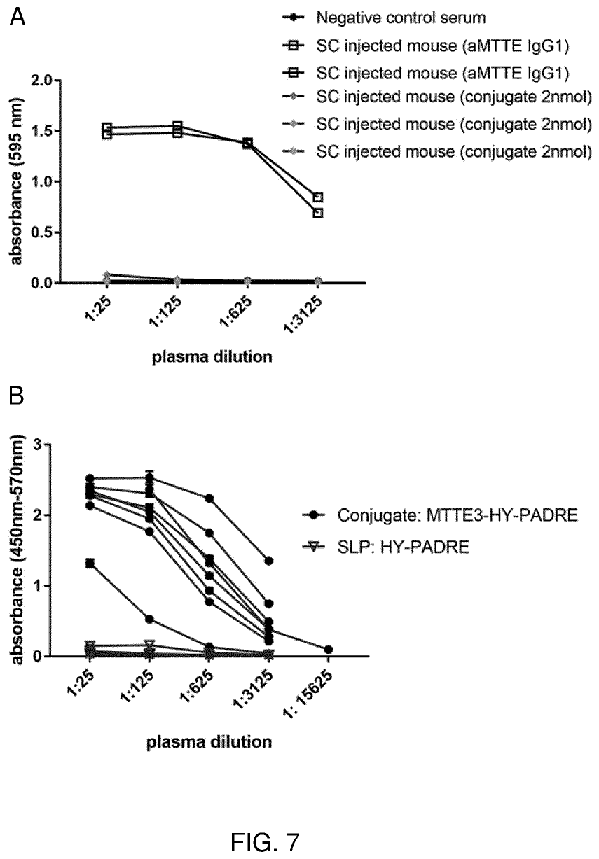

FIG. 7A shows that no endogenous anti-MTTE IgG1 antibodies were induced by a conjugate lacking a CD4+ T-cell epitope (see FIG. 7A, “SC injected mouse (conjugate 2 nmol)”). Anti-MTTE IgG1 injected animals displayed anti-MTTE antibodies in circulation (see FIG. 7A, “SC injected mouse (aMTTE IgG1)”). Thus only the mice injected with a passive transfer of monoclonal anti-MTTE IgG1 antibodies displayed measurable anti-MTTE antibody titres.

### Discussion

The data shown in FIG. 7A demonstrate that administration of a conjugate without a known CD4+ T-cell epitope is not capable of driving the production of anti-MTTE antibodies in mice.

### Example 7b): A Conjugate Comprising a CD4+ Epitope is Capable of Driving an Antibody Response to a B-Cell Epitope in Mice

### Materials and Methods

Mice were administered a conjugate comprising a polypeptide comprising the sequence of a B-cell epitope (MTTE; SEQ ID NO. 7) and a polypeptide comprising the sequence of SEQ ID NO: 55, which contains a non-endogenous, universal CD4+ T-cell epitope, termed “PADRE”.

SEQ ID NO. 55:
ARWWWMHHNMDLIGGAKxVAAWTLKAu

wherein:
x represents Cyclohexyl-Ala;
u represents D-Ala;
ARWW (SEQ ID NO. 56) is a TAP sequence (the TAP sequence is a sequence able to mediate TAP-driven transport of a polypeptide into the endoplasmic reticulum of a host cell);
WMHHNMDLI (SEQ ID NO. 57) is a murine CD8+ T-cell epitope of the minor HY antigen; GG is a proteasome cleavage site; and
AKxVAAWTLKAu (SEQ ID NO. 58) is the PADRE sequence (i.e. a non-endogenous, universal CD4+ T-cell epitope).

Mice were injected SC with 1 nmol of the conjugate or the naked peptide of SEQ ID NO: 55 alone (i.e. without conjugation to the MTTE sequence). Injection took place 3 times separated by 1 week intervals, and 9 days post the last administration of the conjugate or the naked peptide, blood was drawn from the mice and anti-MTTE IgG ELISA was performed with MTTE-coated streptavidin plates as described in Example 1a) to evaluate if the mice had developed anti-MTTE antibodies.

### Results

FIG. 7B shows that a conjugate comprising the universal PADRE sequence (i.e. a CD4+ T-cell epitope) was capable of driving production of anti-MTTE IgG1 antibodies to the conjugate as all the mice exposed to the conjugate had measurable anti-MTTE IgG1 antibody titres (see FIG. 7B, “Conjugate: MTTE3-HY-PADRE”). In contrast, mice exposed to only the naked peptide of SEQ ID NO. 55 containing the PADRE sequence (i.e. without conjugation to the MTTE sequence) had no antibodies to the MTTE sequence (see FIG. 7B, “SLP: HY-PADRE”) (as would be expected as they had not been exposed to the MTTE sequence).

### Discussion

The data shown in FIG. 7B demonstrate that the incorporation of a CD4+ T-cell epitope into the conjugate drives the production of anti-MTTE antibodies in mice. Thus the presence of a CD4+ T-cell epitope (in this example, the PADRE sequence) in the conjugate resulted in elevated anti-MTTE antibody levels, despite the mice not having been previously exposed to a tetanus vaccination. This can be contrasted with Example 7a), where a conjugate without a CD4+ T-cell epitope was not capable of driving the production of anti-MTTE antibodies in mice.

### Example 8: A Vaccine (“TENDU”) Comprising Conjugates Containing a B-Cell Epitope and CD4+ T-Cell Epitopes is Capable of Driving an Antibody Response to the B-Cell Epitope in Rabbits

### Materials and Methods

The TENDU vaccine comprises conjugates each containing three copies of a B-cell epitope (MTTE; SEQ ID NO. 7) and an SLP comprising a CD4+ T-cell epitope (further detail on the structure of the TENDU vaccine is provided in FIG. 8C). It was tested in male rabbits by Meditox (Konarovice, Czech Republic). The rabbits were subcutaneously vaccinated four times (with two-week intervals) with tetanus toxoid (TTd) vaccine (Equip® T vet. ≥30 IU/ml, Orion Pharma Animal Health) to generate circulating anti-MTTE antibodies (FIG. 8A). After another two weeks the rabbits were subcutaneously vaccinated four times (with two-week intervals) with TENDU at a low (10 μg/conjugate, n=5), intermediate (100 μg/conjugate, n=5) or high (240 μl/conjugate, n=8) dose. The two control groups were rabbits that only received the TTd vaccine (n=5) and rabbits that only received the high dose of TENDU (n=5) and not the Equip®T vet. Blood samples were collected for serology analysis to detect MTTE-specific antibody production after the TTd vaccination cycle (week 8) and after the TENDU vaccination cycle (week 15), which was performed by Capra Science (Sweden). The rabbit anti-MTTE titres were measured by Capra Science antibodies AB in an in-house-ELISA where biotinylated MTTE-coated streptavidin plates were used to detect induced rabbit anti-MTTE antibodies pre- and post-TTd vaccination as well as pre- and post-TENDU vaccination. A goat anti-Rabbit IgG conjugated to Alkaline Phosphatase was used as a secondary antibody. The substrate 4-Nitrophenyl phosphate disodium salt hexahydrate (1 mg/ml) was used. Absorbance was measured at 405 nm and curve fitting was used to determine the titre pre- and post-vaccination.

### Results

FIG. 8B shows that the TTd induced low levels of anti-MTTE antibody titres in the rabbits, which were greatly enhanced by TENDU vaccination at the intermediate and high doses of the TENDU vaccine conjugates (FIG. 8B). The rabbits vaccinated only with the TENDU vaccine (FIG. 8B, “no TTd”) had low levels of anti-MTTE antibodies as compared to the group with a pre-existing anti-MTTE antibody response induced by the TTd vaccination. Therefore, the rabbits displayed a difference in their anti-MTTE titre response depending on whether or not they had a pre-existing anti-MTTE response induced by TTd exposure. The rabbits with a pre-existing anti-MTTE response displayed significantly elevated titres of anti-MTTE antibodies pre-versus post- the high dose TENDU vaccination cycle (paired t-test; p=0.0013) which was not noted in the animals exposed to the same high dose of TENDU but that did not have pre-existing antibodies to the MTTE sequence.

### Discussion

The TENDU vaccine is not specifically designed to harbour rabbit-specific T-cell epitopes but rabbit MHC molecules can still present peptides from the synthetic long peptides comprised within the TENDU vaccine conjugates and as such these long peptides can act to drive the production of anti-MTTE antibodies in the rabbits. In the rabbits with pre-existing antibodies, the formation of immune complexes can occur which should induce an immune response as per the mode-of-action as exemplified in FIG. 1. The titre data indicate that an active substance has been administrated in a clinically relevant model and that a vaccine comprising conjugates containing the MTTE B-cell epitope and CD4+ T-cell epitopes can drive the production of anti-MTTE antibodies in the model. The data confirm the mode-of-action with the delivery route and the doses chosen. It also supports the use of a TTd booster vaccination prior to the start of vaccination with a conjugate comprising an (MTTE) B-cell epitope and CD4+ T-cell epitope (in this example, the TENDU vaccine). However, it is possible that further injections of the conjugate would have a similar effect to the use of a TTd booster in terms of antibody production.

### Example 9: Evaluation of Conjugate Binding by Anti-MTTE Antibody

### Materials and Methods

Binding of GMP LUG1-6 Constructs to Human Monoclonal Anti-MTTE IgG1 Antibody An in-house ELISA was used to confirm binding of GMP-produced LUG1-6 conjugates to a recombinant human monoclonal anti-MTTE IgG1 antibody. ELISA plates were coated with 100 μl/well conjugates diluted in Milli-Q water at a range of concentrations (0.000457-1 nmol/ml, a single conjugate per well). The plates were covered and incubated at 4° C. overnight. The plates were subsequently washed four times and blocked with 200 μl/well PBS containing 10% BSA and 0.05% Tween20 and incubated at room temperature (RT) for 1 hour. After washing, the human chimeric anti-MTTE IgG1 antibody (custom made by Evitria AG, Switzerland, >99% monomeric content and <0.1 EU/mg endotoxin), at 0.1 μg/ml in PBS supplemented with 1% BSA and 0.05% Tween20 was added. The plates were washed four times with 250 μl/well PBS containing 0.05% Tween20 and the secondary antibody diluted 1:8000 in PBS supplemented with 1% BSA (anti-human kappa light chain secondary antibody, Thermo Fisher Scientific #A 18853) was added to all wells (100 μl/well). After incubation for 1 hour at RT in the dark the plates were washed and 100 μl TMB was added to the wells. The reaction was stopped with 100 μl/well 1 M H₂SO₄ and the absorbance was measured at 450-570 nm wavelength.

### Binding of GMP LUG1-6 Constructs to Human Polyclonal Anti-MTTE Antibody

The same in-house ELISA as above was used to confirm binding of GMP-produced LUG1-6 constructs to human polyclonal anti-MTTE antibody from plasma from a human donor previously confirmed to have anti-MTTE antibodies. ELISA plates were coated with 100 μl/well conjugate diluted in Milli-Q water at a range of concentrations (0.004, 0.03, 0.4 and 1 nmol/ml, a single conjugate per well). The plates were covered and incubated at RT for 2 hours. The plates were then washed 4 times with 250 μl/well PBS containing 0.05% Tween20. The plates were then blocked 3 times with 200 μl/well Superblock T20 (Thermo Scientific) for 5 mins at RT. Plates were washed 4 times with 250 μl/well PBS containing 0.05% Tween20. Donor human plasma was diluted 1:200 in PBS supplemented with 1% BSA and 0.05% Tween20, and 100 μl/well applied to the plates, which were then incubated for 2 hours at RT. Plates were again washed 4 times with 250 μl/well PBS containing 0.05% Tween20, and the secondary antibody diluted 1:8000 in PBS supplemented with 1% BSA (anti-human kappa light chain secondary antibody, Thermo Fisher Scientific #A 18853) was added to all wells (100 μl/well). After incubation for 1 hour at RT in the dark the plates were washed and 100 μl TMB was added to the wells. The reaction was stopped with 100 μl/well 1 M H₂SO₄ and the absorbance was measured at 450-570 nm wavelength.

### Results and Discussion

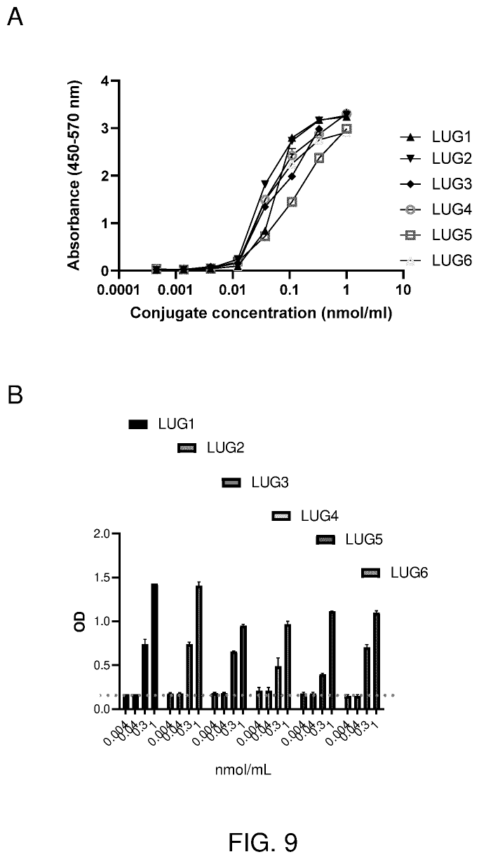

FIGS. 9A and 9B show that conjugates LUG1-6 can each be coated onto ELISA plates and detected by monoclonal anti-MTTE antibodies (FIG. 9A) and polyclonal antibodies from human plasma from a donor with confirmed anti-MTTE antibodies (FIG. 9B). Thus with increasing concentrations, each of the LUG1-6 conjugates was shown to bind monoclonal anti-MTTE antibodies and endogenous polyclonal antibodies from a specific donor. The data demonstrate that anti-MTTE antibodies (monoclonal and polyclonal) are capable of binding the MTTE sequence when comprised within a conjugate.

### Example 10: Administration of the LUG2 Conjugate to Humanised HLA-DR4 Mice

### Materials and Methods

### Evaluation of Epitope-Specific T Cell Responses in Humanised HLA-DR4 Mice

Female HLA-DR4 transgenic mice on a C57BL/6 background (12 weeks old at the start of the study) were acquired from Taconic (Germantown, MD, USA). HLA-DR4 animals were administered a LUG2 construct (20 μg) subcutaneously at the tail base followed by a boost two weeks later. A week later the mice were sacrificed, and the spleens were collected for generation of single cell suspensions for analysis by ELISPOT as described below. Heart bleed was performed to analyze anti-MTTE titers after LUG2 exposure. Tail vein-sampled HLA-DR4 animals that had not been exposed to LUG2 were used as controls for baseline titre assessment (unexposed animals).

### Evaluation of Immune Responses

Antibody titres against the MTTE sequence were determined using an in-house ELISA. Streptavidin plates (Thermo Scientific) were coated with the peptide having the sequence of FIGITELKKLESKINKVFSSAFADVEAA (SEQ ID NO. 142), biotinylated at its C-terminus, overnight at 4° C. The plates were washed with PBS (0.05% Tween) and blocked with PBS (10% BSA and 0.05% Tween) for 1 hour at RT. The mouse serum was serially diluted in PBS (1% BSA and 0.05% Tween), applied to the plates and incubated for 2 hours at RT. Mouse MTTE-specific IgG antibodies were detected with secondary HRP-conjugated antibody: goat anti-mouse IgG (polyclonal antibody from Dako; diluted 1:4000). The secondary HRP-conjugated antibody was diluted in PBS (1% BSA) and incubated on the plates for 1 hour at RT. The reaction was developed with the substrate TMB (Dako) and stopped with 1 M H₂SO₄. The absorbance was read at 450-570 nm using an iMark microplate reader (Bio-Rad).

The immunogenicity of the HLA-DR4 epitope was assessed by stimulating splenocytes with SLPs containing the embedded HLA-DR4 sequence. This was performed using an ex vivo IFNγ ELISpot assay (ELISpot kit for mouse IFNγ/3321-2A, Mabtech, Stockholm, Sweden). The LUG2 SLP with the TAP sequence has the amino acid sequence: ARWWLLHETDSAVAAARQIYVAAFTVQAAAE (SEQ ID NO. 143), and the LUG2 SLP without the TAP sequence has the amino acid sequence of: LLHETDSAVAAARQIYVAAFTVQAAAE (SEQ ID NO. 144); both contain the embedded HLA-DR4 sequence. One day before spleens were harvested, 96-well ELISpot plates (Millipore) for the IFN-γ ELISpot assay were pre-coated with capture antibody according to the manufacturer's protocol. After 5 washes with PBS/Tween and blocking for a minimum of 30 min with T cell medium including RPMI 1640 (Life Technologies/Thermo Fisher Scientific), containing 1% w/v L-Glutamine (SLS/Lonza), 10% v/v FBS (Fisher/GE Healthcare), 2% HEPES (SLS/Lonza), 0.1% v/v Fungizone (Promega), 0.5×10⁶/well freshly isolated splenocytes were seeded in triplicate into the plate along with 100 μl of the respective SLPs at a final concentration of 10 μg/ml. The cells were then incubated at 37° C. in a 5% CO₂ incubator for 48 hours, and the plates then washed 5 times with DPBST. 50 μl/well biotinylated detection antibody (1/1000 dilution) against mouse IFNγ was then added, and the plates incubated for 2 hours at room temperature. Plates were then washed 5 times with DPBST, followed by the addition of 50 μl/well streptavidin alkaline phosphatase (1/1000 dilution). Plates were then incubated for 1 h 30 min at room temperature. After incubation, plates were washed again 6 times with DPBST and then 50 μl/well development solution (BCIP/NBT, BioRad) was added. The plates were left in the dark at room temperature until spots could be seen. Once spots developed, the reaction was stopped by rinsing the plates with tap water. Plates were then left to dry and the spots were quantified using an ELISpot plate reader (Cellular Technology Limited, Shaker Heights, OH, USA). SEB, the staphylococcal enterotoxin-B (at 2.5 μg/ml) was used as positive control, and unstimulated splenocytes (cells alone) were used as a negative control for every ELISpot assay. All experiments were performed in triplicate. Animals were scored as having a positive reaction when the number of spots in the cells-alone wells did not reach more than 20 and when the response in the peptide-containing wells was at least twice that of the standard deviation of the mean of the control wells.

### Results and Discussion

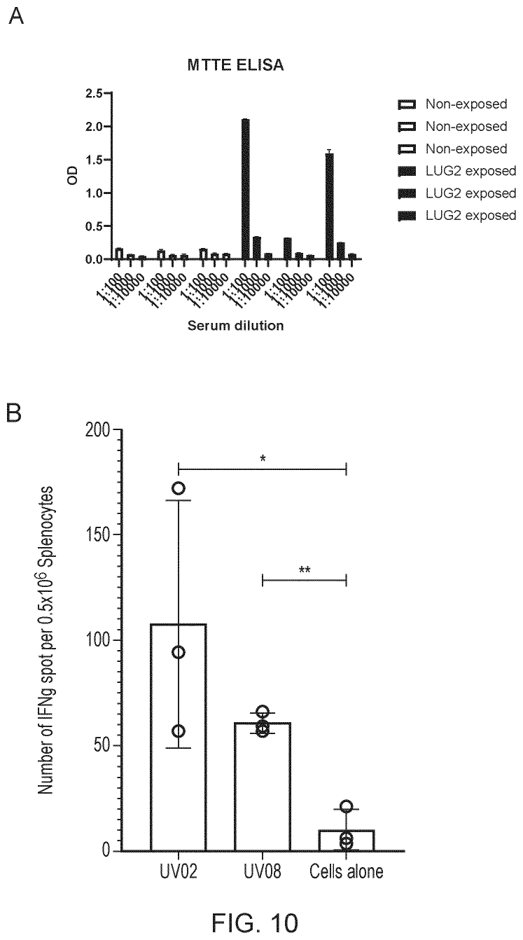

LUG2 includes an HLA-DR4 restricted PSMA epitope and so it was possible to expose animals to LUG2 conjugates and evaluate CD4+ T cell priming. HLA-DR4 mice received a prime/boost vaccination schedule with the LUG2 constructs. FIG. 10A shows that from serum collected from the non-exposed (control) and LUG2 exposed animals, mice exposed to LUG2 showed an increase in their anti-MTTE titre. FIG. 10B shows that upon treatment of splenocytes from the LUG2-vaccinated animals with the SLP contained in the LUG2 construct (UV02, SEQ ID NO: 143) or the SLP without the TAP ARWW sequence (UV08, SEQ ID NO: 144), an increased number of IFN-γ producing T cells was observed. FIG. 10 shows the results obtained from mice vaccinated with 20 μg LUG2; a similar pattern of results was obtained from mice vaccinated with 5 μg of LUG2 (data not shown). Therefore, administration of a conjugate comprising a B-cell epitope and a CD4+ T-cell epitope derived from a prostate cancer-associated antigen to HLA-DR4 mice resulted in elevated titres of antibody specific to the B-cell epitope (anti-MTTE antibody) in exposed versus non-exposed mice and a T-cell response to the CD4+ T-cell epitope of the conjugate. The data also demonstrate that a booster vaccination is not required prior to vaccination with the conjugate in order to observe the increased antibody levels and T-cell response in vivo when a CD4+ T-cell epitope is incorporated in the conjugate. Furthermore, the data demonstrate that a CD4+ T-cell epitope derived from a natural prostate cancer-associated antigen can achieve the induction of anti-MTTE antibodies similar to the non-endogenous PADRE sequence.

### Synthesis Examples 1 to 22

The following Synthesis Examples describe the synthesis of core compounds and conjugates comprising the core compounds in accordance with embodiments of the present invention.

### Core Synthesis—Synthesis Examples 1 to 10

The following Synthesis Examples describe the synthesis of core compounds in accordance with embodiments of the present invention.

### Synthesis Example 1: DBCO-Core A

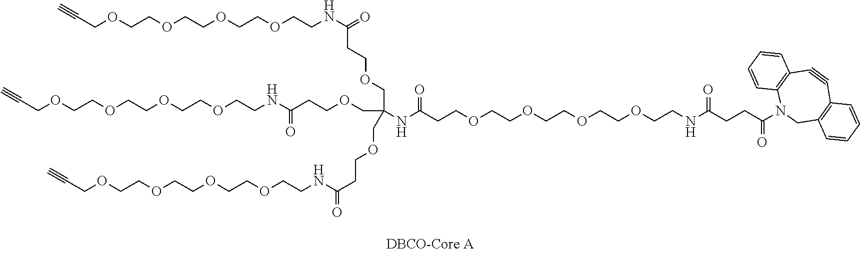 [SMILES: C#CCOCCOCCOCCOCCNC(=O)CCOCC(COCCC(=O)NCCOCCOCCOCCOCC#C)(COCCC(=O)NCCOCCOCCOCCOCC#C)NC(=O)CCOCCOCCOCCOCCNC(=O)CCC(=O)N1Cc2ccccc2C#Cc2ccccc21]

DBCO-acid is activated using PyOxim/Oxyma pure and DIPEA in DMF and reacted with Amino-PEG4-Tris-PEG4-alkyne (Core A) in DMF. The product is purified by flash chromatography (Scheme 1).

LCMS characterisation data: MWₜₕₑₒ=1510.8; MW_{found}=1511.7 [M+H⁺]

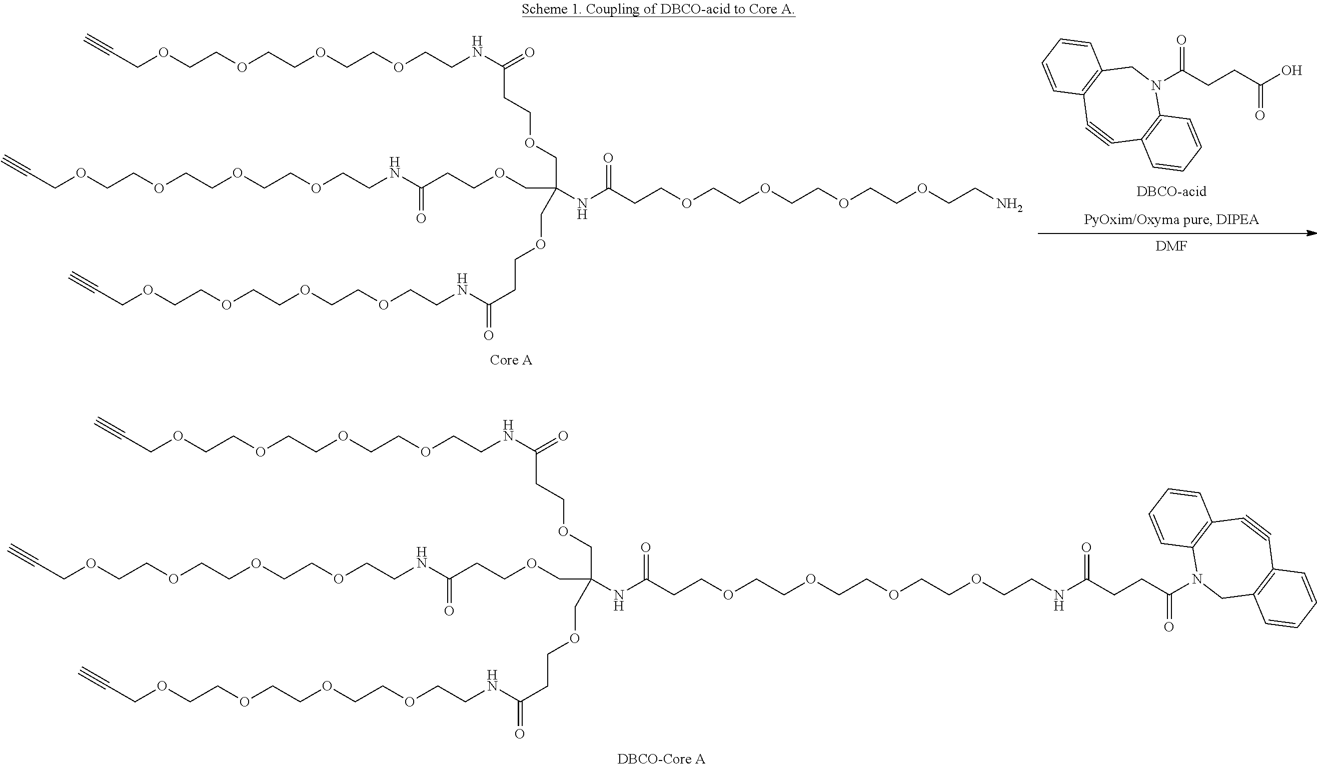

### Synthesis Example 2: PG-[Lys(Br)]₃

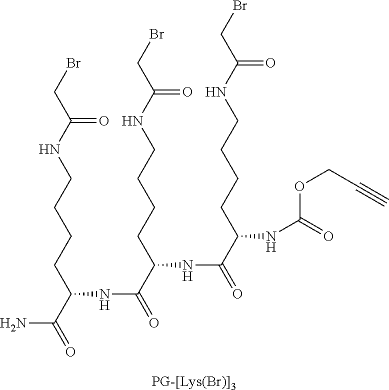 [SMILES: C#CCOC(=O)N[C@@H](CCCCNC(=O)CBr)C(=O)N[C@@H](CCCCNC(=O)CBr)C(=O)N[C@@H](CCCCNC(=O)CBr)C(N)=O]

Starting from Rink amide resin, Fmoc-Lys(Boc)-OH is coupled in series three times using SPPS conditions. After Fmoc-removal, propargyl chloroformate is coupled using DIPEA in DMF and the core is cleaved off from the resin using TFA. Bromoacetic acid is coupled using DIC in DMF and the product is purified by RP-HPLC purification and freeze dried (Scheme 2).

SPPS conditions: Amino acids are coupled using DIC/Oxyma pure and DIPEA in DMF in a 5-fold excess to a linker derivatized standard peptide synthesis resin. Fmoc is removed using 20% piperidine in NMP.

LCMS characterisation data: MWₜₕₑₒ=845.0; MW_{found}=846.0 [M+H⁺]

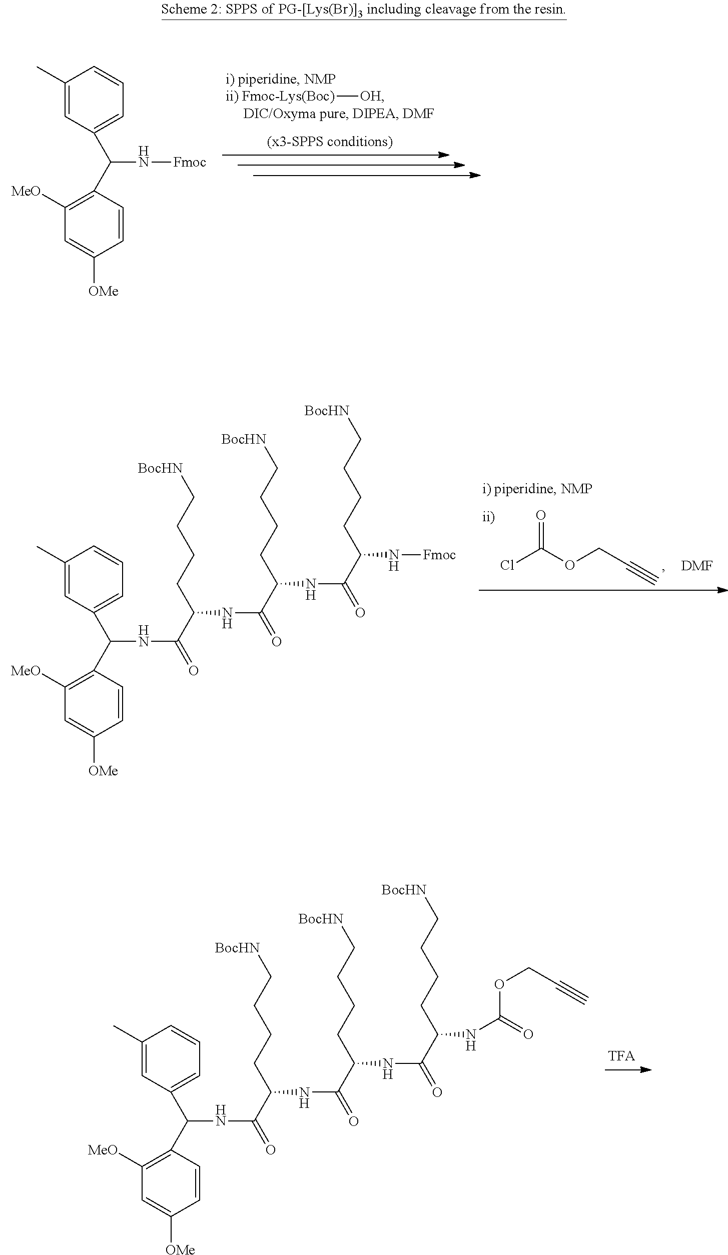

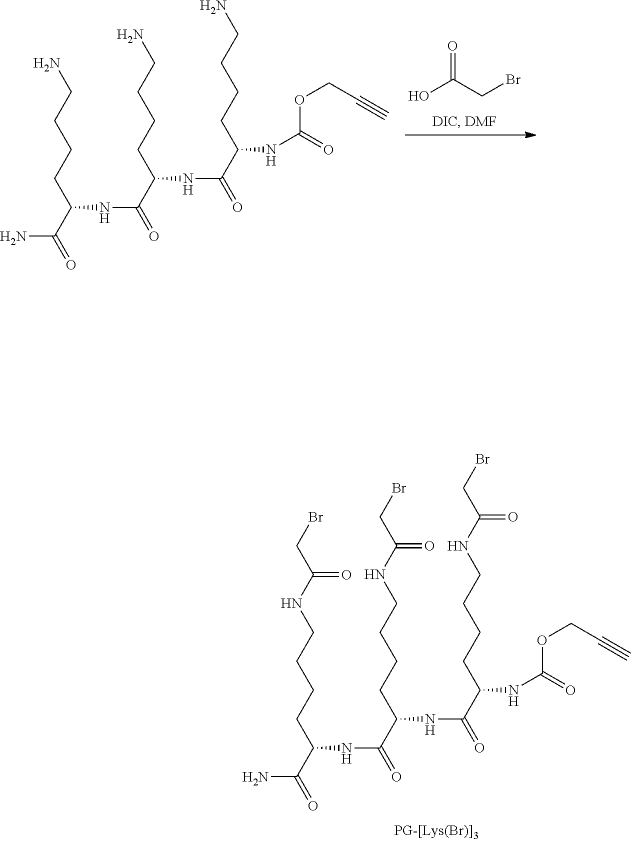

### Synthesis Example 3; PG-[Lys(Mal)]₃

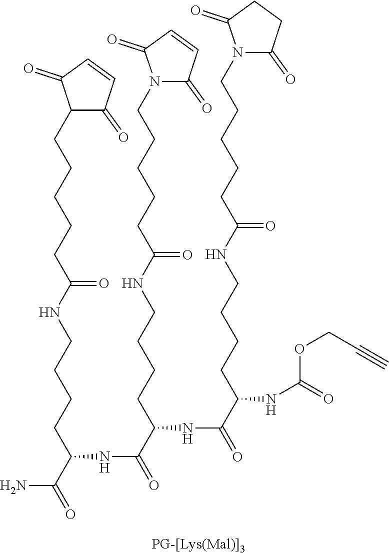 [SMILES: C#CCOC(=O)N[C@@H](CCCCNC(=O)CCCCCN1C(=O)CCC1=O)C(=O)N[C@@H](CCCCNC(=O)CCCCCN1C(=O)C=CC1=O)C(=O)N[C@@H](CCCCNC(=O)CCCCCC1C(=O)C=CC1=O)C(N)=O]

The same procedure as in Synthesis Example 2 was used, except that 8-maleimidohexanoic acid was used instead of bromoacetic acid (Scheme 3).

LCMS characterisation data: MWₜₕₑₒ=1062.5; MW_{found}=1063.8 [M+H⁺]

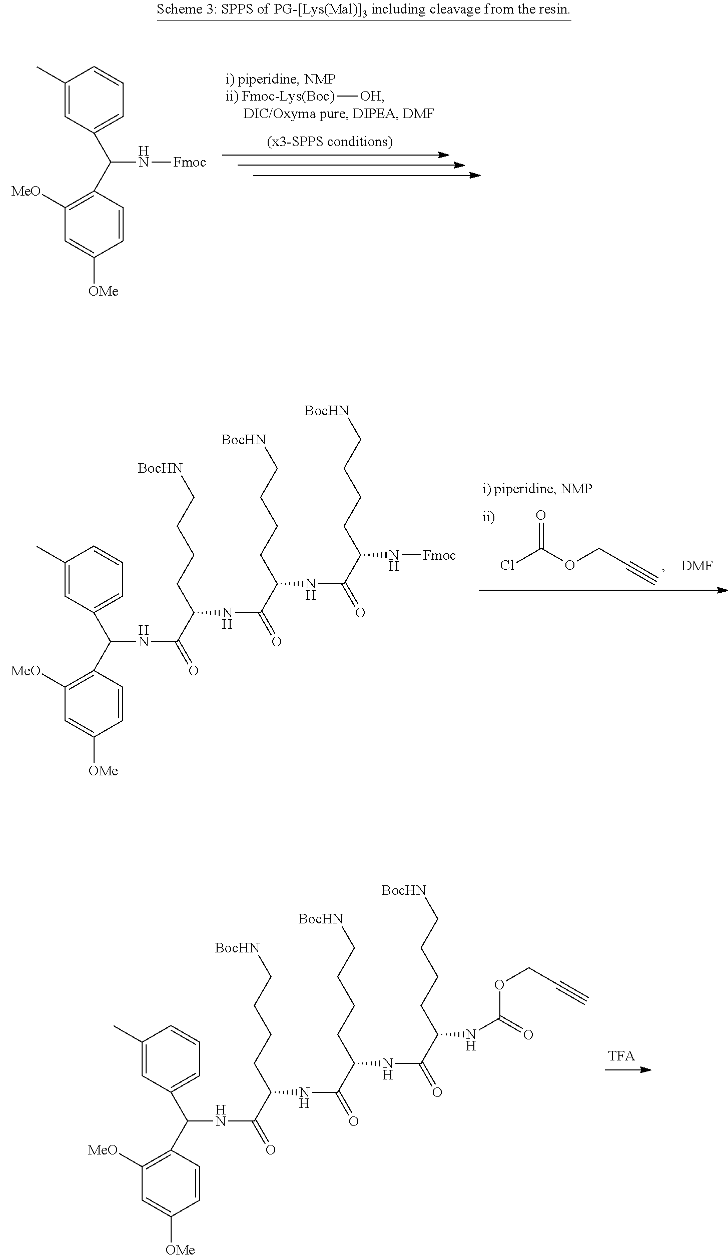

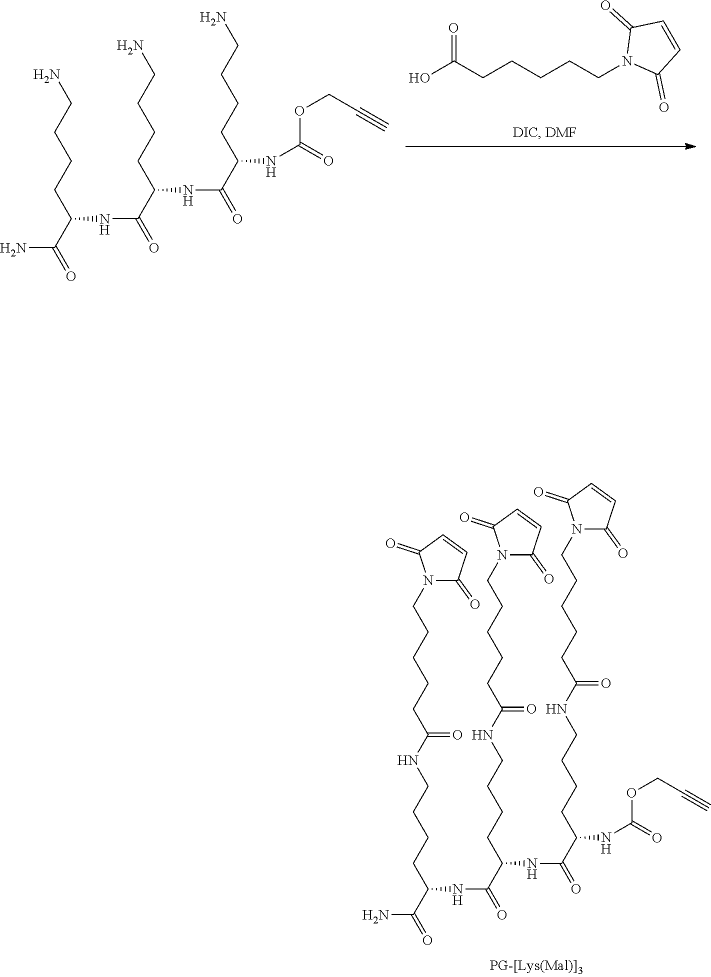 [SMILES: C#CCOC(=O)N[C@@H](CCCCN)C(=O)N[C@@H](CCCCN)C(=O)N[C@@H](CCCCN)C(N)=O ; O=C(O)CCCCCN1C(=O)C=CC1=O ; C#CCOC(=O)N[C@@H](CCCCNC(=O)CCCCCN1C(=O)C=CC1=O)C(=O)N[C@@H](CCCCNC(=O)CCCCCN1C(=O)C=CC1=O)C(=O)N[C@@H](CCCCNC(=O)CCCCCN1C(=O)C=CC1=O)C(N)=O]

### Synthesis Example 4: DBCO-[Lys(Br)]₃

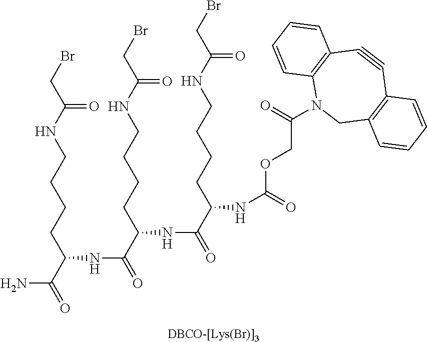 [SMILES: NC(=O)[C@H](CCCCNC(=O)CBr)NC(=O)[C@H](CCCCNC(=O)CBr)NC(=O)[C@H](CCCCNC(=O)CBr)NC(=O)OCC(=O)N1Cc2ccccc2C#Cc2ccccc21]

Starting from Sieber resin, Fmoc-Lys(Mtt)-OH is coupled in series three times using SPPS conditions. After Fmoc-removal, DBCO-acid is coupled using DIC/Oxyma pure and DIPEA in DMF and the core is cleaved off from the resin using TFA. Bromoacetic acid is coupled using DIC in DMF and the product is purified by RP-HPLC purification and freeze dried (Scheme 4).

LCMS characterisation data: MWₜₕₑₒ=1050.0; MW_{found}=1051.0 [M+H⁺]

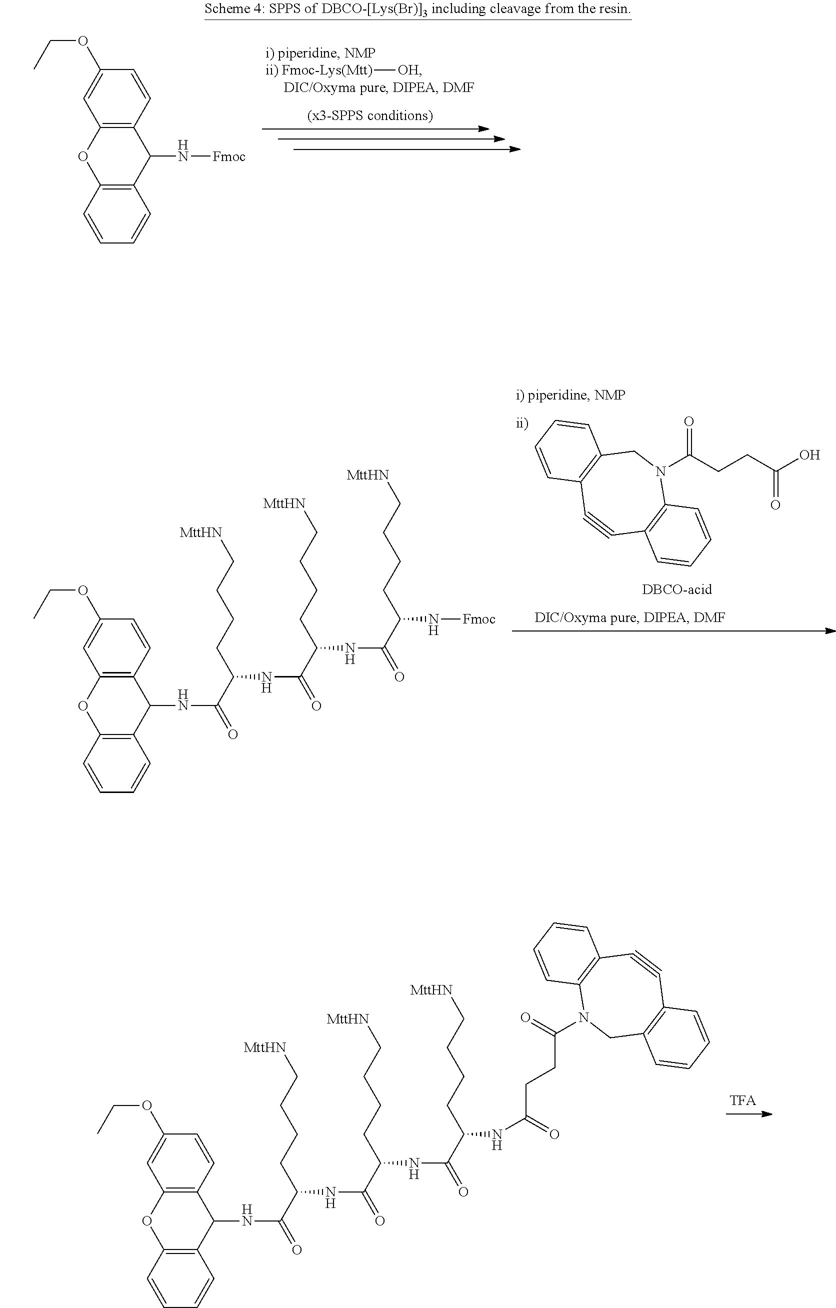

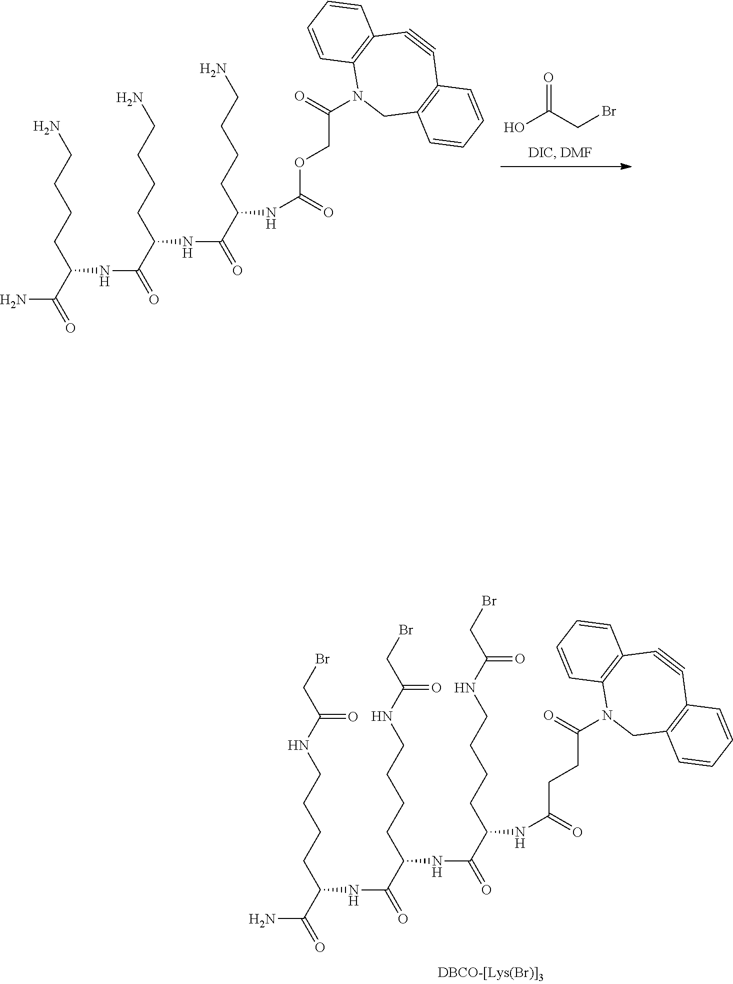

### Synthesis Example 5; DBCO-[Lys(PG)]₃

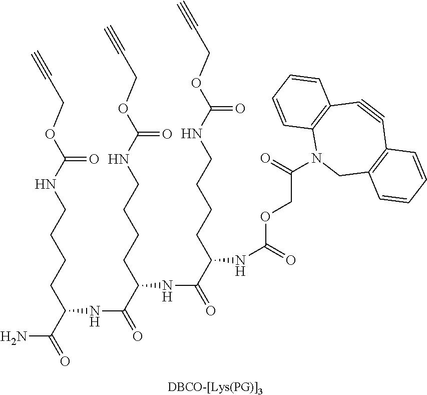 [SMILES: C#CCOC(=O)NCCCC[C@H](NC(=O)[C@H](CCCCNC(=O)OCC#C)NC(=O)[C@H](CCCCNC(=O)OCC#C)NC(=O)OCC(=O)N1Cc2ccccc2C#Cc2ccccc21)C(N)=O]

Starting from Rink amide resin, Fmoc-Lys(Boc)-OH is coupled in series three times using SPPS conditions. The core is cleaved off from the resin and propargyl chloroformate is coupled using DIPEA in DMF. After Fmoc-removal using 20% piperidine in DMF, the intermediate is purified by RP-HPLC purification and freeze dried. DBCO-acid is coupled using DIC/Oxyma pure and DIPEA in DMF and the product is purified by RP-HPLC purification and freeze dried (Scheme 5).

LCMS characterisation data: MWₜₕₑₒ=934.4; MW_{found}=935.5 [M+H⁺]

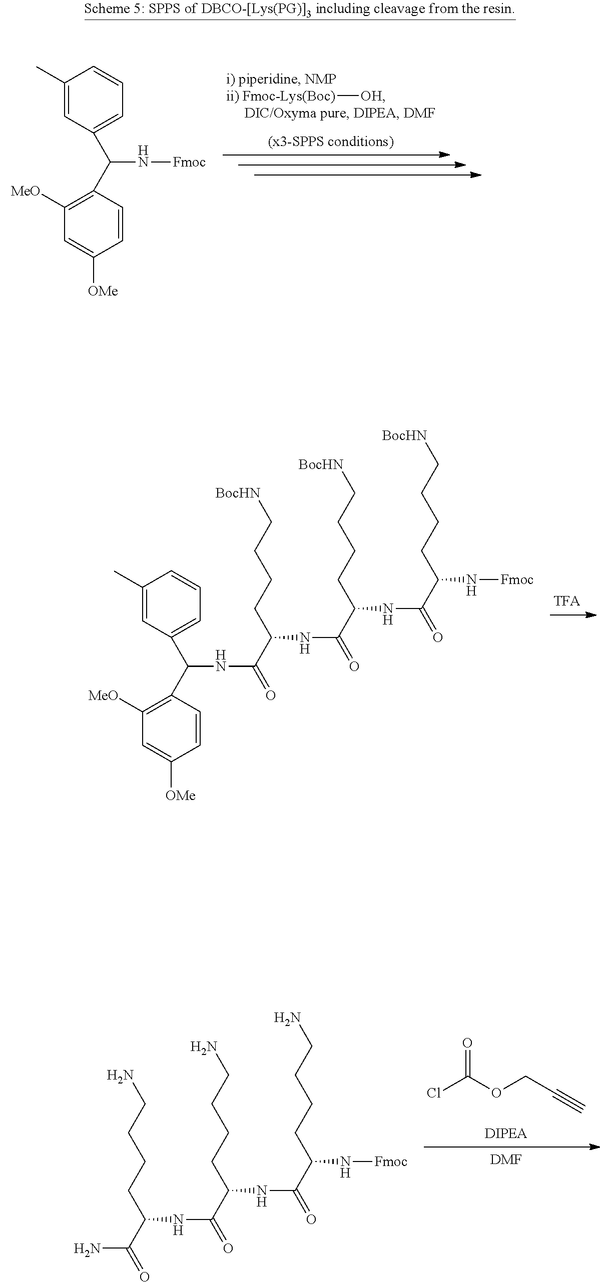

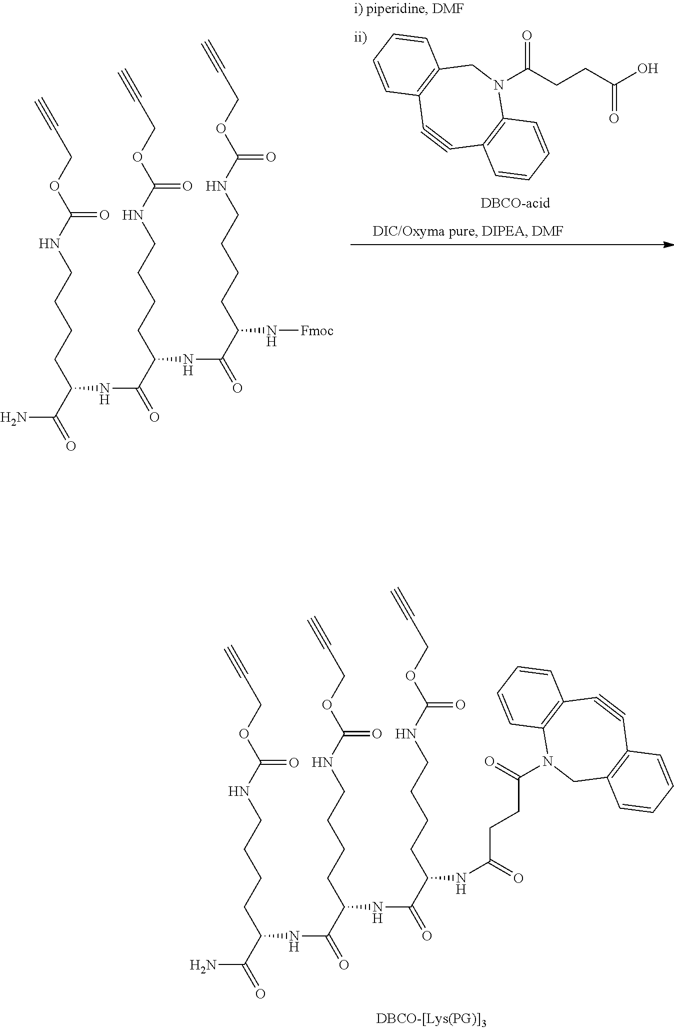

### Reference Synthesis Example 6: Core 1.0

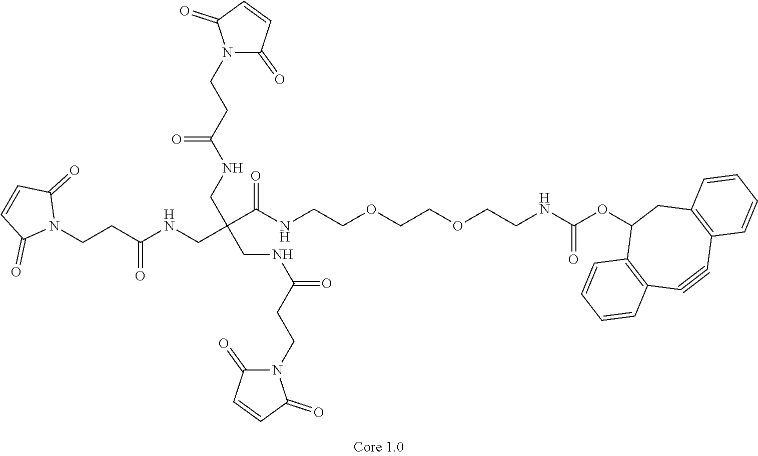 [SMILES: O=C(CCN1C(=O)C=CC1=O)NCC(CNC(=O)CCN1C(=O)C=CC1=O)(CNC(=O)CCN1C(=O)C=CC1=O)C(=O)NCCOCCOCCNC(=O)OC1Cc2ccccc2C#Cc2ccccc21]

Core 1.0 was synthesised as described in [113]-[121] of EP 2547364 B1.

### Synthesis Example 7: DBCO-Core B

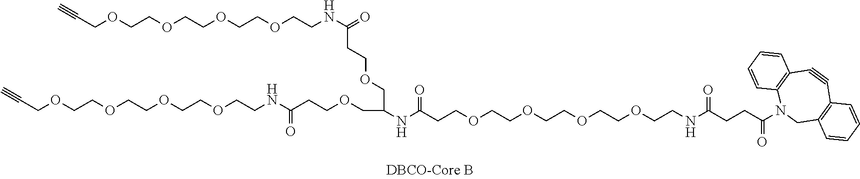 [SMILES: C#CCOCCOCCOCCOCCNC(=O)CCOCC(COCCC(=O)NCCOCCOCCOCCOCC#C)NC(=O)CCOCCOCCOCCOCCNC(=O)CCC(=O)N1Cc2ccccc2C#Cc2ccccc21]

DBCO-acid is activated using PyOxim/Oxyma pure and DIPEA in DMF and reacted with Amino-PEG4-Bis-PEG4-alkyne (Core B) in DMF. The product is purified by flash chromatography (Scheme 6).

LCMS characterisation data: MWₜₕₑₒ=1196.62; MW_{found}=1197.10 [M+H⁺]

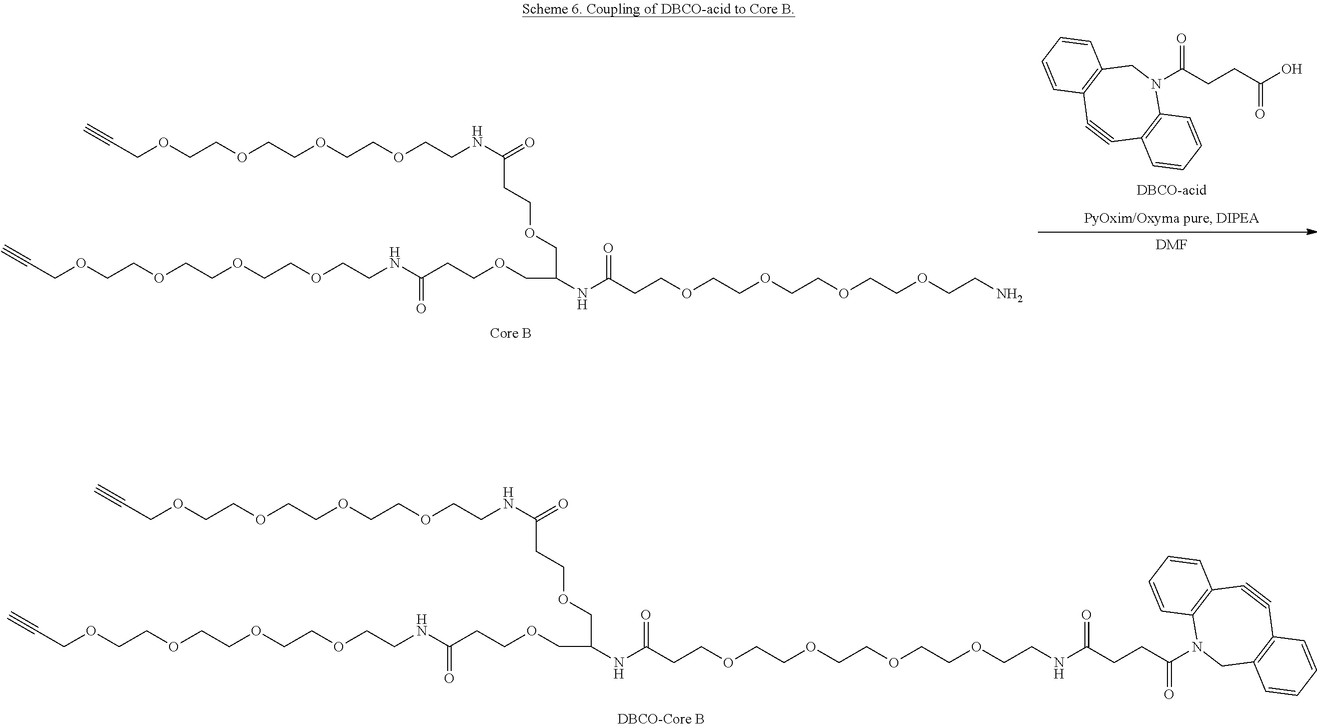

### Synthesis Example 8: DBCO-Lys(Br)-Lys(Br)-Lys(Br)—NH-PG

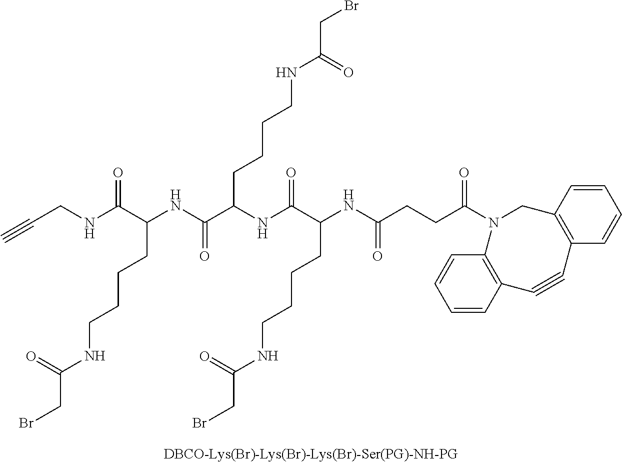 [SMILES: C#CCNC(=O)C(CCCCNC(=O)CBr)NC(=O)C(CCCCNC(=O)CBr)NC(=O)C(CCCCNC(=O)CBr)NC(=O)CCC(=O)N1Cc2ccccc2C#Cc2ccccc21]

Deprotection of Fmoc conditions: MeCN, 1-5 equiv. of Et2NH and/or piperidine.

Coupling conditions: 1 equiv. amino acid, 1 equiv. PyOxim, 1 equiv. Oxyma pure, 2 equiv. in MeCN; 1-2 hours.

Propargylamine is coupled to Fmoc-Lys(Mtt)-OH using coupling conditions, to produce Fmoc-Lys(Mtt)-NH-PG. Fmoc-Lys(Mtt)-NH-PG is deprotected using deprotection of Fmoc conditions, then coupled to Fmoc-Lys(Mtt)-OH using coupling conditions, to produce Fmoc-Lys(Mtt)-Lys(Mtt)-NH-PG. Fmoc-Lys(Mtt)-Lys(Mtt)-NH-PG is deprotected using deprotection of Fmoc conditions, then coupled to Fmoc-Lys(Mtt)-OH using coupling conditions, to produce Fmoc-Lys(Mtt)-Lys(Mtt)-Lys(Mtt)-NH-PG.

Fmoc-Lys(Mtt)-Lys(Mtt)-Lys(Mtt)-NH-PG is then deprotected using deprotection of Fmoc conditions, then coupled to DBCO-acid using coupling conditions, to produce DBCO-Lys(Mtt)-Lys(Mtt)-Lys(Mtt)-NH-PG. Removal of Mtt using excess TFA then provides DBCO-Lys(NH₂)-Lys(NH₂)-Lys(NH₂)—NH-PG, which is purified and freeze-dried.

The purified and freeze-dried DBCO-Lys(NH₂)-Lys(NH₂)-Lys(NH₂)—NH-PG is dissolved in a small amount of DMF. Bromo-acetic acid is dissolved in DMF and DIC is added. The mixture is stirred until it turns cloudy and added to the core. The mixture is stirred until the reaction is complete (overnight). The reaction is diluted with DCM and washed several times with water to remove DMF.

DCM is evaporated and the product is immediately purified by RP-purification and the product containing fractions are freeze dried (Scheme 7).

LCMS characterisation data: MWₜₕₑₒ=1089.19; MW_{found}=1089.05 [M+H⁺]

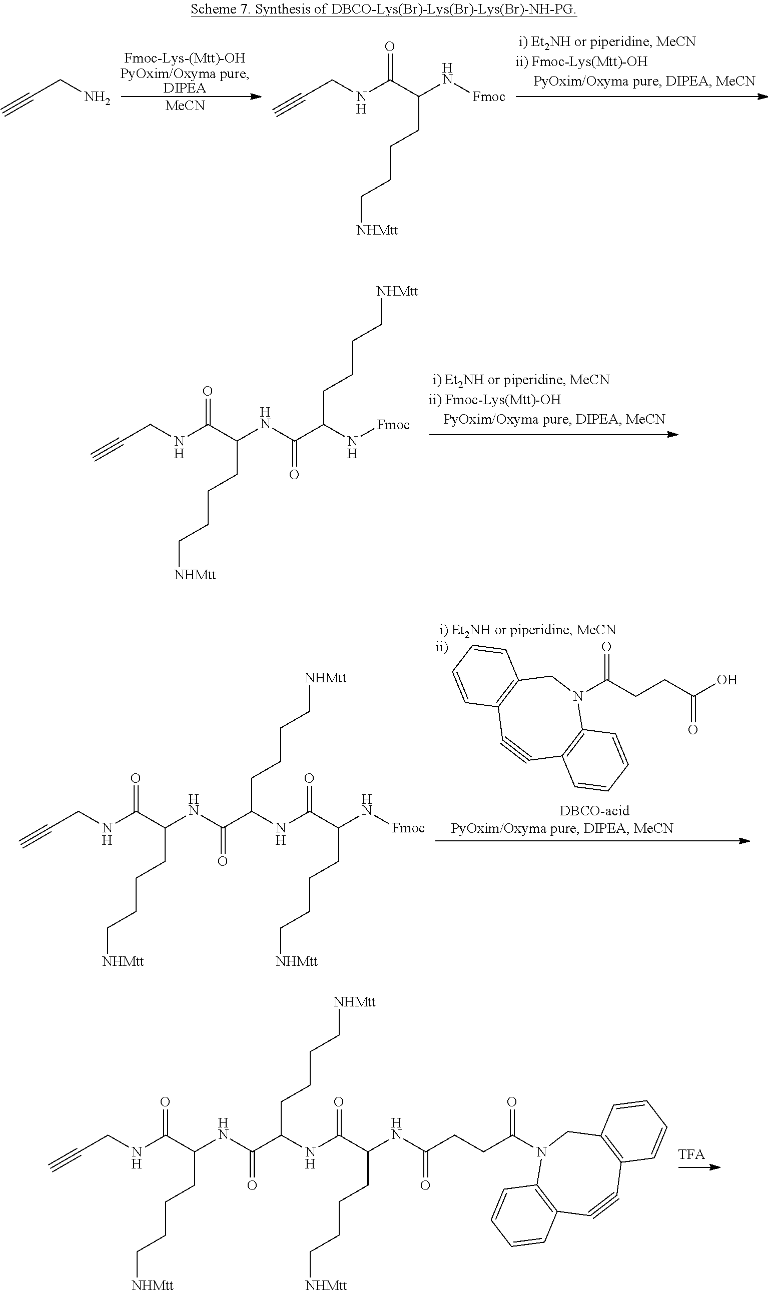

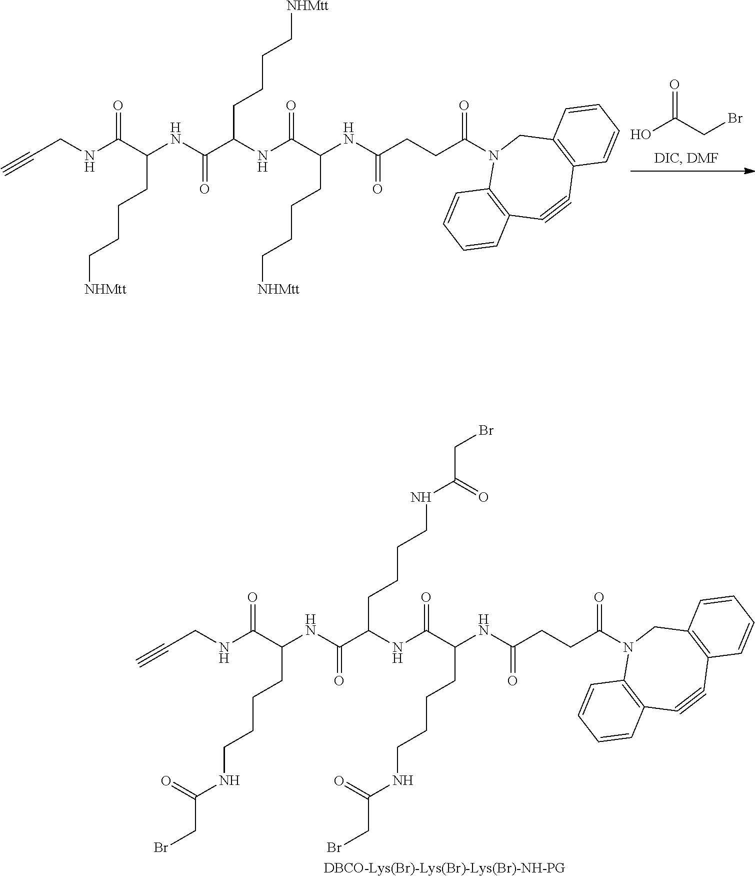

### Synthesis Example 9: DBCO-Lys(Br)-Lys(Br)-Lys(Br)—NH-PEG1-PG

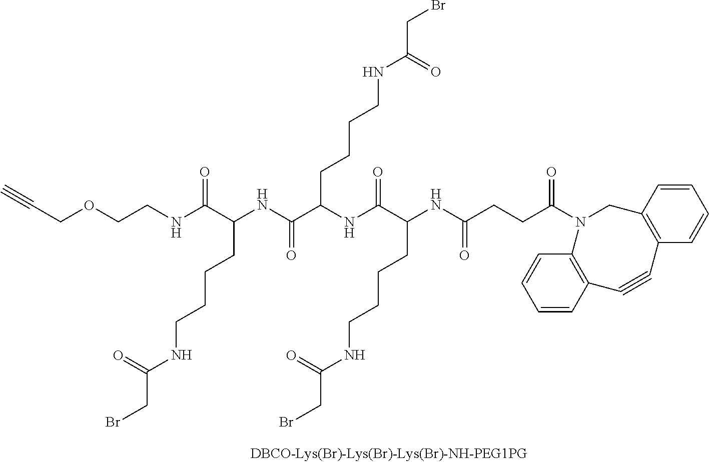 [SMILES: C#CCOCCNC(=O)C(CCCCNC(=O)CBr)NC(=O)C(CCCCNC(=O)CBr)NC(=O)C(CCCCNC(=O)CBr)NC(=O)CCC(=O)N1Cc2ccccc2C#Cc2ccccc21]

The same procedure as in Synthesis Example 8 was used, except that 2-(prop-2-yn-1-yloxy)ethan-1-amine was used instead of propargylamine (Scheme 8).

LCMS characterisation data: MWₜₕₑₒ=1133.22; MW_{found}=1133.13 [M+H⁺]

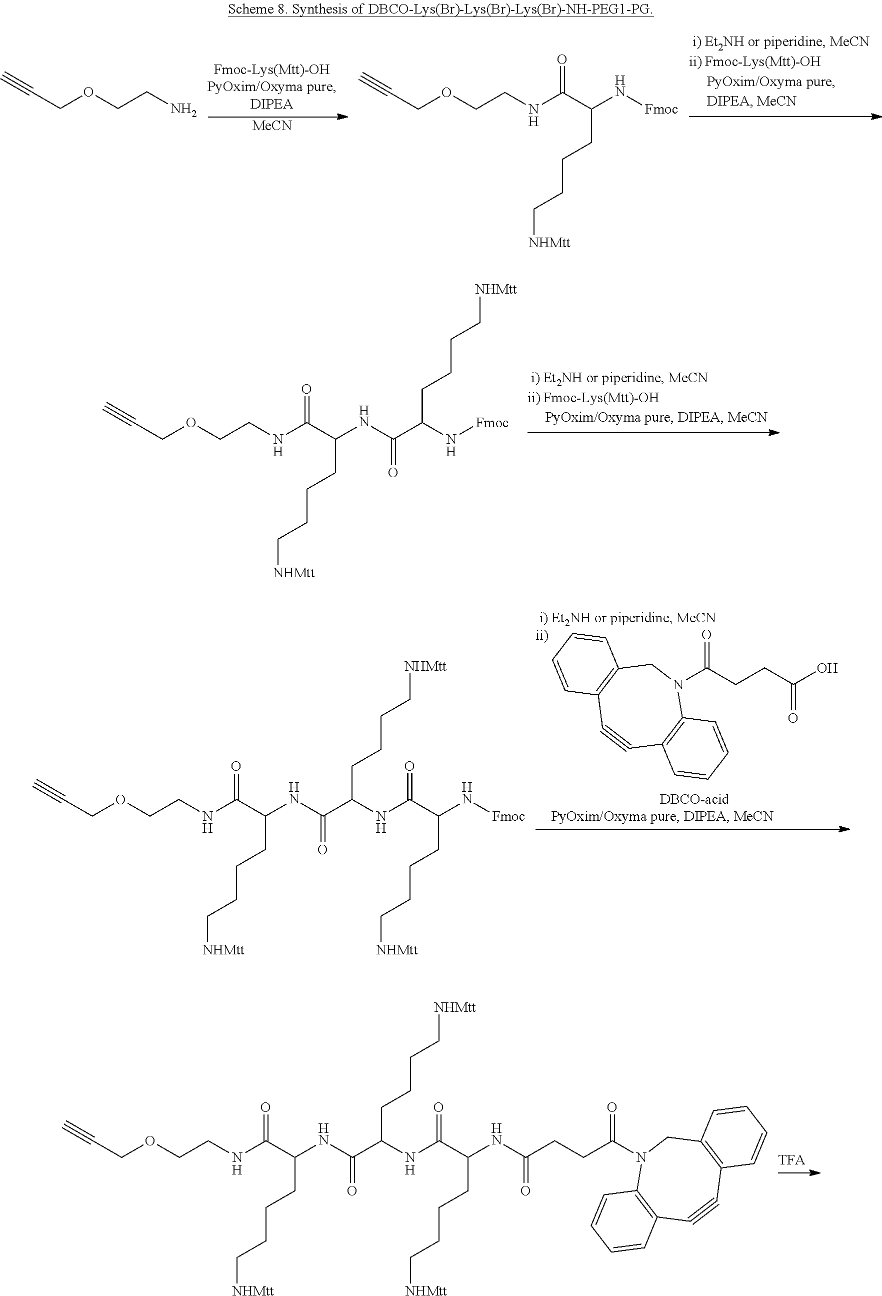

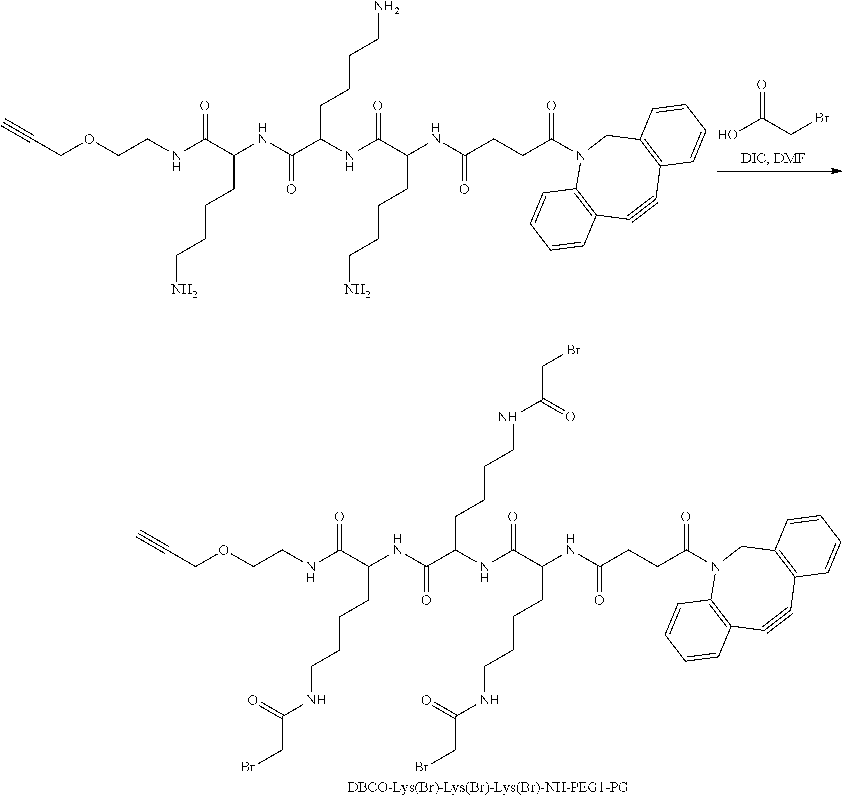

### Synthesis Example 10: DBCO-Lys(Br)-Lys(Br)-Lys(Br)-Ser(PG)-NH₂

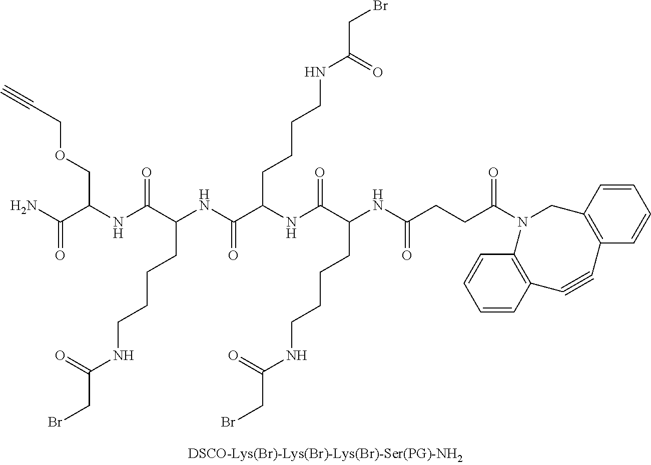 [SMILES: C#CCOCC(NC(=O)C(CCCCNC(=O)CBr)NC(=O)C(CCCCNC(=O)CBr)NC(=O)C(CCCCNC(=O)CBr)NC(=O)CCC(=O)N1Cc2ccccc2C#Cc2ccccc21)C(N)=O]

500 mg (0.3 mmol) Fmoc-Sieber-polystyrene resin (0.61 mmol/g loading) is swollen in DMF for 2 hours. Fmoc is removed with 20% piperidine in NMP and the resin is washed with NMP and DMF. The first amino acid (Fmoc-Ser(PG)-OH) is loaded using DIC/Oxyma pure and DIPEA in DMF in a 5-fold excess. The amino acid is coupled for 6 hours and a second time for 14 hours using a 5-fold excess for both couplings. The Fmoc was cleaved (NMP/piperidine) and followed by coupling the amino acids (Fmoc-Lys(Mtt)-OH) for 6 hours and a second time for 14 hours using a 5-fold excess for both couplings. The step of Fmoc removal and coupling with (Fmoc-Lys(Mtt)-OH) is repeated twice more,

After assembly Fmoc is cleaved and 2 equivalents DBCO acid are coupled using DIC/Oxyma pure and DIPEA in DMF for 6 hours. Additionally, 2 equivalents DBCO acid are coupled a second time using DIC/Oxyma pure and DIPEA in DMF for 14 hours. The side chain protective groups and the sequence are cleaved simultaneously using TFA/TIS/DCM/MeOH (1:2:45:45) for 90 min. The cleavage mixture is concentrated and redissolved in diethyl ether/water (1:1). The fractions are separated, and the aqueous phase is freeze dried. Bromoacetic acid (16 equivalents) is dissolved in DMF at a concentration of 10 mg/mL and DIC (8 equivalents) are added to the mixture. The mixture is stirred to 20-30 minutes until it turned cloudy. DBCO-[Lys(NH₂)]₃Ser(PG)-NH₂ is dissolved in DMF at a concentration of 20 mg/mL and the pre activated bromoacetic acid is added. The reaction is monitored using LCMS. The product is either purified by RP purification using a Biotage® Sfär Bio C18 D-Duo 300 Å 20 μm column and a 0-40% B gradient (A: 0.1% TFA in water, B: 0.1% TFA in ACN). Fractions containing the pure product were combined and freeze dried to give the product in 30% yield (Scheme 9).

LCMS characterisation data: MWₜₕₑₒ=1176.22; MW_{found}=1176.20 [M+H⁺]

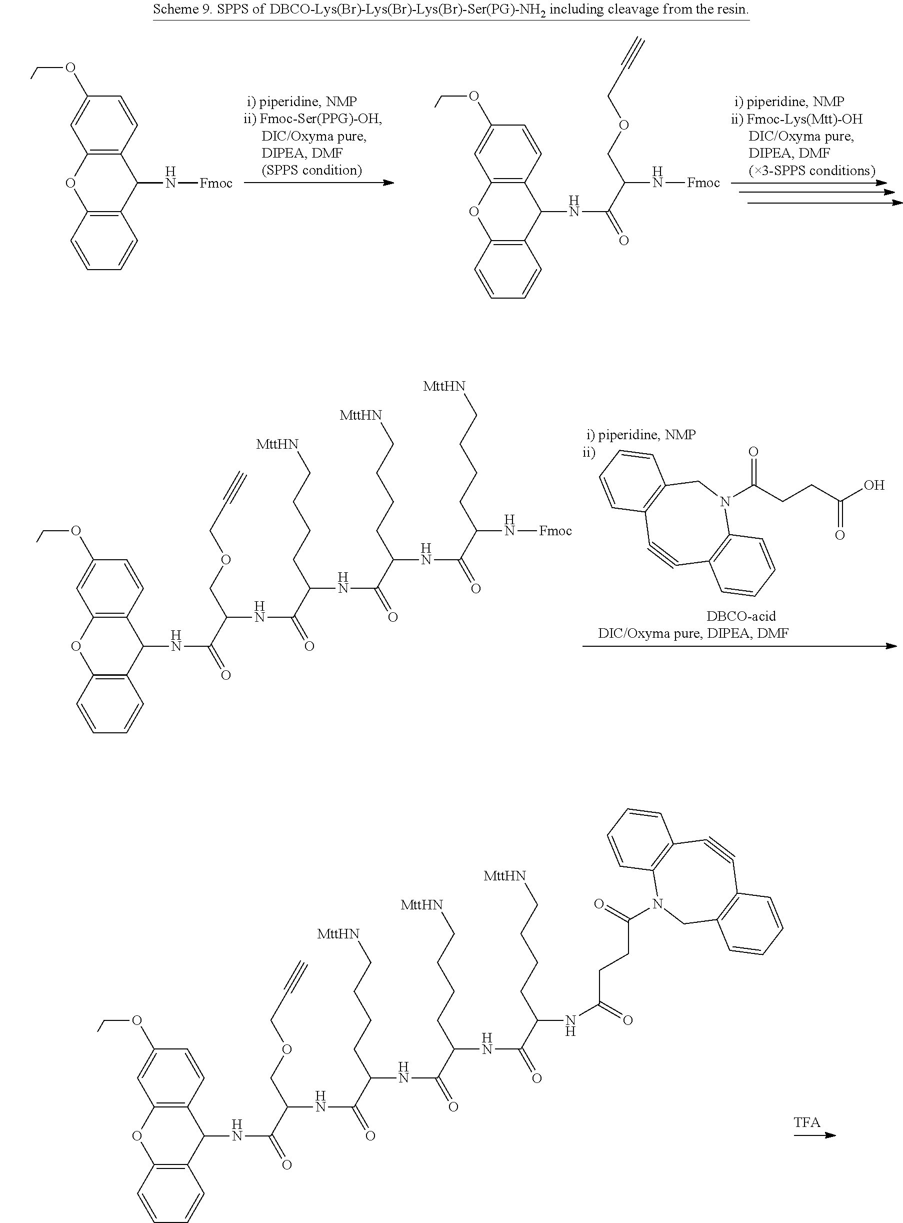

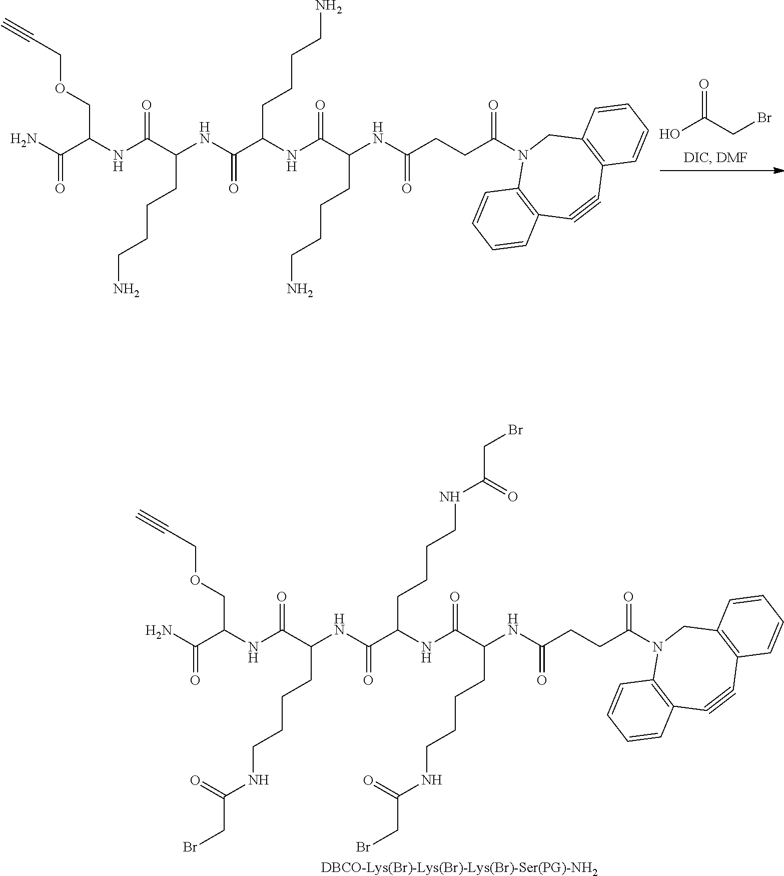

### Conjugate Synthesis—Synthesis Examples 11 to 22

Conjugates were synthesised as detailed below. Tables 10A and 10B summarise the core constructs.

TABLE 10A
Summary of 3+1/2+1 Core Constructs

| Synthesis | | | B-cell epitope | | CD4+ T-cell epitope | |
|---|---|---|---|---|---|---|
| Example | Conjugate | Core | Name | Sequence | Name | Sequence |
| 11 | CD12B | DBCO- | TET001K | (SEQ ID NO: 145) | 719-20K | (SEQ ID NO: 1) |
| | | Core A | | H-FIGITELKKLESKINKVF- | | H-K (N₃)-ALFSVLNYERARRPG |
| | | | | AEKYARVRAK-K(N₃)-NH₂ | | LLGASVLGLDDIHRA-OH |
| 12 | CD31 | DBCO- | TET001K | (SEQ ID NO: 145) | GVExt13M | (SEQ ID NO: 117) |
| | | Core A | | H-FIGITELKKLESKINKVE- | | H-K(N₃)-HREARPALLTSRLRF |
| | | | | AEKYARVRAK-K(N₃)-NH₂ | | IPKPDGLRPIVNMDY-NH₂ |
| 13 | CD28 | DBCO- | TET001K | (SEQ ID NO: 145) | PEP3242 | (SEQ ID NO: 125) |
| | | Core A | | H-FIGITELKKLESKINKVE- | | H- |
| | | | | AEKYARVRAK-K(N₃)-NH₂ | | ALFSVLNYERARRPGLLGASVLGL |
| | | | | | | DDIHRANPILLWQPIPV-K(N₃)-NH₂ |
| 14 | CD20B | DBCO- | TET001C | (SEQ ID NO: 146) | 719-20K | (SEQ ID NO: 1) |
| | | [Lys(Br)]₃ | | H-FIGITELKKLESKINKVE- | | H-K(N₃)-ALFSVLNYERARRPG |
| | | | | AEKYARVRAKC-NH₂ | | LLGASVLGLDDIHRA-OH |
| 15 | CD29 | DBCO- | TET001C | (SEQ ID NO: 146) | GVEx15 | (SEQ ID NO: 116) |
| | | [Lys(Br)]₃ | | H-FIGITELKKLESKINKVE- | | H-K(N₃)-LSEAEVROHREARPALL |
| | | | | AEKYARVRAKC-NH₂ | | TSRLRFIPKPDGL-NH₂ |
| 16 | CD30 | DBCO- | TET001C | (SEQ ID NO: 146) | GVExt13M | (SEQ ID NO: 117) |
| | | [Lys(Br)]₃ | | H-FIGITELKKLESKINKVF- | | H-K(N₃)-HREARPALLTSRLRFIP |
| | | | | AEKYARVRAKC-NH₂ | | KPDGLRPIVNMDY-NH₂ |
| 17 | CD32 | Core 1.0 | TET001C | (SEQ ID NO: 146) | GVEx15 | (SEQ ID NO: 116) |
| | | | | H-FIGITELKKLESKINKVF- | | H-K(N₃)-LSEAEVROHREARPALL |
| | | | | AEKYARVRAKC-NH₂ | | TSRLRFIPKPDGL-NH₂ |
| 18 | CD20 | DBCO- | TET001K | (SEQ ID NO: 145) | 719-20K | (SEQ ID NO: 1) |
| | | [Lys(PG)]₃ | | H-FIGITELKKLESKINKVF- | | H-K(N₃)-ALFSVLNYERARRPGLL |
| | | | | AEKYARVRAK-K(N₃)-NH₂ | | GASVLGLDDIHRA-OH |
| 19 | CD14 | DBCO- | TET005- | (SEQ ID NO: 7) | 719-20-b- | (SEQ ID NO: 1) |
| | | Core B | PEG2- | FIGITELKKLESKINKVE- | 1 | N3-(CH₂)5-C(O)- |
| | | | K(N3)-OH | (PEG2)-K(N₃)-OH | | ALFSVLNYERARRPGLLGASVLGLDD |
| | | | | | | IHRA-OH |

TABLE 10B
Summary of 3+1+1 Core Constructs

| Synthesis | Core | B-cell epitope | | CD4+ T-cell epitope | | Further substance | |
|---|---|---|---|---|---|---|---|
| Example | | Name | Sequence | Name | Sequence | Name | Sequence |
| 20 | DBCO-Lys(Br)- | TET001C | (SEQ ID NO: 146) | 719-20K | (SEQ ID NO: 1) | GVExt5 | (SEQ ID NO: 116) |
| | Lys(Br)-Lys | | H- | | H-K(N₃)- | | H-K (N₃)-LSEAEVRQ |
| | (Br)-NH-PG | | FIGITELKKLESKINKV | | ALFSVLNYERARRPG- | | HREARPALL-TSRLRFI |
| | | | F-AEKYARVRAKC-NH₂ | | LLGASVLGLDDIHRA-OH | | PKPDGL-NH₂ |
| 21 | DBCO-Lys(Br)- | TET001C | (SEQ ID NO: 146) | 719-20K | (SEQ ID NO: 1) | GVExt5 | (SEQ ID NO: 116) |
| | Lys(Br)-Lys | | H- | | H-K(N₃)- | | H-K(N₃)-LSEAEVRQ |
| | (Br)-NH-PEG1- | | FIGITELKKLESKINKV | | ALFSVLNYERARRPG- | | HREARPALL-TSRLRF |
| | PG | | F-AEKYARVRAKC-NH₂ | | LLGASVLGLDDIHRA-OH | | IPKPDGL-NH₂ |
| 22 | DBCO-Lys(Br)- | TET001C | (SEQ ID NO: 146) | 719-20K | (SEQ ID NO: 1) | GVEx15 | (SEQ ID NO: 116) |
| | Lys(Br)-Lys | | H- | | H-K(N₃)- | | H-K(N₃)-LSEAEVRQ |
| | (Br)-Ser(PG)- | | FIGITELKKLESKINKV | | ALFSVLNYERARRPG- | | HREARPALL-TSRLRF |
| | NH₂ | | E-AEKYARVRAKC-NH₂ | | LLGASVLGLDDIHRA-OH | | IPKPDGL-NH₂ |

(N.B. “H—” in Tables 10A and B for the B-cell epitope, the CD4+ T-cell epitope and the further substance refers to hydrogen at the N-terminus.)

It is to be noted that each of the conjugates included in Tables 10A and B includes 2 or 3 copies of the polypeptide comprising the sequence of the B-cell epitope conjugated to the core.

Polypeptide sequences comprising the B-cell epitope and the CD4+ T-cell epitope were synthesised using conventional processes known in the art and/or as described previously (for example, as described in WO 2011/115483, which is incorporated herein by reference).

### Synthesis Example 11: Synthesis of 3+1 Construct CD12B Using DBCO-Core A

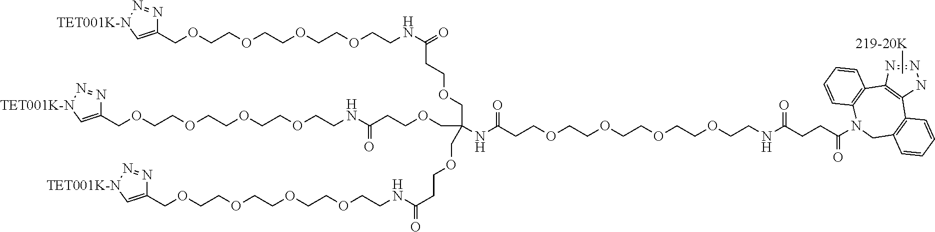

DBCO-Core A and 719-20K are mixed and dissolved in ACN/water. The mixture is stirred until the reaction is complete and the 0+1 intermediate is purified by RP-HPLC and freeze dried. The 0+1 intermediate and TET001K are mixed and dissolved in ACN/water and Cu(I) is added. The mixture is stirred until the reaction is complete and the product is purified by RP-HPLC and freeze dried (Scheme 10).

Deconvoluted HRMS characterisation data: MWₜₕₑₒ=15248.9; MW_{found}=15248.6

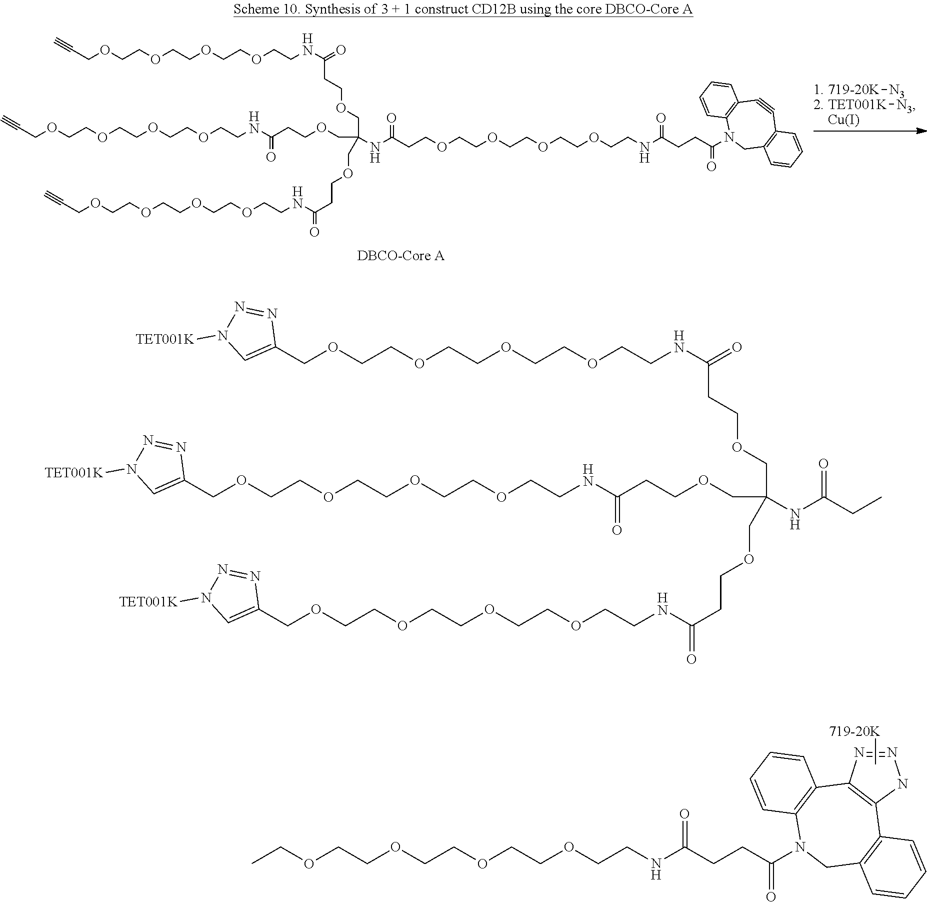

### Synthesis Example 12: Synthesis of 3+1 Construct CD31 Using DBCO-Core A

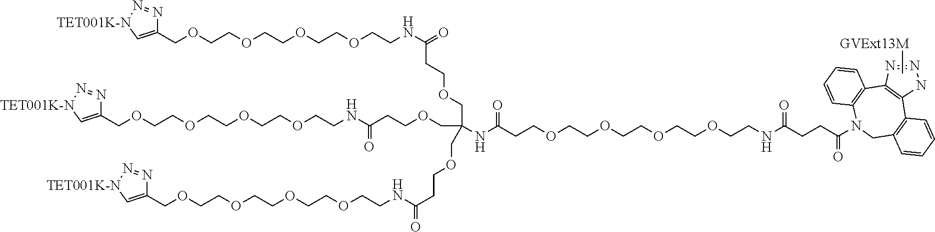

The same procedure as in Synthesis Example 11 was used, except that GVExt13M was used instead of 719-20K (Scheme 11).

Deconvoluted HRMS characterisation data: MWₜₕₑₒ=15500.3; MW_{found}=15499.8

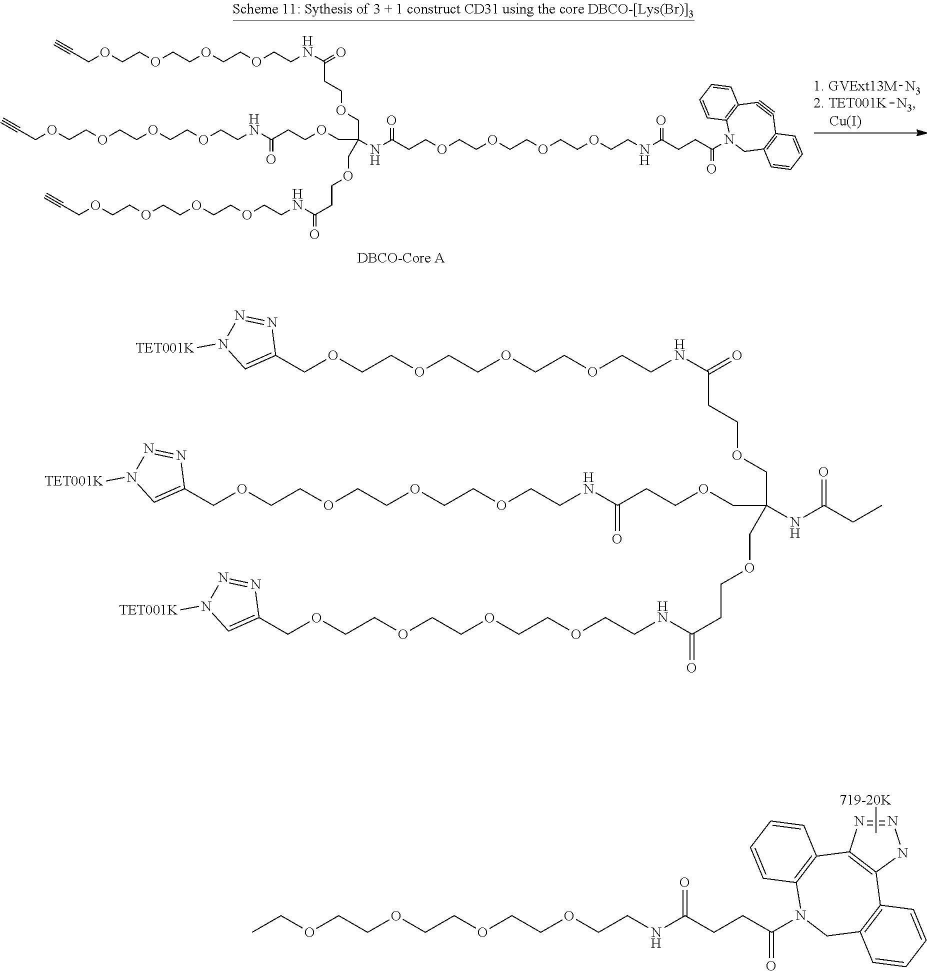

### Synthesis Example 13: Synthesis of 3+1 Construct CD28 Using DBCO-Core A

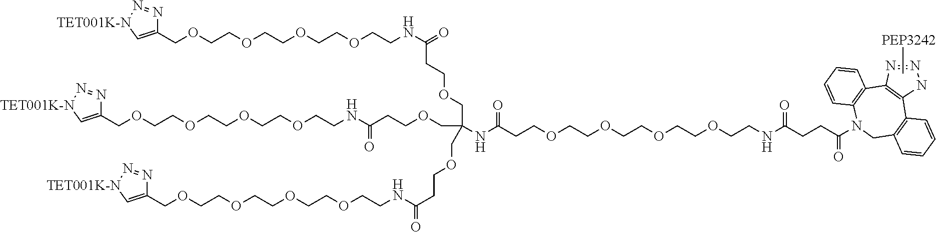

The same procedure as in Synthesis Example 11 was used, except that PEP3242 was used instead of 719-20K (Scheme 12).

Deconvoluted HRMS characterisation data: MWₜₕₑₒ=16519.8; MW_{found}=16519.5

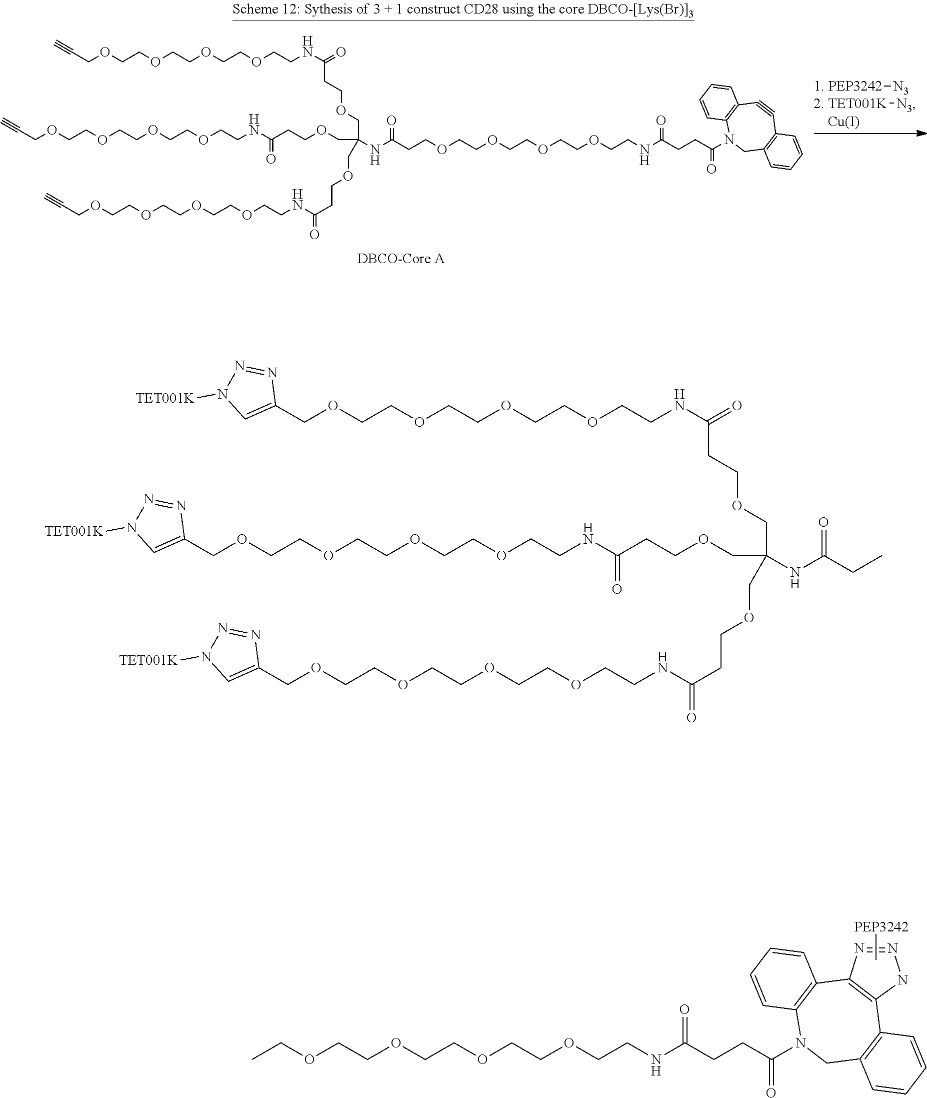

### Synthesis Example 14: Synthesis of 3+1 Construct CD20B Using DBCO-[Lys(Br)]₃

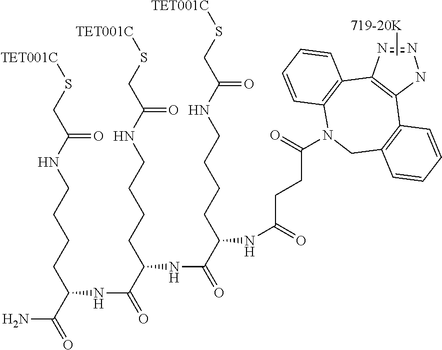

TET001C and DBCO-[Lys(Br)]₃ are mixed and dissolved in ACN/water. The pH is adjusted to 8.5 using 0.5 M NaHCO₃. After the coupling is complete (approximately 15 min) the pH is lowered to 5 with 5% AcOH and 719-20K is added and the reaction is stirred until the coupling is complete. The product is purified by RP-HPLC and freeze dried (Scheme 13).

Deconvoluted HRMS characterisation data: MWₜₕₑₒ=14394.0; MW_{found}=14392.6

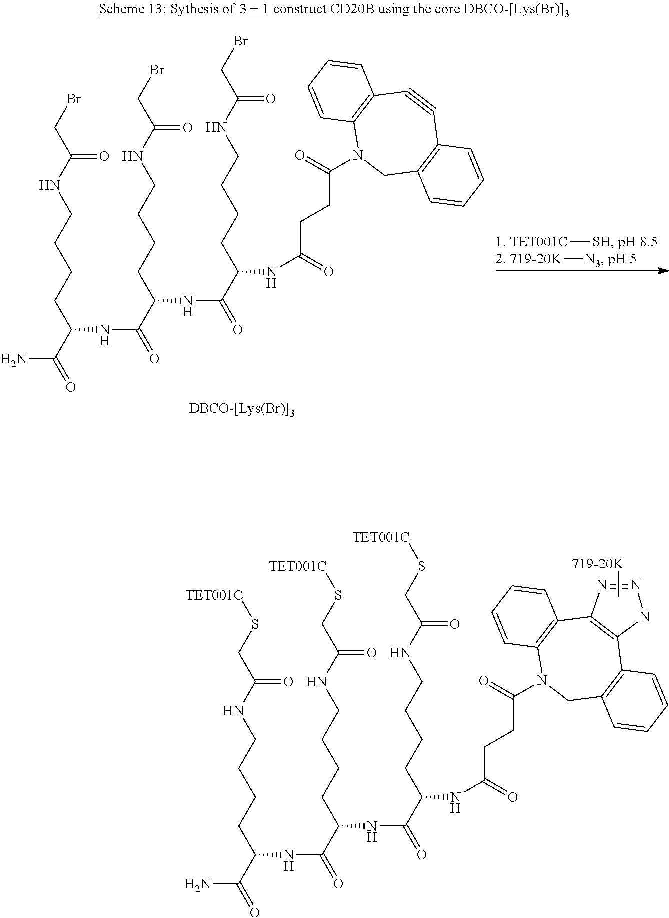

### Synthesis Example 15: Synthesis of 3+1 Construct CD29 Using DBCO-[Lys(Br)]₃

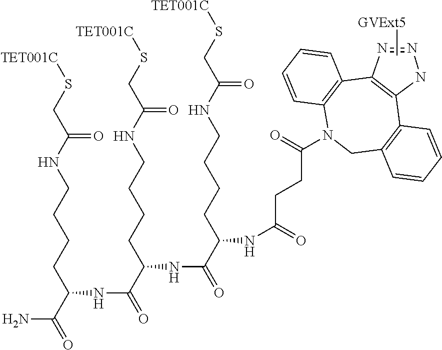

The same procedure as in Synthesis Example 14 was used, except that GVExt5 was used instead of 719-20K (Scheme 14).

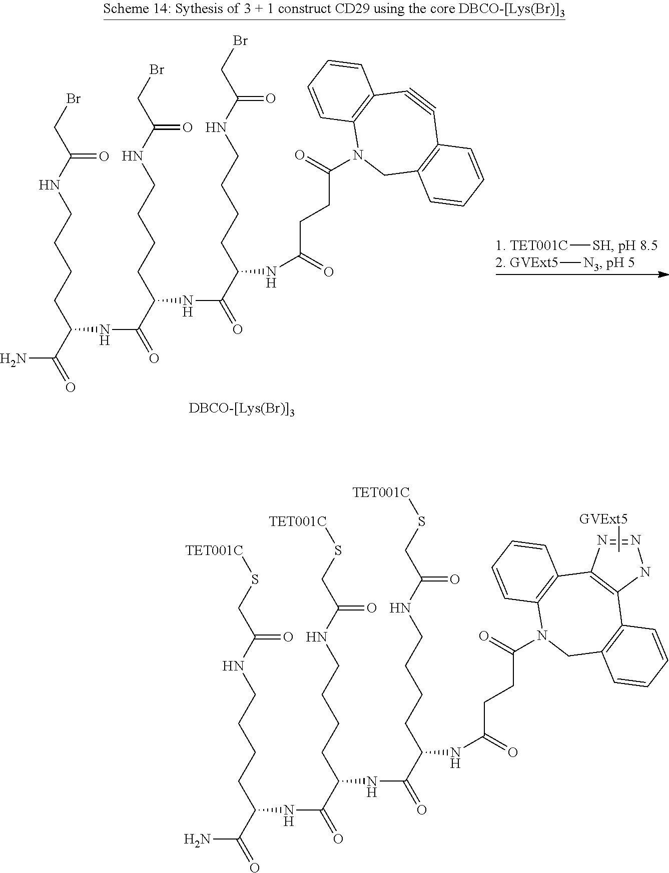

Deconvoluted HRMS characterisation data: MWₜₕₑₒ=14568.2; MW_{found}=14567.4

### Synthesis Example 16: Synthesis of 3+1 Construct CD30 Using DBCO-[Lys(Br)] 3

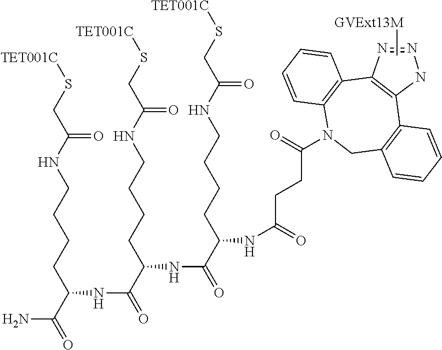

The same procedure as in Synthesis Example 14 was used, except that GVExt13M was used instead of 719-20K (Scheme 15).

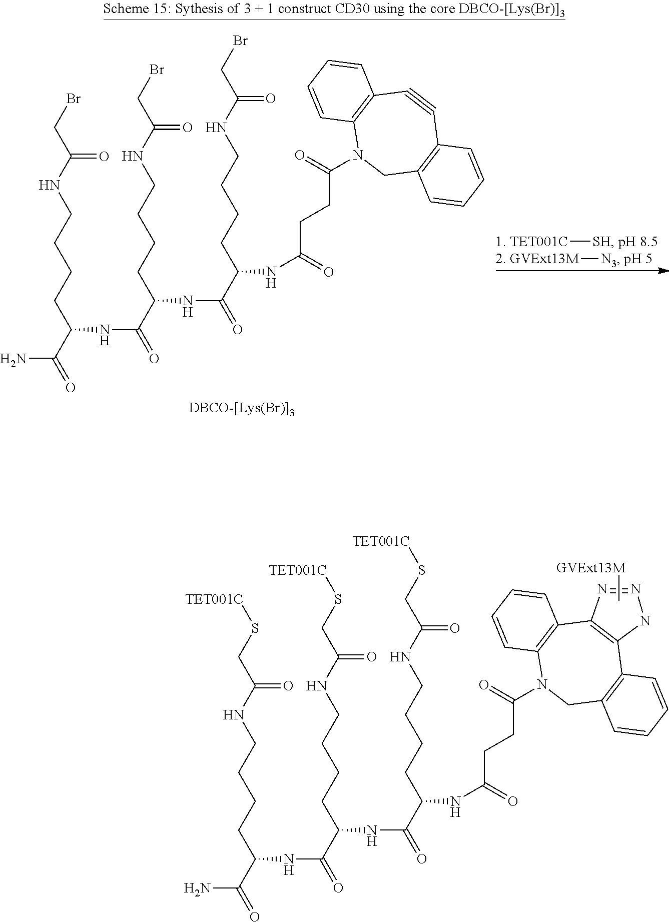

Deconvoluted HRMS characterisation data: MWₜₕₑₒ=14644.4; MW_{found}=14643.4

### Synthesis Example 17: Synthesis of 3+1 Construct CD32 Using Core 1.0

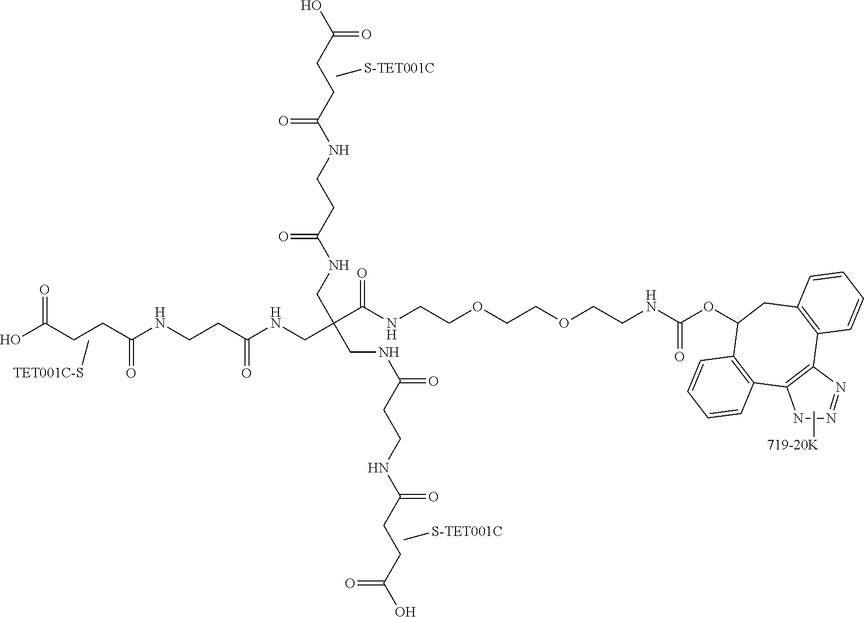

Core 1.0 in acetonitrile was added to a solution of TET001C in degassed water and degassed aqueous 0.5 M NaHCO₃ protected from light. The reaction mixture was stirred at room temperature for 3 h. A solution of peptide 719-20K in DMSO was added and stirring at room temperature was continued overnight. 4.2% aqueous NaHCO₃ was added to the reaction mixture and stirred at 40° C. for 3 days. AcOH and TCEP was added at room temperature and the mixture was stirred for 15 min, transferred to preparative LC-MS vials and purified by reversed phase chromatography and freeze dried (Scheme 16).

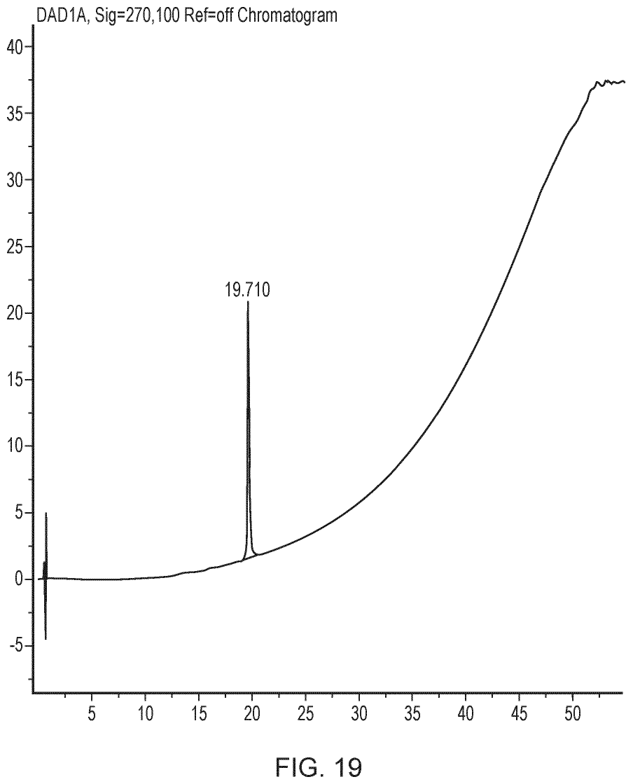

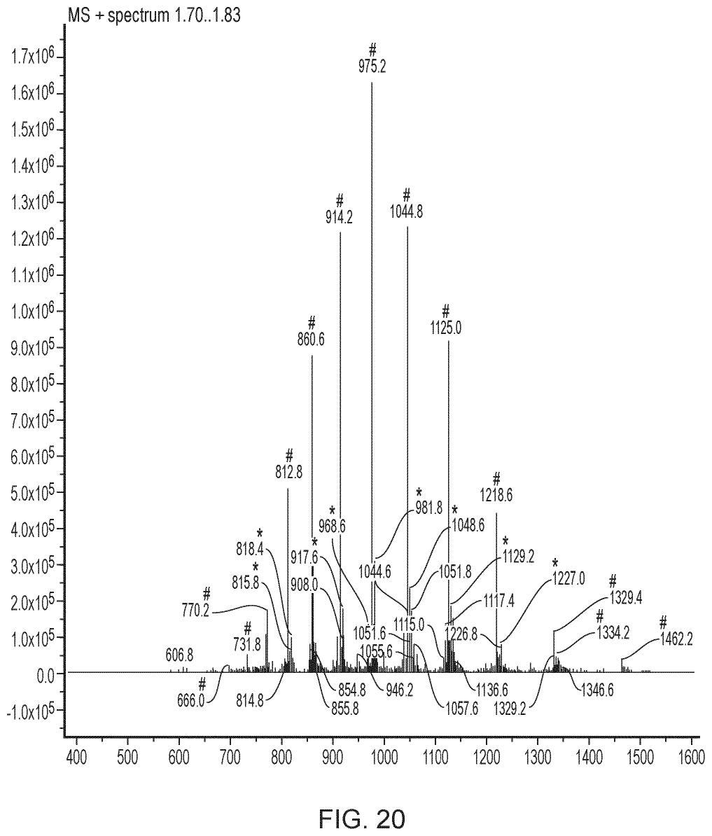

Reverse chromatographic and LCMS traces are shown in FIGS. 19 and 20.

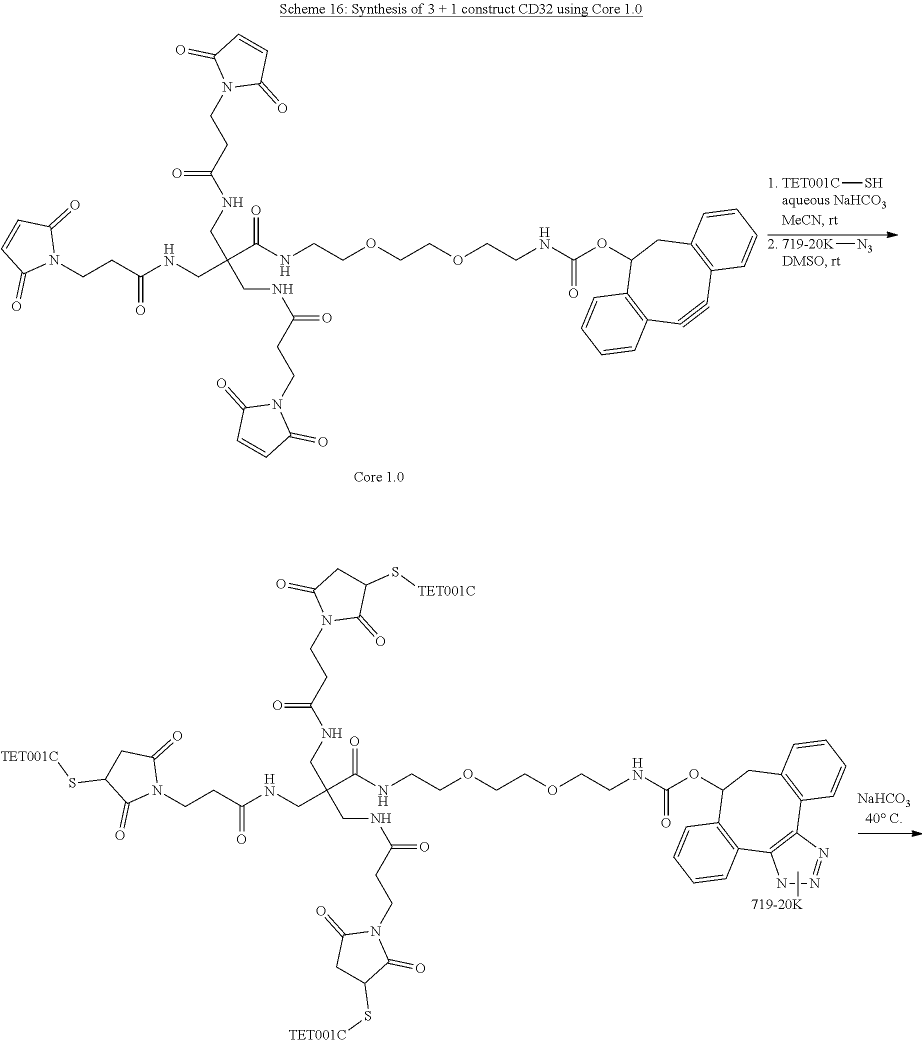

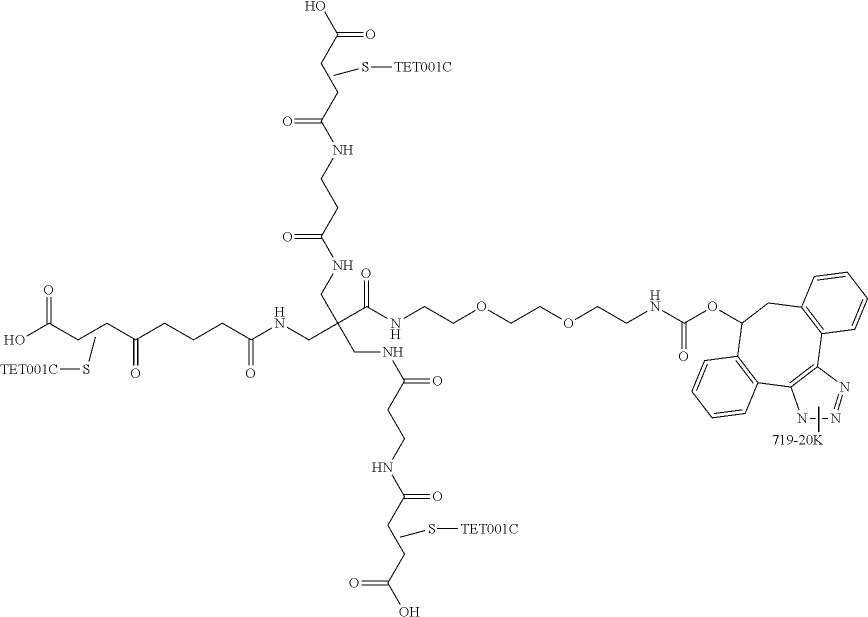

### Synthesis Example 18: Synthesis of 3+1 Construct CD20 Using DBCO-[Lys(PG)]₃

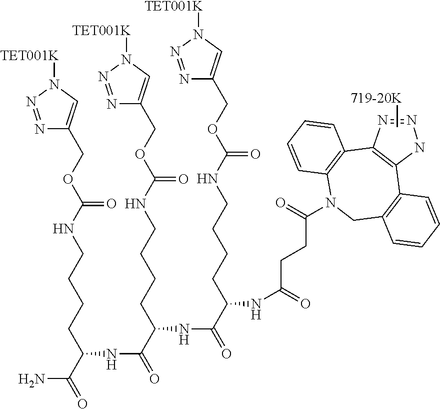

DBCO-[Lys(PG)] 3 core was suspended in 85% ACN in water and transferred to a vial containing 719-20K peptide dissolved in water. Additional ACN was added to clarify the milky solution. After confirmed IPC (LC-MS. 2.5 h), the reaction mixture was purified on a C18 column to yield the intermediate 0+1 construct which was used in the next step without further characterization. To the 0+1 intermediate construct dissolved/suspended in water was added TET001K dissolved in water, followed by aqueous solutions of aminoguanidine hydrochloride, CuSO₄ and THPTA (CAS 760952-88-3) which gave a blue reaction mixture. The reaction was activated by addition of sodium ascorbate solution. After continuous addition of CuSO₄ and sodium ascorbate solutions, full conversion was achieved after 24 h. The 3+1 construct was purified on a C4 RP column (Scheme 17).

Deconvoluted HRMS characterisation data: MWₜₕₑₒ=14647.2; MW_{found}=14646.4

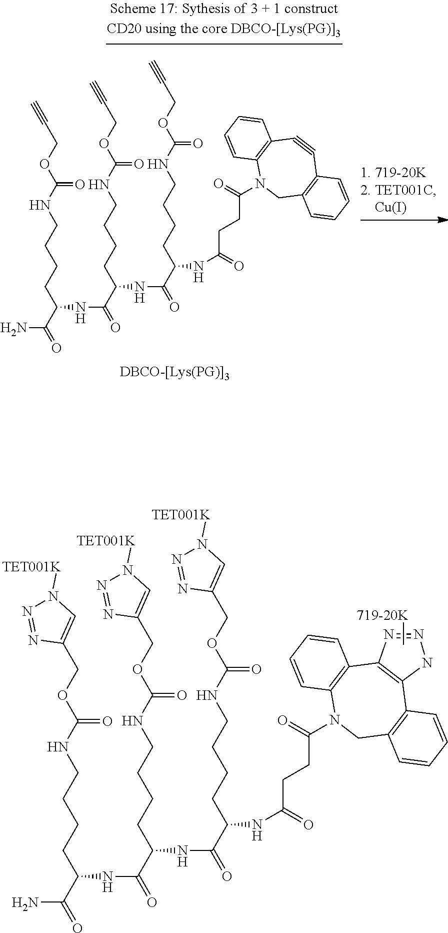

### Synthesis Example 19: Synthesis of 2+1 Construct CD14 Using DBCO-Core B

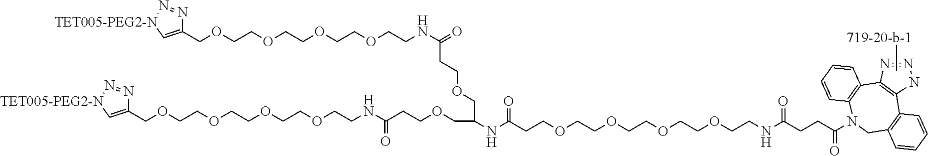

719-20-b-1 is dissolved in ACN/water (1:3) and mixed with DBCO-Core B dissolved in ACN/water (1:1). The reaction is monitored by LCMS and complete in 18 hours. The mixture is diluted with water and purified by RP purification using a Biotage® Sfär Bio C4 D-Duo 300 Å 20 μm column and a 0-30% B gradient (A: 0.1% TFA in water, B: 0.1% TFA in ACN). Fractions containing the pure product were combined and freeze dried and the product was isolated.

719-20-b-1-DBCO-Core B is dissolved in water and mixed with TET005-PEG2-K(N₃)—OH dissolved in ACN/water (4:1). CuBr·SMe₃ and THPTA are dissolved in water and added to the reaction mixture. DIPEA is added to the reaction and the reaction is monitored by LCMS. After completion EDTA (11 equivalents) is added. The mixture is diluted with water and by RP purification using a Biotage® Sfär Bio C4 D-Duo 300 Å 20 μm column and a 0-40% B gradient (A: 0.1% TFA in water, B: 0.1% TFA in ACN). Fractions containing the pure product were combined and freeze dried and the product was isolated (Scheme 18).

Deconvoluted HRMS characterisation data: MWₜₕₑₒ=9430.0; MW_{found}=9429.5

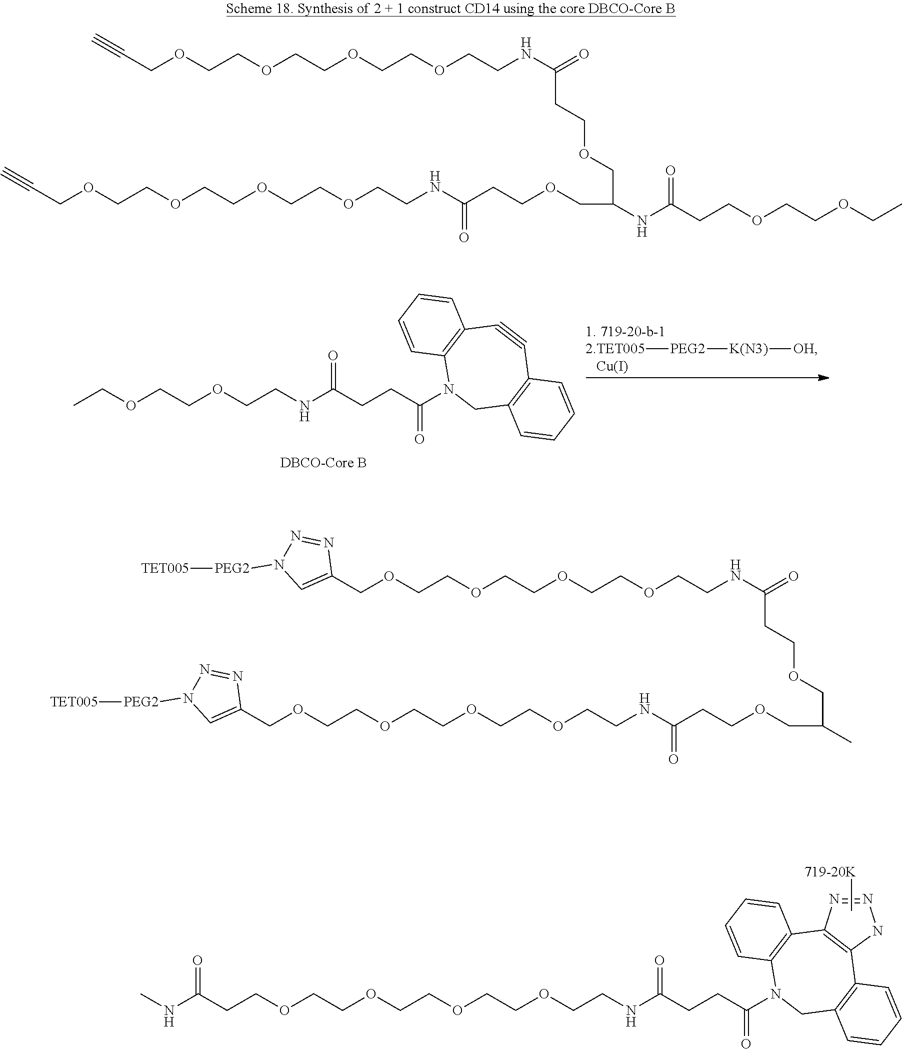

### Synthesis Example 20: Synthesis of 3+1+1 Construct Using DBCO-Lys(Br)-Lys(Br)-Lys(Br)—NH-PG (Batch Number 21E3299/PD00723)

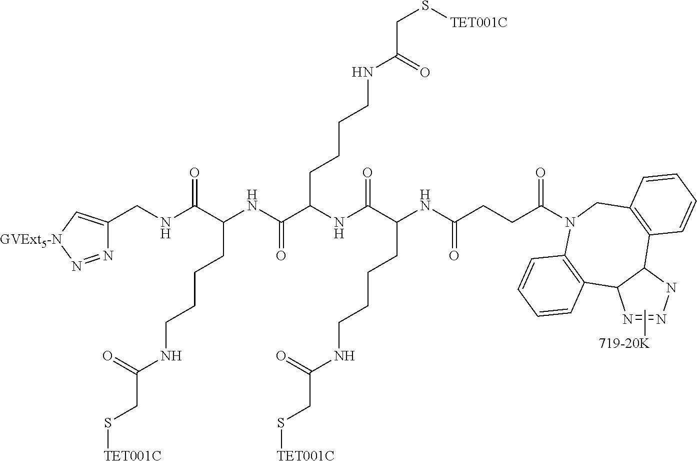

The DBCO-Lys(Br)-Lys(Br)-Lys(Br)—NH-PG core is dissolved in ACN/water (1:1) at a concentration of 2 mg/ml and mixed with TET001C (3.5 equivalents) dissolved in ACN/water (4:1) at a concentration of 5 mg/mL. The pH was adjusted to 8 using 0.5 M NaHCO₃. The reaction is monitored by LCMS. After complete coupling the pH is lowered to 5 using 5% Acetic acid in water and 719-20K (1.1 equivalents) dissolved in ACN/water (4:1) at a concentration of 5 mg/mL. The reaction is monitored by LCMS and after completion TCEP (3 equivalents) is added. The mixture is diluted with water and purified by RP purification using a Biotage® Sfär Bio C4 D-Duo 300 Å 20 μm column and a 0 40% B gradient (A: 0.1% TFA in water, B: 0.1% TFA in ACN). Fractions containing the pure intermediate 3+1+0 product were combined and freeze dried and used for the next step.

The 3+1+0 intermediate and GVExt5 (1.5 equivalents) were dissolved in degassed water under N₂ at a concentration of 2 mg/mL. Copper (I) bromide-dimethyl sulfide complex (20 equivalents) and Tris(3-hydroxypropyltriazolylmethyl)amine (THPTA) (20 equivalents) mixed with degassed water under N₂ at a concentration of 1 mg/ml were added to reaction mixture. Stirred at room temperature for 30 min. The pH was adjusted to 7 using N,N-Diisopropylethylamine (DIPEA) (20 equivalents). The reaction is monitored by LCMS. After complete coupling the reaction mixture was quenched with EDTA (25 equivalents) in water and pH is lowered to 4 using 5% trifluoracetic acid in water. The mixture is diluted with water and purified by RP purification using a Biotage® Sfär Bio C4 D-Duo 300 Å 20 μm column and a 0-40% B gradient (A: 0.1% TFA in water, B: 0.1% TFA in ACN). Fractions containing the pure product were combined and freeze dried (Scheme 19).

Deconvoluted HRMS characterisation data: MWₜₕₑₒ=18042.2; MW_{found}=18041.8

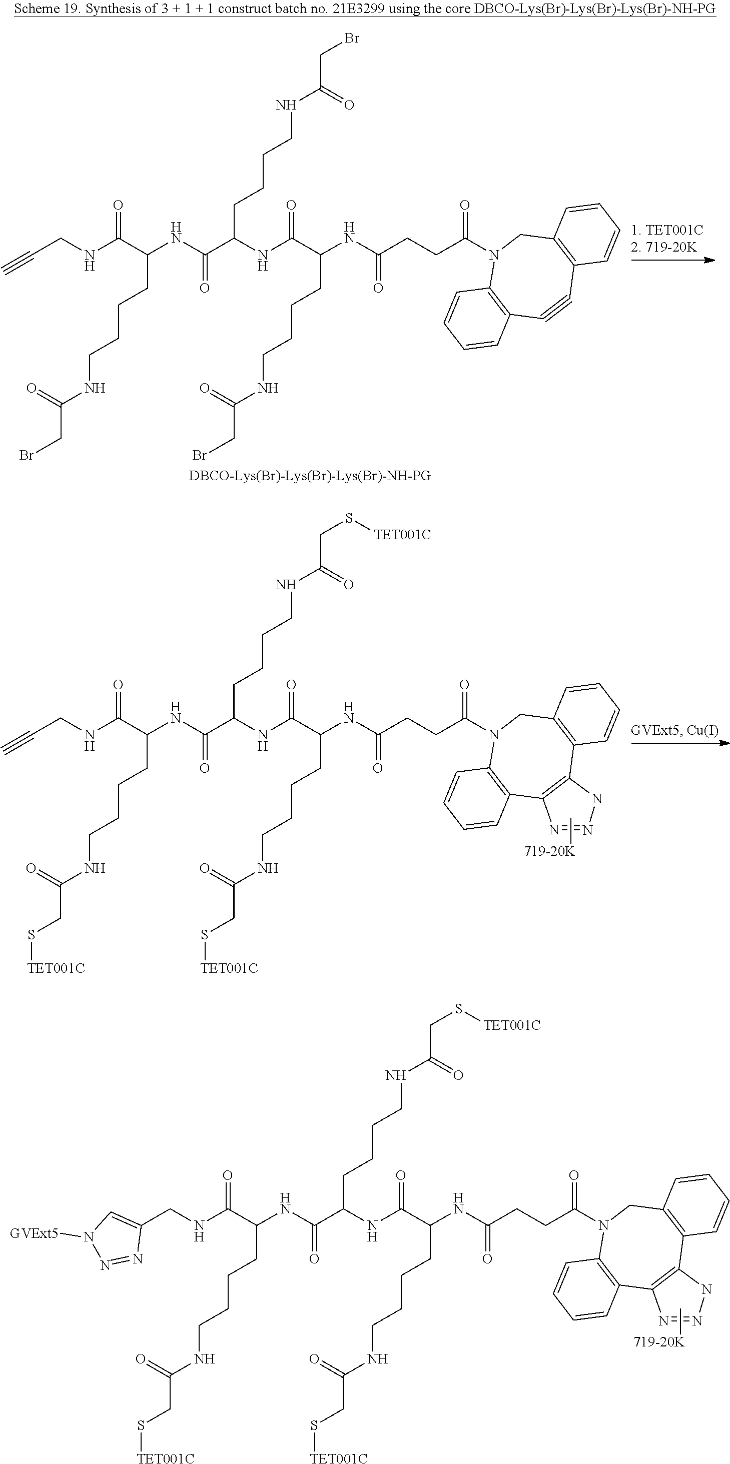

### Synthesis Example 21: Synthesis of 3+1+1 Construct Using DBCO-Lys(Br)-Lys(Br)-Lys(Br)—NH-PEG1-PPG (Batch Number 21E3300/PD00724)

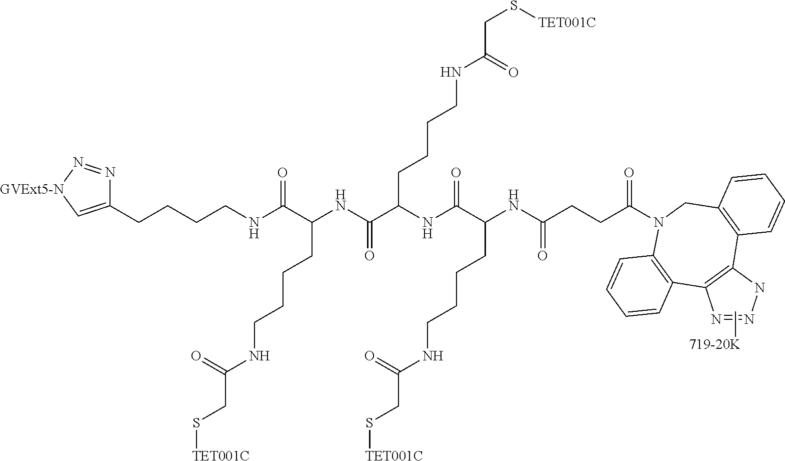

The same procedure as in Synthesis Example 20 was used, except that DBCO-Lys(Br)-Lys(Br)-Lys(Br)—NH-PEG1-PG was used instead of DBCO-Lys(Br)-Lys(Br)-Lys(Br)—NH-PG (Scheme 20).

Deconvoluted HRMS characterisation data: MWₜₕₑₒ=18084.2; MW_{found}=18082.3

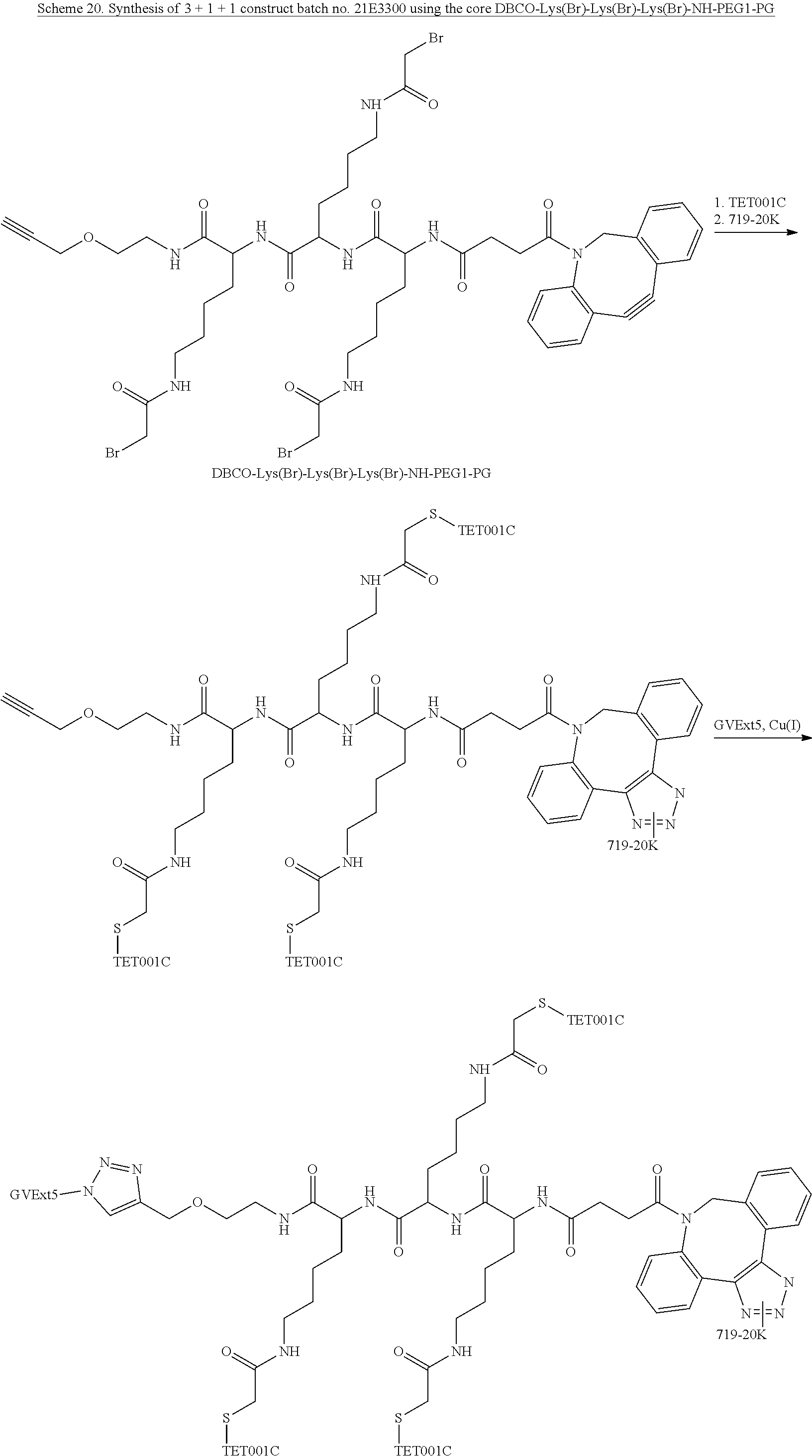

### Synthesis Example 22: Synthesis of 3+1+1 Construct Using DBCO-Lys(Br)-Lys(Br)-Lys(Br)-Ser(PPG)-NH₂ (Batch Number 21E3301/PD00725)

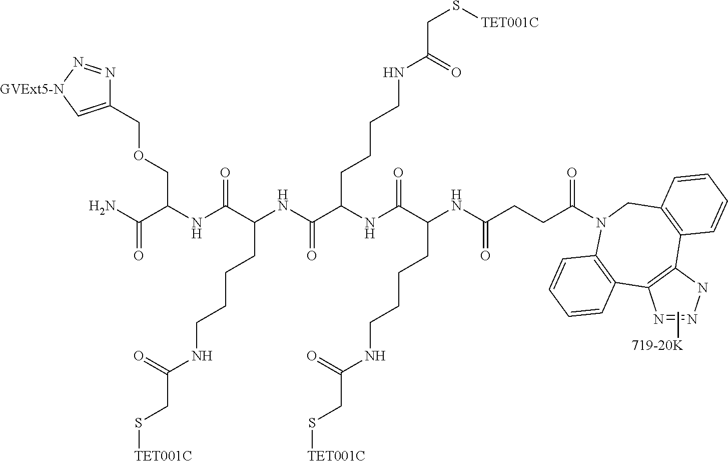

The same procedure as in Synthesis Example 20 was used, except that DBCO-Lys(Br)-Lys(Br)-Lys(Br)-Ser(PG)-NH₂ was used instead of DBCO-Lys(Br)-Lys(Br)-Lys(Br)—NH-PG (Scheme 21).

Deconvoluted HRMS characterisation data: MWₜₕₑₒ=18127.3; MW_{found}=18125.5

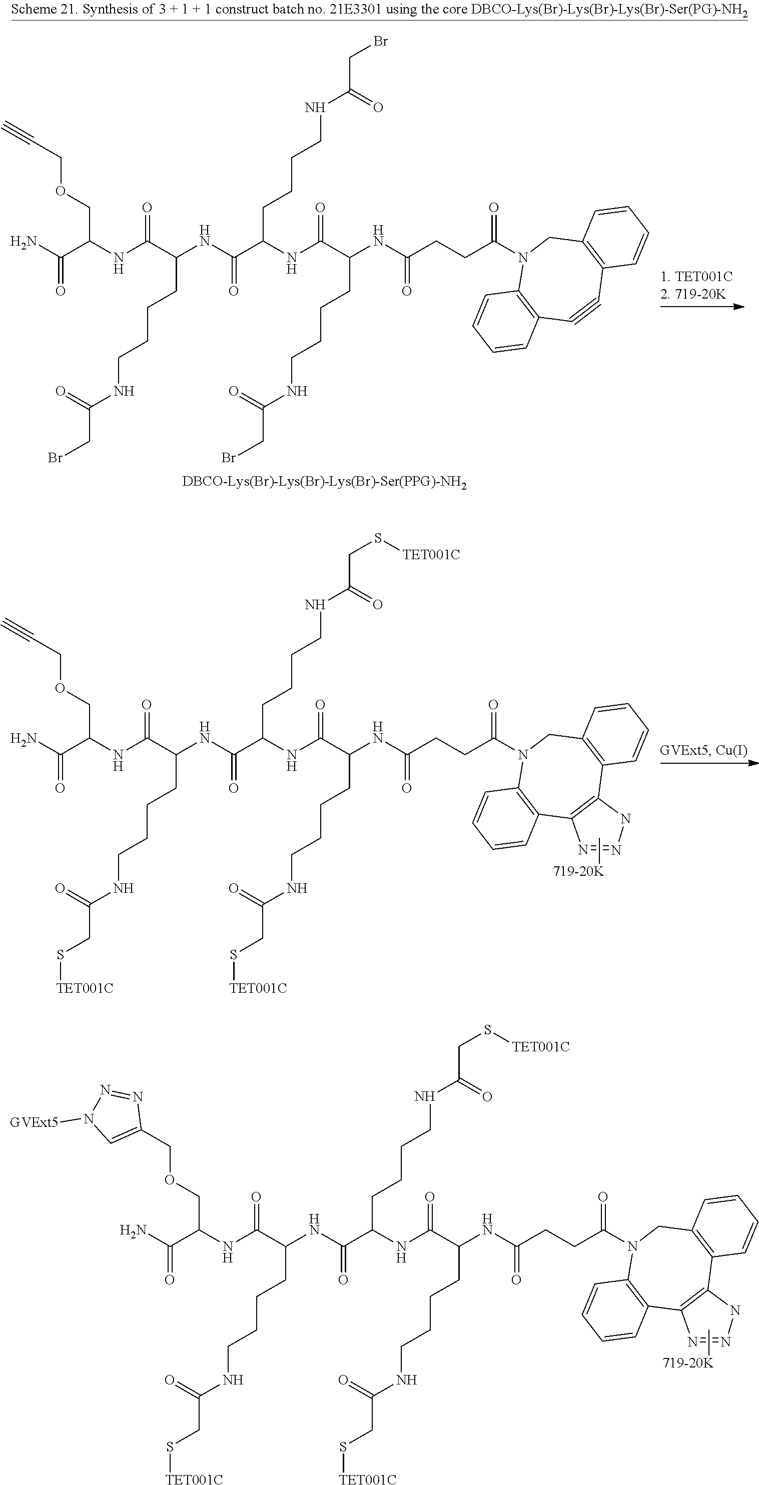

### Example 11: SEQ ID NO: 1 is Immunogenic in Transgenic HLA-DR4 Mice

### Materials and Methods

The study was designed to investigate the immunogenicity of an hTERT polypeptide having the sequence of SEQ ID NO: 1 at a dose of 30 μg (9 nmol) with CpG 1826 at 25 μg mixed with IFA in female B6.129S2-H2-Ab1tm1GruTg(HLADRA/H2-Ea,HLA-DRB1*0401/H2-Eb) 1Kito strain mice. B6.129S2-H2-Ab1tm1GruTg(HLA-DRA/H2-Ea,HLADRB1*0401/H2-Eb) 1Kito strain mice were obtained from Taconic Biosciences A/S. IFA was mixed with the peptide/CpG in a 1:1 ratio with the remaining volume occupied by either the peptide alone or peptide plus CpG.

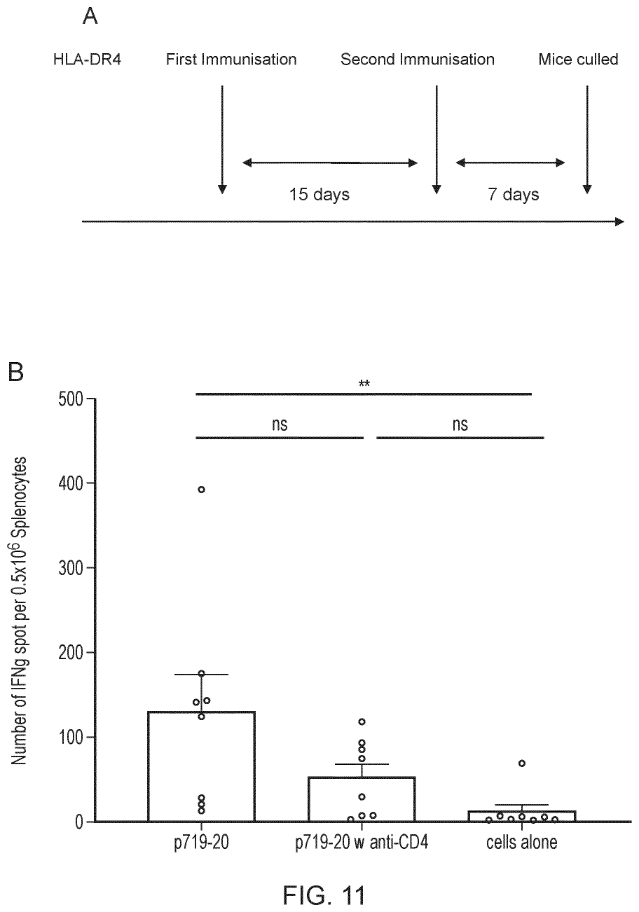

As shown in FIG. 11A, the test material was administered using a prime boost vaccination schedule with approximately 2 weeks between the first and second immunisations and then 1 week after the second immunisation, the animals were sacrificed and necropsy was performed. Spleens were used to perform ex-vivo ELISpots and to show the frequency of responding T-cells and IFN-gamma released into the medium (complete growth medium). Spleens were flushed out using T-cell medium (RPMI 1640, Life Technologies/Thermo Fisher Scientific, containing 1% w/V L-Glutamine (SLS/Lonza), 10% v/v FBS (Fisher/GE Healthcare), 2% HEPES (SLS/Lonza), 0.1% v/v Fungizone (Promega)), after which they were passed through a strainer to remove tissue-debris and centrifuged at 300 g for 10 minutes. Cell pellets were resuspended in T-cell medium for counting and plating. An anti-CD4 antibody from BioXCell was used to block CD4+ T-cell activation during ex-vivo restimulation. All the peptides were used at a final concentration of 10 μg/mL. The frequency of vaccine-induced responsive cells after 48 hours stimulation in vitro was determined using a murine IFN-γ ELISpot kit (Mabtech). For this, transmembrane 96-well Millipore plates were pre-coated with capture antibody according to the manufacturer's protocol. For each experiment, 0.5×10⁶ splenocytes/well were plated in 4 wells+/−10 g/mL of peptides+/−anti-CD4 antibody. Staphylococcal Enterotoxin B (SEB) was used as a positive control. Cells in media only was used as negative control. The cells were then counted.

### Results

FIG. 11B displays the individual mice per group (n=8) and represents mice pooled from 3 different experiments run at separate time-points. Statistical analysis was performed using Kruskal-Wallis test with Dunn's multiple comparisons test; “**” refers to p<0.01 and “ns” is non-significant. FIG. 11B displays data from either cells stimulated with SEQ ID NO: 1, with SEQ ID NO: 1 combined with an anti-CD4 antibody or cells alone as a negative control. SEB as a positive control is not shown, but displayed a strong cell activation. The data show a potent CD4-drived T-cell activation response to the restimulation using SEQ ID NO: 1 as there is a significant increase in the number of spots compared to cells alone. Anti-CD4 antibody affected the activation and thus the response is likely to be CD4+ T-cell mediated.

### Discussion

The data shown in FIG. 11B demonstrate that a polypeptide having the sequence of SEQ ID NO: 1 is immunogenic in female B6.129S2-H2-Ab1tm1GruTg(HLADRA/H2-Ea,HLA-DRB1*0401/H2-Eb)1Kito strain mice at a dose of 9 nmol. Although the response is not uniform at this does level (both non-responders and responders were observed), further experiments demonstrate that all mice appear to respond to a 22 nmol dose (see, for example, Example 15 below). Thus these data demonstrate that transgenic HLA-DR4 mice are suitable to be used for in vivo experiments involving SEQ ID NO: 1 and conjugates comprising SEQ ID NO: 1.

### Example 12: Peptide Conjugates with a Higher Molecular Weight are Processed and Presented Equally Efficiently to T-Cells

The aim was to evaluate whether or not a peptide conjugate with a higher molecular weight (e.g. >14 kDa) can be processed and presented to T-cells to the same degree as a polypeptide having the sequence of SEQ ID NO: 1 in solution. It has been reported that whole protein is processed less efficiently that synthetic long peptides (Rosalia et al., Eur J Immunol. 2013 October; 43 (10): 2554-65). Use was made of an assay based on two stably transfected cells lines.

### Materials and Methods

The Jurkat derivative cell line J76 has been stably transfected with UT1, a TCR specific for SEQ ID NO: 1. J76 lacks expression of TCRa and TCRb chains. The UT1 has been cloned from T-cells from an individual who has shown SEQ ID NO: 1 specific T-cell responses in a clinical trial involving the UV1 cocktail of polypeptides (SEQ ID NOS: 1, 52 and 53). The same cell line was stably transfected with the reporter NFAT-GFP. NFAT is a transcription factor that plays important role in T-cell activation. Upregulation of GFP expression can be used to analyze the specific activation of the resulting cell line J76 1439 907 in response to recognition of SEQ ID NO: 1 by the UT1. The second cell line was an Epstein-Barr Virus (EBV)-Lymphoblastoid Cell Line (LCL) expressing HLA-DQ which is matched for the UT1 and cloned from the same individual (EBV LCL 802).

The polypeptides of SEQ ID NO: 1 or 52 were reconstituted to 500 uM in sterile water. A conjugate comprising the polypeptide of SEQ ID NO: 1 (CD12B or CD20B; see Table 10) was reconstituted to 1 mg/ml (52.9 uM) in sterile water. J76 and the EBV cells were washed by centrifugation and resuspended in RPMI with 25% FBS and added to 96-well tissue culture treated plates to a total volume of 150 μl with 100 000 J76 cells and 200 000 EBV cells per well. J76 and EBV cells only wells were also evaluated as negative controls. The peptides (SEQ ID NO: 1 or 52) and peptide conjugate (CD12B or CD20B) were added to a 5× concentrated stock solution (50 μl per well) in plain RPMI 1640 medium and then the peptides (SEQ ID NO: 1 or 52) and peptide conjugate (CD12B or CD20B) were added to the cells. In one experiment, the conjugate (CD12B) and peptides (SEQ ID NO: 1 or 52) were added to a final concentration of 0.5, 1.5 and 5 μM. In a second experiment, SEQ ID NO: 1 and 52 were added to a final concentration of 0.5, 1.5 and 5 μM and CD12B and CD20B were added to a final concentration of 0.5, 1.5 and 3 μM. The final volume of the cells and peptide co-incubation was 250 μl. Plates were incubated at 37° C. for 24 hours. The polypeptide of SEQ ID NO: 52 was used as a negative control peptide as it should not activate the SEQ ID NO: 1 specific T cells.

After this, cells were transferred to a V-shaped 96-well plate for staining with antibodies and later flow cytometry analysis. Cells were washed by centrifugation and resuspended three times prior staining. Staining was performed in room temperature for 25 min and subsequent to washing, cells were analyzed on a Cytoflex ADP analyzer. A live/dead marker was used to gate away dead cells. Anti-CD3 antibody labeled with allophycocyanin (APC) and anti-CD19 antibody labeled with phycoerythrin (PE) were used to stain the cells. Compensation was performed using single stained beads (using the APC- and PE-labelled antibodies used for the cell staining).

### Results and Discussion

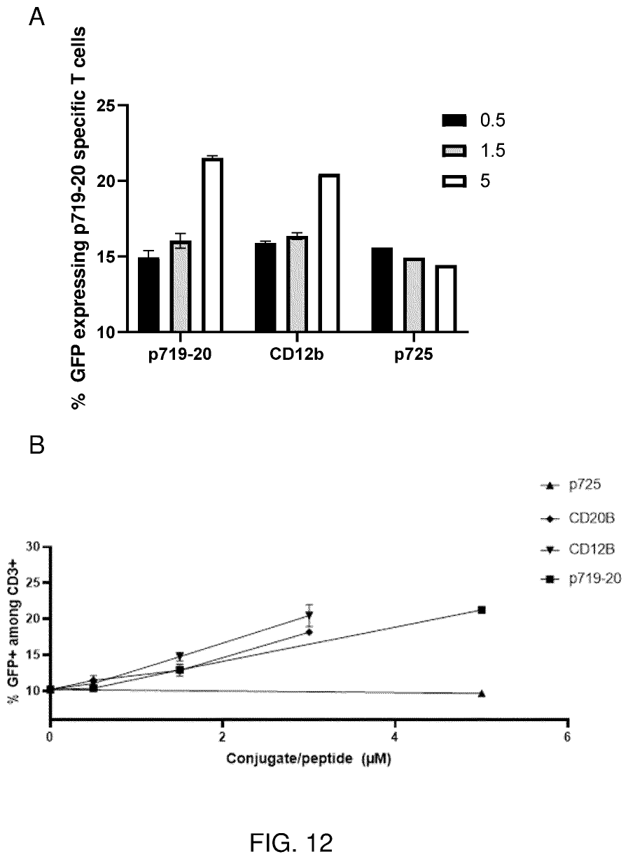

FIG. 12A shows GFP-expressing, CD3-positive cells (identified by gating) when exposed to the three different concentrations of the peptides (SEQ ID NO: 1; “719-20” or SEQ ID NO: 52; “p725”) or peptide conjugate (“CD12b”). Each bar represents the mean value+/−SD of duplicates for SEQ ID NO: 1 (“p719-20”) and the conjugate comprising SEQ ID NO: 1 (“CD12b”), while SEQ ID NO: 52 (“p725”) was run as a single sample for each concentration. The J76 cells become activated when the EBV cells present an epitope derived from SEQ ID NO: 1. The activation results in an increased GFP expression. FIG. 12A shows that a polypeptide comprising the sequence of SEQ ID NO: 1 (“p719-20”) and a conjugate comprising the polypeptide of SEQ ID NO: 1, linked to a B-cell epitope (“CD12b”) can activate the T cells equally well when used in equimolar doses. No activation of T-cells was noted when the antigen-presenting cells (EBV) were not present in the culture (data not shown). FIG. 12B shows that the conjugate CD20B works equally well in the same assay. Thus, the larger molecular weight of the conjugates does not hamper the processing of the polypeptide of SEQ ID NO: 1 and its presentation to T-cells.

### Example 13: Conjugates Comprising a B-Cell Epitope and a CD4+ T-Cell Epitope Derived from hTERT are Bound by Polyclonal Antibodies Specific to the B-Cell Epitope

Conjugates comprising the B-cell epitope of SEQ ID NO: 7 (MTTE) and a CD4+ T-cell epitope derived from hTERT (SEQ ID NO: 1, 116 or 117) were tested by ELISA to evaluate binding of the conjugates by antibodies specific to SEQ ID NO: 7. Further detail on the conjugates comprising the sequences derived from hTERT are presented in Table 10 above.

### Materials and Methods

Indirect and sandwich ELISA were used to evaluate polyclonal antibody binding to the conjugates using the protocols below. It is to be understood that in the indirect ELISA, the conjugates to be tested were coated onto the plates whereas in the sandwich ELISA, conjugates comprising the polypeptide of SEQ ID NO: 1 which were to be tested were captured onto the plate via rabbit polyclonal antibody specific for SEQ ID NO: 1.

Sandwich ELISA: the sandwich ELISA was performed by Capra Science, Sweden, according to the following procedure. Thermo Scientific™ Clear Flat-Bottom Immuno Nonsterile 96-Well Plates were used and coated with a polyclonal rabbit anti-p719-20 (SEQ ID NO: 1) antibody (0.5 μg per well) and in total 50 μl/well and incubated at 4° C. overnight. The plate was blocked using a protein free block solution (Pierce) at 1 hour at room temperature and subsequently washed using PBS and 0.05% Tween washing solution. The conjugates were added to the plate and concentrations indicated and in a serial dilution and incubated for 1 hour at room temperature. Subsequently the plate was washed with the washing solution and then incubated with anti-MTTE antibody based on a Tetaquin® (polyclonal human IgG from high titer anti-tetanus donors or a monoclonal recombinant chimeric anti-MTTE antibody. The plate was washed with washing solution and subsequent it a secondary antibody, goat anti-human kappa HRP was added in a PBS solution with 1% BSA. After 1 hour of incubation the plate was washed again and then the substrate TMB was added to develop the assay and a stop solution of 1 M H2SO4 was added before the plate was read using absorbance.

Indirect ELISA: the indirect ELSIA was performed in-house according to the following procedure. The conjugates were dissolved and diluted in Microplates, 96 well, PS, U-Bottom-Clear-Non-binding plate and 100 μl of the prepared solutions were added to ELISA plates Corning costar 96 well plates and were incubated for 2 hours at room temperature. Plates were washed with 250 μl/well PBS+0.05%+ Tween20 (PBST). The plate was blocked with 200 μl with Superblock T20 (Thermo Scientific) and incubated for min. The plate was emptied, and block solution addition was repeated for a total of 3 times. The plate was washed again with the PBST solution. Detection of MTTE was performed by either diluted serum sampled from a TTd vaccinated individual or a monoclonal recombinant chimeric anti-MTTE antibody, 100 μl per well, and incubated for 2 hours at room temperature. The plates were washed with PBST. Antibodies were detected using a secondary HRP Goat anti-human kappa light chain secondary antibody and incubated for 1 hour at room temperature. After washing TMB was added to the wells and colour developed was monitored and the reaction was stopped by addition of 100 μl of 1M H₂SO₄ and absorbance was read.

### Results

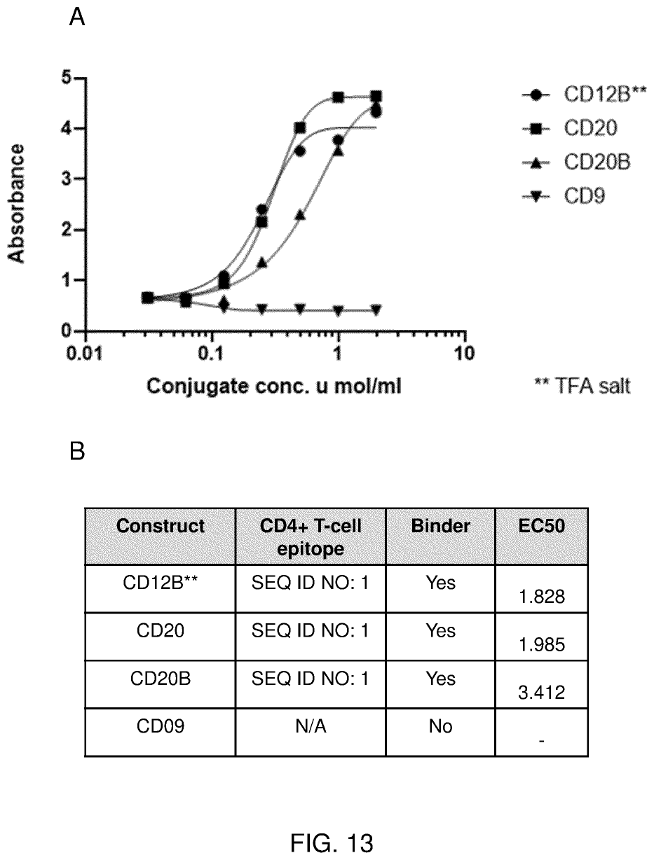

FIGS. 13A and 13B show the results from a sandwich ELISA experiment using conjugates comprising the polypeptide of SEQ ID NO: 1 as the CD4+ T-cell epitope. CD09 did not comprise a CD4+ T-cell epitope and was used as a negative control. As shown in FIGS. 13A and 13B, each of the conjugates of CD12B, CD20 and CD20B which comprise SEQ ID NO: 1 was bound by human polyclonal antibodies (from the blood plasma product TetaQuin) that recognise MTTE (SEQ ID NO: 7).

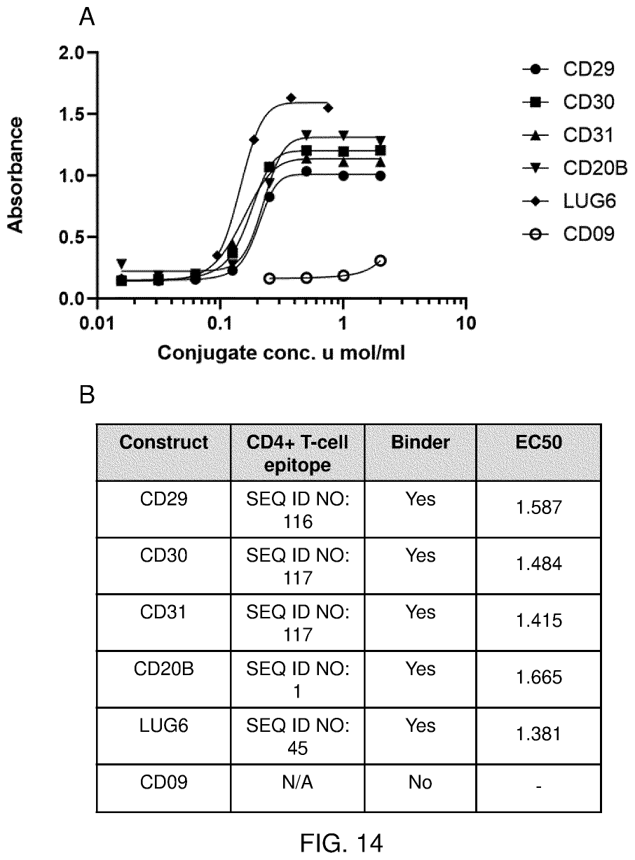

FIGS. 14A and 14B show the results from an indirect ELISA experiment using conjugates comprising the polypeptides of SEQ ID NO: 1 (CD20B), 116 (CD29) or 117 (CD30, CD31) as the CD4+ T-cell epitope. The conjugate LUG6, which comprises SEQ ID NO: 45 derived from NY-ESO-1 was used as a positive control. As shown in FIGS. 14A and 14B, each of the conjugates of CD29 (comprising SEQ ID NO: 116), CD30 and CD31 (comprising SEQ ID NO: 117), or CD20B (comprising SEQ ID NO: 1) was bound by human polyclonal antibodies derived from serum containing polyclonal antibodies that recognise MTTE (SEQ ID NO: 7).

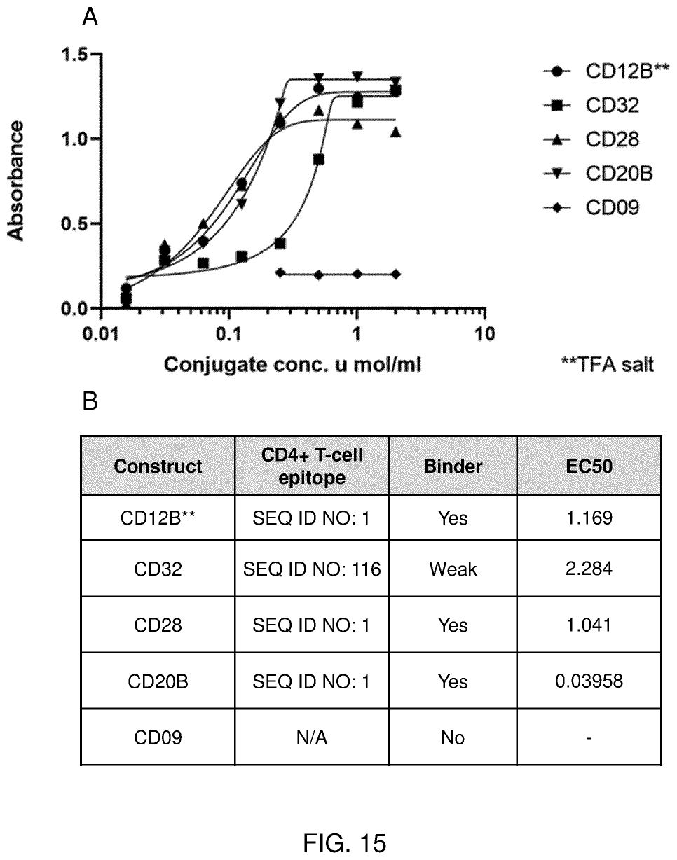

FIGS. 15A and 15B show the results from an indirect ELISA experiment using conjugates comprising the polypeptide of SEQ ID NO: 1 (CD12B, CD28, CD20B) or SEQ ID NO: 116 (CD32) as the CD4+ T-cell epitope. The conjugate CD28 comprises the sequence of SEQ ID NO: 1 Plus a CD8+ T-cell epitope derived from PAP (NPILLWQPIPV; SEQ ID NO: 119). As shown in FIGS. 15A and 15B, each of the conjugates of CD12B, CD28, CD20B (comprising SEQ ID NO: 1) or CD32 (comprising SEQ ID NO: 116) was bound by human polyclonal antibodies derived from serum containing polyclonal antibodies that recognise MTTE (SEQ ID NO: 7). CD32 demonstrated relatively weaker binding to the human polyclonal antibody serum in this experiment.

### Discussion

Conjugates comprising the MTTE sequence (SEQ ID NO: 7) as a B-cell epitope and a CD4+ T-cell epitope derived from hTERT (SEQ ID NO: 1, 116 or 117) are bound by polyclonal antibodies specific for SEQ ID NO: 7. Therefore, anti-MTTE antibodies are capable of recognising the MTTE sequence of SEQ ID NO: 7 when comprised within the above-mentioned conjugates.

### Example 14: A Conjugate Comprising SEQ ID NO: 1 Induces Elevated T-Cell Responses in Transgenic HLA-A2/HLA-DR1 Mice

### Materials and Methods

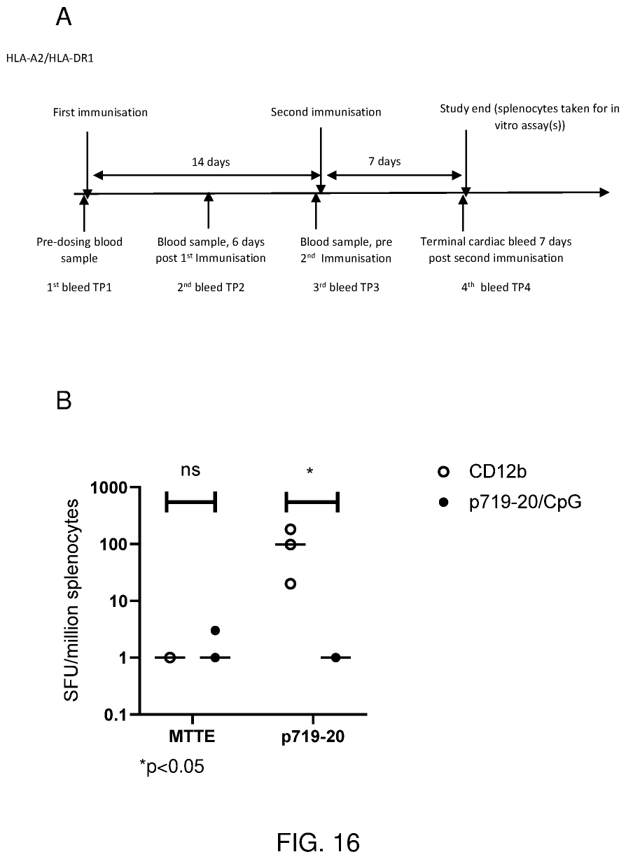

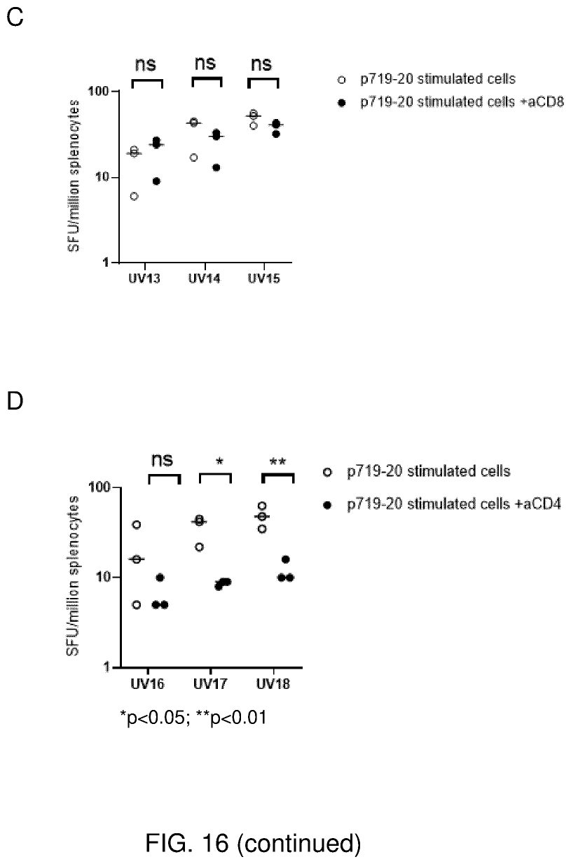

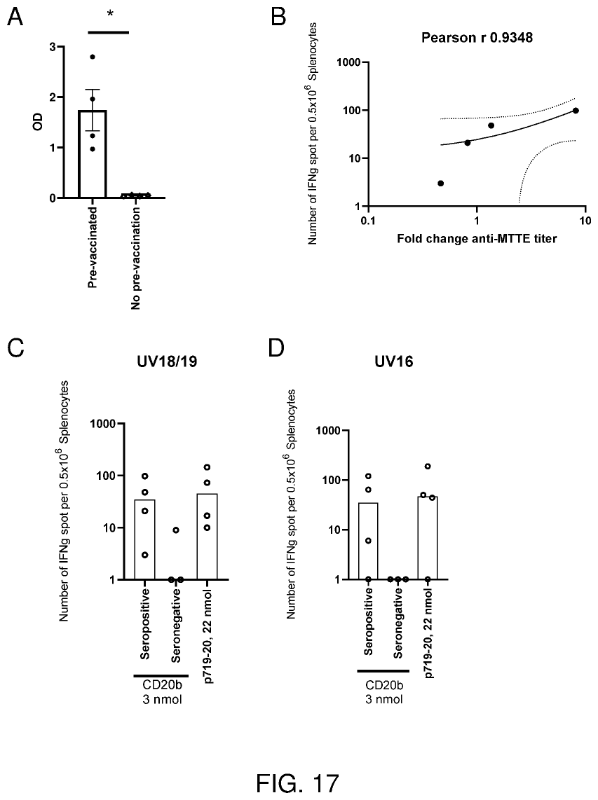

Female mice of strain B2m,Tg(HLA-A/H2-D/B2M)1Bpe,Tg(HLA-DRA*0101,HLA-DRB1*0101)1Dma,H2-Ab1 (EMMA Repository, Orleans, FRANCE) were immunized (prime/boost) at the tail base with either: the polypeptide of SEQ ID NO: 1 mixed with CpG (as a reference adjuvant, a TLR9 agonist) (Group 1: SEQ ID NO: 1 (20 μg) plus CpG (25 μg) (50 μL/mouse); n=3) or the compound CD12B (see Table 10), a conjugate comprising three MTTE (SEQ ID NO: 7) polypeptides and the polypeptide of SEQ ID NO: 1 (Group 2: CD12B, 20 μg/mouse (100 μL/mouse); n=3) according to the schedule shown in FIG. 16A. At the day of termination, the spleen was harvested and single cell suspensions of the splenocytes were prepared.

Spleens were flushed out using T-cell medium (RPMI 1640, Life Technologies/Thermo Fisher Scientific, containing 1% w/v L-Glutamine (SLS/Lonza), 10% v/v FBS (Fisher/GE Healthcare), 2% HEPES (SLS/Lonza), 0.1% v/v Fungizone (Promega)), after which they were passed through a strainer to remove tissue-debris and centrifuged at 300 g for 10 minutes. Cell pellets were resuspended in T-cell medium for counting and plating.

The frequency of vaccine-induced responsive cells after 48 hours stimulation with selected polypeptides in vitro (as shown in Table 11) was determined using a murine IFN-γ ELISpot kit (Mabtech). For this, transmembrane 96-well Millipore plates were pre-coated with capture antibody according to the manufacturer's protocol. For each experiment, 0.5×10⁶ splenocytes/well were plated in 4 wells+/−10 μg/ml of peptides+/−anti-CD4 or CD8 antibody. Staphylococcal Enterotoxin B (SEB) was used as a positive control. Cells in media only was used as negative control.

TABLE 11
Polypeptides used for in vitro stimulation:

| | | Poly- |
|---|---|---|
| | | peptide |
| | Polypeptide sequence | reference |
| SEQ ID NO: | (HLA locus) | name |
| 1 | ALSVLNYERARRPGLLGASVLGDDI | P719-20 |
| | HRA | |
| SEQ ID NO: 147 | LLGASVLGL (HLA-A2) | UV13 |
| SEQ ID NO: 148 | GLLGASVLGL (HLA-A2) | UV14 |
| SEQ ID NO: 149 | VLGLDDIHRA (HLA-A2) | UV15 |
| SEQ ID NO: 150 | FSVLNYERARRPGLL (HLA-DR1) | UV16 |
| SEQ ID NO: 151 | RARRPGLLGASVLGL (HLA-DR1) | UV17 |
| SEQ ID NO: 152 | VLNYERARRPGLLGA (HLA-DR1) | UV18 |
| 7 | FIGITELKKLESKINKVF | UV30 |
| | | (MTTE) |

### Results

FIG. 16B shows that SEQ ID NO: 7 (the MTTE sequence) when used alone for stimulation did not induce a T-cell response in splenocytes from the CD12B exposed animals, indicating that the SEQ ID NO: 7 does not provide a T-cell epitope by itself in these mice. Stimulation with the polypeptide of SEQ ID NO: 1 induced a measurable and significantly elevated T-cell response in the CD12B exposed animals compared to the SEQ ID NO: 1/CpG vaccinated animals at the tested dose using Sidak's multiple comparisons test (*p<0.05).

To assess if the T-cell response was CD4+ or CD8+ derived, the splenocytes from the CD12B exposed animals were stimulated with polypeptides comprising predicted MHC class I epitopes (UV13 to 15, Table 11) or predicted MHC class II epitopes (UV16 to UV18, Table 11). The class I-derived stimulations were performed with or without an anti-CD8 antibody and the class II epitope-derived stimulations were performed using an anti-CD4 derived antibody. The statistical analysis was performed using graph pad and using the Sidak's multiple comparisons test (*p<0.05; **p<0.01; “ns”=non-significant). FIG. 16D shows that the CD12B conjugate induces a DR1-derived CD4+ T-cell response as the anti-CD4 antibody (“+aCD4”) was able to reduce the T-cell response significantly for UV17 and UV18. This was seen with the longer SEQ ID NO: 1 peptide stimulation culture as well (data not shown). FIG. 16C shows that the anti-CD8 antibody (“+aCD8”) did not appear to affect T-cell activation indicating that, on this occasion, there was no induction of a measurable CD8+ T-cell response.

### Discussion

The CD12B compound can induce CD4+ T-cell responses in HLA-A2/HLA-DR1 expressing transgenic mice and results in improved T-cell responses in comparison to administration of SEQ ID NO: 1 in an unlinked form and combined with the adjuvant CpG in solution. The mice had not been pre-vaccinated to induce anti-MTTE antibodies; it is plausible that the immune responses to CD12B could be improved in animals with pre-existing anti-MTTE antibodies. The data indicate that the MTTE sequence in itself does not contain a CD4+ T-cell epitope that can induce DRB*01 linked T-cell responses.

### Example 15: In Vivo Assessment of the Immunogenicity of the CD20B Conjugate in Seropositive or Seronegative HLA-DR4 Animals

### Materials and Methods

The immunogenicity of the CD20B conjugate, which comprises 3 copies of SEQ ID NO: 7 (MTTE) as the B-cell epitope and SEQ ID NO: 1 as the CD4+ T-cell epitope (see Table 10), was investigated in seropositive or seronegative female B6.129S2-H2-Ab1tm1GruTg(HLADRA/H2-Ea,HLA-DRB1*0401/H2-Eb)1Kito strain mice. B6.129S2-H2-Ab1tm1GruTg(HLA-DRA/H2-Ea,HLADRB1*0401/H2-Eb)1Kito strain mice were obtained from Taconic Biosciences A/S. Serological status refers to the presence or absence of pre-existing antibodies to MTTE in the mice. Pre-vaccination was performed using peptide (SEQ ID NO: 7, MTTE) haptenated ovalbumin (OVA) in IFA. Thus MTTE-OVA was used as an immunogen to immunize the mice to achieve antibodies against the MTTE sequence to achieve seropositive animals. The immunization was performed using a solution of 75 μg of MTTE-OVA in PBS emulsified in IFA at a 1:1 ratio. This was administrated as a subcutaneous administration at the tail base of the mice on day 0 and day 14 for the seropositive group while the seronegative group was not exposed to MTTE-OVA/IFA. Six days after the last MTTE-OVA administration both seropositive and seronegative groups were administrated 60 μg (approximately 3 nmol) of CD20B in a sterile buffered solution at the tail base as a primer injection. A booster injection of CD20B was administrated seven days post the first injection of CD20B. Seven days post the last injection mice were sacrificed and splenocytes were isolated for further analyses. Blood samples were taken pre and post the MTTE-OVA vaccination and pre as well as post each CD20B administration along with a cardiac bleed at the endpoint day.

Spleens were used to perform ex-vivo ELISpots and to show the frequency of responding T-cells and IFN-gamma released into the medium (complete growth medium). Spleens were flushed out using T-cell medium (RPMI 1640, Life Technologies/Thermo Fisher Scientific, containing 1% w/v L-Glutamine (SLS/Lonza), 10% v/v FBS (Fisher/GE Healthcare), 2% HEPES (SLS/Lonza), 0.1% v/v Fungizone (Promega)), after which they were passed through a strainer to remove tissue-debris and centrifuged at 300 g for 10 minutes. Cell pellets were resuspended in T-cell medium for counting and plating. All the peptides were used at a final concentration of 10 μg/mL. The frequency of vaccine-induced responsive cells after 48 hours stimulation in vitro was determined using a murine IFN-γ ELISpot kit (Mabtech). For this, transmembrane 96-well Millipore plates were pre-coated with capture antibody according to the manufacturer's protocol. For each experiment, 0.5×10⁶ splenocytes/well were plated in 4 wells+/−10 mg/ml of peptides. Staphylococcal Enterotoxin B (SEB) was used as a positive control. Cells in media only were used as negative control.

The following peptides were used for stimulation in the ELISpot assay:

(P719-20):
SEQ ID NO: 1
ALFSVLNYERARRPGLLGASVLGLDDIHRA (96% pure)
(UV16):
SEQ ID NO: 150
FSVLNYERARRPGLL (95% pure) (HLA-DR4)
&
(UV18):
SEQ ID NO: 152
VLNYERARRPGLLGA (95% pure) (HLA-DR4)
(UV19):
SEQ ID NO: 153
RPGLLGASVLGLDDI (95% pure) (HLA-DR4)

ELISA measurements of anti-MTTE antibody titers was performed according to the following protocol:
1. Streptavidin-coated ELISA plates were coated with 100 μl Biotinylated peptide (MTTE-biotin diluted in PBS 1 nmol/ml) according to the plate layout and incubated at 4° C. overnight.
2. The plates were washed four times with 250 μl PBS/0.05% Tween20.
3. The plates were blocked with 200 μl PBS/10% BSA/0.05% Tween20 and incubated at room temperature (RT) for 1 hour.
4. The plates were washed four times with 250 μl PBS/0.05% Tween20.
5. The plasma was diluted in PBS/1% BSA/0.05% Tween20 (first 1:50, followed by 2× dilutions yielding 100× and 20× dilution, yielding 1000×). 100 μl of diluted supernatants were added per well and incubated for 2 hours at RT.
6. The plates were washed four times with 250 μl PBS/0.05% Tween20.
7. 100 μl of secondary antibody Goat anti-mouse Ig-HRP was added per well, diluted 1:5000 in PBS/1% BSA and incubated for 1 hour in RT in the dark.
8. The plates were washed four times with 250 μl PBS/0.05% Tween20.
9. 100 μl of TMB was added per well and the reaction was stopped with 100 μl 1M H2SO4. Absorbance was read at 450-570 nm. Developing time was 3 min for plates with serum from mice immunized with MTTE-OVA and 4 min for serum with plates from mice immunized with CD20B.

### Results

FIG. 17A shows the anti-MTTE antibody levels in mice at the end of the experiment. The mice had either been pre-vaccinated with MTTE-OVA or had not been pre-vaccinated with MTTE-OVA but all were exposed to 60 μg of CD20b in PBS in a prime boost setting. Significance was assessed by a Mann Whitney test *p=0.0286. As shown in FIG. 16A, mice that had not been exposed to MTTE-OVA did not develop antibodies to the MTTE sequence and were not able to mount an antibody response to the MTTE sequence upon subsequent exposure to the CD20B conjugates. However, mice exposed to MTTE-OVA had high titers of anti-MTTE antibodies post the cycle of the MTTE-OVA/CD20B vaccination.

To evaluate if T-cell responses against peptide fragments from SEQ ID NO: 1 (UV18 and UV19), correlate with anti-MTTE titre increases pre and post CD20B exposure, a correlation analysis was performed, as shown in FIG. 17B. Referring to FIG. 17B, the x-axis displays the fold change of anti-MTTE OD values pre and post CD20B exposure (dilution of serum 1:1000 when assessing the antibody response) for the seropositive animals and its correlation to T-cell responses in response to stimulation with the peptide mix of UV18 and UV19 combined. The correlation is high with a Pearson r of 0.9348. The plotted curve is a simple regression line with the 95% confidence bands of the best-fit line. Analyses were performed using Graph pad prism. Thus there is a strong correlation between an increase in anti-MTTE titres and T-cell responses to the peptides from SEQ ID NO: 1.

FIGS. 17C and 17D show T-cell responses after stimulation with peptides from SEQ ID NO: 1; a combination of UV18/19 (FIG. 17C) or UV16 alone (FIG. 17D). The seronegative group presented an outlier that was removed in the above analysis after an outlier analysis using ROUT (Q=2%). As shown in FIGS. 17C and 17D, T-cell responses were observed in seropositive animals exposed to CD20B at a dose of 3 nmol. In contrast, seronegative animals exposed to CD20B did not mount a T-cell response as measured by responses to the UV18/UV19 or UV16 stimulation. The T-cell responses in the seropositive animals exposed to CD20B were approximately equal to those measured in animals that received a much higher dose (22 nmol) of the peptide of SEQ ID NO: 1 (P719-20) alone (i.e. non-conjugated) formulated in IFA and CpG.

### Discussion

The data support that transgenic HLA-DR4 mice can mount an efficient T-cell response to SEQ ID NO: 1 (P719-20) when vaccinated with the CD20B conjugate. The T-cell responses are only present in seropositive animals suggesting that antigen/antibody complex formation acts as a driver for the cellular (i.e. T-cell) immune responses. Administration of a conjugate comprising SEQ ID NO: 1 (CD20B) is able to promote efficient T-cell responses at a dose approximately 7 times lower than that required for the non-conjugated peptide of SEQ ID NO: 1.

An increase in antibody levels post CD20B exposure (prime/boost vaccination) compared to pre CD20B exposure correlates with T-cell responses in the seropositive mice. This supports that increased antibody levels subsequent to administration of the conjugate, as compared with prior to administration of the conjugate, can be used to indicate the presence of a T-cell immune response. Thus such antibody increases after the conjugate exposure could substitute for more complex cellular assay readouts, and thus simplify immune responder readouts in clinical trials.

### Example 16

This example concerns the in vivo assessment of the immunogenicity of the peptide of SEQ ID NO: 116 (UV 34-GVExt5) in the form of 3+1 conjugate or 3+1+1 conjugate or GVExt5 peptide+IFA in transgenic HLA-A2/HLA-DR1 animals.

### Materials and Methods

The immunogenicity of the GVExt5 peptide in the form of: CD29 (3+1) [comprising 3 copies of UV30 (MTTE) (SEQ ID NO: 7) as the B-cell epitope, UV34 (GVExt5) (SEQ ID NO: 116) T-cell epitope] or, TFA-3+1+1 conjugate [comprising 3 copies of UV30 (MTTE) as the B-cell epitope, p719-20 (SEQ ID NO: 1) and UV34 (GVExt5) as T-cell epitopes]. Serological status refers to the presence or absence of pre-existing antibodies to MTTE in the mice following vaccination with MTTE peptide conjugated to ovalbumin (OVA).

UV34 (GVExt5) naked peptide (SEQ ID NO: 116) was investigated in seroponegative female and male (08-09 weeks old at the start of the study) B2m,Tg(HLA-A/H2-D/B2M)1Bpe,Tg(HLA-DRA*0101,HLA-DRB1*0101)1Dma,H2-Ab1 (EMMA Repository, Orleans, FRANCE) strain mice.

Pre-vaccination to groups of mice, which were scheduled to receive UV34 later in the form of CD29 (4.1 nmol) or 3+1+1 (3.7 nmol) conjugate was performed using MTTE peptide conjugated to ovalbumin (OVA) in IFA. Thus MTTE-OVA was used as an immunogen to immunize the mice to induce antibodies against the MTTE sequence. The immunization was performed using 75 μg of MTTE-OVA in PBS emulsified in IFA at a 1:1 ratio. The emulsion was administrated as a subcutaneous administration at the tail base of the mice on day 0 for the seropositive group. The seronegative control group, scheduled to receive naked UV34 in PBS was administered as in emulsion with IFA at 1:1 ratio plus CpG (25 μg) (as a reference adjuvant) only as pre-vaccination.

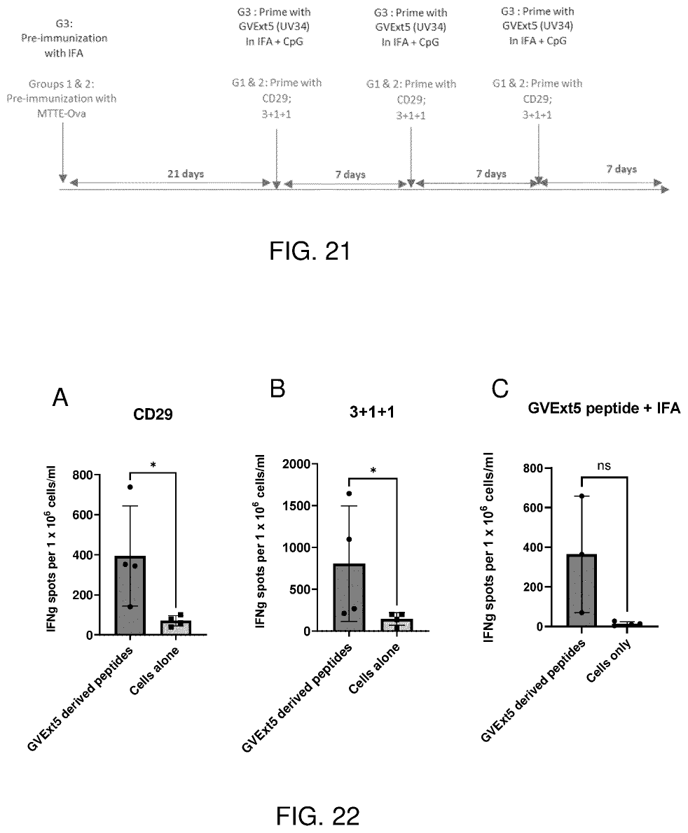

Twenty-one days after MTTE-OVA administration (pre-vaccination) the seropositive group was administrated the conjugate CD29 [60 μg/mouse=4.1 nmol; 100 μL/mouse, n=4] or TFA-3+1+1 [3+1+1, 112.5 μg/mouse=3.7 nmol; 90 μL/mouse, n=4]. The seronegative mice were administered UV34 [14 μg/mouse=4.1 n mol; 100 μL/mouse; n=4]. The conjugates and polypeptide were administered as subcutaneous injections to mice (in the respective groups) at the tail base as primer injections. Two booster injections of CD29, TFA-3+1+1 conjugate and polypeptide UV34 was administered (to mice in the respective groups) with an interval of 1 week between each booster according to the schedule, FIG. 21. On the day of termination, the spleen and the draining lymph nodes (iliac, inguinal, and auxiliary) were harvested and pooled single cell suspensions of these organs were prepared for each mouse separately.

The vaccination schedule is shown schematically in FIG. 21.

Spleens were flushed out using T-cell medium (RPMI 1640, Life Technologies/Thermo Fisher Scientific, containing 1% w/v L-Glutamine (SLS/Lonza), 10% v/v FBS (Fisher/GE Healthcare), 2% HEPES (SLS/Lonza), 0.1% v/v Fungizone (Promega)), after which they were passed through a strainer to remove tissue-debris and centrifuged at 300 g for 10 minutes. Cell pellets were resuspended in T-cell medium for counting and plating.

Lymph nodes were squashed first onto a petri dish containing 2 mL of T-cells media and then through an EASYstrainer 100 μM using the back of a sterile syringe and then rinsing the filter with 1 mL of T-cell media. Cells were spun down at 300 g for 10 min. Pellet was resuspended in 2 mL of T-cells media for counting and plating.

The frequency of vaccine-induced responsive cells after 48 hours stimulation with selected polypeptides in vitro (as shown in Table 12) was determined using a murine IFN-γ ELISpot kit (Mabtech). One day before spleens and lymph nodes were harvested, 96-well ELISpot plates (Millipore) for the IFN-γ ELISpot assay were pre-coated with capture antibody according to the manufacturer's protocol. After 5 washes with PBS/Tween and blocking for a minimum of 30 min with T cell medium including RPMI 1640 (Life Technologies/Thermo Fisher Scientific), containing 1% w/v L-Glutamine (SLS/Lonza), 10% v/v FBS (Fisher/GE Healthcare), 2% HEPES (SLS/Lonza), 0.1% v/v Fungizone (Promega), 0.5×10⁶/well freshly isolated splenocytes+lymph node cells were seeded in triplicate into the plate along with 100 μl of the respective SLPs at a final concentration of 10 μg/ml or 1 μg/ml (for peptides UV64 and UV65). The cells were then incubated at 37° C. in a 5% CO₂ incubator for 48 hours, and the plates then washed 5 times with DPBST. 50 μl/well biotinylated detection antibody (1/1000 dilution) against mouse IFNγ was then added, and the plates incubated for 2 hours at room temperature. Plates were then washed 5 times with DPBST, followed by the addition of 50 μl/well streptavidin alkaline phosphatase (1/1000 dilution). Plates were then incubated for 1 h 30 min at room temperature. After incubation, plates were washed again 6 times with DPBST and then 50 μl/well development solution 5 (BCIP/NBT, BioRad) was added. The plates were left in the dark at room temperature until spots could be seen. Once spots developed, the reaction was stopped by rinsing the plates with tap water. Plates were then left to dry and the spots were quantified using an ELISpot plate reader (Cellular Technology Limited, Shaker Heights, OH, USA). Concanavalin A (ConA), a lectin (isolated from the Jack bean, Canavlia ensiformis) that binds alpha-D-glucose and alpha-D-mannose moieties found in various glycoproteins, glycolipids, and sugars and is a potent leukocyte mitogen, was used as positive control at 1 μl/ml. Unstimulated splenocytes (cells alone) were used as a negative control for every ELISpot assay. All experiments were performed in triplicate.

TABLE 12
Sequence of UV34 (GVExt5) polypeptide and
those polypeptides used in the in vitro
stimulation assay

| | Name | Sequence | Origin | SEQ ID NO |
|---|---|---|---|---|
| 1. | UV34 | LSEAEVRQHREARPALLTSR | | 116 |
| | (GVExt5) | LRFIPKPDGL | | |
| 2. | UV36 | EARPALLTSRLRFIPKPDGL | GVExt5 | 166 |
| 3. | UV57 | QHLKRVQLRELSEAE | GVExt5 | 167 |
| 4 | UV58 | LSEAEVRQHREARPA | GVExt5 | 168 |
| 5. | UV59 | EARPALLTSRLRFIP | GVExt5 | 169 |
| 6. | UV60 | LRFIPKPDGLRPIVN | GVExt5 | 170 |
| 7 | UV64 | LLTSRLRFI | GVExt5 | 171 |
| 8. | UV65 | ALLTSRLRFI | GVExt5 | 172 |
| 9. | UV66 | REARPALLTSRLRFI | GVExt5 | 173 |
| 10. | UV80 | EARPALLTSRLRFIPK | GVExt5 | 126 |

### Results

FIGS. 22A and B shows pooled T cell response of splenocytes-lymph node cultures with the combined results of individual polypeptide culture stimulations. The results demonstrate measurable and significantly elevated T-cell response in the CD29 and TFA-3+1+1 (conjugate) exposed animals compared to the absence of peptides (cells only) using Mann Whitney test *p=0.033. FIG. 22C demonstrates the comparison between sum of splenocytes-lymph node cocultures responses from mice immunized with UV34 polypeptide+IFA towards cultures stimulated with single polypeptides sequences of varying lengths within the UV34 of polypeptide compared to cells only.

### Discussion

The data demonstrate that splenocytes-lymph node cultures from transgenic HLA-DR1 seropositive mice mount a strong and significantly elevated immune response to UV34 (GVExt5) (SEQ ID NO: 116) derived peptides (epitopes), when vaccinated with the CD29 or 3+1+1 conjugates that comprise 3 copies of UV30 (MTTE) (SEQ ID NO: 7) as the B-cell epitope.

### Example 17

This example concerns identifying differences in vaccination with GV1001 (UV80-SEQ ID NO: 126) and with GVExt5 (UV 34-SEQ ID NO: 116).

Mice were administered with a polypeptide of SEQ ID NO: 126 (GV1001-UV80) or SEQ ID NO: 116 (UV 34-GVExt5). The same protocol was used as in Example 16 except that the concentration of peptides administered was 16.4 nmol and the mice were all female (n=4).

### Results

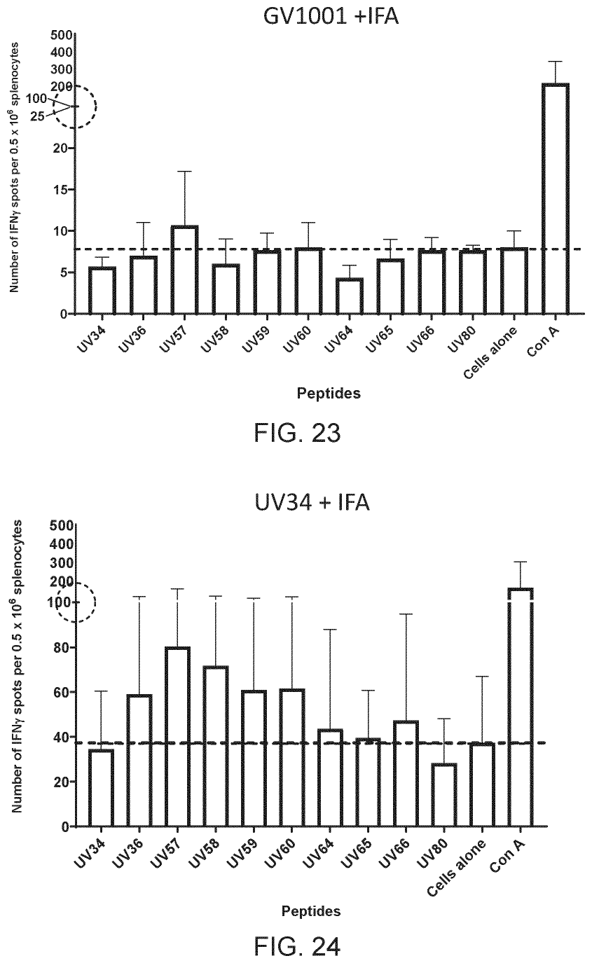

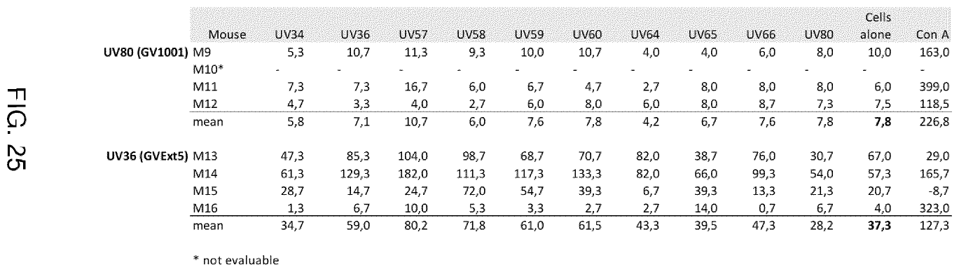

FIGS. 23 and 24 are graphs showing T cell responses against individual peptides. The horizontal dashed line marks the “cut off” which is the response level of “cells alone” which is to be considered the baseline. Bars of peptides above the cut off line/baseline are considered as T cell responses against the specific peptide. The “cells alone” in each group serves as an internal cut off point.

The peptide above the threshold in FIG. 23 (SEQ ID NO: 126-GV1001 administered) is: UV 57. The peptides above the threshold in FIG. 24 (SEQ ID NO: 116-GVExt5 administered) are: UV36, UV57, UV58, UV59, UV60, UV64 and UV66.

The individual results are shown in FIG. 25 demonstrating how the mean T-cell response to each peptide was calculated.

TABLE 13
Summary of Peptides

| Peptide | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| UV34 | L | S | E | A | E | V | R | Q | H | R | E | A | R | P | A | L | L | T | S | R |
| (GVExt5) | | | | | | | | | | | | | | | | | | | | |
| UV80 | — | — | — | — | — | — | — | — | — | — | E | A | R | P | A | L | L | T | S | R |
| (GV1001) | | | | | | | | | | | | | | | | | | | | |
| UV36 | — | — | — | — | — | — | — | — | — | — | E | A | R | P | A | L | L | T | S | R |
| UV57 | L | S | E | A | — | — | — | — | — | — | — | — | — | — | — | — | — | — | — | — |
| UV58 | L | S | E | A | V | R | Q | H | R | E | A | R | P | A | — | — | — | — | — | — |
| UV59 | — | — | — | — | — | — | — | — | — | — | E | A | R | P | A | L | L | T | S | R |
| UV60 | — | — | — | — | — | — | — | — | — | — | — | — | — | — | — | — | — | — | — | — |
| UV64 | — | — | — | — | — | — | — | — | — | — | — | — | — | — | — | L | L | T | S | R |
| UV65 | — | — | — | — | — | — | — | — | — | — | — | — | — | — | A | L | L | T | S | R |
| UV66 | — | — | — | — | — | — | — | — | — | R | E | A | R | P | A | L | L | T | S | R |
| UV30 | F | I | G | I | T | E | L | K | K | L | E | D | K | I | N | K | V | F | — | — |

| | | | | | | | | | | | | | | | | | Includes | |
|---|---|---|---|---|---|---|---|---|---|---|---|---|---|---|---|---|---|---|
| | | | | | | | | | | | | | | | | No. | amino | |
| | | | | | | | | | | | | | | | | of | acids | SEQ |
| | | | | | | | | | | | | | | | | amino | unique to | ID |
| Peptide | 21 | 22 | 23 | 24 | 25 | 26 | 27 | 28 | 29 | 30 | 31 | 32 | 33 | 34 | 35 | acids. | GVExt5 | NO. |
| UV34 | L | R | F | I | P | K | P | D | G | L | — | — | — | — | — | 30 | N/A | 116 |
| (GVExt5) | | | | | | | | | | | | | | | | | | |
| UV80 | L | R | F | I | P | K | — | — | — | — | — | — | — | — | — | 16 | No | 126 |
| (GV1001) | | | | | | | | | | | | | | | | | | |
| UV36 | L | R | F | I | P | K | P | D | G | L | — | — | — | — | — | 20 | Yes | 166 |
| UV57 | — | — | — | — | — | — | — | — | — | — | — | — | — | — | — | NA | Yes | 167 |
| UV58 | — | — | — | — | — | — | — | — | — | — | — | — | — | — | — | 15 | Yes | 168 |
| UV59 | L | R | F | I | P | — | — | — | — | — | — | — | — | — | — | 15 | No | 169 |
| UV60 | L | R | F | I | P | K | P | D | G | L | R | P | I | V | N | 15 | Yes | 170 |
| UV64 | L | R | F | I | — | — | — | — | — | — | — | — | — | — | — | 9 | No | 171 |
| UV65 | L | R | F | I | — | — | — | — | — | — | — | — | — | — | — | 10 | No | 172 |
| UV66 | L | R | F | I | — | — | — | — | — | — | — | — | — | — | — | 15 | Yes | 173 |
| UV30 | — | — | — | — | — | — | — | — | — | — | — | — | — | — | — | — | | 39 |

TABLE 14
Summary of Peptides

| | | | | SEQ |
|---|---|---|---|---|
| | | | | ID |
| | Name | Sequence | Origin | NO. |
| 1 | P719-20 | ALFSVLNYERARRPGLLGASVLGLD | | 1 |
| | | DIHRA | | |
| 2 | UV16 | FSVLNYERARRPGLL (HLA-DR4) | p719-20 | 150 |
| 3 | UV17 | RARRPGLLGASVLGL (HLA-DR4) | p719-20 | 151 |
| 4 | UV18 | VLNYERARRPGLLGA (HLA-DR4) | p719-20 | 152 |
| 5 | UV19 | RPGLLGASVLGLDDI (HLA-DR4) | p719-20 | 153 |
| 6 | UV53 | RAERLTSRVKALFSV | p719-20 | 174 |
| 7 | UV54 | ALFSVLNYERARRPG | p719-20 | 175 |
| 8 | UV55 | ARRPGLLGASVLGLD | p719-20 | 31 |
| 9 | UV56 | VLGLDDIHRAWRTFV | p719-20 | 176 |
| 10 | UV62 | LLGASVLGL | p719-20 | 147 |
| 11 | UV63 | GLLGASVLGL | p719-20 | 148 |
| 12 | UV34 | LSEAEVRQHREARPALLTSRLRFIPK | | 116 |
| | (GVExt5) | PDGL | | |
| 13 | UV36 | EARPALLTSRLRFIPKPDGL | GVExt5 | 166 |
| 14 | UV57 | QHLKRVQLRELSEAE | GVExt5 | 167 |
| 15 | UV58 | LSEAEVRQHREARPA | GVExt5 | 168 |
| 16 | UV59 | EARPALLTSRLRFIP | GVExt5 | 169 |
| 17 | UV60 | LRFIPKPDGLRPIVN | GVExt5 | 170 |
| 18 | UV64 | LLTSRLRFI | GVExt5 | 171 |
| 19 | UV65 | ALLTSRLRFI | GVExt5 | 172 |
| 20 | UV66 | REARPALLTSRLRFI | GVExt5 | 173 |
| 21 | UV80 | EARPALLTSRLRFIPK | GVExt5 | 126 |

### Description of Findings:

### GV 1001 (UV80) Vaccinated Group:

Mouse M10 was not evaluable due to fungus contamination in ELISPOT plate. In the 3 evaluable mice there were generally absent/weak immune responses. M9 and 12 did not respond to any of the tested peptides. M11 demonstrated a marginal immune response against UV57, but as this peptide does not share any sequence homology with GV1001 used for vaccination, the weak reactivity may be a result of background variation.

Conclusion: GV1001 demonstrates marginal immune responses in HLA-A2/DR1 transgenic mice when combined with a standard adjuvant used in mice.

### GV1001 Extension-5 (UV34) Vaccinated Group:

Mouse M16 displayed marginal immune responses against UV57, which has a 5 amino acid overlap with UV34, and with UV65 which is a 10-mer peptide derived embedded in UV34, with an HLA-A2 binding motif.

Mouse M15 responded somewhat more broadly and recognized UV58 which contains a new epitope in Extension 5 N-terminal to the sequence of GV1001 and with a 5 amino acid overlap with GV1001. Cells from this mouse also responded to UV59, which is identical to GV1001 except for the absence of the C-terminal K. Interestingly GV1001 was not recognized indicating that this amino acid (i.e. the C-terminal lysine residue) present in GV1001 may be disadvantageous for binding to HLA-DR1. Peptide UV60 was also recognized. This peptide has a 6 amino acid overlap with GV1001 and 10 amino acid unique to UV34 and thus represents a new epitope in GV Extension 5 (SEQ ID NO: 116).

Mouse M13 and mouse M14 showed quite strong reactivity with similar peptide recognition patterns. They both recognized UV36, UV57, UV58, UV64 and UV66. UV 36 contains the full GV1001 sequence with an additional 4 amino acids unique to UV34. Since these two mice failed to recognize GV1001 (UV80), we interpret these data to mean that combining these 4 amino acids from Extension 5 with the amino acids in GV1001 results in the creation of a novel epitope recognized in the context of HLA-DR1 presentation. UV57, which has a 5 amino acid overlap with UV34 as the only common amino acids indicates that these amino acids may represent a short but particularly strong binding motif. UV58 also stands out as a good epitope, since all 3 responding mice recognize this peptide (see above). UV64 which is a 9-mer HLA-A2 epitope was also recognized by these mice identifying a new HLA-A2 epitope. Finally, both mice recognized UV66 which is overlapping with GV1001 by 14 amino acids the modification being an addition of an N-terminal R residue from Extension 5 and the removal of PK from the C-terminal. Again, it is of interest that these modifications were recognized by the two mice, whereas GV1001 was not. Mouse M14, which displayed the broadest recognition repertoire and the highest number of spot forming cells also recognized UV59 and UV60. UV59 is a version of GV1001 without the C-terminal K residue. Again this indicates that the presence of this amino acid is incompatible with HLA-DR1 presentation. For recognition of UV60, see M15 above.

Conclusion: GV1001 Extension 5 (SEQ ID NO: 116) is highly immunogenic in HLA-DR1/A2 transgenic mice. Strong immunogenicity is obtained by the presence of multiple epitopes within this vaccine peptide as revealed by the reactivity pattern of immunized mice. These epitopes are in part consisting of amino acids present in GV1001 Extension 5 (SEQ ID NO: 116) but absent from GV1001 (SEQ ID NO: 126) and surprisingly also epitopes present in GV1001, exemplified by UV59 and UV66. We interpret the lack of generating immune responses against this latter group of peptides by vaccination with GV1001 to be related to processing of the peptide in the antigen processing cells, most likely the ability to remove the C-terminal basic amino acid Lysine (K) from GV1001 as this may be dependent on further amino acids present in the C-terminal part of GV1001 Extension 5 but absent from GV1001. Importantly, the peptides UV64 (9-mer) and UV65 (10-mer) which were designed as HLA-A2 binding peptides were also recognized after vaccination with GV1001 Extension 5 extending the efficacy of this vaccine peptide to include induction of a CTL response.

### Example 18

This example demonstrates that a 3+1+1 conjugate comprising a B-cell epitope and two CD4+ T-cell epitopes derived from hTERT are bound by polyclonal antibodies specific to the B-cell epitope.

Conjugates comprising the B-cell epitope (MTTE-SEQ ID NO: 7) and two CD4+ T-cell epitopes derived from hTERT (p719-20 (SEQ ID NO: 1) and GVExt5 (UV34; SEQ ID NO: 116) were tested by ELISA to evaluate binding of the conjugates by antibodies specific to MTTE.

### Materials and Methods

Sandwich ELISA technique was used to evaluate human polyclonal antibody binding to the conjugates using the protocols below. It is to be understood that in the indirect ELISA, 30 the conjugates to be tested were coated onto the plates whereas in the sandwich ELISA, conjugates comprising the polypeptide of SEQ ID NO: 1 which were to be tested were captured onto the plate via rabbit polyclonal antibody specific for SEQ ID NO: 1.

Sandwich ELISA: the sandwich ELISA was performed by Capra Science, Sweden, according to the following procedure. Thermo Scientific™ Clear Flat-Bottom Immuno Nonsterile 96-Well Plates were used and coated with a polyclonal rabbit anti-p719-20 or polyclonal rabbit anti-GVExt5 antibody (0.5 μg per well) and in total 50 μl/well and incubated at 4° C. overnight. The plate was blocked using a protein free block solution (Pierce) at 1 hour 5 at room temperature and subsequently washed using PBS and 0.05% Tween washing solution. The conjugates were added to the plate at concentrations indicated and in a serial dilution and incubated for 1 hour at room temperature. Subsequently the plate was washed with the washing solution and then incubated with anti-MTTE antibody based on a TetaQuin® (polyclonal human IgG from high titer anti-tetanus donors or a 10 monoclonal recombinant chimeric anti-MTTE antibody. The plate was washed with washing solution and subsequent it a secondary antibody, goat anti-human kappa ALP was added in a PBS solution with 1% BSA. After one hour of incubation with secondary antibody, the plate was again washed with washing buffer and incubated with the substrate PNPP (1 mg/ml) for one hour at room temperature to develop the assay.

### Results

TABLE 15

| Conjugate | Peptide/s | Binder | EC50 |
|---|---|---|---|
| 3 + 1 + 1 | P719-20 & GVExt5 | Yes | 0.2763 |
| Ba21E3299 (PD00723) | | | |
| 3 + 1 + 1 | P719-20 & GVExt5 | Yes | 0.2258 |
| Ba21E3300 (PD00724) | | | |
| 3 + 1 + 1 | P719-20 & GVExt5 | Yes | 0.2601 |
| Ba21E3301 (PD00725) | | | |
| CD29 (PD00696) | GVExt5 | Yes | 0.1545 |
| CD09 | N/A | No | — |

TABLE 16

| Conjugate | Peptide/s | Binder | EC50 |
|---|---|---|---|
| 3 + 1 + 1 | P719-20 & GVExt5 | Yes | 0.2763 |
| Ba21E3299 (PD00723) | | | |
| 3 + 1 + 1 | P719-20 & GVExt5 | Yes | 0.2258 |
| Ba21E3300 (PD00724) | | | |
| 3 + 1 + 1 | P719-20 & GVExt5 | Yes | 0.2601 |
| Ba21E3301 (PD00725) | | | |
| CD29 (PD00696) | GVExt5 | Yes | 0.1545 |
| CD09 | N/A | No | — |

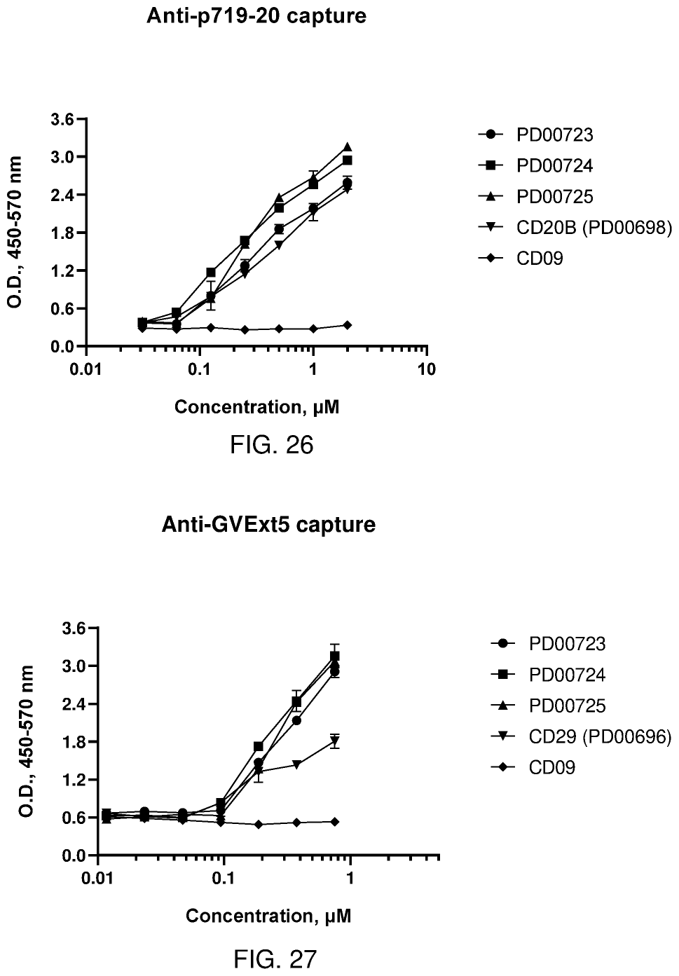

FIGS. 26 and 27 are graphs and Tables 15 and 16 are tables demonstrating the binding of anti-MTTE polyclonal antibody in a sandwich ELISA assay using conjugates comprising the polypeptides p719-20 (SEQ ID NO: 1) and GVExt5 (SEQ ID NO: 116) as the CD4+ T-cell epitopes. For FIG. 26 and Table 15, the capture antibody was anti-p719-20 rabbit polyclonal. For FIG. 27 and Table 16, the capture antibody was anti-GVExt5 rabbit polyclonal. CD09 did not comprise a CD4+ T-cell epitope and was used as a negative control. As shown in FIGS. 26 and 27, 3+1+1 conjugate from each of the 3 batches 21E3299 (Synthesis Example 20), 21E330 (Synthesis Example 21) and 21E3301 (Synthesis Example 22) comprising polypeptides p719-20 (SEQ ID NO: 1) and GVExt5 (SEQ ID NO: 116) was bound by human polyclonal antibodies (from the blood plasma product TetaQuin) that recognise MTTE.

### Example 19

This example concerns binding assays to assess the binding of conjugates of TFA, HCL and acetate salt forms.

### Experimental Procedure

Reconstitution of conjugates UVC2-001 (i.e. CD29, conjugate containing GVExt5 (SEQ ID NO: 116)) & UVC1-017 (i.e. CD20B, conjugate containing p719-20 (SEQ ID NO: 1)): prior use, E-toxate water
Coating Buffer: PBS
1st antibody: CD01 (monoclonal higG1-anti-MTTE) or FF10 (human polyclonal anti-MTTE serum)
2nd antibody: goat-antiHu Kappa light chain-HRP
Read Out: Absorbance (450 nm-570 nm)

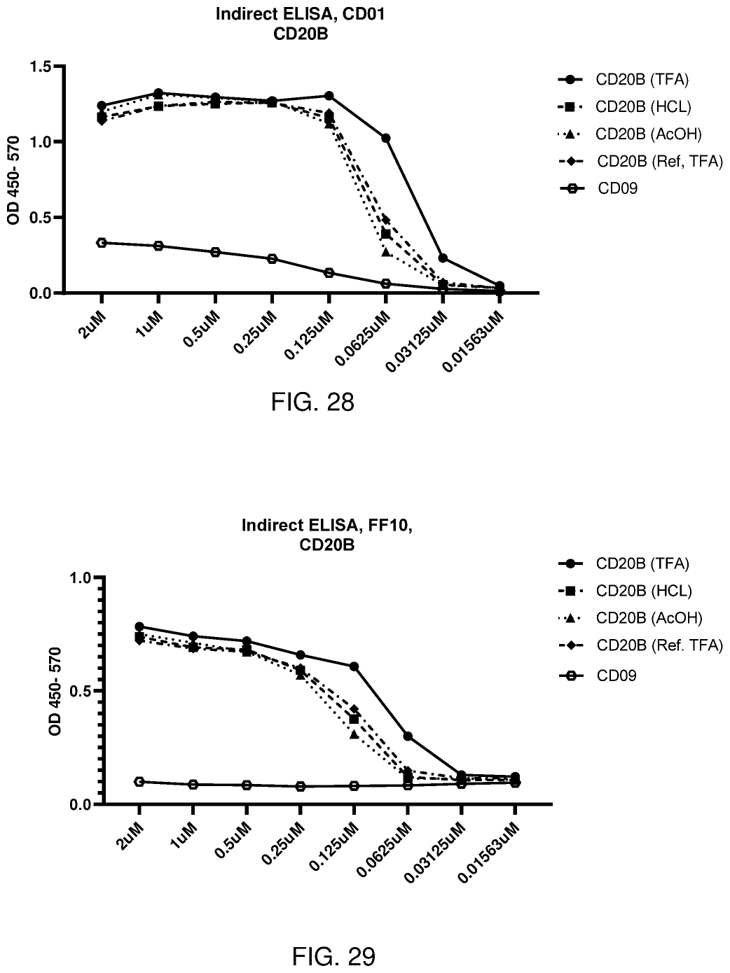

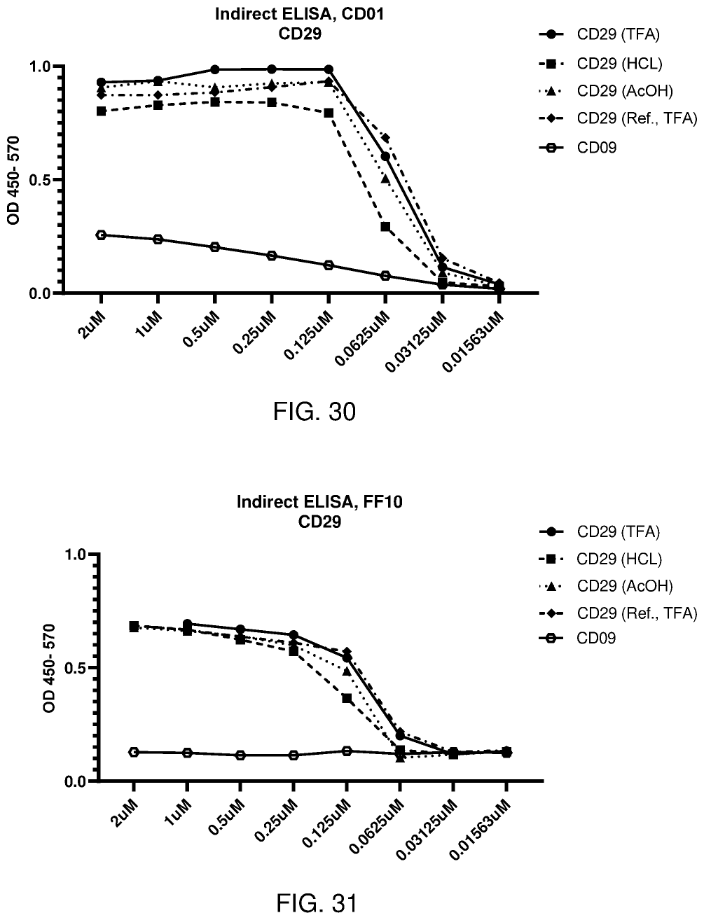

Table 17 shows the pH of the conjugates. The pH of the conjugates after reconstitution in water is the same between the two conjugates (UVC1-017 (CD20B) and UVC2-001 (CD29)) for each salt form. The working solution was diluted in PBS and the pH was 7 for all of the conjugates tested. Results are also shown in FIGS. 28 to 31.

TABLE 17

| | | | Conc. of | | | | Working | |
|---|---|---|---|---|---|---|---|---|
| | | | stock | | | | conc. (μM) | |
| | | | (Dissolved in | | Concentration | | (Diluted | |
| Conjugate | Salt form | Batch | water) | | in μM | pH | in PBS) | pH |
| UVC2-001 (CD29) | TFA | CF20002 | 1 | mg/mL | 43.68 | 4 | 2 | 7 |
| UVC2-001 (CD29) | HCL | CF20008A | 1 | mg/ml | 58.10 | 5 | 2 | 7 |
| UVC2-001 (CD29) | Acetate | CF20008B | 1 | mg/ml | 58.54 | 7 | 2 | 7 |
| UVC2-001 (CD29) | TFA | 21E1862 | 1 | mg/ml | 47.36 | 5 | 2 | 7 |
| | (RISE) | | | | | | | |
| UVC1-017 (CD20B) | TFA | CF20017 | 1 | mg/mL | 45.45 | 4 | 2 | 7 |
| UVC1-017 (CD20B) | HCL | CF20018A | 1 | mg/mL | 57.78 | 5 | 2 | |
| UVC1-017 (CD20B) | Acetate | CF20018B | 1 | mg/ml | 58.08 | 7 | 2 | |
| UVC1-017 (CD20B) | TFA | 21E1469 | 1 | mg/ml | 46.55 | 5 | 2 | 7 |
| | (RISE) | | | | | | | |

### Example 20

This Example concerns anti-MTTE IgG antibodies measured in patients that have received TENDU, a peptide-conjugate vaccine comprising the B-cell epitope MTTE conjugated to synthetic long peptides (SLPs) harboring CD4+ and CD8+ T cell epitopes.

### Materials and Methods

### Vaccination

The TENDU vaccine comprises conjugates each containing three copies of a B-cell epitope (MTTE) and an SLP comprising a CD4+ and CD8+ T-cell epitope. All patients included in the TENDU trial received a dose of Boostrix on the Screening visit, 1-week prior TENDU vaccination start, followed by 4 doses of TENDU vaccine with 2 weeks between every dose. In this Example the TENDU vaccine contained conjugates, each comprising 3 MTTE (SEQ ID NO: 46) peptides as the B-cell epitope and one of the following as the CD4+ T-cell epitope: LUG1 (SEQ ID NO: 47), LUG3 (SEQ ID NO: 49), LUG4 (SEQ ID NO: 50) and LUG 5 (SEQ ID NO: 51) peptides. The trial was a dose escalation trial to advice dose and safety margins of the TENDU vaccine; 40, 400 and 960 μg are administered to cohort #1, cohort #2 and cohort #3 respectively. Samples were collected at the Screening visit, visit 2, visit 4, visit 5 and at safety visit (30 days after the last dose of vaccination). To date only one patient had been enrolled and sampled at all time-points for cohort #3.

### ELISA

Streptavidin-coated plates (Thermo Scientific, cat no 15501) were coated with 1 μM biotinylated MTTE or p725 peptide (SEQ ID NO: 52) (reference negative control peptide) diluted in coating buffer (0.2% HSA, 0.05% Tween-20, PBS) at 4° C. overnight. The next morning, plates were washed four times with PBS/0.05% Tween-20 and coated with 0.5 mg/ml biotin-D (dissolved in 0.2% HSA, 0.05% Tween-20, PBS) for 1 hour in room temperature. Then the plates were emptied (without washing) and blocked with Pierce Protein-free blocking solution (Thermo Scientific, cat no 37572) for 1 hour at room temperature and washed with washing buffer (PBS/0.05% Tween-20) 5 times. The anti-MTTE IgG antibody standard, patient serum samples and controls diluted in plasma diluent buffer (0.2% HSA, 0.05% Tween-20, PBS) were added to the wells in duplicates and incubated for 2 hours at room temperature. After washing, an Fc-specific goat antihuman IgG Peroxidase antibody (Sigma-Aldrich, cat.no. A0170a) diluted in plasma diluent buffer was added and plates were incubated for an hour at room temperature. Plates were washed again, and substrate solution was added to the wells. Absorbance was read at 415 nM after 30 minutes.

A standard curve based on the anti-MTTE IgG antibody was plotted using GraphPad Prism version 9.3.1 software (GraphPad software) and used to calculate total IgG concentrations of all patient samples and controls. For all samples the dilution 1:25 was chosen to calculate serum IgG concentration.

### Results and Discussions

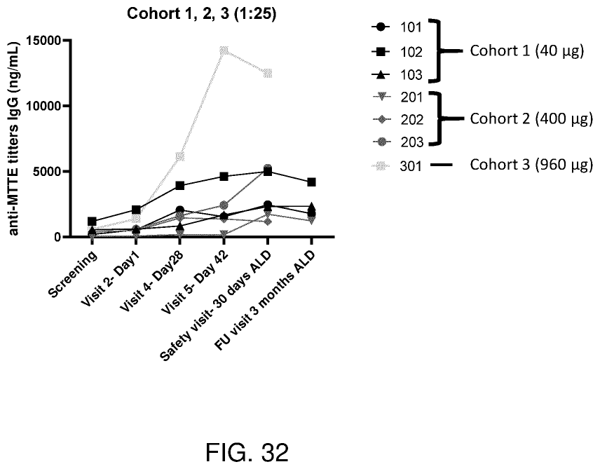

The data (see FIG. 32) show that anti-MTTE concentrations were notably elevated at visit 4 in comparison to concentrations observed at screening and visit 2. No noticeable increase in anti-MTTE concentrations was observed on visit 2, that is at a time point seven days after Boostrix, but before the patient receives the first dose of TENDU vaccine. It can be observed that the anti-MTTE concentration in most patients increased and remained elevated in samples from visit 4 (post start of TENDU vaccination) and later timepoints but concentrations were still low for cohort #1 and #2. For cohort #3, serum anti-MTTE concentration were highly elevated, most likely as a result of the increased dose, leading to sufficient drug exposure for immune activation to occur and thereby allowing for a T cell-mediated (CD4 helper epitope) driven antibody response against the B cell epitope. Data are in line with the rabbit study (Example 8) where all tetanus toxoid exposed rabbits displayed greatly elevated anti-MTTE concentrations at the highest dose, while lower doses, or tetanus naïve rabbits, were not able to mount a consistent increase in anti-MTTE antibody levels compared to levels prior TENDU exposure.

### Example 21

This example concerns the in vivo assessment of the immunogenicity of the GVExt5 peptide (SEQ ID NO: 116) in the form of a conjugate and peptide+IFA in transgenic HLA-A2/HLA-DR1 animals.

### Materials and Methods

The immunogenicity of the GVExt5 peptide (SEQ ID NO: 116) in the form of: CD29 conjugate [comprising 3 copies of UV30 (MTTE) (SEQ ID NO: 7) as the B-cell epitope and UV34 (GVExt5) (SEQ ID NO: 116) T-cell epitope], was investigated in seropositive or in the form of naked peptide+IFA in seronegative female B2m,Tg(HLA-A/H2-D/B2M)1Bpe,Tg(HLA-DRA*0101,HLA-DRB1*0101)1Dma, H2-Ab1 (EMMA Repository, Orleans, FRANCE) strain mice. Serological status refers to the presence or absence of pre-existing antibodies to MTTE in the mice following vaccination with using MTTE peptide conjugated to ovalbumin (OVA).

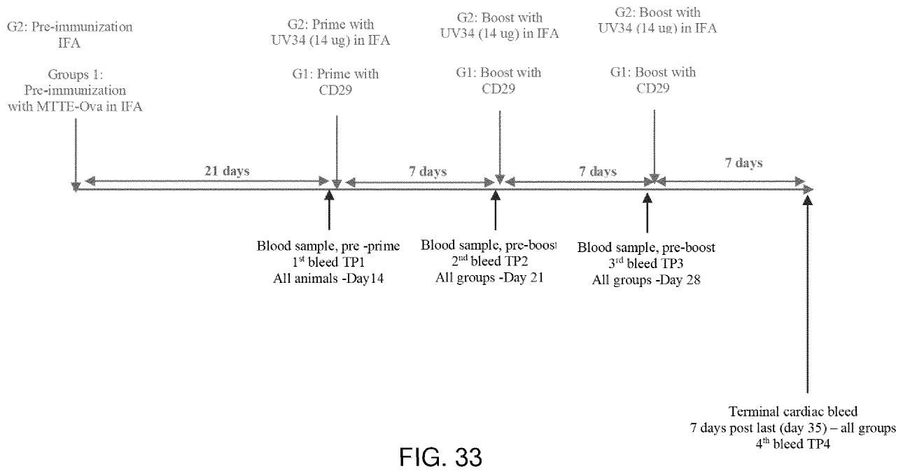

The vaccination schedule is shown schematically in FIG. 33 and is summarized as follows:
4× Group 1: Pre-vaccinated with MTTE-OVA (75 μg) in 1:1 IFA in the scruff of the neck, followed by CD29 (60 μg, 4.1 nmol) in 100 uL/mouse. Immunization at the base of the tail, both sides.
4× Group 2: Pre-vaccinated with IFA in the scruff of the neck followed by UV34 (14 ug/mouse, 4.1 nmol)/mouse in IFA (1:1) in 50 uL/mouse. Immunization at base of the tail, both sides.

Pre-vaccination was performed using MTTE peptide conjugated to ovalbumin (OVA) in IFA. Thus MTTE-OVA was used as an immunogen to immunize the mice to achieve antibodies against the MTTE sequence to achieve seropositive animals. The immunization was performed using a solution of 75 μg of MTTE-OVA in PBS emulsified in IFA at a 1:1 ratio. This was administrated as a subcutaneous administration at the tail base of the mice on day 0 for the seropositive group. The seronegative group was not exposed to MTTE-OVA/IFA.

Twenty-one days after MTTE-OVA administration (pre-vaccination) the seropositive group was administrated the conjugate [Group 1: CD29, 60 μg/mouse=4.1 n mol (100 μL/mouse) n=4]. The seronegative group was administered polypeptide UV34 (equi-moles as the mice in group 1) mixed with IFA (as a reference adjuvant) [Group 2: UV34, 14 ug/mouse=4.1 n mol (100 μL/mouse); n=4]. The conjugate and polypeptide were administered as subcutaneous injections to mice (in the respective groups) at the tail base as primer injections. Two booster injections (two different later occasions) of CD29 conjugate and polypeptide UV34 was administered (to mice in the respective groups) seven (first booster) and fourteen days (second booster) post primer injections, according to the schedule shown in FIG. 33. On the day of termination, the spleen and the draining lymph nodes (iliac, inguinal, and auxiliary) were harvested and single cell suspensions of these organs were prepared.

Spleens were flushed out using T-cell medium (RPMI 1640, Life Technologies/Thermo Fisher Scientific, containing 1% w/v L-Glutamine (SLS/Lonza), 10% v/v FBS (Fisher/GE Healthcare), 2% HEPES (SLS/Lonza), 0.1% v/v Fungizone (Promega)), after which they were passed through a strainer to remove tissue-debris and centrifuged at 300 g for 10 minutes. Cell pellets were resuspended in T-cell medium for counting and plating.

Lymph nodes were squashed first onto a petri dish containing 2 mL of T-cells media and then through an EASYstrainer 100 μM using the back of a sterile syringe and then rinsing the filter with 1 mL of T-cell media. Cells were spun down at 300 g for 10 min. Pellet was resuspended in 2 mL of T-cells media for counting and plating.

The frequency of vaccine-induced responsive cells after 48 hours' stimulation with selected polypeptides in vitro (as shown in Table 13) was determined using a murine IFN-γ ELISpot kit (Mabtech). For this, transmembrane 96-well Millipore plates were pre-coated with capture antibody according to the manufacturer's protocol. For each experiment, 0.5×10⁶ cells (0.25×10⁶ splenocytes+0.25×10⁶ pooled draining LN cell-coculture)/well were plated in 3 wells+/−10 μg/ml of peptides. Concanavalin A (ConA) was used as a positive control. Cells in media only was used as negative control.

ELISA measurements of anti-MTTE antibody titers were performed according to the following protocol:
10. Streptavidin-coated ELISA plates were coated with 100 μl Biotinylated peptide (MTTE-biotin diluted in PBS 1 nmol/ml) according to the plate layout and incubated at 4° C. overnight.
11. The plates were washed four times with 250 μl PBS/0.05% Tween20.
12. The plates were blocked with 200 μl PBS/10% BSA/0.05% Tween20 and incubated at room temperature (RT) for 1 hour.
13. The plates were washed four times with 250 μl PBS/0.05% Tween20.
14. The plasma was diluted in PBS/1% BSA/0.05% Tween20 (first 1:50, followed by 2× dilutions yielding 100× and 20× dilution, yielding 1000×). 100 μl of diluted supernatants were added per well and incubated for 2 hours at RT.
15. The plates were washed four times with 250 μl PBS/0.05% Tween20.
16. 100 μl of secondary antibody Goat anti-mouse Ig-HRP was added per well, diluted 1:5000 in PBS/1% BSA and incubated for 1 hour in RT in the dark.
17. The plates were washed four times with 250 μl PBS/0.05% Tween20.
18. 100 μl of TMB was added per well and the reaction was stopped with 100 μl 1M H2SO4. Absorbance was read at 450-570 nm. Developing time was 90 seconds for plates with serum from mice immunized with MTTE-OVA and 150 seconds for serum with plates from mice immunized with UV34.

### Results

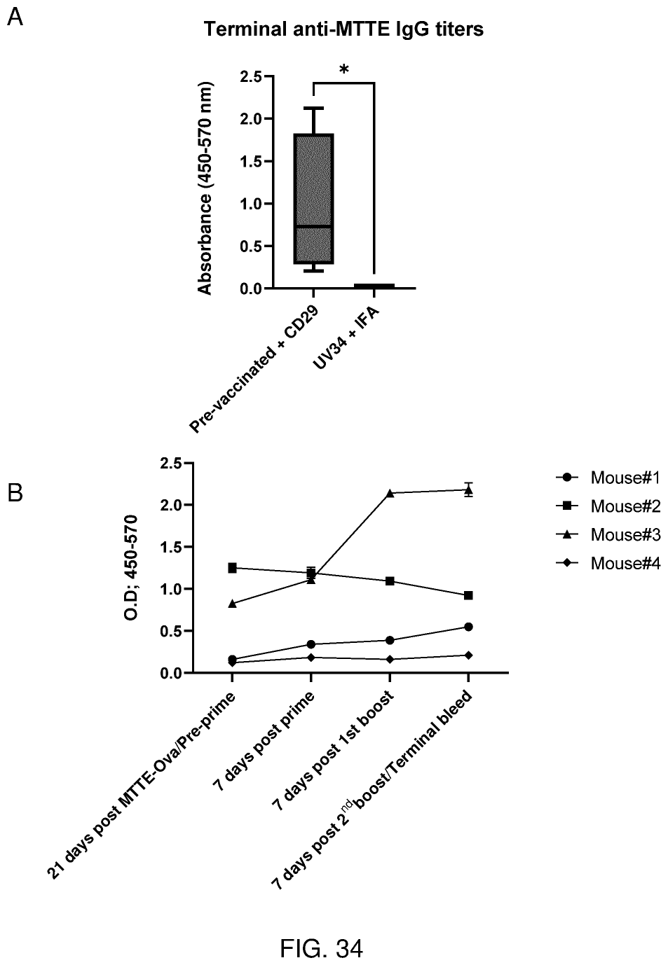

FIG. 34A represents anti-MTTE antibody titers in mice at the end of the experiment. Mice had either been pre-vaccinated with MTTE-OVA followed by immunization with conjugate CD29 (group 1) or had not been pre-vaccinated with MTTE-OVA and were only exposed to equi-mole quantity of polypeptide UV34 (SEQ ID NO: 116) in IFA (1:1 v/v). Immunization scheme of the conjugate and peptide+IFA was in a prime-boost-boost setting. Mice that had not been exposed to MTTE-OVA followed by conjugate CD29 did not develop antibodies to the MTTE sequence and were not able to mount an antibody response to the MTTE sequence upon exposure to UV34+IFA. However, mice exposed to MTTE-OVA had high titers of anti-MTTE antibodies post the cycle of the MTTE-OVA/CD29 vaccination, significance was assessed by a Mann Whitney test *p=0.0286. FIG. 34B shows anti-MTTE antibody titers in individual mice from group 1. During the experimental period mice 1 and 3 showed gradual increase in anti-MTTE antibody titers with administration of CD29 (Prime-boost-boost) on three occasions, although of varying degree of increase in antibody titers. In mouse 2 the initial high antibody titer marginally decreased with time.

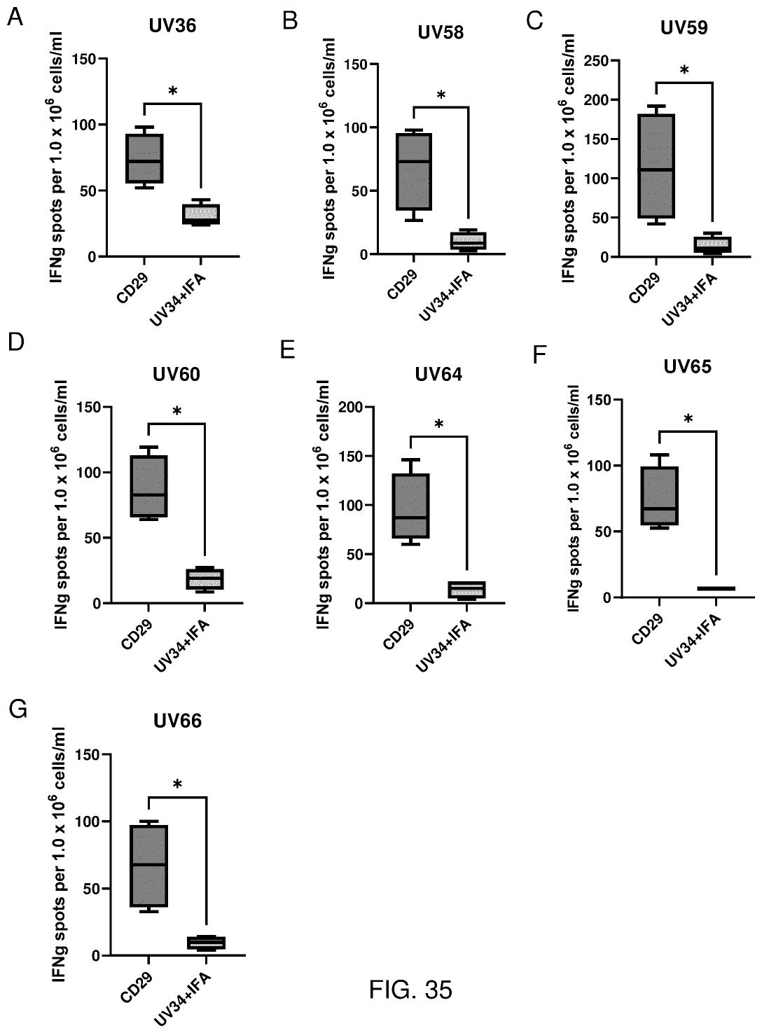

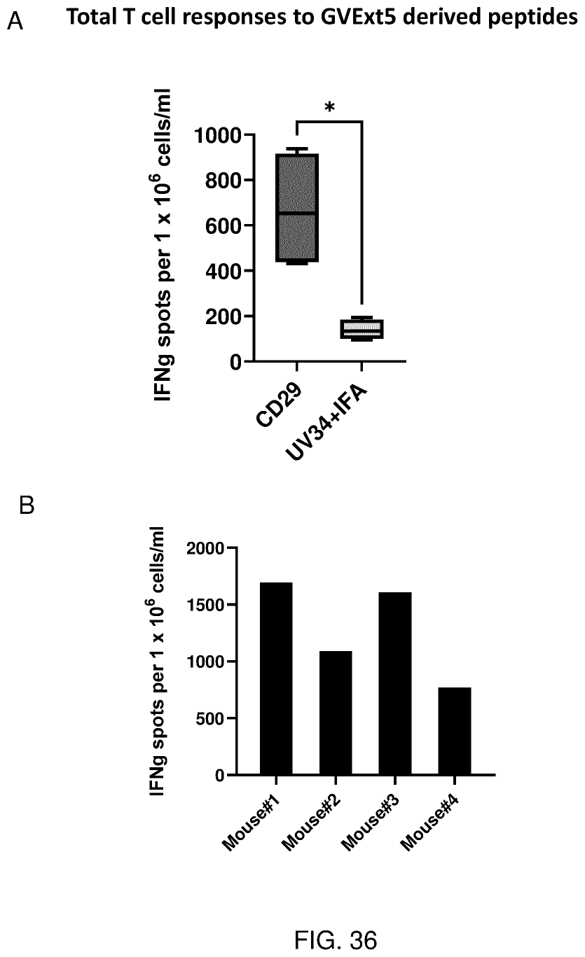

FIG. 35 shows that stimulation of splenocytes-LN coculture with seven individual polypeptides UV36, UV58, UV59, UV60, UV64, UV65 and UV66 (see Table 13) induced a measurable and significantly elevated T-cell response in the CD29 (conjugate) exposed animals (seropositive) compared to polypeptide UV34+IFA vaccinated animals (seronegative) at the tested dose using Mann Whitney test *p=0.0286. FIG. 36A demonstrates the sum of T cell responses of mice exposed to CD29 towards polypeptides sequences of varying lengths within the UV36 peptide is significantly elevated compared to that of the sum of T cell responses of mice in exposed to polypeptide UV34+IFA using Mann Whitney test *p=0.0286.

### Discussion

The data demonstrates that transgenic HLA-DR1 seropositive mice mount a superior T-cell response to UV34 (GVExt5-SEQ ID NO: 116) derived peptides (epitopes), when vaccinated with a UV34 conjugate (CD29) comprising 3 copies of MTTE B-cell epitope in comparison to seronegative mice vaccinated with equi. mol of UV34 peptide with IFA as an adjuvant.

GVext5 (SEQ ID NO: 116) is an extended version of GV1001 (SEQ ID NO: 126) with nine N-terminal amino acids and 4 C-terminal amino acids derived from the hTERT sequence. GVExt5 is 81% longer in length than GV1001.

T cell responses were observed when cells from vaccinated animals were stimulated with seven specific individual peptides of lengths 20, 15, 14, 10 or 9 amino acids, see Table 13. For every peptide tested a superior T cell response was seen in animals vaccinated with the Extension 5 conjugate as compared to Extension 5 in IFA. Interestingly, these experiments also identify a core binding motif in the GV1001 sequence, which is represented by UV64 (Table 13), and is present in all peptides tested except UV58 and UV60 (Table 13). Importantly these two peptides contain the 10 N-terminal amino acids (UV58) and the 4 C-terminal amino acids (UV60) present in Extension 5, which are absent from GV1001. Recognition of these two peptides after vaccination with the Extension 5 conjugate demonstrate that Extension 5 contains novel epitopes defined by the N- and C-terminal flanking amino acids respectively. These data provide further evidence for a superior immunogenicity of Extension 5 over GV1001. Interestingly, the recognition of UV60 implies that the addition of 4 C-terminal amino acids PDGL (SEQ ID NO: 178) to GV1001 results in recognition of a peptide that contains an additional C-terminal “tail” of 5 amino acids (RPIVN (SEQ ID NO: 177)), which is present in the hTERT sequence, but is absent from Extension 5 peptide sequence used for vaccination. This represents a further mechanism whereby vaccination design may broaden the repertoire of specific T cells that can recognize the target antigen.

This Example demonstrates elevated anti MTTE antibody titer at the termination of the experiment only in mice that received OVA-MTTE pre-vaccine followed by CD29 conjugate, FIG. 36A. Anti-MTTE antibody titers in individual mice (FIG. 34B) corresponds to total T cell response elicited by each mouse, FIG. 36B.

### Schedule of Sequence Listing

| SEQ ID NO. | | |
|---|---|---|
| in Sequence | | |
| Listing | Sequence | Notes |
| 1 | ALESVLNYERARRPGLLGASVLGLDDIHRA | Corresponds to amino acid |
| | | positions 660-689 of human |
| | | telomerase reverse |
| | | transcriptase (hTERT)- |
| | | p719-20 |
| 2 | MPRAPRCRAVRSLLRSHYREVLPLATFVRRIGPQGWRLVQRGDPAAFRALVAQCLVCVPW | hTERT |
| | DARPPPAAPSFRQVSCLKELVARVLQRLCERGAKNVLAFGFALLDGARGGPPEAFTTSVR | |
| | SYLPNTVTDALRGSGAWGLLLRRVGDDVLVHLLARCALFVLVAPSCAYQVCGPPLYQLGA | |
| | ATQARPPPHASGPRRRLGCERAWNHSVREAGVPLGLPAPGARRRGGSASRSLPLPKRPRR | |
| | GAAPEPERTPVGQGSWAHPGRTRGPSDRGFCVVSPARPAEEATSLEGALSGTRHSHPSVG | |
| | RQHHAGPPSTSRPPRPWDTPCPPVYAETKHFLYSSGDKEQLRPSFLLSSLRPSLTGARRL | |
| | VETIFLGSRPWMPGTPRRLPRLPQRYWQMRPLFLELLGNHAQCPYGVLLKTHCPLRAAVT | |
| | PAAGVCAREKPQGSVAAPEEEDTDPRRLVQLLRQHSSPWQVYGFVRACLRRLVPPGLWGS | |
| | RHNERRFLRNTKKFISLGKHAKLSLQELTWKMSVRDCAWLRRSPGVGCVPAAEHRLREEI | |
| | LAKFLHWLMSVYVVELLRSFFYVTETTFQKNRLFFYRKSVWSKLQSIGIRQHLKRVQLRE | |
| | LSEAEVRQHREARPALLTSRLRFIPKPDGLRPIVNMDYVVGARTFRREKRAERLTSRVKA | |
| | LESVLNYERARRPGLLGASVLGLDDIHRAWRTFVLRVRAQDPPPELYFVKVDVTGAYDTI | |
| | PQDRLTEVIASIIKPQNTYCVRRYAVVQKAAHGHVRKAFKSHVSTLTDLQPYMRQFVAHL | |
| | QETSPLRDAVVIEQSSSLNEASSGLEDVELREMCHHAVRIRGKSYVQCQGIPQGSILSTL | |
| | LCSLCYGDMENKLFAGIRRDGLLLRLVDDFLLVTPHLTHAKTFLRTLVRGVPEYGCVVNL | |
| | RKTVVNFPVEDEALGGTAFVQMPAHGLFPWCGLLLDTRTLEVQSDYSSYARTSIRASLTF | |
| | NRGFKAGRNMRRKLFGVLRLKCHSLFLDLQVNSLQTVCTNIYKILLLQAYRFHACVLQLP | |
| | FHQQVWKNPTFFLRVISDTASLCYSILKAKNAGMSLGAKGAAGPLPSEAVQWICHQAFLL | |
| | KLTRHRVTYVPLLGSLRTAQTQLSRKLPGTTLTALEAAANPALPSDFKTILD | |
| 3 | PITINNFRYSDPVNNDTIIMMEPPYCKGLDIYYKAFKITDRIWIVPERYEFGTKPEDEN | Tetanus toxin (TTx) |
| | PPSSLIEGASEYYDPNYLRTDSDKDRFLQTMVKLENRIKNNVAGEALLDKIINAIPYLGN | |
| | SYSLLDKEDTNSNSVSFNLLEQDPSGATTKSAMLTNLIIFGPGPVINKNEVRGIVLRVDN | |
| | KNYFPCRDGFGSIMQMAFCPEYVPTFDNVIENITSLTIGKSKYFQDPALLLMHELIHVLH | |
| | GLYGMQVSSHEIIPSKQEIYMQHTYPISAEELFTFGGQDANLISIDIKNDLYEKTLNDYK | |
| | AIANKLSQVTSCNDPNIDIDSYKQIYQQKYQFDKDSNGQYIVNEDKFQILYNSIMYGFTE | |
| | IELGKKENIKTRLSYFSMNHDPVKIPNLLDDTIYNDTEGENIESKDLKSEYKGQNMRVNT | |
| | NAFRNVDGSGLVSKLIGICKKIIPPTNIRENLYNRTASLTDLGGELCIKIKNEDLTFIAE | |
| | KNSFSEEPFQDEIVSYNTKNKPLNFNYSLDKIIVDYNLQSKITLPNDRTTPVTKGIPYAP | |
| | EYKSNAASTIEIHNIDDNTIYQYLYAQKSPTTLQRITMINSVDDALINSTKIYSYFPSVI | |
| | SKVNQGAQGILFLQWVRDIIDDFTNESSQKTTIDKISDVSTIVPYIGPALNIVKQGYEGN | |
| | FIGALETTGVVLLLEYIPEITLPVIAALSIAESSTQKEKIIKTIDNFLEKRYEKWIEVYK | |
| | LVKAKWLGTVNTQFQKRSYQMYRSLEYQVDAIKKIIDYEYKIYSGPDKEQIADEINNLKN | |
| | KLEEKANKAMININIEMRESSRSFLVNQMINEAKKQLLEFDTQSKNILMQYIKANSKFIG | |
| | ITELKKLESKINKVESTPIPFSYSKNLDCWVDNEEDIDVILKKSTILNLDINNDIISDIS | |
| | GENSSVITYPDAQLVPGINGKAIHLVNNESSEVIVHKAMDIEYNDMENNFTVSFWLRVPK | |
| | VSASHLEQYGTNEYSIISSMKKHSLSIGSGWSVSLKGNNLIWTLKDSAGEVRQITERDLP | |
| | DKENAYLANKWVFITITNDRLSSANLYINGVLMGSAEITGLGAIREDNNITLKLDRCNNN | |
| | NQYVSIDKFRIFCKALNPKEIEKLYTSYLSITFLRDEWGNPLRYDTEYYLIPVASSSKDV | |
| | QLKNITDYMYLTNAPSYINGKLNIYYRRLYNGLKFIIKRYTPNNEIDSFVKSGDFIKLYV | |
| | SYNNNEHIVGYPKDGNAFNNLDRILRVGYNAPGIPLYKKMEAVKLRDLKTYSVQLKLYDD | |
| | KNASLGLVGTHNGQIGNDPNRDILIASNWYFNHLKDKILGCDWYFVPTDEGWIND | |
| 4 | PITINNFRYSDPVNNDTIIMMEPPYCKGLDIYYKAFKITDRIWIVPERYEFGTKPEDENP | TTx light chain (α chain) |
| | PSSLIEGASEYYDPNYLRTDSDKDRFLQTMVKLENRIKNNVAGEALLDKIINAIPYLGNS | |
| | YSLLDKFDTNSNSVSENLLEQDPSGATTKSAMLTNLIIFGPGPVLNKNEVRGIVLRVDNK | |
| | NYFPCRDGFGSIMQMAFCPEYVPTEDNVIENITSLTIGKSKYFQDPALLLMHELIHVING | |
| | LYGMQVSSHEIIPSKQEIYMQHTYPISAEELFTFGGQDANLISIDIKNDLYEKTLNDYKA | |
| | IANKLSQVISCNDPNIDIDSYKQIYQQKYQFDKDSNGQYIVNEDKEQILYNSIMYGFTEI | |
| | ELGKKENIKTRLSYFSMNHDPVKIPNLLDDTIYNDTEGFNIESKDLKSEYKGQNMRVNTN | |
| | AFRNVDGSGLVSKLIGLCKKIIPPTNIRENLYNRTA | |
| 5 | SLTDLGGELCIKIKNEDLTFIAEKNSFSEEPFQDEIVSYNTKNKPLNFNYSLDKIIVDYN | TTx heavy chain (β chain) |
| | LQSKITLPNDRTTPVTKGIPYAPEYKSNAASTIEIHNIDDNTIYQYLYAQKSPTTLQRIT | |
| | MTNSVDDALINSTKIYSYFPSVISKVNQGAQGILFLQWVRDIIDDETNESSQKTTIDKIS | |
| | DVSTIVPYIGPALNIVKQGYEGNFIGALETTGVVLLLEYIPEITLPVIAALSIAESSTQK | |
| | EKIIKTIDNFLEKRYEKWIEVYKLVKAKWLGTVNTQFQKRSYQMYRSLEYQVDAIKKIID | |
| | YEYKIYSGPDKEQIADEINNLKNKLEEKANKAMININIFMRESSRSFLVNQMINEAKKQL | |
| | LEFDTQSKNILMQYIKANSKFIGITELKKLESKINKVESTPIPESYSKNLDCWVDNEEDI | |
| | DVILKKSTILNLDINNDIISDISGENSSVITYPDAQLVPGINGKAIHLVNNESSEVIVHK | |
| | AMDIEYNDMENNFTVSFWLRVPKVSASHLEQYGTNEYSIISSMKKHSLSIGSGWSVSLKG | |
| | NNLIWTLKDSAGEVRQITFRDLPDKFNAYLANKWVFITITNDRLSSANLYINGVLMGSAE | |
| | ITGLGAIREDNNITLKLDRCNNNNQYVSIDKFRIFCKALNPKEIEKLYTSYLSITFLRDE | |
| | WGNPLRYDTEYYLIPVASSSKDVQLKNITDYMYLTNAPSYINGKLNIYYRRLYNGLKFII | |
| | KRYTPNNEIDSFVKSGDFIKLYVSYNNNEHIVGYPKDGNAFNNLDRILRVGYNAPGIPLY | |
| | KKMEAVKLRDLKTYSVQLKLYDDKNASLGLVGTHNGQIGNDPNRDILIASNWYFNHLKDK | |
| | ILGCDWYFVPTDEGWIND | |
| 6 | GITELKKL | Fragment of SEQ ID NO. 5- |
| | | corresponds to amino acid |
| | | positions 383 to 390 of TTx |
| | | heavy chain |
| 7 | FIGITELKKLESKINKVE | Fragment of SEQ ID NO. 5- |
| | | corresponds to amino acid |
| | | positions 381 to 398 of TTx |
| | | heavy chain-UV30 (MTTE) |
| 8 | MGAPTLPPAWQPFLKDHRISTFKNWPFLEGCACTPERMAEAGFIHCPTENEPDLAQCEFC | Survivin |
| | FKELEGWEPDDDPIEEHKKHSSGCAFLSVKKQFEELTLGEFLKLDRERAKNKIAKETNNK | |
| | KKEFEETAKKVRRAIEQLAAMD | |
| 9 | MEVSPLQPVNENMQVNKIKKNEDAKKRLSVERIYQKKTQLEHILLRPDTYIGSVELVTQQ | DNA topoisomerase 2-alpha |
| | MWVYDEDVGINYREVTFVPGLYKIFDEILVNAADNKQRDPKMSCIRVTIDPENNLISIWN | |
| | NGKGIPVVEHKVEKMYVPALIFGQLLTSSNYDDDEKKVTGGRNGYGAKLCNIFSTKFTVE | |
| | TASREYKKMFKQTWMDNMGRAGEMELKPENGEDYTCITFQPDLSKFKMQSLDKDIVALMV | |
| | RRAYDIAGSTKDVKVFLNGNKLPVKGFRSYVDMYLKDKLDETGNSLKVIHEQVNHRWEVC | |
| | LTMSEKGFQQISFVNSIATSKGGRHVDYVADQIVTKLVDVVKKKNKGGVAVKAHQVKNHM | |
| | WIFVNALIENPTFDSQTKENMTLQPKSFGSTCQLSEKFIKAAIGCGIVESILNWVKFKAQ | |
| | VQLNKKCSAVKHNRIKGIPKLDDANDAGGRNSTECTLILTEGDSAKTLAVSGLGVVGRDK | |
| | YGVFPLRGKILNVREASHKQIMENAEINNIIKIVGLQYKKNYEDEDSLKTLRYGKIMIMT | |
| | DQDQDGSHIKGLLINFIHHNWPSLLRHRFLEEFITPIVKVSKNKQEMAFYSLPEFEEWKS | |
| | STPNHKKWKVKYYKGLGTSTSKEAKEYFADMKRHRIQFKYSGPEDDAAISLAFSKKQIDD | |
| | RKEWLTNFMEDRRQRKLLGLPEDYLYGQTTTYLTYNDFINKELILFSNSDNERSIPSMVD | |
| | GLKPGQRKVLFTCEKRNDKREVKVAQLAGSVAEMSSYHHGEMSLMMTIINLAQNFVGSNN | |
| | LNLLQPIGQFGTRLHGGKDSASPRYIFTMLSSLARLLFPPKDDHTLKFLYDDNQRVEPEW | |
| | YIPIIPMVLINGAEGIGTGWSCKIPNFDVREIVNNIRRLMDGEEPLPMLPSYKNFKGTIE | |
| | ELAPNQYVISGEVAILNSTTIEISELPVRTWTQTYKEQVLEPMINGTEKTPPLITDYREY | |
| | HTDTTVKFVVKMTEEKLAEAERVGLHKVFKLQTSLTCNSMVLFDHVGCLKKYDTVLDILR | |
| | DFFELRLKYYGLRKEWLLGMLGAESAKLNNQARFILEKIDGKIIIENKPKKELIKVLIQR | |
| | GYDSDPVKAWKEAQQKVPDEEENEESDNEKETEKSDSVTDSGPTFNYLLDMPLWYLTKEK | |
| | KDELCRLRNEKEQELDTLKRKSPSDLWKEDLATFIEELEAVEAKEKQDEQVGLPGKGGKA | |
| | KGKKTQMAEVLPSPRGQRVIPRITIEMKAEAEKKNKKKIKNENTEGSPQEDGVELEGLKQ | |
| | RLEKKQKREPGTKTKKQTTLAFKPIKKGKKRNPWSDSESDRSSDESNFDVPPRETEPRRA | |
| | ATKTKFTMDLDSDEDESDEDEKTDDEDFVPSDASPPKTKTSPKLSNKELKPQKSVVSDLE | |
| | ADDVKGSVPLSSSPPATHFPDETEITNPVPKKNVTVKKTAAKSQSSTSTTGAKKRAAPKG | |
| | TKRDPALNSGVSQKPDPAKTKNRRKRKPSTSDDSDSNFEKIVSKAVTSKKSKGESDDFHM | |
| | DFDSAVAPRAKSVRAKKPIKYLEESDEDDLE | |
| 10 | MGTSLSPNDPWPLNPLSIQQTTLLLLLSVLATVHVGQRLLRQRRRQLRSAPPGPFAWPLI | Cytochrome P450 1B1 |
| | GNAAAVGQAAHLSFARLARRYGDVFQIRLGSCPIVVLNGERAIHQALVQQGSAFADRPAF | |
| | ASFRVVSGGRSMAFGHYSEHWKVQRRAAHSMMRNFFTRQPRSRQVLEGHVLSEARELVAL | |
| | LVRGSADGAFLDPRPLTVVAVANVMSAVCFGCRYSHDDPEFRELLSHNEEFGRTVGAGSL | |
| | VDVMPWLQYFPNPVRTVFREFEQLNRNFSNFILDKFLRHCESLRPGAAPRDMMDAFILSA | |
| | EKKAAGDSHGGGARLDLENVPATITDIFGASQDTLSTALQWLLLLFTRYPDVQTRVQAEL | |
| | DQVVGRDRLPCMGDQPNLPYVLAFLYEAMRFSSFVPVTIPHATTANTSVLGYHIPKDTVV | |
| | FVNQWSVNHDPLKWPNPENEDPARFLDKDGLINKDLTSRVMIFSVGKRRCIGEELSKMQL | |
| | FLFISILAHQCDFRANPNEPAKMNFSYGLTIKPKSFKVNVTLRESMELLDSAVQNLQAKE | |
| | TCQ | |
| 11 | MCNTNMSVPTDGAVTTSQIPASEQETLVRPKPLLLKLLKSVGAQKDTYTMKEVLFYLGQY | E3 ubiquitin-protein |
| | IMTKRLYDEKQQHIVYCSNDLLGDLFGVPSFSVKEHRKIYTMIYRNLVVVNQQESSDSGT | ligase Mdm2 |
| | SVSENRCHLEGGSDQKDLVQELQEEKPSSSHLVSRPSTSSRRRAISETEENSDELSGERQ | |
| | RKRHKSDSISLSFDESLALCVIREICCERSSSSESTGTPSNPDLDAGVSEHSGDWLDQDS | |
| | VSDQFSVEFEVESLDSEDYSLSEEGQELSDEDDEVYQVTVYQAGESDTDSFEEDPEISLA | |
| | DYWKCTSCNEMNPPLPSHCNRCWALRENWLPEDKGKDKGEISEKAKLENSTQAEEGEDVP | |
| | DCKKTIVNDSRESCVEENDDKITQASQSQESEDYSQPSTSSSIIYSSQEDVKEFEREETQ | |
| | DKEESVESSLPLNAIEPCVICQGRPKNGCIVHGKTGHLMACFTCAKKLKKRNKPCPVCRQ | |
| | PIQMIVITYFP | |
| 12 | ALFSVLNYERARRP | Fragment of SEQ ID NO. 1- |
| | | corresponds to amino acid |
| | | positions 660-673 of hTERT |
| 13 | LFSVLNYERARRPG | Fragment of SEQ ID NO: 1- |
| | | Corresponds to amino acid |
| | | positions 661-674 of hTERT |
| 14 | FSVLNYERARRPGL | Fragment of SEQ ID NO: 1- |
| | | Corresponds to amino acid |
| | | positions 662-675 of hTERT |
| 15 | SVLNYERARRPGLL | Fragment of SEQ ID NO: 1 |
| | | Corresponds to amino acid |
| | | positions 663-676 of hTERT |
| 16 | VLNYERARRPGLLG | Fragment of SEQ ID NO: 1- |
| | | Corresponds to amino acid |
| | | positions 664-677 of hTERT |
| 17 | LNYERARRPGLLGA | Fragment of SEQ ID NO: 1- |
| | | Corresponds to amino acid |
| | | positions 665-678 of hTERT |
| 18 | NYERARRPGLIGAS | Fragment of SEQ ID NO: 1- |
| | | Corresponds to amino acid |
| | | positions 666-679 of hTERT |
| 19 | YERARRPGLLGASV | Fragment of SEQ ID NO: 1- |
| | | Corresponds to amino acid |
| | | positions 667-680 of hTERT |
| 20 | ERARRPGLLGASVL | Fragment of SEQ ID NO: 1- |
| | | Corresponds to amino acid |
| | | positions 668-681 of hTERT |
| 21 | RARRPGLLGASVLG | Fragment of SEQ ID NO: 1- |
| | | Corresponds to amino acid |
| | | positions 669-682 of hTERT |
| 22 | ARRPGLLGASVLGL | Fragment of SEQ ID NO: 1- |
| | | Corresponds to amino acid |
| | | positions 670-683 of hTERT |
| 23 | RRPGLLGASVLGLD | Fragment of SEQ ID NO: 1- |
| | | Corresponds to amino acid |
| | | positions 671-684 of hTERT |
| 24 | RPGLLGASVLGLDD | Fragment of SEQ ID NO: 1- |
| | | Corresponds to amino acid |
| | | positions 672-685 of hTERT |
| 25 | PGLLGASVLGLDDI | Fragment of SEQ ID NO: 1- |
| | | Corresponds to amino acid |
| | | positions 673-686 of hTERT |
| 26 | GLLGASVLGLDDIH | Fragment of SEQ ID NO: 1- |
| | | Corresponds to amino acid |
| | | positions 674-687 of hTERT |
| 27 | LLGASVLGLDDIHR | Fragment of SEQ ID NO: 1- |
| | | Corresponds to amino acid |
| | | positions 675-688 of hTERT |
| 28 | LGASVLGLDDIHRA | Fragment of SEQ ID NO: 1- |
| | | Corresponds to amino acid |
| | | positions 676-689 of hTERT |
| 29 | SVLNYERARRPGLLG | Fragment of SEQ ID NO: 1- |
| | | Corresponds to amino acid |
| | | positions 663-677 of hTERT |
| 30 | FSVLNYERARRPGLL | Fragment of SEQ ID NO: 1- |
| | | Corresponds to amino acid |
| | | positions 662-676 of hTERT |
| 31 | ARRPGLLGASVLGLD | Fragment of SEQ ID NO: 1- |
| | | Corresponds to amino acid |
| | | positions 670-684 of hTERT |
| 32 | RARRPGLLGASVLGL | Fragment of SEQ ID NO: 1- |
| | | Corresponds to amino acid |
| | | positions 669-683 of hTERT |
| 33 | VLNYERARRPGLLGA | Fragment of SEQ ID NO: 1- |
| | | Corresponds to amino acid |
| | | positions 664-678 of hTERT |
| 34 | RPGLLGASVLGLDDI | Fragment of SEQ ID NO: 1- |
| | | Corresponds to amino acid |
| | | positions 671-685 of hTERT |
| 35 | VLNYERARRPGLLGA | Fragment of SEQ ID NO: 1- |
| | | Corresponds to amino acid |
| | | positions 664-678 of hTERT |
| 36 | MQAEGRGTGGSTGDADGPGGPGIPDGPGGNAGGPGEAGATGGRGPRGAGAARASGPGGGA | Cancer/testis antigen 1 |
| | PRGPHGGAASGLNGCCRCGARGPESRLLEFYLAMPFATPMEAELARRSLAQDAPPLPVPG | (NY-ESO-1) |
| | VLLKEFTVSGNILTIRLTAADHRQLQLSISSCLQQLSLIMWITQCFLPVFLAQPPSGQRR | |
| 37 | MRAAPLLLARAASLSLGFLFLLFFWLDRSVLAKELKFVTLVERHGDRSPIDTFPTDPIKE | Prostatic acid phosphatase |
| | SSWPQGFGQLTQLGMEQHYELGEYIRKRYRKFINESYKHEQVYIRSTDVDRTLMSAMTNL | (PAP) |
| | AALFPPEGVSIWNPILLWQPIPVHTVPLSEDQLLYLPFRNCPRFQELESETLKSEEFQKR | |
| | LHPYKDFIATLGKLSGLHGQDLEGIWSKVYDPLYCESVHNFTLPSWATEDTMTKLRELSE | |
| | LSLLSLYGIHKQKEKSRLQGGVLVNEILNHMKRATQIPSYKKLIMYSAHDTTVSGLQMAL | |
| | DVYNGLLPPYASCHLTELYFEKGEYFVEMYYRNETQHEPYPLMLPGCSPSCPLERFAELV | |
| | GPVIPQDWSTECMTTNSHQGTEDSTD | |
| 38 | MWNLLHETDSAVATARRPRWLCAGALVLAGGFFLLGFLFGWFIKSSNEATNITPKHNMKA | Glutamate carboxypeptidase 2 |
| | FLDELKAENIKKFLYNFTQIPHLAGTEQNFQLAKQIQSQWKEFGLDSVELAHYDVLLSYP | (GCPII) / prostate-specific |
| | NKTHPNYISIINEDGNEIFNTSLFEPPPPGYENVSDIVPPFSAFSPQGMPEGDLVYVNYA | membrane antigen (PSMA) |
| | RTEDFFKLERDMKINCSGKIVIARYGKVERGNKVKNAQLAGAKGVILYSDPADYFAPGVK | |
| | SYPDGWNLPGGGVQRGNILNLNGAGDPLTPGYPANEYAYRRGIAEAVGLPSIPVHPIGYY | |
| | DAQKLLEKMGGSAPPDSSWRGSLKVPYNVGPGFTGNFSTQKVKMHIHSTNEVTRIYNVIG | |
| | TLRGAVEPDRYVILGGHRDSWVEGGIDPQSGAAVVHEIVRSFGTLKKEGWRPRRTILFAS | |
| | WDAEEFGLIGSTEWAEENSRLLQERGVAYINADSSIEGNYTLRVDCTPLMYSLVHNLTKE | |
| | LKSPDEGFEGKSLYESWTKKSPSPEFSGMPRISKLGSGNDFEVFFQRLGIASGRARYTKN | |
| | WETNKFSGYPLYHSVYETYELVEKFYDPMFKYHLTVAQVRGGMVFELANSIVLPFDCRDY | |
| | AVVLRKYADKIYSISMKHPQEMKTYSVSFDSLFSAVKNFTEIASKFSERLQDEDKSNPIV | |
| | LRMMNDQLMFLERAFIDPLGLPDRPFYRHVIYAPSSHNKYAGESFPGIYDALFDIESKVD | |
| | PSKAWGEVKRQIYVAAFTVQAAAETLSEVA | |
| 39 | GQDLFGIWSKVYDPL | Fragment of SEQ ID NO. 37 |
| 40 | TEDTMTKLRELSELS | Fragment of SEQ ID NO. 37 |
| 41 | GKVFRGNKVKNAQLA | Fragment of SEQ ID NO. 38 |
| 42 | TGNESTQKVKMHIHS | Fragment of SEQ ID NO. 38 |
| 43 | NYTLRVDCTPLMYSL | Fragment of SEQ ID NO. 38 |
| 44 | RQIYVAAFTVQAAAE | Fragment of SEQ ID NO. 38 |
| 45 | GARGPESRLLEFYLAMPFATPMEAELA | Fragment of SEQ ID NO. 36 |
| 46 | FIGITELKKLESKINKVEAEKYARVRAKC | Sequence comprising SEQ ID |
| | | NO. 7 and a spacer sequence |
| 47 | ARWWNYARTEDFFQQQPPPGQDLFGIWSKVYDPL | TENDU SLP |
| 48 | ARWWLLHETDSAVAAARQIYVAAFTVQAAAE | TENDU SLP |
| 49 | ARWWSLSLGFLFLAAAGKVERGNKVKNAQLA | TENDU SLP |
| 50 | ARWWGMPEGDLVYTGNFSTQKVKMHIHS | TENDU SLP |
| 51 | ARWWKVERGNKVKNYTLRVDCTPLMYSL | TENDU SLP |
| 52 | RTFVLRVRAQDPPPE | Corresponds to amino acid |
| | | positions 691-705 of hTERT |
| 53 | AERLTSRVKALFSVL | Corresponds to amino acid |
| | | positions 651-665 of hTERT |
| 54 | AVGALEGSRNQDWLGVPRQL | SLP containing murine CD8+ |
| | | T-cell epitope of mgp100 |
| 55 | ARWWWMHHNMDLIGGAKxVAAWTLKAu | SLP containing murine CD4+ |
| | Wherein x represents Cyclohexyl-Ala; u represents D-Ala | T-cell epitope (“PADRE”) |
| 56 | ARWW | TAP sequence |
| 57 | WMHHNMDLI | Murine CD8+ T-cell epitope |
| | | of the minor HY antigen |
| 58 | AKxVAAWTLKAU | Murine ″PADRE″ sequence |
| | Wherein x represents Cyclohexyl-Ala; u represents D-Ala | |
| 59 | FIGITELKKLES | |
| 60 | GITELKKLESKI | |
| 61 | SKFIGITELKKLES | |
| 62 | FIGITELKKLESKI | |
| 63 | ANSKFIGITELKKLES | |
| 64 | SKFIGITELKKLESKI | |
| 65 | IKANSKFIGITELKKLES | |
| 66 | ANSKFIGITELKKLESKI | |
| 67 | QYIKANSKFIGITELKKLES | |
| 68 | IKANSKFIGITELKKLESKI | |
| 69 | QYIKANSKFIGITELKKLESKI | |
| 70 | FIGITELKKLESKINKVEST | |
| 71 | FIGITELKKLESKINKVES | |
| 72 | FIGITELKKLESKINKVE | UV30 |
| 73 | FIGITELKKLESKINKV | |
| 74 | FIGITELKKLESKINK | |
| 75 | FIGITELKKLESKIN | |
| 76 | IGITELKKLESKIN | |
| 77 | GITELKKLESKINK | |
| 78 | FIGITELKKLESKI | |
| | Wherein Lys8 is formylated | |
| 79 | FIGITELKKLESKI | |
| | Wherein Lys9 is formylated | |
| 80 | FIGITELKKLESKI | |
| | Wherein Lys13 is formylated | |
| 81 | FIGITELKKLESKI | |
| | Wherein Lys8 and Lys9 are formylated | |
| 82 | FIGITELKKLESKI | |
| | Wherein Lys9 and Lys13 are formylated | |
| 83 | AIGITELKKLESKINKVE | |
| 84 | FAGITELKKLESKINKVE | |
| 85 | FIAITELKKLESKINKVE | |
| 86 | FIGITALKKLESKINKVE | |
| 87 | FIGITEAKKLESKINKVE | |
| 88 | FIGITELAKLESKINKVE | |
| 89 | FIGITELKALESKINKVE | |
| 90 | FIGITELKKAESKINKVE | |
| 91 | FIGITELKKLASKINKVE | |
| 92 | FIGITELKKLEAKINKVE | |
| 93 | FIGITELKKLESAINKVE | |
| 94 | FIGITELKKLESKANKVE | |
| 95 | FIGITELKKLESKIAKVE | |
| 96 | FIGITELKKLESKINAVE | |
| 97 | FIGITELKKLESKINKAF | |
| 98 | FIGITELKKLESKINKVA | |
| 99 | YIGITELKKLESKINKVE | |
| 100 | FLGITELKKLESKINKVE | |
| 101 | FISITELKKLESKINKVE | |
| 102 | FIGLTELKKLESKINKVE | |
| 103 | FIGISELKKLESKINKVE | |
| 104 | FIGITDLKKLESKINKVE | |
| 105 | FIGITEIKKLESKINKVE | |
| 106 | FIGITELRKLESKINKVE | |
| 107 | FIGITELKRLESKINKVE | |
| 108 | FIGITELKKIESKINKVE | |
| 109 | FIGITELKKLETKINKVE | |
| 110 | FIGITELKKLESRINKVE | |
| 111 | FIGITELKKLESKLNKVE | |
| 112 | FIGITELKKLESKIQKVE | |
| 113 | FIGITELKKLESKINRVE | |
| 114 | FIGITELKKLESKINKLE | |
| 115 | FIGITELKKLESKINKVY | |
| 116 | LSEAEVRQHREARPALLTSRLRFIPKPDGL | UV34 (GVExt5) |
| 117 | HREARPALLTSRLRFIPKPDGLRPIVNMDY | |
| 118 | LYFVKVDVTGAYDTIPQDRLTRVIASIIKP | |
| 119 | NPILLWQPIPV | |
| 120 | HREARPALLTSRLRFIPKPDGLRPIVNMDYVVGARTFRREK | |
| 121 | EARPALLTSRLRFIPKPDGLRPIVNMDY | |
| 122 | EARPALLTSRLRFIPKPDGLRPIVNMDYV | |
| 123 | EARPALLTSRLRFIPKPDGLRPIVNMD | |
| 124 | AEVRQHREARPALLTSRLRFIPKPDGL | |
| 125 | ALFSVLNYERARRPGLLGASVLGLDDIHRANPILLWQPIPV | |
| 126 | EARPALLTSRLRFIPK | UV80 (GV1001) |
| 127 | AEKYARVRAK | |
| 128 | AAKYARVRAK | |
| 129 | AAKYARVRAKC | |
| 130 | LKRVQLRELSEAEVRQ | |
| 131 | EAEVRQHREARPALL | |
| 132 | RPALLTSRLRFIPKP | |
| 133 | TSRLRFIPKPDGLRP | |
| 134 | FIPKPDGLRPIVNMD | |
| 135 | PDGLRPIVNMDYVVG | |
| 136 | FVLRVRAQDPPPELY | |
| 137 | PELYFVKVDVTGAY | |
| 138 | ELYFVKVDVTGAYDT | |
| 139 | LYFVKVDVTGAYDTI | |
| 140 | VTGAYDTIPQDRLTE | |
| 141 | PQDRLTEVIASIIKP | |
| 142 | FIGITELKKLESKINKVESSAFADVEAA | |
| 143 | ARWWLLHETDSAVAAARQIYVAAFTVQAAAE | |
| 144 | LLHETDSAVAAARQIYVAAFTVQAAAE | |
| 145 | FIGITELKKLESKINKVF-AEKYARVRAK | |
| 146 | FIGITELKKLESKINKVF-AEKYARVRAKC | |
| 147 | LLGASVIGL | UV13 |
| 148 | GLLGASVLGL | UV14 |
| 149 | VLGLDDIHRA | UV15 |
| 150 | FSVLNYERARRPGLL | UV16 |
| 151 | RARRPGLLGASVLGL | UV17 |
| 152 | VLNYERARRPGLLGA | UV18 |
| 153 | RPGLLGASVLGLDDI | UV19 |
| 154 | SSAFADVEAA | |
| 155 | NYARTEDEF | CD8 epitope LUG1 |
| 156 | LLHETDSAV | CD8 epitope LUG2 |
| 157 | SLSLGFLFL | CD8 epitope LUG3 |
| 158 | GMPEGDLVY | CD8 epitope LUG4 |
| 159 | KVERGNKVK | CD8 epitope LUG5 |
| 160 | gcactgttca gcgtgctcaa ctacgagcgg gcgcggcgcc ccggectect | |
| | gggcgcctct gtgctgggcc tcgacgatat ccacagggcc | |
| 161 | ctgtcggaag cagaggtcag gcagcategg gaagccaggc ccgccctgct | |
| | gacgtccaga ctccgcttca tccccaagcc tgacgggctg cgg | |
| 162 | catcgggaag ccaggcccgc cctgctgacg tccagactcc gottcatccc | |
| | caagcctgac gggctgcggc cgattgtgaa catggactac | |
| 163 | NYERARRPG | |
| 164 | RRPGLLGAS | |
| 165 | LLGASVLGLDDI | |
| 166 | EARPALLTSRLRFIPKPDGL | UV36 |
| 167 | QHLKRVQLRELSEAE | UV57 |
| 168 | LSEAEVRQHREARPA | UV58 |
| 169 | EARPALLTSRLRFIP | UV59 |
| 170 | LRFIPKPDGLRPIVN | UV60 |
| 171 | LLTSRLRFI | UV64 |
| 172 | ALLTSRLRFI | UV65 |
| 173 | REARPALLTSRLRFI | UV66 |
| 174 | RAERLTSRVKALFSV | UV53 |
| 175 | ALFSVLNYERARRPG | UV54 |
| 176 | VLGLDDIHRAWRTFV | UV56 |
| 177 | RPIVN | |
| 178 | PDGL | |

1.-41. (canceled)

42. A conjugate comprising:
(a) at least one polypeptide comprising a sequence of a B-cell epitope; and
(b) at least one polypeptide comprising a sequence of a CD4+ T-cell epitope, wherein the CD4+ T-cell epitope comprises a region of at least 12 amino acids of a universal tumour antigen or a sequence having at least 80% sequence identity to the region and wherein the CD4+ T-cell epitope is immunogenic in at least 50% of the population, wherein the at least one polypeptide comprising the sequence of the CD4+ T-cell epitope is equal to or less than 500 amino acids in length,
wherein the sequence of the B-cell epitope is different from the sequence of the CD4+ T-cell epitope, and
wherein an antibody specific for the B-cell epitope binds to the conjugate.

43. A conjugate according to claim 42, wherein the at least one polypeptide comprising a sequence of a B-cell epitope and the at least one polypeptide comprising a sequence of a CD4+ T-cell epitope are linked via a core,
wherein the core comprises, prior to linkage:
a body portion;
one or more first linking groups attached to the body portion; and
one or more second linking groups attached to the body portion,
wherein the first linking group and second linking group are orthogonal to each other;
and wherein the first linking group is linked to the at least one polypeptide comprising a sequence of a B-cell epitope to form a first connecting element, and the second linking group is linked to the at least one polypeptide comprising a sequence of a CD4+ T-cell epitope to form a second connecting element; preferably wherein:
(i) the first linking group and the second linking group are independently selected from an alkyne (e.g. a terminal alkyne), an alkene (e.g. a terminal alkene, norbornene), a cycloalkyne, a trans-cycloalkene, a tetrazine, a conjugated diene, a maleimide, an α-halocarbonyl, a thiol and an azide, and/or
(ii) the first connecting element and the second connecting element are independently selected from a 1,2,3-triazole linkage, a dihydropyridazine linkage, a pyridazine linkage and a sulfide linkage.

44. A method of determining the presence of a CD4+ T-cell response to a CD4+ T-cell epitope in a subject to whom a conjugate comprising at least one polypeptide comprising a sequence of a B-cell epitope and at least one polypeptide comprising a sequence of the CD4+ T-cell epitope has been administered, comprising the steps of:
(a) detecting a quantity or an absence of an antibody specific to the conjugate in a sample derived from the subject prior to an administration of the conjugate, wherein the quantity or the absence of the antibody is detected at a first level; and
(b) detecting a quantity or an absence of an antibody specific to the conjugate in a sample derived from the subject subsequent to one or more administrations of the conjugate, wherein the quantity or the absence of the antibody is detected at a second level, and
wherein an increase in the quantity of the antibody at the second level relative to the quantity or absence of the antibody at the first level is indicative of the presence of a CD4+ T-cell response to the CD4+ T-cell epitope in the subject.

45. The method according to claim 44, wherein step (b) comprises detecting a quantity or absence of an antibody specific to the conjugate in a sample derived from a subject subsequent to two or more administrations of the conjugate, preferably three or more administrations of the conjugate, more preferably four administrations of the conjugate.

46. The method according to claim 44, wherein the antibody specific to the conjugate is an antibody specific to the B-cell epitope.

47. The conjugate according to claim 42, wherein the at least one polypeptide comprising a sequence of a B-cell epitope is a first and a second polypeptide comprising a sequence of a B-cell epitope, preferably a first, a second and a third polypeptide comprising a sequence of a B-cell epitope.

48. The conjugate according to claim 42, wherein the B-cell epitope comprises a sequence selected from:
(i) a sequence comprising at least 10 amino acids which are contiguous in SEQ ID NO: 3, preferably a sequence comprising at least 10 amino acids which are contiguous in SEQ ID NO: 5; or
(ii) a sequence having at least 70% sequence identity to (i).

49. The conjugate according to claim 48, wherein the B-cell epitope comprises a sequence selected from:
(i) a sequence comprising at least 10 amino acids which are contiguous in SEQ ID NO: 5 and comprise the amino acid sequence GITELKKL as represented by SEQ ID NO: 6 in the Sequence Listing; or
(i) a sequence having at least 70% sequence identity to (i).

50. The conjugate according to claim 49, wherein the B-cell epitope comprises a sequence selected from:
(i) SEQ ID NO: 7; or
(ii) a sequence having at least 70% sequence identity to (i).

51. The method according to claim 44, wherein the CD4+ T-cell epitope comprises a region of at least 12 amino acids of a self-antigen or a tumour-associated antigen, or a sequence having at least 80% sequence identity to the region and wherein the at least one polypeptide comprising the sequence of the CD4+ T-cell epitope is equal to or less than 500 amino acids in length, preferably, wherein the self-antigen or the tumour-associated antigen is a universal tumour antigen.

52. The conjugate according to claim 42, wherein the universal tumour antigen is selected from the group consisting of telomerase reverse transcriptase, survivin, DNA topoisomerase 2-alpha, cytochrome P450 1B1 and E3 ubiquitin-protein ligase Mdm2.

53. The conjugate according to claim 52, wherein the universal tumour antigen is telomerase reverse transcriptase and wherein the CD4+ T-cell epitope comprises one or more sequences selected from:
(i) SEQ ID NO: 1;
(ii) SEQ ID NO: 116;
(iii) SEQ ID NO: 117;
(iv) the sequence of an immunogenic fragment of (i), (ii) or (iii) comprising at least 12 amino acids; and/or
(v) a sequence having at least 80% sequence identity to (i), (ii), (iii) or (iv).

54. The conjugate according to claim 42, wherein the at least one polypeptide comprising the sequence of the CD4+ T-cell epitope comprises the sequence of a further T-cell epitope, wherein the further T-cell epitope is a further CD4+ T-cell epitope and/or a CD8+ T-cell epitope.

55. The conjugate according to claim 42, wherein the conjugate comprises a further substance, preferably the further substance is a further polypeptide comprising the sequence of an epitope, more preferably the epitope is a further T-cell epitope, more preferably the further T-cell epitope is a further CD4+ T-cell epitope and/or a CD8+ T-cell epitope.

56. The conjugate according to claim 54, wherein the CD4+ T-cell epitope comprises a sequence selected from:
(i) SEQ ID NO: 1;
(ii) the sequence of an immunogenic fragment of (i) comprising at least 12 amino acids; or
(iii) a sequence having at least 80% sequence identity to (i) or (ii),
and wherein the further CD4+ T-cell epitope comprises a sequence selected from:
(iv) SEQ ID NO: 116;
(v) the sequence of an immunogenic fragment of (iv) comprising at least 12 amino acids; or
(vi) a sequence having at least 80% sequence identity to (iv) or (v).

57. A cocktail of conjugates comprising first and second different conjugates, wherein the first conjugate and/or the second conjugate is a conjugate according to claim 42.

58. The cocktail of conjugates according to claim 57, wherein the first conjugate comprises a CD4+ T-cell epitope comprising a sequence selected from:
(i) SEQ ID NO: 1;
(ii) the sequence of an immunogenic fragment of (i) comprising at least 12 amino acids; or
(iii) a sequence having at least 80% sequence identity to (i) or (ii),
and wherein the second conjugate comprises a CD4+ T-cell epitope comprising a sequence selected from:
(iv) SEQ ID NO: 116;
(v) the sequence of an immunogenic fragment of (iv) comprising at least 12 amino acids; or
(vi) a sequence having at least 80% sequence identity to (iv) or (v).

59. A nucleic acid molecule comprising a first nucleotide sequence encoding a polypeptide as defined in claim 42, part (a) and a second nucleotide sequence encoding a polypeptide as defined in claim 42, part (b).

60. A polypeptide comprising a sequence selected from:
(i) SEQ ID NO: 116;
(ii) a sequence having at least 91% sequence identity to (i);
(iii) SEQ ID NO: 117; or
(iv) a sequence having at least 95% sequence identity to (iii),
wherein the polypeptide comprising the sequence selected from (i) or (ii) is equal to or less than 100 amino acids in length and wherein the polypeptide comprising the sequence selected from (iii) or (iv) is equal to or less than 40 amino acids in length.

61. The polypeptide according to claim 60, wherein the polypeptide comprising the sequence selected from (i) or (ii) is equal to or less than 75 or 50 amino acids in length.

62. The polypeptide according to claim 60, wherein the polypeptide comprising the sequence selected from (iii) or (iv) is equal to or less than 35, 34, 33, 32 or 31 amino acids in length.

63. A nucleic acid molecule consisting of a nucleotide sequence encoding a polypeptide according to claim 42.

64. A vector comprising a nucleic acid molecule according to claim 59.

65. A combination comprising a first product and a second product wherein the first product is selected from the following (i) to (v):
(i) a polypeptide comprising the sequence of SEQ ID NO: 1;
(ii) a polypeptide comprising an immunogenic fragment of (i) comprising at least eight amino acids;
(iii) a polypeptide comprising a sequence having at least 80% sequence identity to (i) or (ii);
(iv) a conjugate comprising the polypeptide defined in any one of (i) to (iii);
(v) a nucleic acid molecule consisting of a nucleotide sequence encoding a polypeptide as defined in any one of (i) to (iii),
and wherein the second product is selected from the following (vi) to (x):
(vi) a polypeptide comprising the sequence of SEQ ID NO: 116;
(vii) a polypeptide comprising an immunogenic fragment of (vi) comprising at least seventeen amino acids;
(viii) a polypeptide comprising a sequence having at least 80% sequence identity to (vi) or (vii);
(ix) a conjugate comprising the polypeptide defined in any one of (vi) to (viii);
(x) a nucleic acid molecule consisting of a nucleotide sequence encoding a polypeptide as defined in any one of (vi) to (viii),
wherein the first product and the second product are optionally a single product subject to the following provisos:
(a) where the first product and the second product are a single polypeptide and the first product is as defined in any one of (i) to (iii) and the second product is as defined in any one of (vi) to (viii) then the single polypeptide is equal to or less than 170 amino acids in length;
(b) where the first product and the second product are a single product and the first product is as defined in (v) and the second product is as defined in (x) then the single nucleic acid molecule is less than 1500 nucleotides in length.

66. The combination according to claim 65, comprising a cocktail of polypeptides wherein the first product is a polypeptide as defined in claim 65, part (i), (ii) or (iii) and wherein the second product is a polypeptide as defined in claim 65, part (vi), (vii) or (viii).

67. The combination according to claim 65, comprising a cocktail of nucleic acid molecules wherein the first product is a nucleic acid molecule as defined in claim 65, part (v) and the second product is a nucleic acid molecule as defined in claim 65, part (x).

68. The polypeptide according to claim 60, wherein the polypeptide is linked to a further substance.

69. A method of treating a disease in a subject comprising administering to the subject a first product, wherein the first product is administered simultaneously, separately or sequentially with a second product, wherein the first product is selected from the following (i) to (v):
(i) a polypeptide comprising the sequence of SEQ ID NO: 1;
(ii) a polypeptide comprising an immunogenic fragment of (i) comprising at least eight amino acids;
(iii) a polypeptide comprising a sequence having at least 80% sequence identity to (i) or (ii);
(iv) a conjugate comprising the polypeptide defined in any one of (i) to (iii);
(v) a nucleic acid molecule consisting of a nucleotide sequence encoding a polypeptide as defined in any one of (i) to (iii),
and wherein the second product is selected from the following (vi) to (x):
(vi) a polypeptide comprising the sequence of SEQ ID NO: 116;
(vii) a polypeptide comprising an immunogenic fragment of (vi) comprising at least seventeen amino acids;
(viii) a polypeptide comprising a sequence having at least 80% sequence identity to (vi) or (vii);
(ix) a conjugate comprising the polypeptide defined in any one of (vi) to (viii);
(x) a nucleic acid molecule consisting of a nucleotide sequence encoding a polypeptide as defined in any one of (vi) to (viii),
wherein the first product and the second product are optionally a single product subject to the following provisos:
(a) where the first product and the second product are a single polypeptide and the first product is as defined in any one of (i) to (iii) and the second product is as defined in any one of (vi) to (viii) then the single polypeptide is equal to or less than 170 amino acids in length;
(b) where the first product and the second product are a single product and the first product is as defined in (v) and the second product is as defined in (x) then the single nucleic acid molecule is equal to or less than 1500 nucleotides in length.

70. A method of preventing or treating a disease in a subject comprising administering to the subject a second product, wherein the second product is administered simultaneously, separately or sequentially with a first product, wherein the second product is selected from the following (i) to (v):
(i) a polypeptide comprising the sequence of SEQ ID NO: 116;
(ii) a polypeptide comprising an immunogenic fragment of (i) comprising at least seventeen amino acids;
(iii) a polypeptide comprising a sequence having at least 80% sequence identity to (i) or (ii);
(iv) a conjugate comprising the polypeptide defined in any one of (i) to (iii);
(v) a nucleic acid molecule consisting of a nucleotide sequence encoding a polypeptide as defined in any one of (i) to (iii),
and wherein the first product is selected from the following (vi) to (x):
(vi) a polypeptide comprising the sequence of SEQ ID NO: 1;
(vii) a polypeptide comprising an immunogenic fragment of (vi) comprising at least eight amino acids;
(viii) a polypeptide comprising a sequence having at least 80% sequence identity to (vi) or (vii);
(ix) a conjugate comprising the polypeptide defined in any one of (vi) to (viii);
(x) a nucleic acid molecule consisting of a nucleotide sequence encoding a polypeptide as defined in any one of (vi) to (viii),
wherein the second product and the first product are optionally a single product subject to the following provisos:
(a) where the second product and the first product are a single polypeptide and the second product is as defined in any one of (i) to (iii) and the first product is as defined in any one of (vi) to (viii) then the single polypeptide is equal to or less than 170 amino acids in length;
(b) where the second product and the first product are a single product and the second product is as defined in (v) and the first product is as defined in (x) then the single nucleic acid molecule is equal to or less than 1500 nucleotides in length.

71. A method of preventing or treating a disease in a subject comprising administering to the subject a conjugate according to claim 42.

72. A method of preventing or treating a disease in a subject according to claim 71, wherein the disease is cancer.

73. A pharmaceutical composition comprising a conjugate according to claim 42, and a pharmaceutically acceptable diluent, excipient or adjuvant, and optionally another therapeutic ingredient.

74. A core comprising:
a body portion;
one or more first linking groups attached to the body portion; and
one or more second linking groups attached to the body portion,
wherein the first linking group and second linking group are orthogonal to each other, at least one of the first linking group and the second linking group comprises two or more first linking groups or second linking groups, and
the first linking group and the second linking group are independently selected from an alkyne (e.g. a terminal alkyne), an alkene (e.g. a terminal alkene, norbornene), a cycloalkyne, a trans-cycloalkene, a tetrazine, a conjugated diene, a maleimide, an α-halocarbonyl, a thiol and an azide; preferably wherein the first linking group and the second linking group are independently selected from an alkyne (e.g. a terminal alkyne), a cycloalkyne, a maleimide and an α-halocarbonyl; more preferably wherein the first linking group and the second linking group are independently selected from an alkyne (e.g. a terminal alkyne), a cycloalkyne and an α-halocarbonyl;
wherein the core is not:

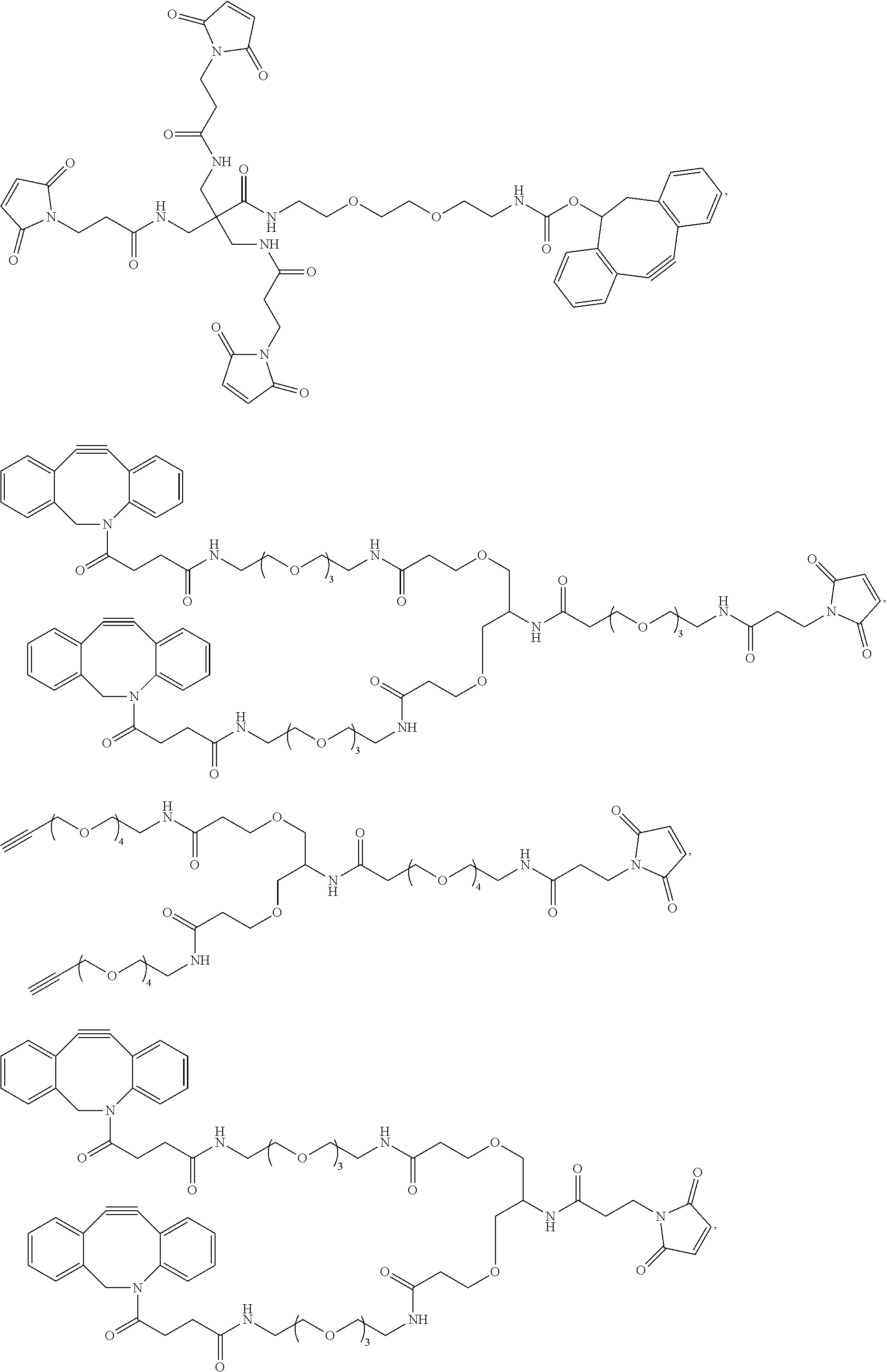

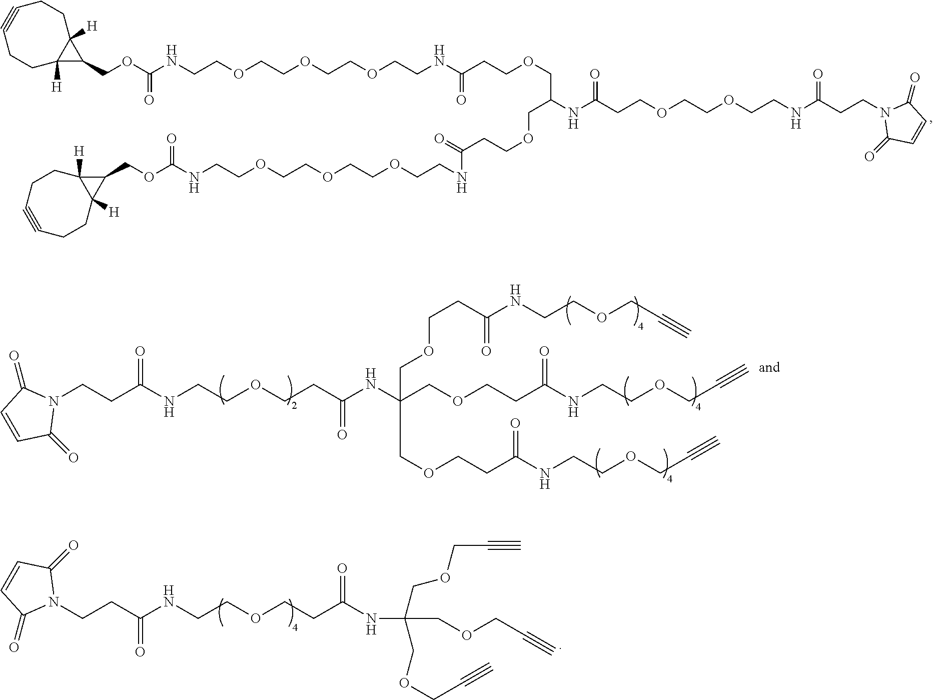

75. The conjugate according to claim 43, wherein the body portion is represented by one of Formulae 1 and 2: 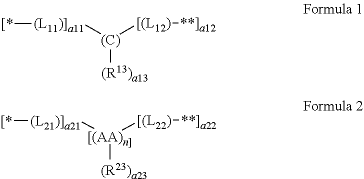
wherein in Formula 1:
L₁₁ and L₁₂ are linkers;
R₁₃ is selected from hydrogen, hydroxy, optionally substituted amino, halogen, optionally substituted alkyl, —S-(optionally substituted alkyl), optionally substituted cycloalkyl, optionally substituted heterocycloalkyl, optionally substituted alkoxy, optionally substituted alkanoyl, optionally substituted aryl and optionally substituted heteroaryl;
a11 represents the number of [(L₁₁)-*] groups attached to the carbon atom and is selected from 1, 2 or 3;
a12 represents the number of [(L₁₂)-**] groups attached to the carbon atom and is selected from 1, 2 or 3;
a13 represents the number of R₁₃ groups attached to the carbon atom and is selected from 0 or 1;
a11+a12+a13 is 4;
* represents a connection point to the first linking group;
** represents a connection point to the second linking group; and
wherein in Formula 2:
AA represents an amino acid;
n represents the number of independently selected AA groups and is selected from 1 to 12;
L₂₁ and L₂₂ are linkers;
R₂₃ is selected from hydrogen, hydroxy, optionally substituted amino, optionally substituted alkyl, —S-(optionally substituted alkyl), optionally substituted cycloalkyl, optionally substituted heterocycloalkyl, optionally substituted alkoxy, optionally substituted alkanoyl, optionally substituted aryl and optionally substituted heteroaryl;
a21 represents the number of [(L₂₁)-*] groups attached to [(AA)] n and is selected from 1, 2 or 3;
a22 represents the number of [(L₂₂)-**] groups attached to [(AA)] n and is selected from 1, 2 or 3;
a23 represents the number of R₂₃ groups attached to [(AA)], at the C-terminus and/or the N-terminus, and is selected from 0, 1 or 2;
* represents a connection point to the first linking group via the N-terminus, C-terminus or a side-chain of one of the AA groups;
** represents a connection point to the second linking group via the N-terminus, C-terminus or a side-chain of one of the AA groups.

76. The conjugate according to claim 43, wherein L₁₁, L₁₂, L₂₁ and L₂₂ are independently selected from -(optionally substituted alkylene)-, —O—, —(CONH)—, —(NHCO)—, —(CH₂CH₂O)_{w}—, —(CO)—, -(optionally substituted alkylene)-O—, -(optionally substituted alkylene)-(CONH)—, -(optionally substituted alkylene)-(NHCO)—, -(optionally substituted alkylene)-(CH₂CH₂O)_{w}-, -(optionally substituted alkylene)-(CO)—, —O-(optionally substituted alkylene)-, —O—(CONH)—, —O—(NHCO)—, —O—(CH₂CH₂O)_{w}—, —O—(CO)—, —(CONH)-(optionally substituted alkylene)-, —(CONH)—O—, —(CONH)—(NHCO)—, —(CONH)—(CH₂CH₂O)_{w}—, —(CONH)—(CO)—, —(NHCO)-(optionally substituted alkylene)-, —(NHCO)—O—, —(NHCO)—(CONH)—, —(NHCO)—(CH₂CH₂O)_{w}—, —(NHCO)—(CO)—, —(CH₂CH₂O)_{w}-(optionally substituted alkylene)-, —(CH₂CH₂O)_{w}—O—, —(CH₂CH₂O)_{w}—(CONH)—, —(CH₂CH₂O)—(NHCO)—, —(CH₂CH₂O)_{w}—(CO)—, —(CO)-(optionally substituted alkylene), —(CO)—O—, —(CO)—(CONH)—, —(CO)—(NHCO)—, —(CO)—(CH₂CH₂O)_{w}—, —(CO)-(optionally substituted alkylene)-(CO)—, -(optionally substituted alkylene)-(NHCO)-(optionally substituted alkylene)-, -(optionally substituted alkylene)-(CONH)-(optionally substituted alkylene)-, -(optionally substituted alkylene)-(NHCO)—(CH₂CH₂O)_{w}-(optionally substituted alkylene)-, -(optionally substituted alkylene)-(CONH)—(CH₂CH₂O)_{w}-(optionally substituted alkylene)-, -(optionally substituted alkylene)-O-(optionally substituted alkylene)-(NHCO)—(CH₂CH₂O)_{w}-(optionally substituted alkylene)-, -(optionally substituted alkylene)-O-(optionally substituted alkylene)-(CONH)—(CH₂CH₂O)_{w}-(optionally substituted alkylene)-, —(CONH)—(CH₂CH₂O)_{w}-(optionally substituted alkylene)-(CONH)—O—, —(NHCO)—(CH₂CH₂O)_{w}-(optionally substituted alkylene)-(CONH)—O—, —(CONH)—(CH₂CH₂O)_{w}-(optionally substituted alkylene)-(NHCO)—O—, —(NHCO)—(CH₂CH₂O)_{w}-(optionally substituted alkylene)-(NHCO)—O—, —(CONH)—(CH₂CH₂O)_{w}-(optionally substituted alkylene)-(CONH)-(optionally substituted alkylene)-(CO)—, —(NHCO)—(CH₂CH₂O)_{w}-(optionally substituted alkylene)-(CONH)-(optionally substituted alkylene)-(CO)—, (CONH)—(CH₂CH₂O)_{w}-(optionally substituted alkylene)-(NHCO)-(optionally substituted alkylene)-(CO)—, —(NHCO)—(CH₂CH₂O)_{w}-(optionally substituted alkylene)-(NHCO)-(optionally substituted alkylene)-(CO)—.

77. The conjugate according to claim 43, wherein the body portion is represented by Formula 2.

78. The conjugate according to claim 43, wherein the core further comprises a third linking group attached to the body portion, wherein the third linking group is orthogonal to the first linking group and the second linking group; preferably wherein the third linking group is independently selected from an alkyne (e.g. a terminal alkyne), an alkene (e.g. a terminal alkene, norbornene), a cycloalkyne, a trans-cycloalkene, a tetrazine, a conjugated diene, a maleimide, an α-halocarbonyl, a thiol and an azide.

79. A conjugate comprising:
(a) at least one polypeptide comprising a sequence of a B-cell epitope; or
(b) at least one polypeptide comprising a sequence of a CD4+ T-cell epitope;
wherein the at least one polypeptide comprising a sequence of a B-cell epitope or the at least one polypeptide comprising a sequence of a CD4+ T-cell epitope is linked to a core, wherein the core comprises, prior to linkage:
a body portion;
one or more first linking groups attached to the body portion; and
one or more second linking groups attached to the body portion,
wherein the first linking group and second linking group are orthogonal to each other,
wherein the core is not: 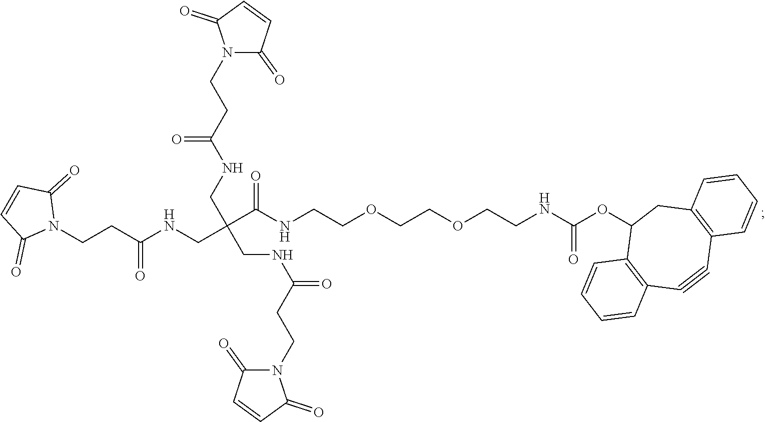 [SMILES: O=C(CCN1C(=O)C=CC1=O)NCC(CNC(=O)CCN1C(=O)C=CC1=O)(CNC(=O)CCN1C(=O)C=CC1=O)C(=O)NCCOCCOCCNC(=O)OC1Cc2ccccc2C#Cc2ccccc21]
and wherein the first linking group is linked to the at least one polypeptide comprising a sequence of a B-cell epitope to form a first connecting element, or the second linking group is linked to the at least one polypeptide comprising a sequence of a CD4+ T-cell epitope to form a second connecting element;
preferably wherein the core is as defined according to claim 74.

80. A conjugate according to claim 79, wherein the conjugate comprises:
(a) at least one polypeptide comprising a sequence of a B-cell epitope; and
(b) at least one polypeptide comprising a sequence of a CD4+ T-cell epitope;
and wherein the first linking group is linked to the at least one polypeptide comprising a sequence of a B-cell epitope to form a first connecting element, and the second linking group is linked to the at least one polypeptide comprising a sequence of a CD4+ T-cell epitope to form a second connecting element.

81. A process for manufacturing a conjugate, comprising the steps of:
(a) providing a core comprising:
a body portion;
one or more first linking groups attached to the body portion; and
one or more second linking groups attached to the body portion,
wherein the first linking group and second linking group are orthogonal to each other,
at least one of the first linking group and the second linking group comprises two or more first linking groups or second linking groups, and
the first linking group and the second linking group are independently selected from an alkyne (e.g. a terminal alkyne), an alkene (e.g. a terminal alkene, norbornene), a cycloalkyne, a cycloalkene, a tetrazine, a conjugated diene, a maleimide, an α-halocarbonyl, a thiol and an azide; preferably wherein the first linking group and the second linking group are independently selected from an alkyne (e.g. a terminal alkyne), a cycloalkyne, a maleimide and an α-halocarbonyl; more preferably wherein the first linking group and the second linking group are independently selected from an alkyne (e.g. a terminal alkyne), a cycloalkyne and an α-halocarbonyl;
wherein the core is not: 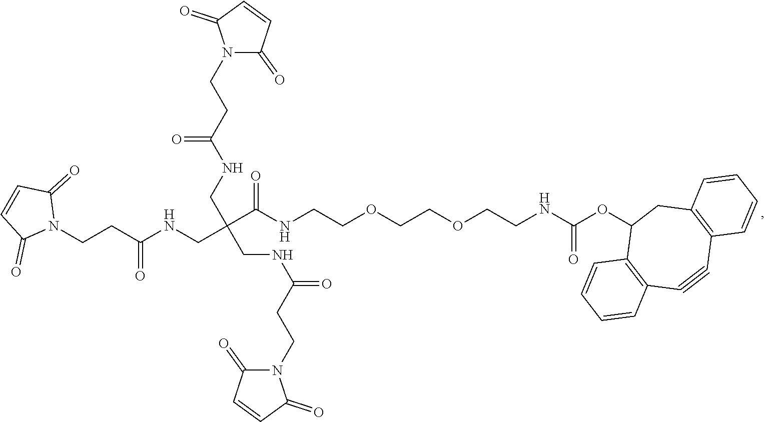 [SMILES: O=C(CCN1C(=O)C=CC1=O)NCC(CNC(=O)CCN1C(=O)C=CC1=O)(CNC(=O)CCN1C(=O)C=CC1=O)C(=O)NCCOCCOCCNC(=O)OC1Cc2ccccc2C#Cc2ccccc21]
preferably wherein the core is as defined according to claim 74;
(b) providing at least one polypeptide comprising a sequence of a B-cell epitope, or at least one polypeptide comprising a sequence of a CD4+ T-cell epitope; and reacting the core with the at least one polypeptide comprising a sequence of a B-cell epitope to form a first connecting element, or reacting the core with the at least one polypeptide comprising a sequence of a CD4+ T-cell epitope to form a second connecting element.

82. The process according to claim 81, further comprising the step of:
(c) providing the other of at least one polypeptide comprising a sequence of a CD4+ T-cell epitope, or at least one polypeptide comprising a sequence of a B-cell epitope, not provided in step (b); and reacting the core with the at least one polypeptide comprising a sequence of a CD4+ T-cell epitope to form a second connecting element if the first connecting element was formed in step (b); or reacting the core with the at least one polypeptide comprising a sequence of a B-cell epitope to form a first connecting element if the second connecting element was formed in step (b).
